(19) United States
(12) Patent Application Publication
(10) Pub. No.: US 2010/0055093 A1
(43) Pub. Date: Mar. 4, 2010

(54) PAN-CELL SURFACE RECEPTOR-SPECIFIC THERAPEUTICS

(75) Inventors: H. Michael Shepard, San Francisco, CA (US); Pei Jin, Palo Alto, CA (US); Louis E. Burton, San Mateo, CA (US); Malgorzata Beryt, Malibu, CA (US)

Correspondence Address: MORRISON & FOERSTER LLP, 755 PAGE MILL RD, PALO ALTO, CA, 94304-1018, US

(73) Assignee: RECEPTOR BIOLOGIX INC., Palo Alto, CA (US)

(21) Appl. No.: 12/304,467

(22) PCT Filed: Jun. 12, 2007
(86) PCT No.: PCT/US2007/071041

§ 371 Date: Nov. 16, 2009

Related U.S. Application Data
(60) Provisional application No. 60/813,260, filed on Jun. 12, 2006.
(60) Provisional application No. 60/848,542, filed on Sep. 29, 2006.
(60) Provisional application No. 60/878,941, filed on Jan. 5, 2007.

(51) Int. Cl. A61K 39/395; C07K 14/00; C07K 16/00; C07H 21/00; C12N 15/63; C12N 5/00; A61K 38/16; A61K 31/7052; A61K 35/12; A61P 35/00; A61P 9/00; A61P 13/10; A61P 11/00; A61P 29/00

(52) U.S. Cl. 424/133.1; 530/350; 530/391.1; 530/402; 536/23.5; 435/320.1; 435/325; 514/12; 514/44 R; 424/93.7; 424/130.1; 514/44 A; 424/158.1; 424/178.1; 424/142.1; 530/324; 436/86; 530/387.9

(57) ABSTRACT

Provided are pan-cell surface receptor-specific therapeutics, methods for preparing them and methods of treatment using them. Among the pan-cell surface receptor-specific therapeutics are pan-HER-specific therapeutics that interact with at least two different HER receptor ligands and/or dimerize with or interact with two or more HER cell surface receptors. By virtue of these properties, the therapeutics modulate the activity of at least two cell surface receptors and are useful for therapeutic purposes.

### Related Applications

The present patent application claims priority to U.S. Provisional Application Ser. No. 60/813,260, filed on Jun. 12, 2006; U.S. Provisional Application Ser. No. 60/848,542, filed on Sep. 29, 2006; and U.S. Provisional Application Ser. No. 60/848,941, filed on Jan. 5, 2007.

The subject matter of each of the above-referenced related applications and the sequence listing pertainting thereto is incorporated by reference in its entirety.

### FIELD OF THE INVENTION

Pan-cell surface receptor-specific therapeutics, including pan-HER-specific therapeutics, and methods of making and using them are provided.

### BACKGROUND

Cell signaling pathways involve a network of molecules including polypeptides and small molecules that interact to relay extracellular, intercellular and intracellular signals. Such pathways interact, handing off signals from one member of the pathway to the next. Modulation of one member of the pathway can be relayed through the signal transduction pathway, resulting in modulation of activities of other pathway members and in modulating outcomes of such signal transduction such as affecting phenotypes and responses of a cell or organism to a signal. Diseases and disorders can involve misregulated or changes in modulation of signal transduction pathways. A goal of drug development is to target such misregulated pathways to restore more normal regulation in the signal transduction pathway.

Receptor tyrosine kinases (RTKs) are a family of cell signaling molecules that are among the polypeptides involved in many signal transduction pathways. RTKs play a role in a variety of cellular processes, including embryogenesis, cell division, proliferation, differentiation, migration and metabolism. RTKs can be activated by ligands. Such activation, in turn, usually results in receptor dimerization or oligomerization as a requirement for the subsequent activation of the signaling pathways. Activation of the signaling pathway, such as by triggering autocrine or paracrine cellular signaling pathways, for example, activation of second messengers, results in specific biological effects. Ligands for RTKs specifically bind to the cognate receptors.

RTKs also are involved in or play a role in a number of disease processes, including cancer, autoimmune diseases and other chronic diseases (see, e.g., Hynes et al. (2005) Nature Reviews Cancer 5:341-35) Cancers in which RTKs have been implicated include breast and colorectal cancers, gastric carcinomas, gliomas and mesodermal-derived tumors. Disregulation of RTKs has been noted in several cancers. For example, breast cancer can be associated with amplified expression of p185-HER2. RTKs also have been associated with diseases of the eye, including diabetic retinopathies and macular degeneration. RTKs also are associated with regulating pathways involved in angiogenesis, including physiologic and tumor blood vessel formation. RTKs also are implicated in the regulation of cell proliferation, migration and survival.

Among the RTKs associated with disease is the HER (Human EGFR family, also referred to as the ErbB or EGFR) family of receptors (see, e.g., Hynes et al. (2005) Nature Reviews Cancer 5:341-354, for a discussion of their role cancer). These receptors, referred to as the Class I receptors, include HER1/EGFR, HER2, HER3 and HER4. Nomenclature varies: HER1 also is referred to as EGFR and ERBB1; HER2, also is referred to as ERBB2 and NEU; HER3 also is referred to ERBB3; and HER4 also is referred to as ERBB4. All members of this family have an extracellular ligand-binding region, a single membrane-spanning region and a cytoplasmic tyrosine-kinase-containing domain. The HERs are expressed in various tissues of epithelial, mesenchymal and neuronal origin.

Under normal physiological conditions, activation of the HERs is controlled by the spatial and temporal expression of their ligands, which are members of the EGF family of growth factors. Ligand binding induces the formation of receptor homo- and heterodimers leading to activation of the intrinsic kinase domain, resulting in phosphorylation on specific tyrosine residues in the cytoplasmic tail, ultimately leading to activation of intracellular signalling pathways.

Each of these receptors has been shown to have a role in cancer (see, e.g., Slamon et al. (1989) Science 244:707-712; Bazley et al., (2005) Endocr. Relat. Cancer Jul12 Suppl. 1:S17-S27). For example, HER1 (ErbB1) and HER2 (ErbB2) have been implicated in the development and pathology of many human cancers; and alterations in these receptors have been associated with more aggressive disease and disease associated with poor clinical outcome. The following table summarizes roles of HER receptor family members and their cognate ligands in certain cancers:

TABLE 1
Role of HERs and their cognate ligands in Cancer*

| | Nature of | Type of | |
|---|---|---|---|
| Molecule | Disregulation | Cancer | Role |
| Ligands | | | |
| TGF-α | Overexpression | Prostate | Expressed by stroma in early androgen- |
| | | | dependent cancer and by tumors in |
| | | | advanced androgen-independent cancer |
| | Overexpression | Pancreatic | Correlates with tumor size and decreased |
| | | | patient survival; possibly due to over- |
| | | | expression of Ki-Ras, which also drives |
| | | | expression of HB-EGF and NRG1 |
| | Overexpression | Lung, ovary, | Correlates with poor prognosis when co- |
| | | colon | expressed with HER1 |
| NRG1 | Overexpression | Mammary | Necessary, but not sufficient for tumori- |
| | | adenocarcinomas | genesis in animal models |
| Receptors | | | |
| HER1 | Overexpression | Head and neck, | Significant indicator for recurrence in |
| | | breast, bladder, | operable breast tumors; associated with |
| | | prostate, kidney, | shorter disease-free time and overall |
| | | non-small-cell | survival in advanced breast cancer; |
| | | lung cancer | prognostic marker for bladder, prostate |
| | | | and non-small-cell lung cancers |
| | Overexpression | Gliomas | Amplification occurs in 40% of gliomas; |
| | | | overexpression correlates with higher |
| | | | grade and reduced survival |
| | Mutation | Glioma, lung, | Deletion of part of the extracellular |
| | | ovary, breast | domain yields a constitutively active |
| | | | receptor |
| HER2 | Overexpression | Breast, lung, | Overexpression resulting from gene |
| | | pancreas, colon, | amplification in 15-30% of all invasive |
| | | esophagus, endo- | ductal breast cancers; overexpression |
| | | metrium, cervix | correlates with tumor size, spread to |
| | | | lymph nodes, high greade, high |
| | | | percentage of S-phase cells, aneuploidy |
| | | | and lack of steroid hormone receptors |
| HER3 | Expression | Breast, colon | Coexpression with HER2 |
| | | gastric, prostate; | |
| | | other carcinomas | |
| | Overexpression | Oral squamous | Overexpression correlates with lymph |
| | | cell cancer | node involvement and patient survival |
| HER4 | Reduced | Breast, prostate | Correlates with a differentiated |
| | expression | | phenotype |
| | Expression | Childhood | Coexpression with HER2 |
| | | medulloblastoma | |

*Yarden et al. (2001) Mol. Cell. Biol., 2: 127

Because of their roles in cancers and other diseases, HER receptors are therapeutic targets. There are two classes of anti-HER therapeutics: antibodies targeted to the extracellular (or ectodomain), referred to herein as the ECD, and small-molecule tyrosine kinase inhibitors. Anti-HER drugs exhibit limited efficacy and limited duration of response. For example, Herceptin® (Trastuzimab) is a humanized version of a murine monoclonal antibody, and targets the extracellular domain of HER2. Effectiveness requires high expression (at least 3- to 5-fold overexpression) of HER2. Consequently fewer than 25% of breast cancer patients qualify for treatment. Among this population, a large proportion fail to respond to treatment (Piccart-Gebhart et al. 2005; Romond et al., 2005). In addition, small molecule tyrosine kinase inhibitors often lack specificity. Thus, with the exception of preselected highly expressing HER2 patients treated with Herceptin in combination with chemotherapy, the efficacy observed with single-targeted anti-HER agents, antibody or small molecule tyrosine kinase inhibitors, is in the range of 10-15%.

Because of the limited effectiveness of the available therapies, there remains a need to develop alternative strategies for addressing these targets. Accordingly, it is among the objects herein to provide alternative strategies for targeting the HER receptor family, including provision of more effective therapeutics than the anti-HER antibodies and small molecules.

### BRIEF SUMMARY OF THE INVENTION

As part of this specification, a list of sequences is used as part of the invention is appended. The sequences are incorporated as part of the specification.

Provided herein are therapeutics and candidate therapeutics and methods for identifying or discovering candidate therapeutics. Methods of treatment using such therapeutics are provided. The therapeutics are designed to be pan cell surface receptor therapeutics in that they specifically target more than one cell surface receptor, such as via binding to ligands for one or more receptors and/or interacting with one or more cell surface receptors, as long as the activity of more than one cell surface receptor is modulated. The therapeutics include those that target more than one HER receptor as well as those that target one or more HER receptors and additional receptors, such as a HER receptor that contributes or participates in development of resistance to anti-HER therapies. In particicular embodiments, the therapeutics and candidate therapeutics are designed to addess problems, including limited efficacy and development of resistance, associated with limitations on the effectiveness of anti-HER therapeutics.

Provided herein are multimers of an extracellular domain (ECD), or portion(s) thereof, of two cell surface receptors. The components of the multimer include a first ECD polypeptide and a second ECD polypeptide where the first and second polypeptide are separately linked directly or indirectly via a linker to a multimerization domain. In multimers provided herein, the first chimeric polypeptide can be a full-length ECD of HER1; or the first chimeric polypeptide can contain less than the full-length ECD of HER1, HER2, HER3, or HER4 where the ECD portion at least contains a sufficient portion of subdomains I and III to bind to a ligand of the HER receptor and a sufficient portion of the ECD to dimerize with a cell surface receptor, including a sufficient portion of subdomain II, unless the all or a portion of the ECD is from HER2 in which case at least part of domain IV, typically a sufficient portion of modules 2-5, of domain IV must be present to effect dimerization of the HER2 ECD. The second component of the polypeptide is a second chimeric polypeptide that contains at least a sufficient portion of an ECD of a cell surface receptor (CSR) to bind to ligand and/or to dimerize with a cell surface receptor. The CSR of the second chimeric polypeptide can be any ECD, or portion thereof, or a CSR that is desired. If, however, the first chimeric polypeptide is a full-length HER1 ECD, then the second chimeric polypeptide cannot be a full-length HER2, although a full-length HER1 can be combined with a truncated HER2 so long as the truncated HER2 contains a sufficient portion of domain IV to effect dimerization. The first and second chimeric ECD polypeptides form a multimer through interactions of their multimerization domains. The resulting multimer provided herein binds to additional ligands as compared to the first chimeric polypeptide or a homodimers thereof and/or dimerizes with more cell surface receptors than the first chimeric polypeptide or homodimers thereof.

In other multimers, at least one of the ECD domains or portion thereof, includes a mutation that alters ligand binding or other activity compared to the form lacking such mutation. In such multimers, a second ECD portion can be the same ECD domain, wildtype or mutated form, or the ECD from any other cell surface receptor. As above, the ECD or portion thereof of each monomer is linked to a multimerization domain or is linked to a second ECD or portion thereof directly or via a linker. Exemplary of such multimers, are multimers that contain at least one HER1 ECD that contains a mutation in subdomain III that increases its affinity for a ligand other than EGF. Such increase in affinity is at least 10-fold, typically 100, 1000, 10⁴, 10⁵, 10⁶ or more.

In particular, also provided are multimers that contain modified ECDs, such as an ECD or plurality thereof whose ligand binding affinity is altered. For example, EGFR1, which is activated by EGF and generally is not stimulated by NRG-2β, has been modified so that both ligands interact with the EGFR ECD to promote receptor dimerization/receptor signaling (see, Gilmore et al. (2006) Biochem J. 396:79-88, who show that NRG2β is a more potent stimulus of the EFGR mutant than of wild-type. The sequence of an exemplary modified EGFR, EGFR-S442F, is set forth in SEQ ID No. 414 in which the ECD begins at amino acid 25. The ECD (25-645 of SEQ ID No. 414; the position of the modification is at locus 442 with reference to a sequence of the ECD that includes the first 25 amino acid signal sequence and is at 418 when referencing the mature form) or a portion thereof or a corresponding portion of an allelic or species variant thereof containing at least a sufficient portion of domains I-III to bind to EGFR1 and NRG-2β (or at least a sufficient portion of modified domain III for binding to NRG-2β can be employed in the multimers provided herein as well as in the chimeras and other PAN-cell surface therapeutics provided herein. The ECDs provided herein or known to those of skill in the art can be modified to alter ligand binding specificity, such as with a modification corresponding that the exemplified modification. The ECD from EGFR-S442F, as well as from other ECDs modified to interact with ligands specific for different ECDs, can be employed as Pan-cell surface receptor therapeutics, particularly when linked to a multimerization domain, such as an Fc domain. These modified ECDs can be employed in all embodiments described herein. Hence provided herein are homo-multimers of modified ECDS of receptors that interact with at least two ligands, where each ligand interacts with a different wild-type ECD.

The multimer provided herein can be one where the ECD of one or both of the first and second chimeric polypeptide is a hybrid ECD that contains subdomains from at least two different cell surface receptor ECDs. Also included herein, are multimers where the first chimeric polypepide can contain less than the full-length of the ECD of HER2, HER3, or HER4. Most often, the first chimeric polypeptide contains less than the full-length of the ECD of HER3 or HER4.

Additionally, the ECD portion of the second polypeptide in the multimer provided herein includes those where the ECD portion of the second polypeptide is not HER1, but contains all or a portion of an ECD of another CSR. In some instances, the other ECD portion includes those where the ECD domain of the second chimeric polypeptide is from HER3 or HER4.

Also included among ECD multimers provided herein are those where the second chimeric polypeptide includes an ECD polypeptide that is a full-length ECD. Alternatively, the ECD domain of the second chimeric polypeptide is truncated and contains at least a sufficient portion of subdomains I, II, and III to bind to its ligand and to dimerize with a cell surface receptor. In some cases, the truncated ECD domain of the second chimeric polypeptide includes a sufficient portion of domains I and III to bind ligand. In other cases, the truncated ECD domain of the second chimeric polypeptide includes a sufficient portion of the ECD to dimerize with a cell surface receptor.

Also included are multimer that contain an ECD domain that is modified to alter ligand binding or other activity of the ECD or full-length receptor containing such ECD compared to the unmodified ECD or full-length receptor. Alteration includes elimination or addition of ligand binding. For example, the ECD can be modified to bind to additional ligands compared to the unmodified ECD. Such modification includes a modification a S442 (e.g., SEQ ID. No.2) or a corresponding position of an HER receptor, whereby the ECD binds to ligands for HER3, such as NRG-2β, as well ligands, such as EGF, for HER1.

These multimers can include an ECD or portion thereof from HER1 and from HER3 or HER4, whereby the resulting multimer interacts with ligands for at least two, three, four, five, six or seven HER receptors. Dimers are included among the multimers. The multimerization domains include any known to those of skill in the art, including any listed above or below, such as an Fc domain or variant thereof.

The multimerization domain of the first and second polypeptide in the multimer provided herein include any multimerization domain from among an immunoglobulin constant domain (Fc), a leucine zipper, complementary hydrophobic regions, complementary hydrophilic regions, compatible protein-protein interaction domains, free thiols that form an intermolecular disulfide bond between two molecules, and a proturberance-into-cavity and a compensatory cavity of identical or similar size that form stable multimers. In some embodiments, the multimerization domain is an Fc domain or a variant thereof that effects multimerization. The Fc domain can be from any immunoglobulin molecule including from an IgG, IgM, or IgE.

Typically, for the multimer provided herein, the cell surface receptor (CSR) of or cell surface protein from which the second chimeric polypeptide is derived and/or from which the multimer dimerizes is a cognate receptor to an ECD,or portion thereof, of the multimer. Examples of CSRs include HER2, HER3, HER4, IGF1-R, a VEGFR, a FGFR, a TNFR, a PDGFR, MET, Tie (i.e. Tie-1 or TEK (Tie-2)), RAGE, an Eph receptor, and a T cell receptor. In some embodiments, the ECD of the second chimeric polypeptide is from VEGFR1, FGFR2, FGFR4, IGF1-R, or Tie1. In other instances, the ECD or portion thereof of the second chimeric polypeptide is an intron fusion protein that is linked directly or indirectly via a linker to a multimerization domain. In some cases, the intron fusion protein is a herstain. In one aspect, the multimer provided herein binds to at least seven different ligands. In some embodiments, the second chimeric polypeptide of the multimer provided herein is another receptor tyrosine kinase (RTK) that is not all or a part of an ECD of HER1.

Such an ECD multimer can interact with any of HER ligands EGF, TGF-α, amphiregulin, HB-EGF, β-cellulin, epiregulin, and any additional ligand that binds to the ECD of a cell surface receptor other than HER1. For example, the additional ligand can include a neuregulin, such as any of a neuregulin-1, neuregulin-2, neuregulin-3, and neuregulin-4.

In some examples, the multimer provided herein includes as a first chimeric polypeptide one that contains either a i) a full-length ECD from a HER1 receptor, or ii) a portion thereof sufficient to bind ligand and/or dimerize and as a second chimeric polypeptide all or a portion of the ECD of HER3 of HER4 sufficient to bind to ligand and/or to dimierize.

Any of the multimers provided herein include component chimeric polypeptides linked to a multimerization domain where the multimerization domain can be any of a immunoglobulin constant region (Fc), a leucine zipper, complementary hydrophobic regions, complementary hydrophilic regions, compatible protein-protein interactions domains, free thiols that forms an intermolecular disulfide bond between two molecules, and a proturberance-into-cavity and a compensatory cavity of identical or similar size that form stable multimers. Such multimers, through interactions of their multimerization domain, are oriented in a back-to-back configuration where the ECD of both chimeric polypeptides are avaiblabe for dimerization with a cell surface receptor. In one example, the multimerization domain is an Fc domain. The Fc domain can be from any immunoglobulin molecule, such as from an IgG, IgM, or IgE.

Included among the multimers provided herein are those having at least two chimeric polypeptides. In one example, a multimer includes one that has at least two chimeric polypeptides where the first chimeric polypeptide contains all or part of HER1 and the second chimeric polypeptide contains all or part of HER3 or HER4.

Also included among the multimers provided herein are those where one of the constituent chimeric polypeptides is a fusion polypeptide. In some embodiments, both of the first chimeric polypeptide and second chimeric polypeptide are fusion polypeptides. In other examples, a constituent chimeric polypeptide is formed by chemical conjugation. In one embodiment, both of the first chimeric polypeptide and second chimeric polypeptide are formed by chemical conjugation. In additional examples, the multimerization domain of at least one of the chimeric polypeptides is linked directly to the ECD. Alternatively, the multimerization domain of one of the chimeric polypeptides is linked via a linker to an ECD polypeptide. In some embodiments of this, the multimerization domain of each of the first and second chimeric polypeptides are linked to each respective ECD via a linker. The linker can be a chemical linker or a polypeptide linker.

The multimer provided herein can be a heterodimer. The heterodimer can be one where the component chimeric polypeptides are in a back-to-back configuration, such that the ECD in each chimeric polypeptide is available for dimerization with a cell surface receptor.

Provided herein are heteromultimers that include an extracellular domain (ECD) from one HER receptor (i.e. HER1, HER2, HER3, or HER4), and an ECD from a second receptor such that at least one of the ECDs is a HER ECD and contains subdomains I, II, and III and part (including at least module 1) but not all of subdomain IV, of the ECD. In such a heteromultimer, the ECDs of the first and second receptor are different. In some instances, the ECDs of the first and second receptor are both HER ECDs. Thus, a heteromultimer provided herein includes one where one HER is HER1 and the other is HER3 or HER4. In other instances, the ECD of the second receptor is from a cell surface receptor. The dimerization arm of the ECD of the first or second receptor in the heteromultimer is available for dimerization with a cell surface receptor.

Included among heteromultimers provided herein are those where each ECD is linked directly or via a linker to a multimerization domain such that the multimerization domain of at least two ECDs interact to form a heteromultimer. The multimerization domain of each of the ECDs in the heteromultimer include any of an immunoglobulin constant (Fc) domain, a leucine zipper, complementary hydrophobic regions, complementary hydrophilic regions, compatible protein-protein interaction domains, free thiols that from an intermolecular disulfide bond between two molecules, or a proturberance-into-cavity and a compensatory cavity of identical or similar size that form stable multimers. In some embodiments, the multimerization domain is an Fc domain. The Fc domain can be from any immunoglobulin molecule including from an IgG, IgM, or IgE.

The cell surface receptor (CSR) of the second receptor of the heteromultimer provided herein is a cognate receptor to an ECD, or portion thereof, that is a component of the heteromultimer. Examples of CSRs include HER2, HER3, HER4, IGF1-R, a VEGFR, a FGFR, a TNFR, a PDGFR, MET, a Tie (i.e. Tie-1 or Tie-2 (TEK)), RAGE, and EPH receptor, or a T cell receptor. In some embodiments, the CSR is any of a VEGFR1, FGFR2, FGFR4, IGF1-R, or Tie-1.

Also contemplated are such heteromultimer in which a domain or part thereof from an ECD contains a mutation in the domain that alters ligand binding or specificity or other activity. The mutation alters ligand binding or other activity of the ECD or full-length receptor containing such ECD compared to the unmodified ECD or full-length receptor, whereby the heteromultimer exhibits the altered ligand binding or specificity. Exemplary of such heteromultimers are that include a HER1 ECD modified to bind to two ligands, such as a HER1 and a HER3 ligand. For example, modification of the HER ECD by replacement of S442, such as with F, or a corresponding position of an HER receptor modifies ligand binding. Such modification results in a HER1 ECD that intereacts with NRG-2β. Such heteromultimers can contain an ECD or portion thereof from HER1 and from HER3 or HER4, whereby the resulting ECD can interact with ligands for at least two or more, such as three, four, five, six and seven, HER receptors.

Provided herein are hybrid ECDs that each contain all or a part of at least domain I, II, and III of an ECD of one or more CSR such that at least two of the domains are from ECDs of different cell surface receptors and the hybrid ECD contains a sufficient portion of an ECD of a cell surface receptor, including a sufficient portion of domain II, to dimerize with a cell surface receptor when the hybrid ECD is linked to a multimerization domain and/or sufficient portions of ligand binding domains to interact with the ligand for the ECD from which the ECD domain or portion thereof is derived. In some embodiments, the cell surface receptor is a member of the HER family. Thus, for example, domain I is from HER1, domain II is from HER2, and domain III is from HER3. In another embodiment domains I and III are from an ECD containing a mutation in domain III that renders domain III able to bind to a ligand for HER3 or HER4.

The hybrid ECDs include, for example, those that contain a subdomain or portion thereof from an ECD that contains a mutation in the subdomain that alters ligand binding or specificity. Exemplary of such mutations are those described above, and below, such as a modification of HER1 whereby the modified HER1 interacts with two or more ligands, such as EGF and NRG-2β.

Also provided herein are chimeric polypeptide of a hybrid ECD provided herein linked directly or via a linker to a multimerization domain. The multimerization domain includes any of an immunoglobulin constant (Fc) domain, a leucine zipper, complementary hydrophobic regions, complementary hydrophilic regions, compatible protein-protein interaction domain, free thiols that form an intermolecular disulfide bond between two molecules, and a proturberance-into-cavity and a compensatory cavity of identical or similar size that form stable multimers. In some instances, the multimerization domain is an Fc domain. The Fc domain can be from any immunoglobulin molecule, including from an IgG, IgM, or IgE. Provided herein, is a multimer formed between at least two chimeric hybrid ECD polypeptides provided herein.

Provided herein is a heteromultimer that contains all or part of an ECD from HER1 and all or part of an ECD from HER3 or HER4 such that if the heteromultimer contains a truncated part of an ECD of HER1, HER3, or HER4, the part includes at least subdomains I, II and III.

Provided herein are chimeric polypeptides containing an ECD or portion thereof sufficient for ligand binding and/or dimerization linked to a multimerization domain. The ECD or portion thereof of the chimeric polypeptide provided herein can be from any of a HER2, HER3 or HER4 ECD or modified form thereof. Exemplary of such are: HER2-530 (SEQ ID NO:14), HER2-595 (SEQ ID NO:16), HER2-650 (SEQ ID NO:18), Her3-500 (SEQ ID NO:20), p85Her3 (SEQ ID NO:22), HER3-519 (SEQ ID NO:24), HER3-621 (SEQ ID NO:26), HER4-485 (SEQ ID NO:28), HER4-522 (SEQ ID NO:30), HER4-650 (SEQ ID NO:32), a polypeptide set forth in any or SEQ ID NOS: 32, 34, 127, 141, 146, 159, and 54-125 and allelic and species variants of any of the aforementioned ECDs as well a modified forms thereof, such as forms modified to alter an activity (see, e.g., residues 25-645, or a portion thereof that includes residue 442F, of SEQ ID No. 414, which sets forth the sequence of a modified HER1 (EGFR1) in which S at 442 is replaced by F to yield an ECD that binds to NRG2βas well as EGF). Also provided is a heteromultimer containing two or more chimeric polypeptides from any of a HER1-501 (SEQ ID NO:10), HER1-621 (SEQ ID NO:12) HER1 S442F (SEQ ID No. 414, residues 25-645) or a portion of any of the preceding HER1 polypeptides sufficient for ligand binding (for HER1 S442F containing the S442F mutation) and/or receptor dimerization, HER2-530 (SEQ ID NO:14), HER2-595 (SEQ ID NO:16), HER2-650 (SEQ ID NO:18), Her3-500 (SEQ ID NO:20), p85Her3 (SEQ ID NO:22), HER3-519 (SEQ ID NO:24), HER3-621 (SEQ ID NO:26), HER4-485 (SEQ ID NO:28), HER4-522 (SEQ ID NO:30), HER4-650 (SEQ ID NO:32), a polypeptide set forth in any or SEQ ID NOS: 32, 34, 127, 141, 146, 159, and 54-125, and allelic or species variants thereof of any of the aforementioned polypeptides where the ECD, or portions thereof, in the heteromultimer are linked directly or indirectly via linkers to a multimerization domain.

Provide are chimeric polypeptides that contain an ECD or portion thereof of a HER1 receptor linked to a multimerization domain, such as any listed above, where ECD or portion thereof includes a modification(s), whereby the ECD binds to an additional ligand compared to the unmodified ECD or portion thereof. Exemplary of such polypeptides are chimeric polypeptides containing all or a portion of a contiguous sequence of amino acids from residues 25-645 of SEQ ID No. 414 or having at least about 70, 80, 90, 95% sequence identity thereto and including a mutation, such as Ser to Phe at a position corresponding to 442 of SEQ ID No.414, that alters ligand binding, linked to a multimerization domain. The alteration in ligand binding includes a modification such that the ECD of HER1 also binds to HER3 ligands, such as NRG-2β. For example, chimeric polypeptides containing a multimerization domain and a sufficient portion of the ECD of a modified HER1 to interact with EGF and NRG-2β.

Included among chimeric polypeptides in the multimers and heteromultimers are chimeric polypeptides that contain a multimerization domain linked directly or indirectly via a linker to the polypeptide set forth as amino acids 25-645 of SEQ ID No. 414 or a portion thereof sufficient to effect ligand binding to at least two different ligands. These chimeric polypeptides also are provided.

In some embodiments, the multimerization domain of the chimeric polypeptide or of the heteromultimer can be any of an immunoglobulin constant region (Fc), a leucine zipper, complementary hydrophobic regions, complementary hydrophilic regions, compatible protein-protein interaction domains, free thiols that form an intermolecular disulfide bond between two molecules, and a protuberance-into-cavity and a compensatory cavity of identical or similar sixe that form stable dimers such that the chimeric polypeptides in the heteromultimer interact in a back-to-back configuration where the ECD of both chimeric polypeptides are available for dimerization with a cell surface receptor. In some cases, the multimerization domain is an Fc domain. The Fc domain can be from any immunoglobulin molecule including an IgG, IgM, or an IgE.

Also provided herein isolated polypeptide containing a sequence of amino residues set forth in any of SEQ ID NOS: 127, 141, 146, 153, 155, 157, 159, 297, or 299. Such an isolated polypeptide can be linked to a multimerization domain to provide for a chimeric polypeptide. Also provided is a heteromultimer that contains a chimeric polypeptide having an amino acid sequence set forth in any of SEQ ID NOS:127, 141, 146, 153, 155, 157, 159, 297, or 299 and a sequence for a multimerization domain. The heteromultimer can contain as a second polypeptide a HER ECD or portion thereof sufficient for ligand binding and/or receptor dimerization.

Provided herein are nucleic acid molecules encoding a chimeric polypeptide provided herein or at least one chimeric polypeptide in the multimers or heteromultimers provided herein, including the hybrid ECDs provided herein. Provided herein are vectors containing the nucleic acid molecules. Also provided are cells containing a vector as described herein.

Provided herein are pharmaceutical compositions containing a multimer, heteromultimer, or chimeric polypeptide provided herein, or encoding nucleic acid molecule. Also provide are pharmaceutical compositions containing an isolated cell that contains a nucleic acid provided herein or a vector provided herein. In some embodiments, the pharmaceutical composition is formulated for single dosage administration. In some cases, the pharmaceutical compositions also can be formulated for local, topical or systemic administration.

Provided herein are methods of treating a disease or condition by administering any of the pharmaceutical compositions described herein. Diseases or conditions treated include cancer, inflammatory disease, an angiogenic disease, or a hyperproliferative disease. Exemplary of cancers include pancreatic, gastric, head and neck, cervical, lung, colorectal, endometrial, prostate, esophageal, ovarian, uterine, glioma, bladder, renal, or breast cancer. Included among diseases to be treated is a proliferative disease. Exemplary of proliferative diseases include those that involve proliferation and/or migration of smooth muscle cells, or a disease of the anterior eye, a diabetic retinopathy, or psoriasis. Other exemplary diseases to be treated include restenosis, ophthalmic disorders, stenosis, atherosclerosis, hypertension from thickening of blood vessels, bladder diseases, and obstructive airway diseases. Other exemplary diseases include diseases or conditions associated with, e.g., caused by, or aggravated by, exposure to one or more Neuregulin (“NRG”), such as NRG1, including type I, II, and III, NRG2, NRG3, and/or NRG4. Examples of NRG-associated diseases include neurological or neuromuscular diseases, including schizophrenia and Alzheimer's disease.

Provided herein is a method of treating cancer by administering any of the pharmaceutical compositions provided herein in combination with another anti-cancer agent. The anti-cancer agent includes radiation and/or a chemotherapeutic agent. In one example, the anti-cancer agent includes a tyrosine kinase inhibitor or an antibody. Exemplary of anti-cancer agents include a quinazoline kinase inhibitor, an antisense or siRNA or other double-stranded RNA molecule, an antibody that interacts with a HER receptor, and antibody conjugated to a radionuclide, or a cytotoxin. Other exemplary anti-cancer agents include Gefitinib, Tykerb, Panitumumab, Eroltinib, Cetuximab, Trastuzimab, Imatinib, a platinum complex or nucleoside analog.

Provided herein is a method of treatment of a HER-mediated disease including testing a subject with the disease to identify which HER receptors are expressed or overexpressed and based on the results, selecting a multimer that targets at least one, typically, two of the HER receptors. In one embodiment, the disease is a cancer. Exemplary of cancers include pancreatic, gastric, head and neck, cervical, lung, colorectal, endometrial, prostate, esophaegeal, ovarian, uterine, glioma, bladder or breast cancer.

Provided herein is a polypeptide having a sequence of amino acids set forth in any one of SEQ ID NOS: 54-125, or 405.

Provided herein is a method of identifying candidate thereapeutic molecules that interact with HER receptors by first contacting a test molecule or collection thereof with a polypeptide of at least 6 amino acids or 6 amino acids up to about 50 amino acids or 50 amino acids based upon regions in domains II and IV or I and III that are involved in any of dimerization, ligand binding, and/or tethering and then identifying and selecting any test molecule that interacts with one or more of the polypeptides. In one embodiment, the polypeptides are contained within a library that is a combinatorial library of polypeptides based upon the HER receptors. Exemplary of polypeptides for which the test molecule can be contacted include any of having a sequence of amino acids set forth in any of SEQ ID NOS: 54-125, and portions of any of the polypeptides that have 4, 5, 6, 8, 10, 12, or more amino acid residues thereof, or SEQ ID NO:405, and portions thereof that have 6, 8, 10, 12, 14,1 5, 18, 20, 25, 30, 35, 40, 45, or 50 or more amino acid residues thereof. Among the library of molecules are those that contain polypeptides on a solid support or on the surface of a virus. In one example, the polypeptides are contained within a phage display library.

In one embodiment, the test molecules are a library of molecules. Thus, in one example, the test molecules include those in a phage display library. In another embodiment, the molecules are small organic compounds or polypeptides.

In the method provided herein, test molecules are selected that bind to a domain I and/or domain III, or to domain II or to domain IV. In one aspect of the method, a heterodimer of two or more polypeptide test molecules identified is made where one of the peptides of the heterodimer binds to domain II and the other binds to domain IV.

Provided herein is an isolated antibody that interacts with any of the polypeptides having a sequence of amino acids set forth in any of SEQ ID NOS: 54-125, or 405. In one embodiment, the antibody is a multiclonal antibody that interacts with two or more of the polypeptides provided herein. In some examples, the antibody is a receptabody dimer or multimer that contains at least two different polypeptides each linked to a multimerization domain. The multimerization domain is any of a immunoglobulin constant region (Fc), a leucine zipper, complementary hydrophobic regions, complementary hydrophilic regions, compatible protein-protein interaction domain, free thiols that form an intermolecular disulfide bond between two molecules, and a protuberance-into-cavity and a compensatory cavity of identical or similar size that form stable multimers. In one example, the multimerization domain is an Fc domain. The Fc domain can be from any immunoglobulin molecule such as from an IgG, IgM, or an IgE.

Among the heteromultimers are those in which a subdomain or part thereof of an ECD contains a mutation in the domain that alters ligand binding or specificity or other activity. For example, the mutation alters ligand binding or other activity of the ECD or full-length receptor containing such ECD compared to the unmodified ECD or full-length receptor, whereby the heteromultimer exhibits the altered ligand binding or specificity. Such modifications include any that eliminate or add or enhance an activity, such as binding to an additional ligand, such as interaction of an ECD of a HER1 with a ligand for HER3, such as NRG-213 ligand. Examplary of such modifications is a modification that corresponds to modification at position S442, such as S442F, of HER1 or a corresponding position of a HER receptor. The resulting ECD binds to or interacts with at least two ligands, one for HER1, such as the ligand EGF, and a second for HER3, such as NRG-2β.

These heteromultimer can contain and ECD or portion thereof from HER1 and from HER3 or HER4, whereby the resulting hybrid can interact with ligands for at least three HER receptors. These heteromultimers and contain a multimerization domain, such as any described herein or known to those of skill in the art, such as an Fc multimerization domain or variant thereof (i.e. a variant whose T cell interactions are altered).

The invention also provides for compositions comprising a mixture of heteromultimers and homomultimers wherein the heteromultimer comprises an ECD or portion thereof from HER1 and another ECD or portion thereof from HER3 and wherein the homomultimers comprise an ECD or portion thereof from HER1 or an ECD or portion thereof from HER3. In some aspects, the HER1 portion has been enhanced for ligand binding and/or biological activity. In other aspects, the HER3 portion has been enhanced for ligand binding and/or biological activity. In yet another aspect, both HER1 and HER3 portions have been enhanced for ligand binding and/or biological activity.

The invention also provides for pharmaceutical compositions comprising the compositions above formulated for topical, oral, systemic, or local administration.

In another aspect, the invention provides for methods for treating cancer, an inflammatory disease, an angiogenic disease or a hyperproliferative disease, comprising administering a therapeutically effective amount of a composition listed above. In some aspects, the cancer is pancreatic, gastric, head and neck, cervical, lung, colorectal, endometrial, prostate, esophageal, ovarian, uterine, glioma, bladder, renal or breast cancer. In other aspects, the disease is a proliferative disease. In other aspects, the proliferative disease involves proliferation and/or migration of smooth muscle cells, or is a disease of the anterior eye, or is a diabetic retinopathy, or psoriasis. In other aspects, the disease is restenosis, ophthalmic disorders, stenosis, atherosclerosis, hypertension from thickening of blood vessels, bladder diseases, and obstructive airway diseases.

### BRIEF DESCRIPTION OF THE FIGURES

Since interactions are dynamic, amino acid positions noted are for reference and exemplification. The noted positions reflect a range of loci that vary by 2, 3, 4, 5 or more amino acids. Variations also exist among allelic variants and species variants. Those of skill in the art can identify corresponding sequences by visual comparison or other comparisons including readily available algorithms and software.

FIG. 1(a) depicts a schematic of of the Human EGF Receptor 1 (HER1; ErbB1; EGFR) and sets forth the loci for various features with reference to HER1, but such structures are also conserved among other family members (i.e. HER2, 3, 4). The ECD of HER (ErbB) family members contains four subdomains, designated domains I (L1), II (S1), III (L2), and IV (S2). Subdomains I and III cooperate for ligand binding; domain II contains sequences required for dimerization (the ‘dimerization arm’); and domain IV contains sequences which allow domain II/IV tethering (except for HER2 which does not undergo a tethered conformation). The small disulfide-bonded modules within domains II and IV are represented by individual boxes. The β-hairpin/loop (also called the dimerization arm) in domain II (corresponding to amino acids 240-260 of full length mature HER1) is indicated. The shorter β-hairpin/loops in domain IV that facilitate tethering (corresponding to amino acids 561-569 and to amino acids 572-585 of full length mature HER1) are indicated. Some amino acid residues within the loop regions that participate in dimerization and/or tethering of the receptor are specified. HER full-length receptors also contain a transmembrane domain (shaded region), juxtamembrane (JM) domain, kinase domain, and cytosolic tail (CT).

FIG. 1(b) depicts the mechanism of ligand induced HER dimerization. Domains I, II, III, and IV are depicted. Most (about 95%) of HER receptors exist in a tethered conformation where domain II and IV form an intramolecular interaction. The remaining 5% of monomeric receptors on the cell surface are in an untethered or open configuration. Ligands (E) bind to domains I and/or III of HER family receptors. Ligand binding stabilizes the untethered conformation in which the dimerization arm in domain II is exposed. The domain II dimerization arm interacts with regions in domain II of another HER family receptor to yield homo- and hetero-dimers. Ligand binding and dimerization of HER receptors induces activation of the intrinsic kinase domain, resulting in phosphorylation on specific tyrosine residues within the cytoplasmic tail and subsequent downstream signaling.

FIG. 2(a) depicts alignment and domain organization of HER1 (EGFR) ECD isoforms as compared to the mature form (lacking the signal sequence) of the full-length EGFR (NP_{—}005219, corresponding to amino acids 25-1210 of SEQ ID NO:2). Aligned HER1 (EGFR) ECD isoforms (lacking a signal sequence) include HF100 (SEQ ID NO:12), HF110 (SEQ ID NO: 10), HF120 (ERRP, SEQ ID NO:34), HE R1 (EGFR) isoform b (NP_{—}958439, corresponding to amino acids 25-628 of SEQ ID NO:12), HER1 (EGFR) isoform c (NP_{—}958440, corresponding to amino acids 25-405 of SEQ ID NO:133), and HER1 (EGFR) isoform d (NP_{—}958441, corresponding to amino acids 25-705 of SEQ ID NO:131). Domain I (corresponding to amino acids 1-165 of full-length mature HER1 (EGFR)) and domain III (corresponding to amino acids 313-481 of full-length mature HER1 (EGFR)) are denoted in bold. Domain II (corresponding to amino acids 166-312 of full-length mature HER1 (EGFR)) and domain IV (corresponding to amino acids 482-621 of full-length mature HER1 (EGFR)) are denoted in regular font, with cysteine modules highlighted. Non-ECD portions of full-length mature HER1 (EGFR)) are denoted in light grey. Amino acids showing no alignment to amino acid sequences in the mature full-length HER1 (EGFR) are depicted by italics.

FIG. 2(b) depicts alignment and domain organization of HER2 ECD isoforms as compared to the mature form (lacking the signal sequence) of the full-length HER2 (AAA75493.1, corresponding to amino acids 23-1255 of SEQ ID NO:4). Aligned HER2 ECD isoforms (lacking a signal sequence) include HF200 (SEQ ID NO:18), ErbB2.1e (corresponding to amino acids 23-633 of SEQ ID NO:137), HF210 (SEQ ID NO:16), HF220 (SEQ ID NO:14), ErbB2.1d (corresponding to amino acids 25-680 of SEQ ID NO:136), ErbB2.1f (corresponding to amino acids 23-575 of SEQ ID NO:138), HER2-int11 (corresponding to amino acids 23-438 of SEQ ID NO:141), herstatin (AAD56009, corresponding to amino acids 23-419 of SEQ ID NO:135), and ErbB2.a (corresponding to amino acids 23-90 of SEQ ID NO:139). Domain I (corresponding to amino acids 1-172 of full-length mature HER2) and domain III (corresponding to amino acids 320-488 of full-length mature HER2) are denoted in bold. Domain II (corresponding to amino acids 173-319 of mature full-length HER2) and domain IV (corresponding to amino acids 489-628 of full-length mature HER2) are denoted in regular font, with cysteine modules highlighted. Non-ECD portions of full-length mature HER1 (EGFR) are denoted in light grey. Amino acids showing no alignment to amino acid sequences in the mature full-length HER2 are depicted by italics.

FIG. 2(c) depicts alignment and domain organization of HER3 ECD isoforms as compared to the mature form (lacking the signal sequence) of the full-length HER3 (NP_{—}001973.1, corresponding to amino acids 20-1342 of SEQ ID NO:6). Aligned HER3 ECD isoforms (lacking a signal sequence) include HF300 (SEQ ID NO:26), HF310 (SEQ ID NO:20), p85HER3 (corresponding to amino acids 20-562 of SEQ ID NO:22), HER3-519 (SEQ ID NO:24), HER3 isoform (AAH02706, corresponding to amino acids 20-331 of SEQ ID NO:143), HER3-int10 (corresponding to amino acids 20-403 of SEQ ID NO:146), p75sHER3 (corresponding to amino acids 20-534 of SEQ ID NO:150), HER3-int11 (corresponding to amino acids 20-425 of SEQ ID NO:148), p45sHER3 (corresponding to amino acids 20-331 of SEQ ID NO:149), p50sHER3 (corresponding to amino acids 20-400 of SEQ ID NO:151), and HER3 isoform 2 (P21860-2, corresponding to amino acids 20-183 of SEQ ID NO:144). Domain I (corresponding to amino acids 1-159 of full-length mature HER3) and domain III (corresponding to amino acids 312-480 of full-length mature HER3) are denoted in bold. Domain II (corresponding to amino acids 160-311 of full-length mature HER3) and domain IV (corresponding to amino acids 481-621 of full-length mature HER3) are denoted in regular font, with cysteine modules highlighted. Non-ECD portions of full-length mature HER3 are denoted in light grey. Amino acids showing no alignment to amino acid sequences in the mature full-length HER3 are depicted by italics.

FIG. 2(d) depicts alignment and domain organization of HER4 (ErbB4) ECD isoforms as compared to the mature form (lacking the signal sequence) of the full-length HER4 (ErbB4) (NP_{—}005226, corresponding to amino acids 26-1308 of SEQ ID NO:8). Aligned ErbB4 ECD isoforms (lacking a signal sequence) include ErbB4-522 (SEQ ID NO:30), HF400 (SEQ ID NO: 32), ErbB4-int11 (corresponding to amino acids 26-430 of SEQ ID NO: 157), ErbB4-int12 (corresponding to amino acids 26-506 of SEQ ID NO:159), HF410 (SEQ ID NO:28), ErbB4-int9 (corresponding to amino acids 26-391 of SEQ ID NO:153), and ErbB4-int10 (corresponding to amino acids 26-421 of SEQ ID NO:155). Domain I (corresponding to amino acids 1-163 of full-length mature ErbB4) and domain III (corresponding to amino acids 309-477 of full-length mature ErbB4) are denoted in bold. Domain II (corresponding to amino acids 164-308 of full-length mature ErbB4) and domainIV (corresponding to amino acids 478-625 of full-length mature ErbB4) are denoted in regular font, with cysteine modules highlighted. Non-ECD portions of full-length mature HER1 (EGFR) are denoted in light grey. Amino acids showing no alignment to amino acid sequences in the mature full-length ErbB4 are depicted by italics.

FIG. 3(a) shows the synergistic growth inhibitory effect observed when MDA MB 468 cells were treated with RB200h and tyrosine kinase inhibitor AG825.

FIG. 3(b) shows the synergistic growth inhibitory effect observed when A 431 cells were treated with RB200h and Gefitinib (Iressa).

FIG. 4 shows a schematic of RB200h, a Pan-Her ligand trap.

FIG. 5 shows the purity of hermodulin constructs (RB600, HFD100, HDF300, and RB200h) as analyzed by reverse-phase HPLC.

FIG. 6a shows that engineered dimers retain specificity to ¹²⁵I-EGF and ¹²⁵I-HRGβ: Lane 1: HFD100=HER1-621/Fc, Lane 2: HFD200=HER2-628/Fc, Lane 3: HFD300=HER3-621/Fc, and Lane 4:HFD400=HER4-625/Fc.

FIG. 6b shows that engineered dimers of RB200h retain specificity to ¹²⁵I-EGF and ¹²⁵I-HRG1β.

FIG. 7a shows EU-NRG1β1 binding to RB200h.

FIG. 7b shows binding of EU-EGF to RB200h.

FIG. 7c shows competition Eu-EGF binding by other HER ligands.

FIG. 7d shows competition of Eu-NRG1-b1 binding by other HER ligands.

FIGS. 8a-c show inhibition of EGF ligand-stimulated HER family protein phosphorylation by RB200h, Herceptin, or Erbitux in A431 epidermoid cancer cells.

FIGS. 8d-f show inhibition of NRG1β1 ligand-stimulated HER family protein phosphorylation by RB200h, Herceptin, or Erbitux in A431 epidermoid cancer cells.

FIG. 9a-c show inhibition of EGF ligand stimulated HER family protein phosphorylation by RB200h, Herceptin, or Erbitux in ZR-75-1 breast cancer cells.

FIG. 9d-f show inhibition of NRG1β1 ligand stimulated HER family protein phosphorylation by RB200h, Herceptin, or Erbitux in ZR-75-1 breast cancer cells.

FIG. 10a shows RB600 is more potent than RB200h in inhibiting receptor phosphorylation stimulated by EGF.

FIG. 10b shows RB600 is more potent than RB200h in inhibiting receptor phosphorylation stimulated by NRG1β1.

FIG. 11a shows RB200h inhibits proliferation of cultured tumor cells, A431 cells.

FIG. 11b shows RB200h inhibits proliferation of cultured tumor cell, MDA-MB-468 breast cancer cells.

FIG. 12 a-b show RB200h inhibits both ligand stimulated and unstimulated Soft Agar colony growth of ZR-75-1 (FIG. 11a) and A549 (FIG. 11b) tumor cells.

FIG. 13a shows RB200h inhibits ligand-induced proliferation of breast cancer cells induced by EGF.

FIG. 13b shows RB200h inhibits ligand-induced proliferation of breast cancer cells induced by NRG1β1.

FIG. 13c shows RB200h inhibits ligand-induced proliferation of breast cancer cells induced by LPA.

FIG. 14a shows RB200h Inhibits ligand-induced proliferation of SUM149 breast cancer cells by EGF.

FIG. 14b shows RB200h Inhibits ligand-induced proliferation of SUM149 breast cancer cells by LPA.

FIG. 15a-d show synergistic growth inhibition of RB200h with tyrosine kinase inhibitors: AG-825, Gefitinib, and Erlotinib.

FIG. 16 shows synergistic growth inhibition of RB200h with tyrosine kinase inhibitors: Gefitinib.

FIG. 17 shows RB200h has synergistic antiproliferative effect with AG 825 tyrosine kinase inhibitor.

FIG. 18 shows RB200h produces potent synergistic antiproliferative response with Iressa in A431 epidermal cancer cells.

FIG. 19 shows synergism between RB200h and Iressa in BT474 breast cancer cells.

FIG. 20 shows therapeutic evaluation of RB200h in A431 s.c. model. Mean tumor volume of s.c. A431 tumor in nude mice. Dosing was initiated at day 10. Two-way ANOVA with Bonferroni's post test. In the figue, * Statistical significant indicates a p<0.05, ** indicates p<0.01, and *** indicates p<0.001.

FIG. 21 shows a schematic of the method used for producing HFD100 mutants by PCR from HFD 100.

FIG. 22 shows HFD100-T39S has enhanced affinity for EGF (FIG. 22a), HB-EGF (FIG. 22b), and TGF-α (FIG. 22c).

FIG. 23 shows binding affinity of HFD100 mutants for EGF, HB-EGF, and TGF-α and relative expression levels.

FIG. 24 shows the mean bodyweights (panel A) and final tumor volume (panel B) for a pilot toxicity study.

FIG. 25 shows the mean tumor volume of s.c. A431 tumor in nude mice. The dosing was initiated at day 10. Statistical significant of *p<0.05, **p<0.01, ***p<0.001 was calculated using Two way ANOVA with Bonferroni's post test/

FIG. 26 shows the mean tumor weights of s.c. A431 tumors. Statistical significance was calculated using One way ANOVA.

FIG. 27 shows the mouse bodyweights during therapeutic study.

### DETAILED DESCRIPTION

A. Definitions

B. Pan-Cell Surface Receptor-Specific Therapeutics

C. HER receptor and other cell surface receptor structure and activities
1. HER1 ECD structure and domain organization
2. HER2 ECD structure and domain organization
3. HER3 ECD structure and domain organization
4. HER4 ECD structure and domain organization
5. HER Family Ligands, Ligand specificity, and Ligand-Mediated Receptor activation
6. Dimerization versus Tethering and Generation of Active Homo- and Heterodimers
7. HER Family Receptor Activity
   a. Cell Proliferation
   b. Cell Survival
   c. Angiogenesis
   d. Migration and Invasion
8. Other CSR ECDs
   a. VEGFR1 (Flt-1) and VEGFR2 (KDR)
   b. FGFR1-FGFR4
   c. IGF-1R
   d. RAGE and other CSRs

D. Components of ECD multimers and Formation of ECD multimers
1. ECD polypeptides
   a. HER family full length ECD
      i. HER1 ECD
      ii. HER2 ECD
      iii. HER3 ECD
      iv. HER4 ECD
   b. HER family truncated ECD
      i. Truncated HER1 ECD
      ii. Truncated HER2 ECD
      iii. Truncated HER3 ECD
      iv. Truncated HER4 ECD
   c. Hybrid ECD
   d. Other CSR or RTK ECDs, or portions thereof
   e. Alternatively spliced polypeptide isoforms
2. Formation of Multimers
   a. Peptide Linkers
   b. Heterobifunctional linking agents
   c. Polypeptide Multimerization domains
      i. Immunoglobulin domain
         (a). Fc domain
         (b). Protuberances-into-cavity (i.e. knobs and holes)
      ii. Leucine Zipper
         (a) fos and jun
         (b) GCN4
      iii. Other multimerization domains
         (a) R/PKA-AD/AKAP
3. Chimeric ECD Polypeptides
   a. Exemplary Chimeric HER ECD polypeptides

E. ECD multimers
a. Full-length HER1 ECD and all or part of an ECD of another

CSR
b. Two or more truncated ECD components
c. Hybrid ECD multimers
d. ECD components that are the same or derived from the same

CSR

F. Methods of Producing Nucleic Acid Encoding Chimeric ECD polypeptide fusions and Production of the Resulting ECD Multimers
1. Synthetic genes and polypeptides
2. Methods of cloning and isolating ECD polypeptides
3. Methods of Generating and Cloning ECD Polypeptide Chimeras
4. Expression Systems
   a. Prokaryotic expression
   b. Yeast
   c. Insect cells
   d. Mammalian cells
   e. Plants
5. Methods of Transfection and Transformation
6. Recovery and Purification of ECD Polypeptides, Chimeric Polypeptides, and the Resulting ECD multimers

G. Assays to Assess or Monitor ECD Multimer Activities
1. Kinase/Phosphorylation Assays
2. Complexation/Dimerization
3. Ligand Binding
4. Cell Proliferation Assays
5. Cell Disease Model Assays
6. Animal Models

H. Preparation, Formulation and Administration of ECD multimers and ECD multimer Compositions

I. Exemplary Methods of Treatment with ECD multimers
1. HER-mediated Diseases or Disorders
   a. Cancer
   b. Angiogenesis
   c. Neuregulin-associated disease
   d. Smooth Muscle Proliferative-related diseases and conditions
2. RTK-mediated disorders or diseases
   a. Angiogeneis-related ocular conditions
   b. Angiogenesis-related atherosclerosis
   c. Additional Angiogenesis-related Treatments
   d. Cancers
3. Other CSR-mediated Diseases or Disorders
4. Selection of the ECD Polypeptide Components of an ECD multimer
5. Patient Selection
6. Combination Therapies

J. Methods for the Identifying, Screening and creating Pan-HER Therapeutics
1. Targets for Pan-HER Therapeutics
2. Screening methods to Identify Pan-HER Therapeutics
   a. Phage Display
      i. Peptide Libraries
      ii. Multimeric Polypeptides (Heterodimeric peptides)
   b. Exemplary Screening Assays

K. Examples

### A. Definitions

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, a “pan-cell surface receptor therapeutic” or “pan-cell surface receptor-specific therapeutic” is a molecule, including peptide based compounds and small molecules, that can modulate the activity of two or more cell surface receptors.

As used herein, “pan-HER therapeutics” or “pan-HER-specific therapeutics” are pan-cell surface receptor therapeutics (molecules, including peptide based compounds and small molecules), that can modulate the activity of two or more HER (ErbB) receptors. Generally a Pan-HER therapeutic targets at least two different HER receptors, such as via ligand binding and/or interaction with the receptors.

As used herein, an anti-cancer agent includes any cancer treatment and drug therefor and includes radiation therapy, surgery, anti-cancer compounds, including small molecules, chemotherapeutic agents, such as cisplatin and gencytinbine, and monoclonal antibodies.

As used herein, a cell surface receptor is a protein that is expressed on the surface of a cell and typically includes a transmembrane domain or other moiety that anchors it to the surface of a cell. As a receptor it binds to ligands that mediate or participate in an activity of the cell surface receptor, such as signal transduction or ligand internalization. Cell surface receptors include, but are not limited to, single transmembrane receptors and G-protein coupled receptors. Receptor tyrosine kinases, such as growth factor receptors, also are among such cell surface receptors.

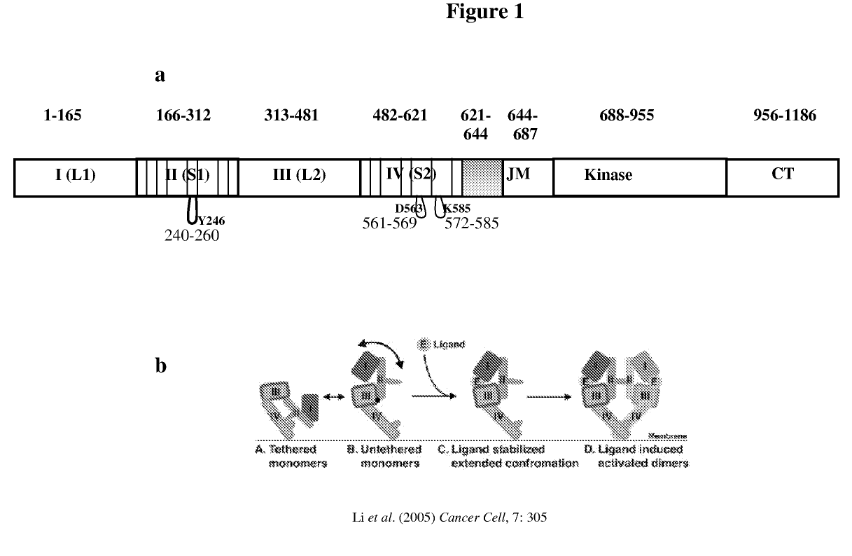

As used herein, a domain refers to a portion (a sequence of three or more, generally 5 or 7 or more amino acids) of a polypeptide that is a structurally and/or functionally distinguishable or definable. For example, a domain includes those that can form an independently folded structure within a protein made up of one or more structural motifs (e.g. combinations of alpha helices and/or beta strands connected by loop regions) and/or that is recognized by virtue of a functional activity, such as kinase activity. A protein can have one, or more than one, distinct domain. For example, a domain can be identified, defined or distinguished by homology of the sequence therein to related family members, such as homology and motifs that define an extracellular domain. In another example, a domain can be distinguished by its function, such as by enzymatic activity, e.g. kinase activity, or an ability to interact with a biomolecule, such as DNA binding, ligand binding, and dimerization. A domain independently can exhibit a function or activity such that the domain independently or fused to another molecule can perform an activity, such as, for example proteolytic activity or ligand binding. A domain can be a linear sequence of amino acids or a non-linear sequence of amino acids from the polypeptide. Many polypeptides contain a plurality of domains. For example, the domain structure of HER1 (EGFR) is set forth in FIG. 1: it includes an ECD, a transmembrane domain, a juxtamembrane domain, a kinase domain, and a C-terminal cytoplasmic domain. For HER1 (EGFR) the ECD includes four subdomains referred to as I (or L1), II (or S1), III (or L2) and IV (or S2). The “L” subdomains (I and III) participate in ligand interactions, the II (S1) and IV (S2) domains interact via the tethering region; subdomain II (S1) includes the dimerization loop. Those of skill in the art are familiar with domains and can identify them by virtue of structural and/or functional homology with other such domains.

As used herein, a cytoplasmic domain is a domain that participates in signal transduction.

As used herein, an extracellular domain (ECD) is the portion of the cell surface receptor that occurs on the surface of the receptor and includes the ligand binding site(s). For purposes herein, reference to an ECD includes any ECD-containing molecule, or portion thereof, so long as the ECD polypeptide does not contain any contiguous sequence associated with another domain (i.e. Transmembrane, protein kinase domain, or others) of a cognate receptor. Thus, for example, an ECD polypeptide includes alternative spliced isoforms of CSRs where the isoform has an ECD-containing portion, but lacks any other domains of a cognate CSR, and also has additional sequences not associated or aligned with another domain sequence of a cognate CSR. These additional sequences can be intron-encoded sequences such as occur in intron fusion protein isoforms. Typically, the additional sequences do not inhibit or interfere with the ligand binding and/or receptor dimerization activities of a CSR ECD polypeptide. An ECD polypeptide also includes hybrid ECDs.

As used herein, a hybrid ECD refers to an ECD that contains a portion of an ECD from different cell surface receptors. Typically, a hybrid ECD contains at least two ECD subdomains from different cell surface receptors.

As used herein, a chimeric polypeptide refers to a polypeptide that contains portions from at least two different polypeptides or from two non-contiguous portions of a single polypeptide. Thus, a chimeric polypeptide generally includes a sequence of amino acid residues from all or part of one polypeptide and a sequence of amino acids from all or part of another different polypeptide. The two portions can be linked directly or indirectly and can be linked via peptide bonds, other covalent bonds or other non-covalent interactions of sufficient strength to maintain the integrity of a substantial portion of the chimeric polypeptide under equilibrium conditions and physiologic conditions, such as in isotonic pH 7 buffered saline. For purposes herein, chimeric polypeptides include those containing all or part of an ECD portion of a CSR linked directly or indirectly to a multimerization domain. Chimeric polypeptides can include additional sequences as well, such as for example, epitope tags.

As used herein, a fusion construct refers to a nucleic acid molecule containing coding sequence from one nucleic acid molecule and the coding sequence from another nucleic acid molecule in which the coding sequences are in the same reading frame such that when the fusion construct is transcribed and translated in a host cell, the protein is produced containing the two proteins. The two molecules can be adjacent in the construct or separated by a linker polypeptide that contains, 1, 2, 3, or more, typically few than 10, 9, 8, 7, 6 amino acids. The protein product encoded by a fusion construct is referred to as a fusion polypeptide. The spacer can encode a polypeptide that alters the properties of the polypeptide, such as solubility or intracellular trafficking.

As used herein, a fusion protein refers to a chimeric protein containing two or portions from two more proteins or peptides that are linked directly or indirectly via peptide bonds.

As used herein, a multimerization domain refers to a sequence of amino acids that promotes stable interaction of a polypeptide molecule with another polypeptide molecule containing a complementary multimerization domain, which can be the same or a different multimerization domain to forms a stable multimer with the first domains. Generally, a polypeptide is joined directly or indirectly to the multimerization domain. Exemplary multimerization domains include the immunoglobulin sequences or portions thereof, leucine zippers, hydrophobic regions, hydrophilic regions, compatible protein-protein interaction domains such as, but not limited to an R subunit of PKA and an anchoring domain (AD), a free thiol that forms an intermolecular disulfide bond between two molecules, and a protuberance-into-cavity (i.e., knob into hole) and a compensatory cavity of identical or similar size that form stable multimers. The multimerization domain, for example, can be an immunoglobulin constant region. The immunoglobulin sequence can be an immunoglobulin constant domain, such as the Fc domain or portions thereof from IgG1, IgG2, IgG3 or IgG4 subtypes, IgA, IgE, IgD and IgM.

As used herein, “knobs into holes” (also referred to herein as protuberance-into-cavity) refers to particular multimerization domains engineered such that steric interactions between and/or among such domains, not only promote stable interaction, but also promote the formation of heterodimers (or multimers) over homodimers (or homomultimers) from a mixture of monomers. This can be achieved, for example by constructing proturberances and cavities. Protuberances can be constructed by replacing small amino acid side chains from the interface of the first polypeptide with larger side chains (e.g. tyrosine or tryptophan). Compensatory “cavities” of identical or similar size to the protuberances optionally are created on the interface of a second polypeptide by replacing large amino acid side chains with smaller ones (e.g., alanine or threonine).

As used herein, complementary multimerization domains refer to two or more multimerization domains that interact to form a stable multimers of polypeptides linked to each such domain. Complementary multimerization domains can be the same domain or a member of a family of domains, such as for example, Fc regions, leucine zippers, and knobs and holes.

As used herein, “Fc” or “Fc region” or “Fc domain” refers to a polypeptide containing the constant region of an antibody heavy chain, excluding the first constant region immunoglobulin domain. Thus, Fc refers to the last two constant region immunoglobulin domains of IgA, IgD, and IgE, or the last three constant region immunoglobulin domains of IgE and IgM. Optionally, an Fc domain can include all or part of the flexible hinge N-terminal to these domains. For IgA and IgM, Fc can include the J chain. For an exemplary Fc domain of IgG, Fc contains immunoglobulin domains Cγ2 and Cγ3, and optionally all or part of the hinge between Cγ1 and Cγ2. The boundaries of the Fc region can vary, but typically, include at least part of the hinge region. An exemplary sequences of IgG Fc domain is set forth in SEQ ID NOS:167. In addition, Fc also includes any allelic or species variant or any variant or modified form, such as any variant or modified form that alters the binding to an FcR or alters an Fc-mediated effector function. Exemplary sequences of other Fc domains, including modified Fc domains, are set forth in SEQ ID NOS: 168 or 169.

As used herein, “Fc chimera” refers to a chimeric polypeptide in which one or more polypeptides is linked, directly or indirectly, to an Fc region or a derivative thereof. Typically, an Fc chimera combines the Fc region of an immunoglobulin with another polypeptide, such as for example an ECD polypeptide. Derivatives of or modified Fc polypeptides are known to those of skill in the art.

As used herein, the polypeptides that contain at least two chimeric polypeptides that include an ECD portion and a multimerization domain, also are referred to as “ECD multimers” (also termed homo- or heteromultimer or homo- or heterodimer.) In instances in which the multimerization domain is from an antibody or portion thereof, the polypeptides can be referred to as immunoadhesins or receptabody dimers or multimers. The constituent polypeptides of the multimers also are referered to herein as chimeric polypeptides. Linkage of a multimerization domain to an ECD can be direct or indirect and can be effected using recombinant nucleic acid methods to produce fusion proteins. Linkage also can be effected using chemical coupling methods, such as using heterobifunctional reagents. Exemplary coupling agents include N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2, 4-dinitrobenzene).

As used herein, an antibody refers to an immunoglobulin molecule that has a specific amino acid sequence that recognizes a specific antigen unique to its target. Immunoglobulins are glycoproteins that structurally appear as a “Y”-shaped molecule containing two identical heavy chains (from any of the five classes of heavy chains: γ, δ, α, μ, ε) and two identical light chains connected by disulfide bonds. Each heavy chain has a constant region, which is the same for all immunoglobulins of the same class (C_{H}), and a variable region (V_{H}), which serves as the antigen binding site and differs between immunoglobulins depending on the antigen specificity. Heavy chains γ, δ, α have a constant region composed of three domains (C_{H}1, C_{H}2, and C_{H}3) and have a hinge region, while the constant region of heavy chains μ, ε are composed of four domains (C_{H}1, C_{H}2, C_{H}3, C_{H}4). The light chain has one constant (C_{L}) and one variable (V_{L}) domain. For purposes herein, reference to an antibody refers to a molecule containing all or part of an immunoglobulin molecule containing one or more domains thereof. For example, a Fab fragment is part of an antibody molecule composed of one constant and one variable domain of each of the heavy and light chains. The Fc fragment is composed of two to three contant domains, and optionally all or part of the hinge region (depending on the class of antibody) of the heavy chain. Thus, reference to an antibody refers to polyclonal antibodies, monoclonal antibodies, or any molecule containing part of an antibody portion, such as for example, a receptabody dimer or multimer where the multimerization domain linking two polypeptides (i.e. the ECD, or portion thereof, of at least two CSRs) together is an antibody, or portion thereof, such as an Fc fragment.

As used herein, a monoclonal antibody refers to a highly specific antibody produced in the laboratory by clones of a single hybrid cell by the fusion of a B cell with a tumor cell.

As used herein, conjugate refers to the joining, pairing, or association of two or more molecules. For example, two or more polypeptides (or fragments, domains, or active portions thereof) that are the same or different can be joined together, or a polypeptide (or fragment, domain, or active portion thereof) can be joined with a synthetic or chemical molecule or other moiety. The association of two or more molecules can be through direct linkage, such as by joining of the nucleic acid sequence encoding one polypeptide with the nucleic acid sequence encoding another polypeptide, or can be indirect such us by noncovalent or covalent coupling of one molecule with another. For example, conjugation of two or more molecules or polypeptides can be achieved by chemical linkage.

As used herein, a “tag” or an “epitope tag” refers to a sequence of amino acids, typically added to the N- or C-terminus of a polypeptide. The inclusion of tags fused to a polypeptide can facilitate polypeptide purification and/or detection. Typically a tag or tag polypeptide refers to polypeptide that has enough residues to provide an epitope recognized by an antibody or can serve for detection or purification, yet is short enough such that it does not interfere with activity of chimeric polypeptide to which it is linked. The tag polypeptide typically is sufficiently unique so an antibody that specifically binds thereto does not substantially cross-react with epitopes in the polypeptide to which it is linked Suitable tag polypeptides generally have at least 5 or 6 amino acid residues and usually between about 8-50 amino acid residues, typically between 9-30 residues. The tags can be linked to one or more chimeric polypeptides in a multimer and permit detection of the multimer or its recovery from a sample or mixture. Such tags are well known and can be readily synthesized and designed. Examplary tag polypeptides include those used for affinity purification and include, His tags, the influenza hemagglutinin (HA) tag polypeptide and its antibody 12CA5, (Field et al. (1988) Mol. Cell. Biol. 8:2159-2165); the c-myc tag and the 8F9, 3C7, 6E10, G4, B7 and 9E10 antibodies thereto (see, e.g., Evan et al. (1985) Molecular and Cellular Biology 5 :3610-3616); and the Herpes Simplex virus glycoprotein D (gD) tag and its antibody (Paborsky et al. (1990) Protein Engineering 3:547-553 (1990).

As used herein, a fusion tagged polypeptide refers to a chimeric polypeptide containing an ECD polypeptide fused to a tag polypeptide.

As used herein, tethering refers to the interaction between two domains of a receptor monomer whereby the monomer occurs in a conformation that renders it less available for interaction. For example, subdomain II (S1) can interact in HER1, HER3 and HER4, with its subdomain IV (S2) domain, forming a tethered inactive structure. When in a tethered state, a receptor or isoform thereof is less available or unavailable for dimerization and/or receptor binding. The ECDs of the monomeric forms of HER1, HER3 and HER4 occur in a tethered form that exhibits lower ligand affinity than the untethered form. HER2, which lacks certain residues in subdomain IV, occurs in an untethered form and is available for dimerization with HER1, HER3 and HER4. Upon ligand binding to a tethered (monomeric) form, the tethering interaction is released and the ECD (or receptor) is in a conformation available for dimerization which involves interactions between domains II of two ECDs.

As used herein, reference herein to modulating the activity of a CSR or HER receptor, means that any activity of such receptor, such as ligand binding or other signal-transduction-related activity is altered.

As used herein, a back-to-back configuration refers to the configuration of two ECDs such that each is available for dimerization with a cell surface receptor. When in a back-to-back configuration, each ECD part of of a chimeric polypeptide that contains a multimerization domain is oriented upon formation of an ECD multimer such that that each ECD or portion thereof is available for dimerization with a cell surface receptor.

As used herein, dimer and dimerize with reference to two chimeric polypeptides refers to the interaction between the two chimeric polypeptides. When appropriately dimerized, the ECDs in each or at least one of the chimeric polypeptides is/are available for dimerization with a cell surface receptor.

As used herein, “dimerization with a cell surface receptor” refers to the interaction of a cell surface receptor with an ECD in a multimer provided herein or with another cell surface receptor. The “dimer” or “dimerization” to which the language refers to will be clear from the context.

As used herein, a “polypeptide comprising a domain” refers to a polypeptide that contains a complete domain with reference to the corresponding domain of a cognate receptor. A complete domain is determined with reference to the definition of that particular domain within a cognate polypeptide. For example, a receptor isoform comprising a domain refers to an isoform that contains a domain corresponding to the complete domain as found in the cognate receptor. If a cognate receptor, for example, contains a transmembrane domain of 21 amino acids between amino acid positions 400-420, then a receptor isoform that comprises such transmembrane domain, contains a 21 amino acid domain that has substantial identity with the 21 amino acid domain of the cognate receptor. Substantial identity refers to a domain that can contain allelic variation and conservative substitutions as compared to the domain of the cognate receptor. Domains that are substantially identical do not have deletions, non-conservative substitutions or insertions of amino acids compared to the domain of the cognate receptor.

As used herein, an allelic variant or allelic variation references to a polypeptide encoded by a gene that differs from a reference form of a gene (i.e. is encoded by an allele). Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide.

As used herein, species variants refer to variants of the same polypeptide between and among species. Generally, interspecies variants have at least about 60%, 70%, 80%, 85%, 90%, or 95% identity or greater with a wildtype and/or predominant form from another species, including 96%, 97%, 98%, 99% or greater identity with a wildtype and/or predominant form of a polypeptide.

As used herein, modification in reference to modification of a sequence of amino acids of a polypeptide or a sequence of nucleotides in a nucleic acid molecule and includes deletions, insertions, and replacements of amino acids and nucleotides, respectively.

As used herein, an open reading frame refers to a sequence of nucleotides or ribonucleotides in a nucleic acid molecule that encodes a functional polypeptide or a portion thereof, typically at least about fifty amino acids. An open reading frame can encode a full-length polypeptide or a portion thereof. An open reading frame can be generated by operatively linking one or more exons or an exon and intron, when the stop codon is in the intron and all or a portion of the intron is in a transcribed mRNA.

As used herein, a polypeptide refers to two or more amino acids covalently joined. The terms “polypeptide” and “protein” are used interchangeably herein.

As used herein, truncation or shortening with reference to the shortening of a nucleic acid molecule or protein, refers to a sequence of nucleotides or ribonucleotides in a nucleic acid molecule or a sequence of amino acid residues in a polypeptide that is less than full-length compared to a wildtype or predominant form of the protein or nucleic acid molecule.

As used herein, a reference gene refers to a gene that can be used to map introns and exons within a gene. A reference gene can be genomic DNA or portion thereof, that can be compared with, for example, an expressed gene sequence, to map introns and exons in the gene. A reference gene also can be a gene encoding a wildtype or predominant form of a polypeptide.

As used herein, a family or related family of proteins or genes refers to a group of proteins or genes, respectively that have homology and/or structural similarity and/or functional similarity with each other.

As used herein, a premature stop codon is a stop codon occurring in the open reading frame of a nucleic acid molecule before the stop codon used to produce or create a full-length form of a protein, such as a wildtype or predominant form of a polypeptide. The occurrence of a premature stop codon can be the result of, for example, alternative splicing and mutation.

As used herein, a kinase is a protein that catalyzes phosphorylation of a molecule, typically a biomolecule, including macromolecules and small molecules. For example, the molecule can be a small molecule, or a protein. Phosphorylation includes auto-phosphorylation. Some kinases have constitutive kinase activity. Other kinases require activation. For example, many kinases that participate in signal transduction are phosphorylated. Phosphorylation activates their kinase activity on another biomolecule in a pathway. Some kinases are modulated by a change in protein structure and/or interaction with another molecule. For example, complexation of a protein or binding of a molecule to a kinase can activate or inhibit kinase activity.

As used herein, modulate and modulation refer to a change of an activity of a molecule, such as a protein. Exemplary activities include, but are not limited to, biological activities, such as signal transduction. Modulation can include an increase in the activity (i.e., up-regulation or agonist activity) a decrease in activity (i.e., down-regulation or inhibition) or any other alteration in an activity (such as a change in periodicity, frequency, duration, kinetics or other parameter). Modulation can be context dependent and typically modulation is compared to a designated state, for example, the wildtype protein, the protein in a constitutive state, or the protein as expressed in a designated cell type or condition.

As used herein, inhibit and inhibition refer to a reduction in an activity relative to the uninhibited activity.

As used herein, a composition refers to any mixture. It can be a solution, a suspension, liquid, powder, a paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related. A kit is a packaged combination that optionally includes instructions for use of the combination or elements thereof.

As used herein, a pharmaceutical effect or therapeutic effect refers to an effect observed upon administration of an agent intended for treatment of a disease or disorder or for amelioration of the symptoms thereof.

As used herein, angiogenesis refers to the formation of new blood vessels from existing ones; neovascularization refers to the formation of new vessels. Physiologic angiongenesis is tightly regulated and is essential to reproduction and embryonic development. During post natal and adult life, angiogenesis occurs in wound repair and in exercised muscle and is generally restricted to days or weeks. In contrast, pathologic angiogenesis (or aberrant angiogenesis) can be persistent for months or years supporting the growth of solid tumors and leukemias, for example. It provides a conduit for the entry of inflammatory cells into sites of chronic inflammation (e.g., Crohn's disease and chronic cysititis). It is the most common cause of blindness; it destroys cartilage in rheumatoid arthritis and contributes to the growth and hemorrhage of atherosclerotic plaques. It leads to intraperitoneal bleeding in endometriosis. Tumor growth is angiogenesis-dependent. Tumors recruit their own blood supply by releasing factors that stimulate angiogenesis. Such factors include, VEGF, FGF, PDGF, TGF-β, Tek, EPHA2, AGE and others. AGE-RAGE interactions can elicit angiogenesis through transcriptional activation of the VEGF gene via NF-κB and AP-1 factors. VEGF is overproduced in a large number of human cancers, including breast, lung, colorectal.

As used herein, angiogenic diseases (or angiogenesis-related diseases) are diseases in which the balance of angiogenesis is altered or the timing thereof is altered. Angiogenic diseases include those in which an alteration of angiogenesis, such as undesirable vascularization, occurs. Such diseases include, but are not limited to cell proliferative disorders, including cancers, diabetic retinopathies and other diabetic complications, inflammatory diseases, endometriosis and other diseases in which excessive vascularization is part of the disease process, including those noted above.

As used herein, HER (ErbB)-related diseases or HER receptor-mediated disease are any diseases, conditions or disorders in which a HER receptor and/or ligand is implicated in some aspect of the etiology, pathology or development thereof. In particular, involvement includes, for example, expression or overexpression or activity of a HER receptor family member or ligand. Diseases, include, but are not limited to proliferative diseases, including cancers, such as, but not limited to, pancreatic, gastric, head and neck, cervical, lung, colorectal, endometrial, prostate, esophageal, ovarian, uterine, glioma, bladder or breast cancer. Other conditions, include those involving cell proliferation and/or migration, including those involving pathological inflammatory responses, non-malignant hyperproliferative diseases, such as ocular conditions, skin conditions, conditions resulting from smooth muscle cell proliferation and/or migration, such as stenoses, including restenosis, atheroscelerosis, muscle thickening of the bladder, heart or other muscles, endometriosis, or rheumatoid arthritis.

As used herein, treatment means any manner in which the symptoms of a condition, disorder or disease or other indication, are ameliorated or otherwise beneficially altered.

As used herein therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, the term “subject” refers to an animals, including a mammal, such as a human being.

As used herein, a “patient” refers to a human subject.

As used herein, an “individual” can be a subject.

As used herein, normal levels or values can be defined in a variety of ways known to one of skill in the art. Typically, normal levels refer to the expression levels of a CSR or CSR ligand across a healthy population. The normal levels (or reference levels) are based on measurements of healthy subjects, such as from a specified source (i.e. blood, serum, tissue, or other source). Often, a normal level will be specified as a “normal range”, which typically refers to the range of values of the median 95% of the healthy population. Reference value is used interchangeably herein with normal level but can be different from normal levels depending on the subjects or the source. For example, a normal level of a CSR or ligand can differ between a patient that is 2-years old versus a patient that is 50-years old. Thus, the reference levels are typically dependent on the normal levels of a particular segment of the population. Thus, for purposes herein, a normal or reference level is a predetermined standard or control by which a test patient can be compared.

As used herein, elevated level refers to the any level of expression of a CSR or CSR ligand that is increased about the normal or reference levels. Expression of a CSR or CSR ligand in a test subject can be compared to the normal or control levels of the CSR or ligand to determine if the level is elevated.

As used herein, an activity refers to a function or functioning or changes in or interactions of a biomolecule, such as polypeptide. Exemplary, but not limiting of such activities are: complexation, dimerization, multimerization, receptor-associated kinase activity or other enzymatic or catalytic activity, receptor-associated protease activity, phosphorylation, dephosphorylation, autophosphorylation, ability to form complexes with other molecules, ligand binding, catalytic or enzymatic activity, activation including auto-activation and activation of other polypeptides, inhibition or modulation of another molecule's function, stimulation or inhibition of signal transduction and/or cellular responses such as cell proliferation, migration, differentiation, and growth, degradation, membrane localization, membrane binding, and oncogenesis. An activity can be assessed by assays described herein and by any suitable assays known to those of skill in the art, including, but not limited to in vitro assays, including cell-based assays, in vivo assays, including assays in animal models for particular diseases.

As used herein, complexation refers to the interaction of two or more molecules such as two molecules of a protein to form a complex. The interaction can be by noncovalent and/or covalent bonds and includes, but is not limited to, hydrophobic and electrostatic interactions, Van der Waals forces and hydrogen bonds. Generally, protein-protein interactions involve hydrophobic interactions and hydrogen bonds. Complexation can be influenced by environmental conditions such as temperature, pH, ionic strength and pressure, as well as protein concentrations.

As used herein, dimerization refers to the interaction of two molecules, such as two molecules of a receptor. Dimerization includes homodimerization where two identical molecules interact. Dimerization also includes heterodimerization in which two different molecules, such as two different receptor molecules, interact. Typically, dimerization involves two molecules that interact with each other through interaction of a dimerization domain or multimerization domain contained in each molecule. Similarly multimerization, refers to interaction of a plurality of molecules to form dimers, trimers, or higher ordered oligomers, where the molecules are of the same type or are different.

Dimerization with reference to two chimeric polypeptides refers to the dimerization that occurs by virtue of interaction between multimerization domains of each. Receptor dimerization refers to the dimerization between two receptors leading to activation thereof, or between a receptor and an ECD portion capable of dimerizing with the receptor, such as an ECD multimer, that would then modulate the activation of the receptor thereof.

As used herein, in silico refers to research and experiments performed using a computer. In silico methods include, but are not limited to, molecular modeling studies, biomolecular docking experiments, and virtual representations of molecular structures and/or processes, such as molecular interactions.

As used herein, biological sample refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or to sample that is processed For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived thereform. Also included are soil and water samples and other environmental samples, viruses, bacteria, fungi algae, protozoa and components thereof.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof. Nucleic acid can refer to polynucleotides such as deoxyribonucleic acid (DNA) and ribonucleic acid (RNA). The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phophorothioate bond, a thioester bond, or a peptide bond (peptide nucleic acid). The term “oligonucleotide” also is used herein essentially synonymously with “polynucleotide,” although those in the art recognize that oligonucleotides, for example, PCR primers, generally are less than about fifty to one hundred nucleotides in length.

Polynucleotides include nucleotide analogs, include, for example, mass modified nucleotides, which allow for mass differentiation of polynucleotides; nucleotides containing a detectable label such as a fluorescent, radioactive, luminescent or chemiluminescent label, which allow for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid molecules can be prepared using well-known methods (see, for example, Weiler et al. Nucleic acids Res. 25: 2792-2799 (1997)).

As used herein, oligonucleotides refer to polymers that include DNA, RNA, nucleic acid analogues, such as PNA, and combinations thereof. For purposes herein, primers and probes are single-stranded oligonucleotides or are partially single-stranded oligonucleotides.

As used herein, synthetic, with reference to, for example, a synthetic nucleic acid molecule or a synthetic gene or a synthetic peptide refers to a nucleic acid molecule or polypeptide molecule that is produced by recombinant methods and/or by chemical synthesis methods.

As used herein, production by recombinant techniques or methods using recombinant DNA methods means the use of the well-known methods of molecular biology for expressing proteins encoded by cloned DNA.

As used herein, the term “vector” refers to a nucleic acid molecule capable of transporting another nucleic acid to which it has been linked. One type of vector is an episome, i.e., a nucleic acid capable of extra chromosomal replication. Vectors include those capable of autonomous replication and/or expression of nucleic acids to which they are linked. Vectors capable of directing the expression of genes to which they are operatively linked are referred to herein as “expression vectors.” In general, expression vectors often are in the form of “plasmids,” which are generally circular double stranded DNA loops that, in their vector form are not bound to the chromosome. “Plasmid” and “vector” are used interchangeably as the plasmid is the most commonly used form of vector. Other such other forms of expression vectors that serve equivalent functions and that become known in the art subsequently hereto.

As used herein, the phrase “operatively linked” in reference to nucleic acid sequences generally means the nucleic acid molecules or segments thereof are covalently joined into one piece of nucleic acid such as DNA or RNA, whether in single or double stranded form. The segments are not necessarily contiguous, rather two or more components are juxtaposed so that the components are in a relationship permitting them to function in their intended manner. For example, segments of RNA (exons) can be operatively linked such as by splicing, to form a single RNA molecule. In another example, DNA segments can be operatively linked, whereby control or regulatory sequences on one segment control permit expression or replication or other such control of other segments. Thus, in the case of a regulatory region operatively linked to a reporter or any other polynucleotide, or a reporter or any polynucleotide operatively linked to a regulatory region, expression of the polynucleotide/reporter is influenced or controlled (e.g., modulated or altered, such as increased or decreased) by the regulatory region. For gene expression, a sequence of nucleotides and a regulatory sequence(s) are connected in such a way to control or permit gene expression when the appropriate molecular signal, such as transcriptional activator proteins, are bound to the regulatory sequence(s). Operative linkage of heterologous nucleic acid, such as DNA, to regulatory and effector sequences of nucleotides, such as promoters, enhancers, transcriptional and translational stop sites, and other signal sequences, refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, operative linkage of heterologous nucleic to regulatory and effector sequences of nucleotides, such as promoters, enhancers, transcriptional and translational stop sites, and other signal sequences refers to the relationship between such nucleic acid, such as DNA, and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA. Thus, operatively linked or operationally associated refers to the functional relationship of nucleic acid, such as DNA, with regulatory and effector sequences of nucleotides, such as promoters, enhancers, transcriptional and translational stop sites, and other signal sequences. In order to optimize expression and/or in vitro transcription, it can be necessary to remove, add or alter 5′ untranslated portions of the clones to eliminate extra, potentially inappropriate alternative translation initiation (i.e., start) codons or other sequences that can interfere with or reduce expression, either at the level of transcription or translation. Alternatively, consensus ribosome binding sites (see, e.g., Kozak J. Biol. Chem. 266:19867-19870 (1991)) can be inserted immediately 5′ of the start codon and can enhance expression. The desirability of (or need for) such modification can be empirically determined.

As used herein, the term “operatively linked” in reference to polypeptides, for example, such as when used in the context of the phrase “at least one subdomain or portion thereof of a cell surface receptor is operatively operatively linked to another subdomain or portion thereof” means that they are the two amino acid sequences are joined by a peptide bond between a terminal amino acid residue in each sequence, to form a single amino acid residue sequence.

As used herein, the phrase “generated from a nucleic acid” in reference to the generating of a polypeptide, such as an isoform and intron fusion protein, includes the literal generation of a polypeptide molecule and the generation of a polypeptide by translation of a nucleic acid molecule.

As used herein, production with reference to a polypeptide refers to expression and recovery of expressed protein (or recoverable or isolatable expressed protein). Factors that can influence the production of a protein include the expression system and host cell chosen, the cell culture conditions, the secretion of the protein by the host cell, and ability to detect a protein for purification purposes. Production of a protein can be monitored by assessing the secretion of a protein, such as for example, into cell culture medium.

As used herein, secretion refers to the process by which a protein is transported into the external cellular environment or, in the case of gram-negative bacteria, into the periplasmic space. Generally, secretion occurs through a secretory pathway in a cell, for example, in eukaryotic cells this involves the endoplasmic reticulum and golgi apparatus.

As used herein, homologous with reference to a molecule, such as a nucleic acid molecule or polypeptide, from different species refers to a corresponding molecule (i.e. a species variant). Such molecules typically are similar and generally share about 45% sequence identity or homology. One of skill in the art can identify homologs among species.

As used herein, heterologous nucleic acid is nucleic acid that is not normally produced in vivo by the cell in which it is expressed or that is produced by the cell but is at a different locus or expressed differently or that mediates or encodes mediators that alter expression of endogenous nucleic acid, such as DNA, by affecting transcription, translation, or other regulatable biochemical processes. Heterologous nucleic acid is generally not endogenous to the cell into which it is introduced, but has been obtained from another cell or prepared synthetically. Heterologous nucleic acid can be endogenous, but is nucleic acid that is expressed from a different locus or altered in its expression. Generally, although not necessarily, such nucleic acid encodes RNA and proteins that are not normally produced by the cell or in the same way in the cell in which it is expressed. Heterologous nucleic acid, such as DNA, also can be referred to as foreign nucleic acid, such as DNA. Thus, heterologous nucleic acid or foreign nucleic acid includes a nucleic acid molecule not present in the exact orientation or position as the counterpart nucleic acid molecule, such as DNA, is found in a genome. It also can refer to a nucleic acid molecule from another organism or species (i.e., exogenous). Heterologous nucleic acid with reference to an isolated nucleic acid molecule can refer to a portion of such molecule that is derived from a different source or locus from the another portion of such molecule. Exemplary of heterologous secrection signals include any presequence (i.e. signal sequence) or preprosequence that in not the endogenous signal sequence of an encoded molecules, such as, but not limited to, a tPA preprosequence, a preprogastrin sequence, and any other sequence known to one of skill in the art.

Similarly, heterologous with reference to a portion of polypeptide, refers to one portion of a chimeric polypeptide compared to the other. Hence in a hybrid ECD that contains subdomain I from HER1, subdomain II from HER2 and subdomain III from HER3, each subdomain is heterologous to each of the other subdomains.

A heterologous molecule can be derived from a different genetic source or species. Thus, molecules heterologous to a particular CSR ECD or isoform thereof include any molecule containing a sequence that is not derived from or endogenous to the CSR ECD or isoform thereof. Examples of heterologous molecules include secretion signals from a different polypeptide of the same or different species, a tag such as a fusion tag or label, or all or part of any other molecule. A heterologous molecule can be fused to a nucleic acid or polypeptide sequence of interest for the generation of a fusion or chimeric molecule or can be chemically linked via covalent or non-covalent linkages.

As used herein, a heterologous secretion signal refers to a signal sequence from a polypeptide, from the same or different species, that is different in sequence from the endogenous signal sequence. A heterologous secretion signal can be used in a host cell from which it is derived or it can be used host cells that differ from the cells from which the signal sequence is derived.

As used herein, an active portion a polypeptide, such as with reference to an active portion of an ECD, refers to a portion of polypeptide that has an activity.

As used herein, purification of a protein refers to the process of isolating a protein, such as from from a homogenate, which can contain cell and tissue components, including DNA, cell membrane and other proteins. Proteins can be purified in any of a variety of ways known to those of skill in the art, such as for example, according to their isolectric points by running them through a pH graded gel or an ion exchange column, according to their size or molecular weight via size exclusion chromatography or by SDS-PAGE (sodium dodecyl sulfate-polyacrylamide gel electrophoresis) analysis, or according to their hydrophobicity. Other purification techniques include, but are not limited to, precipitation or affinity chromatography, including immuno-affinity chromatography, and others and methods that include combination of any of these methods. Furthermore, purification can be facilitated by including a tag on the molecule, such as a his tag for affinity purification or a detectable marker for identification.

As used herein, “isolated,” with reference to a molecule, such as a nucleic acid molecule, oligonucleotide, polypeptide or antibody, indicates that the molecule has been altered by the hand of man from how it is found in its natural environment. For example, a molecule produced by and/or contained within a recombinant host cell is considered “isolated ” Likewise, a molecule that has been purified, partially or substantially, from a native source or recombinant host cell, or produced by synthetic methods, is considered “isolated.” Depending on the intended application, an isolated molecule can be present in any form, such as in an animal, cell or extract thereof; dehydrated, in vapor, solution or suspension; or immobilized on a solid support.

As used herein, a substantially pure polypeptide or an isolated polypeptide (or other molecule) are used interchangeably and mean the polypeptide has been purified from a source or sample homogeneity as detected by chromatographic techniques or other such techniques, such as SDS-PAGE under non-reducing or reducing conditions using, for example Coomassie blue or silver stain. Homogeneity tpyically means less than about 5% or less than 5% contamination with other source proteins.

As used herein, detection includes methods that permit visualization (by eye or equipment) of a protein. A protein can be visualized using an antibody specific to the protein.

Detection of a protein can also be facilitated by fusion of a protein with a tag including an epitope tag or label.

As used herein, a label refers to a detectable compound or composition which is conjugated directly or indirectly to a polypeptide so as to generate a labeled polypeptide. The label can be detectable by itself (e.g., radioisotope labels or fluorescent labels) or, in the case of an enzymatic label, can catalyze chemical alteration of a substrate compound composition which is detectable. Non-limiting examples of labels included fluorogenic moieties, green fluorescent protein, or luciferase.

As used herein, expression refers to the process by which a gene's coded information is converted into the structures present and operating in the cell. Expressed genes include those that are transcribed into mRNA and then translated into protein and those that are transcribed into RNA but not translated into protein (e.g., transfer and ribosomal RNA). For purposes herein, a protein that is expressed can be retained inside the cells, such as in the cytoplasm, or can be secreted from the cell.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans-acting factors. Promoters, depending upon the nature of the regulation, can be constitutive or regulated.

As used herein, regulatory region means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Exemplary of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to an including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA and, stop codons, leader sequences and fusion partner sequences, internal ribosome binding sites (IRES) elements for the creation of multigene, or polycistronic, messages, polyadenylation signals to provide proper polyadenylation of the transcript of a gene of interest and stop codons and can be optionally included in an expression vector.

As used herein, the “amino acids,” which occur in the various amino acid sequences appearing herein, are identified according to their well-known, three-letter or one-letter abbreviations (see Table 2). The nucleotides, which occur in the various DNA fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§1.821-1.822, abbreviations for amino acid residues are shown in Table 2:

TABLE 2
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

All sequences of amino acid residues represented herein by a formula have a left to right orientation in the conventional direction of amino-terminus to carboxyl-terminus. In addition, the phrase “amino acid residue” is defined to include the amino acids listed in the Table of Correspondence modified, non-natural and unusual amino acids. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and generally can be made without altering an activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p.224).

Such substitutions can be made, for example, in accordance with those set forth in TABLE 3 as follows:

TABLE 3

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions, including non-conservative changes, also are permissible and can be determined empirically or in accord with other known conservative or non-conservative substitutions.

As used herein, a peptidomimetic is a compound that mimics the conformation and certain stereochemical features of the biologically active form of a particular peptide. In general, peptidomimetics are designed to mimic certain desirable properties of a compound, but not the undesirable properties, such as flexibility, that lead to a loss of a biologically active conformation and bond breakdown. Peptidomimetics can be prepared from biologically active compounds by replacing certain groups or bonds that contribute to the undesirable properties with bioisosteres. Bioisosteres are known to those of skill in the art. For example the methylene bioisostere CH2S has been used as an amide replacement in enkephalin analogs (see, e.g., Spatola (1983) pp. 267-357 in Chemistry and Biochemistry of Amino Acids, Peptides, and Proteins, Weinstein, Ed. volume 7, Marcel Dekker, New York). Morphine, which can be administered orally, is a compound that is a peptidomimetic of the peptide endorphin. For purposes herein, cyclic peptides are included among peptidomimetics as are polypeptides in which one or more peptide bonds is/are replaced by a mimic. The heteromultimers and multimers and hybrid ECDs and chimeric polypeptides provided herein can be modified by replacing bonds with mimetics and such molecules are provided herein.

As used herein, “similarity” between two proteins or nucleic acids refers to the relatedness between the amino acid sequences of the proteins or the nucleotide sequences of the nucleic acids. Similarity can be based on the degree of identity and/or homology of sequences and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

“Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptide sequences, the term “identity” is well known to skilled artisans (Carillo, H. & Lipton, D., SIAM J Applied Math 48:1073 (1988)).

As used herein, sequence identity compared along the full length of each SEQ ID to the full length of a an isoform refers to the percentage of identity of an amino acid sequence of an isoform polypeptide along its full-length to a reference polypeptide, designated by a specified SEQ ID, along its full length. For example, if a polypeptide A has 100 amino acids and polypeptide B has 95 amino acids, identical to amino acids 1-95 of polypeptide A, then polypeptide B has 95% identity when sequence identity is compared along the full length of a polypeptide A compared to full length of polypeptide B. Typically, where an isoform polypeptide or a reference polypeptide is a mature polypeptide lacking a signal sequence, sequence identity is compared along the full length of the polypeptides, excluding the signal sequence portion. For example, if an isoform lacks a signal peptide but a reference polypeptide contains a signal peptide, comparison along the full length of both polypeptides for determination of sequence identity excludes the signal sequence portion of the reference polypeptide. As discussed below, and known to those of skill in the art, various programs and methods for assessing identity are known to those of skill in the art. For example, a global alignment, such as using the Needleman-Wunsch global alignment algorithm, can be used to find the optimum alignment and identity of two sequences when considering the entire length. High levels of identity, such as 90% or 95% identity, readily can be determined without software.

As used herein, by homologous (with respect to nucleic acid and/or amino acid sequences) means about greater than or equal to 25% sequence homology, typically greater than or equal to 25%, 40%, 60%, 70%, 80%, 85%, 90% or 95% 90% or 95% sequence homology; the precise percentage can be specified if necessary. For purposes herein the terms “homology” and “identity” often are used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence homology, the number of conserved amino acids is determined by standard alignment algorithms programs, and can be used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

Whether any two nucleic acid molecules have nucleotide sequences that are at least 60%, 70%, 80%, 85%, 90%, 95%, 96%, 97%, 98% or 99% “identical” or “homologous” can be determined using known computer algorithms such as the “FAST A” program, using for example, the default parameters as in Pearson et al. (1988) Proc. Natl. Acad. Sci. USA 85:2444 (other programs include the GCG program package (Devereux, J., et al., Nucleic Acids Research 12(1):387 (1984)), BLASTP, BLASTN, FASTA (Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Hence, as used herein, the term “identity” or “homology” represents a comparison between a test and a reference polypeptide or polynucleotide.

As used herein, the term at least “90% identical to” refers to percent identities from 90 to 99.99 relative to the reference nucleic acid or amino acid sequences. Identity at a level of 90% or more is indicative of the fact that, assuming for exemplification purposes a test and reference polypeptide length of 100 amino acids are compared. No more than 10% (i.e., 10 out of 100) amino acids in the test polypeptide differs from that of the reference polypeptide. Similar comparisons can be made between test and reference polynucleotides. Such differences can be represented as point mutations randomly distributed over the entire length of an amino acid sequence or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, a polypeptide comprising a specified percentage of amino acids set forth in a reference polypeptide refers to the proportion of contiguous identical amino acids shared between a polypeptide and a reference polypeptide. For example, an isoform that comprises 70% of the amino acids set forth in a reference polypeptide having a sequence of amino acids set forth in SEQ ID NO:XX, which recites 147 amino acids, means that the reference polypeptide contains at least 103 contiguous amino acids set forth in the amino acid sequence of SEQ ID NO:XX.

As used herein, “primer” refers to an oligonucleotide containing two or more deoxyribonucleotides or ribonucleotides, generally more than three, from which synthesis of a primer extension product can be initiated. A primer can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. Experimental conditions conducive to synthesis include the presence of nucleoside triphosphates and an agent for polymerization and extension, such as DNA polymerase, and a suitable buffer, temperature and pH.

Certain nucleic acid molecules can serve as a “probe” and as a “primer.” A primer, however, as a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application.

As used herein, an effective amount is the quantity of a therapeutic agent necessary for preventing, curing, ameliorating, arresting or partially arresting a symptom of a disease or disorder.

As used herein, unit dose form refers to physically discrete units suitable for human and animal subjects and packaged individually as is known in the art.

As used herein, a single dosage formulation refers to a formulation for direct administration.

As used herein, the singular forms “a,” “an” and “the” include plural referents unless the context clearly dictates otherwise. Thus, for example, reference to compound, comprising “an extracellular domain”” includes compounds with one or a plurality of extracellular domains.

As used herein, ranges and amounts can be expressed as “about” a particular value or range. About also includes the exact amount. Hence “about 5 bases” means “about 5 bases” and also “5 bases.’

As used herein, “optional” or “optionally” means that the subsequently described event or circumstance does or does not not occur, and that the description includes instances where said event or circumstance occurs and instances where it does not. For example, an optionally substituted group means that the group is unsubstituted or is substituted.

### B. Pan-Cell Surface Receptor-Specific Therapeutics

Provided herein are compounds that are therapeutics or candidate therapeutics that interact with with one or more, typically two or more, cell surface receptors, such as members of the HER family, particularly HER1, HER3 and HER4, insulin-like growth factor-1 receptors (IGF-1R or IGF1R), particularly IFG1R, and vascular endothelial cell growth factor receptor (VEGFR) family members. These therapeutics and candidate therapeutics act by specifically targeting at least one or more receptors and/or their ligands that cooperate in the activating of a disease pathway. Such therapeutics overcome or address problems associated with therapeutics targeted to a single receptor.

For example, a problem with anti-HER drugs, such as Herceptin® (Trastuzimab), has been limited efficacy because HER2 overexpression, which occurs in only subset of breast cancers, and also limited duration of response because resistance develops to the drug can develop, such as by virtue of activity of other receptors. Similar problems are observed with drugs that target receptors other than HER family members. A mechanism for Herceptin® (Trastuzimab) resistance is co-expression of additional HER family members. Other mechanisms of resistance, include co-expression of the IGF-1R; metalloprotease-mediated activation of HER2 (by ‘clipping’ of the extracellular domain); and upregulation of the P13K-AKT (phosphatidylinositol-3-kinase-Protein Kinase B) pathway, often mediated by loss of PTEN (phosphatase and tensin homology, which is mutated in cancers; see, e.g., Nahta et al. (2006) Cancer Lett. 8:123-38; Hynes et al. (2005) Nature Reviews Cancer 5:341-354). Mechanisms of resistance to HER1/EGFR therapeutics are similar to those for resistance to Herceptin®

(Trastuzimab). Data show that 60% of patients (88/145 patients) express one or two HER family members; 18.6% (27/145) co-express three HER family members. The data also show that cumulative receptor expression predicts a much more severe disease (p<0.0001). Additional data indicate that about 40% of breast cancers co-express two HER family members. The frequency of co-expression of HER family members in other cancers is comparable to that in breast cancer, with up to ˜50% of patients predicted to simultaneously express HERs, and thus can be resistant to single agent targeted therapeutics, perhaps as a result of the constitutive activation of AKT (protein kinase B) and other cell pathways that stimulate cell proliferation (Hynes et al. (2005) Nature Reviews Cancer 5:341-354). Simultaneous co-expression of HER family members also leads to induction of survivin (an anti-apoptotic factor; Xia et al., (2006) Oncogene 24:6213-6221) as well as mediating production of distinct growth factors important in tumor progression (e.g., vascular endothelial cell growth factor; VEGF).

It is concluded herein that resistance to any particular HER-directed therapeutic is frequently mediated through expression of other HER family members, or through expression of related receptor tyrosine kinases, such as the IGF1R, VEGFR, FGFR and others. For example, IGF-1R directly inhibits the activity of Herceptin® (Trastuzimab) via heterodimerization with HER2 (Nahta et al. (2006) Cancer Lett. 8:123-38).

In addition to co-overexpression, the frequency of overexpression of any particular HER family member varies among cancers. It is found herein that the most commonly overexpressed of the HER family are HER1 and HER3, and the least commonly overexpressed member is HER4. TGF-α is the most commonly expressed ligand. The following Table provides an estimated disease incidence and estimated distribution of overexpression frequencies of HER family members (determined from literature sources; all data based upon immunohistochemistry):

TABLE 4
Percent Patients Overexpressing

| | Mortality** | | | | |
|---|---|---|---|---|---|
| Disease | (U.S.) | HER1 | HER2 | HER3 | HER4 |
| NSCLC* | 113,000 | 60 | 20-50 | 84 | Pos |
| Breast | 40,580 | 16 | 25 | 18 | 12 |
| Colorectal | 56,730 | 70 | Pos | 50 | |
| Pancreatic | 31,270 | 33 | 25 | 50 | Pos |
| Liver | 14,270 | 68 | 21 | 84 | 61 |
| Gastro- | 24,000 | 30-50 | 10-20 | 81 | Pos |
| Esophageal | | | | | |

*non-small cell lung cancer
**Cancer Facts and Figures, 2003

Co-expression of HER family members, which results in lack of response, or in development of resistance through compensatory upregulation of alternative HER family members, creates a challenge for treatment. The observations that different HER family members contribute to tumor development and progression in an overlapping and synergistic fashion is recognized herein and exploited herein to provide therapeutics that can be designed to avoid the problems of resistance and that can be designed for particular tumors based upon receptor expression in the tumor. The therapeutics and candidate therapeutics provided herein address these problems, including those identified herein and others, by targeting at least one or more cell surface receptors, typically two or more cell surface receptors such as a plurality of HER family members, and/or HER family members and any other cell surface receptor that participates in or is involved in resistance to drugs targeted to a single cell surface receptor.

Based upon the structure, functioning and interaction of HER family members, as well as other cell surface receptors, provided herein are a number of therapeutic loci for targeting and intervention. These include regions of the receptors involved in ligand binding and regions involved in receptor dimerization, and regions involved in tethering. These regions can be targeted in a plurality of receptors simultaneously so that one therapeutic interferes with ligand binding and/or receptor dimerization of two or more receptors. Provided herein are several approaches and candidate therapeutics molecules.

Methods for targeting regions of receptors, including the domains responsible for dimerization, ligand binding, and/or tethering are provided. In particular, receptor dimerization is blocked by therapeutics that interact with a plurality of receptors. These therapeutics include heteromultimers provided herein and described in detail below.

Also, provided are methods for producing therapeutics that interact with targeted regions. For example, subdomains II and IV are targeted to interfere with receptor dimerization and or to stabilize or promote tethering. As a first step in these methods, peptides that bind specifically to DII and IV homologous regions are respectively identified, such as by phage display selection. Subsequently, high-affinity, suitable peptide pairs that bind D II and IV are identified and hetero-dimers are constructed using one of the available methods such as chemical synthesis or PEGylation. The identified high affinity hetero-dimeric peptides that bind DII and IV simultaneously may tightly hold the receptors in their autoinhibited configuration. Additionally, the peptide binders selected can target the homologous regions in domain II and domain IV of HER family receptors. The peptides targeted using this method can cross-link interdomain regions (e.g., stabilize the DII/IV interaction) in tethered, inactive, HER family members; or can bind distinct sites, for example on DII of a single receptor, thereby sterically inhibiting its ability to dimerize.

Methods for targeting ligands with therapeutics that bind to a plurality of ligands also are provided. Receptor ligands can be screened to identify molecules that bind thereto. Heteromultimers containing two or more of such molecules can be produced.

Methods for stabilizing the tethered conformation of the receptors are provided. HER1, 3, and 4 exist in a tethered and open form. The tether is formed upon interaction of subdomains II and IV. In this form, the principal dimerization arm (in DII) is unable to interact with other receptors, and so cannot form receptor dimers or heterodimers. The HER receptors on the cell surfaces, except for HER2, which is proposed to be constitutively ‘ready for dimerization’, are estimated to occur in the tethered form about 95% of the time on cells (even when stimulated with ligand). Stabilization of the tethered form of the receptor, so that it cannot assume an open configuration, inhibits receptor activity.

Hence provided herein are therapeutics and methods that address one or all of the considerations and the problems noted above. Therapeutics that target a plurality of receptors, particularly members of the HER family, are provided herein. In particular, provided herein are Pan-cell surface receptor therapeutics, including pan-HER therapeutics, methods for making and using such therapeutics for treatment of diseases and disorders that involve the HER family of receptors and their ligands. Also provided are methods for identifying Pan-Her therapeutic candidate molecule, and screening assays therefor. Such methods are described herein in Section J and in the Examples.

In some embodiments, Pan cell surface receptor-specific therapeutics are designed to interact with ligands for one or more more receptors and/or to interact with one or more receptors to modulate, generally inhibit, the activity of two more receptors. This is achieved by forming heteromultimers of two or more ECDs or fragments thereof from at least one HER and another RTK or other CSR, which may or many not be a member of the HER family. In particular, at least one of the ECDs is from a HER receptor and includes portions of at least domains I, II and III to permit ligand binding and dimerization with cell surface receptors. The heteromultimers typically are linked so that the dimerization domains are positioned for interaction with a cell surface receptor. Typically, the ECDs can include a multimerization domain that facilitates dimerization or multimerization of two or more ECDS. Included among the ECDS are hybrid ECDs that contain domains from two or more different receptors.

At least one of the ECDs in the heteromultimer contains sufficient portions of domains I-III and, if needed, domain IV, such that the heteromultimer interacts with ligand and/or is available for dimerization with a cell surface receptor, such that the heteromultimer modulates the activity of at least two cell surface receptors. The at least two cell surface receptors generally includes at least one HER receptor family member.

The Pan-Her therapeutics, which contain at least two ECDs or portions from two different HER family members, can block activity of two or more members of the HER family by attaching the extracellular domain portion of the receptors, such as similar to Herceptin and Erbitux, and/or by binding ligand that activates one or more receptors. The Pan-Her therapeutics modulate the activity of two or more cell surface receptors, including at least one cell surface receptor that is a HER receptor.

Also provided are multimers in which two or more of the ECDS are derived from the same HER receptor. In dimmers of such multimers, the ECDS, however, contain different ECD portions.

The following sections describe exemplary therapeutics, methods of making them, screening for them, and using them.

### C. HER Receptor and Other Cell Surface Receptor Structures and Activities

Provided herein are multimers that contain ECDs from different cell surface receptors, including members of the HER family of receptors. The multimers include combinations of receptor domains and subdomains linked to multimerization domains. To design such ECD multimers as provided herein, an appreciation of the receptor structure and function is advantageous. This section provides such description.

The receptor tyrosine kinases are a large family of cell signaling molecules that participate in embryogenesis, cell growth and differentiation, and in several disease processes, including diseases as diverse as cancer, autoimmune disorders and other chronic human diseases (reviewed in Hynes and Lane (2005) Nat Rev Cancer 5: 341-54). The best characterized of these is the human EGF Receptor family (HER) of receptor tyrosine kinases. These are referred to as the Class I receptors. The HER family of receptors belong to the receptor tyrosine kinase (RTK) family, and possess protein tyrosine kinase activity (except for HER3; for reviews, see, e.g., Jorissen et al. (2003) Exptl. Cell Res. 284:31-53; Dawson et al. (2005) Mol. Cell Biol. 25:7734-7742, which sets forth nomenclature used herein; and Bazley et al. (2005) Endocrine-Related Cancer 12:S17-S27). There are four receptor genes that encode HER family members: the HER1(EGFR or ErbB1), HER2 (or c-erbB-2 or ErbB2 or NEU), HER3 (c-erbB3 or ErbB3) and HER4 (c-erbB4 or ErbB4). The encoding genes can be alternatively spliced to produce a number of variants, including truncated variants, and variants that are intron fusion proteins. Some of the receptors play a role in normal development, differentiation, migration, wound healing and apoptosis, which are essential activities. Aberrant function and activity play a role in a variety of disease states, including cancers.

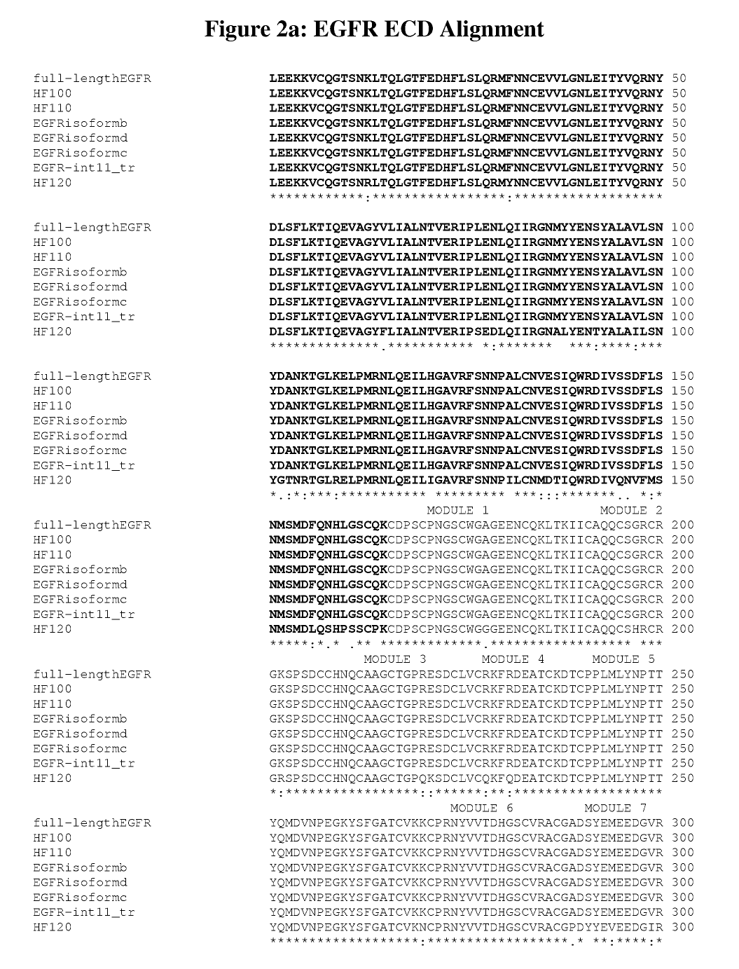

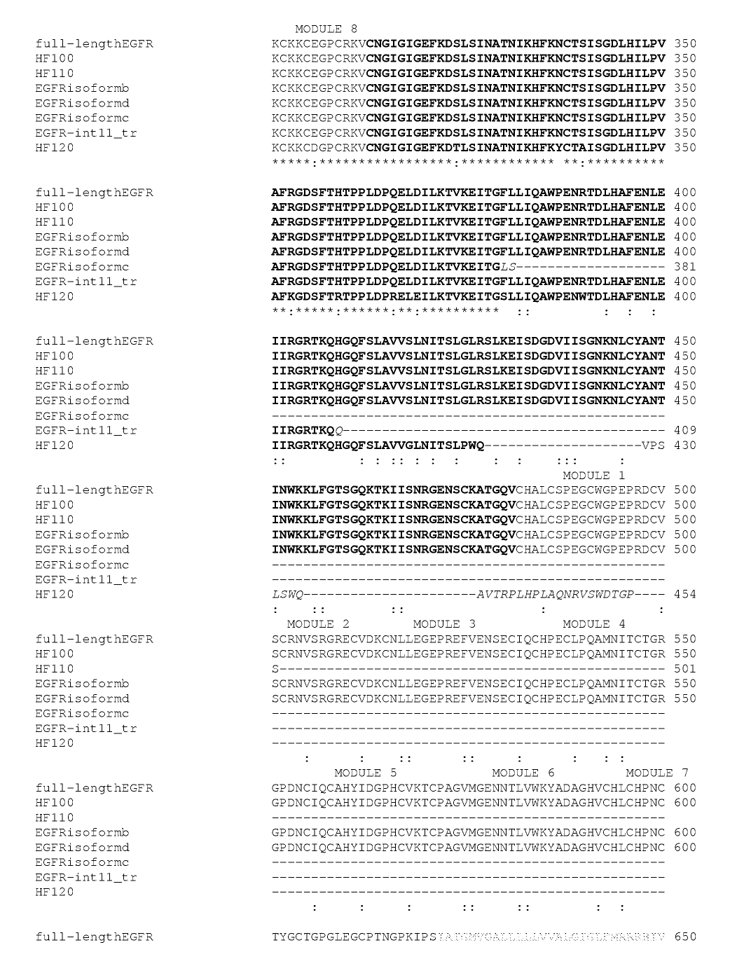

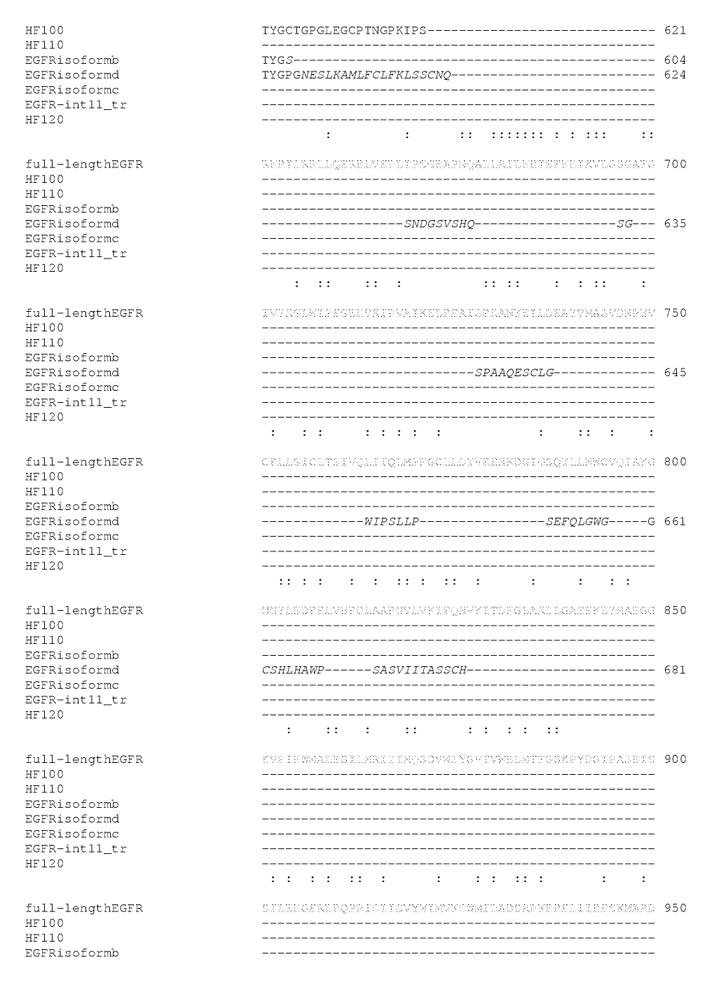

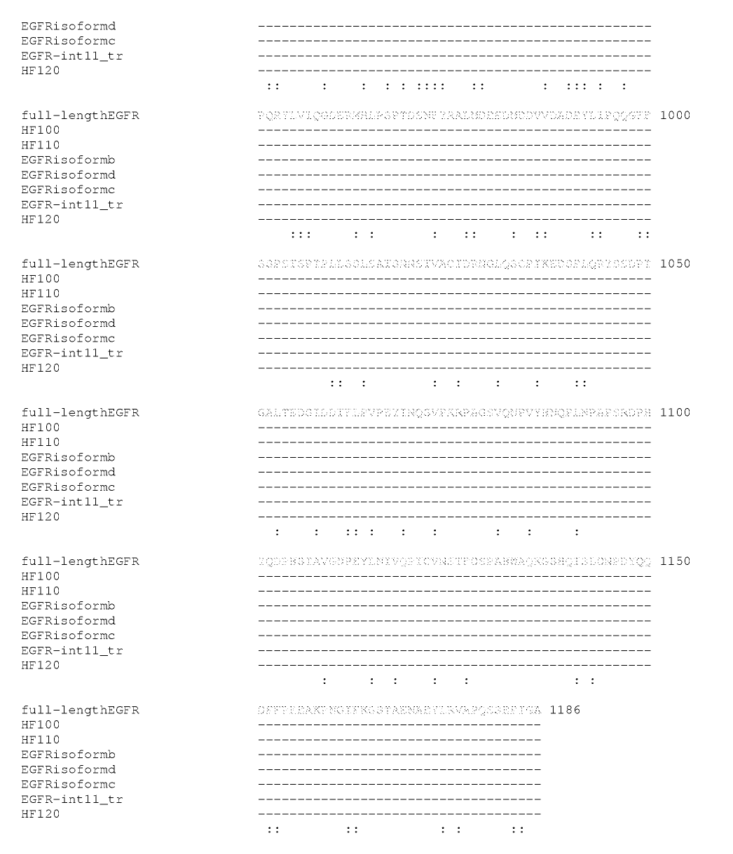

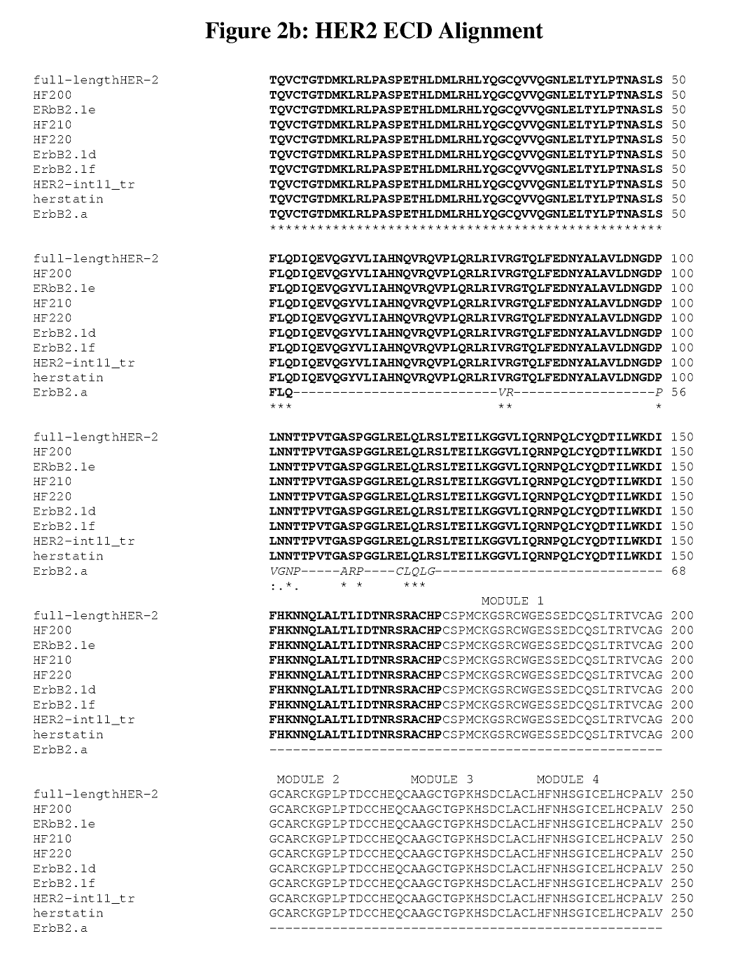

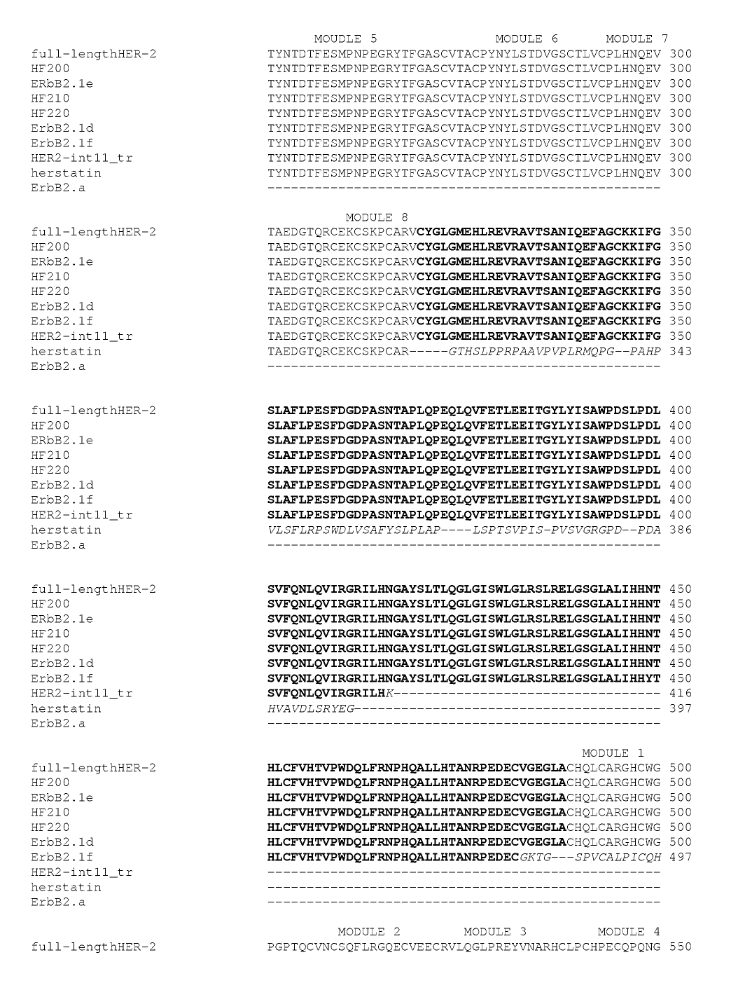

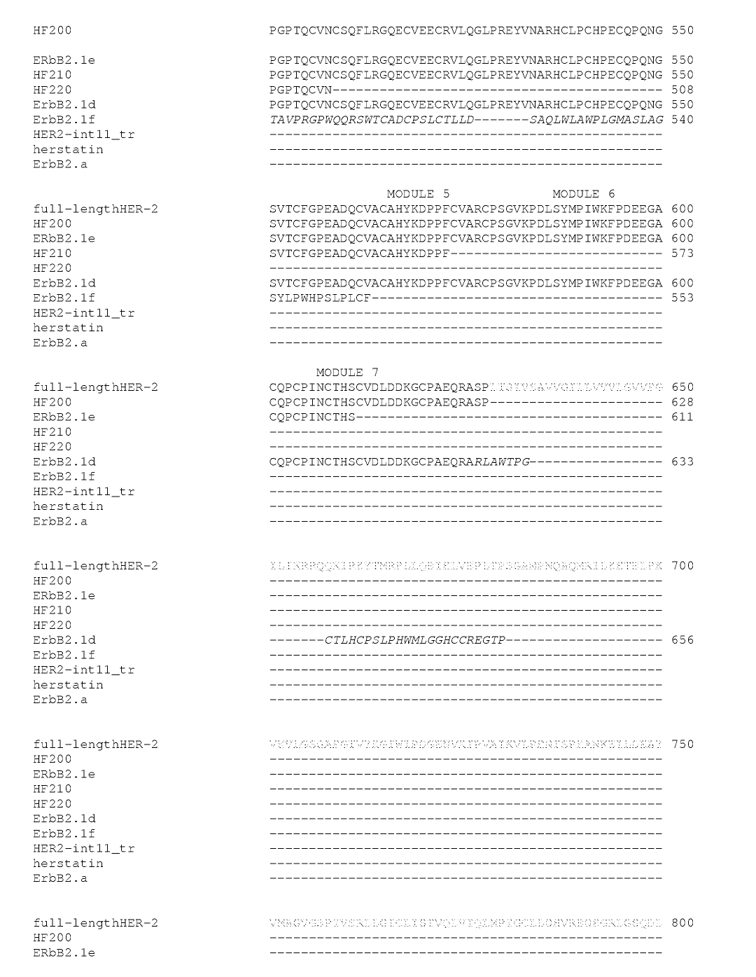

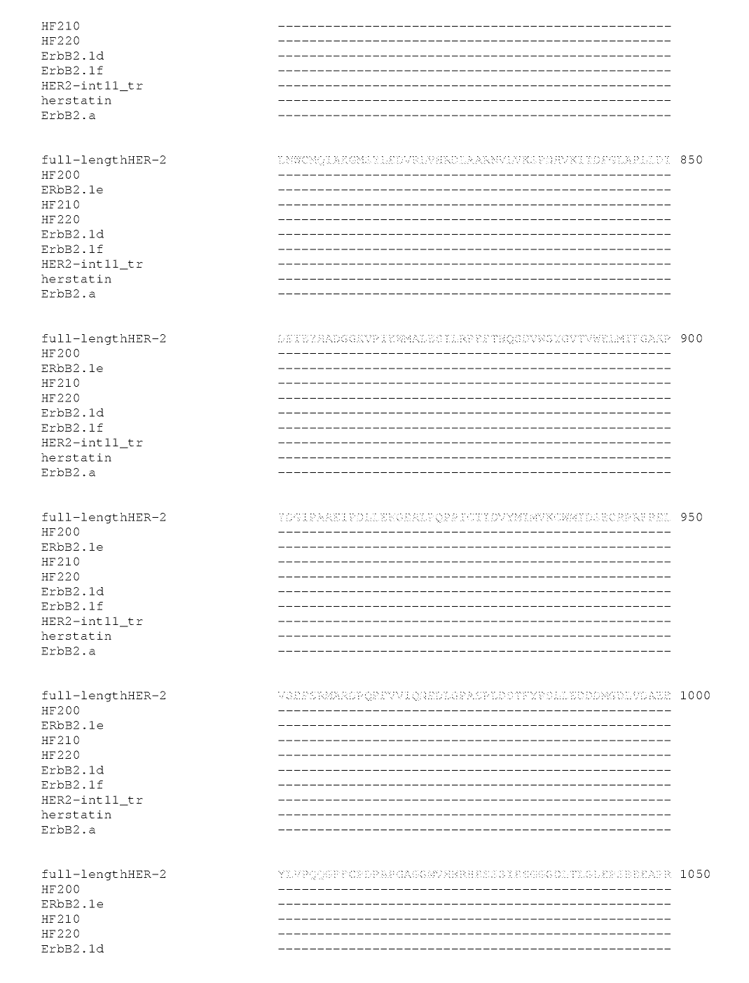

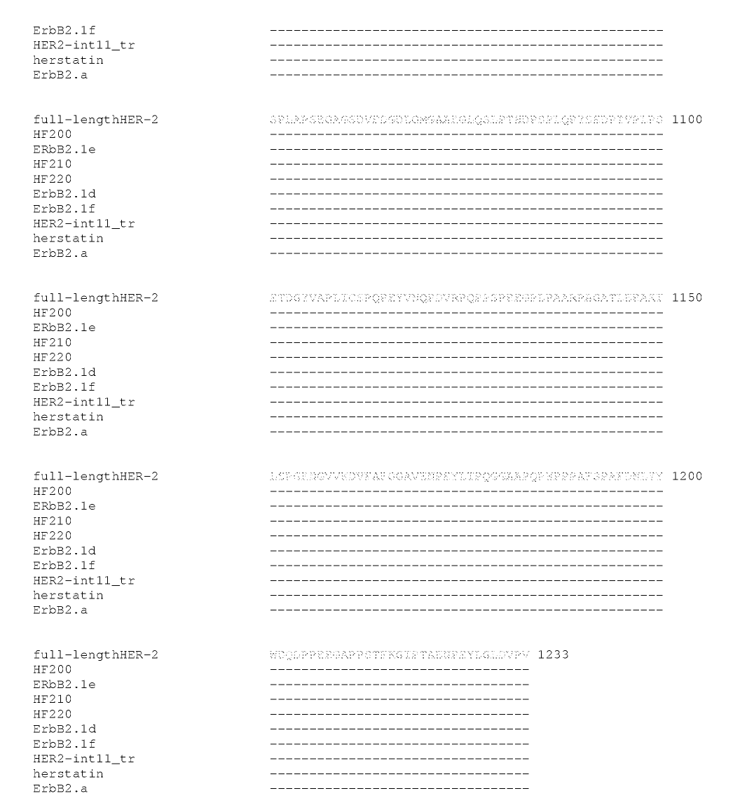

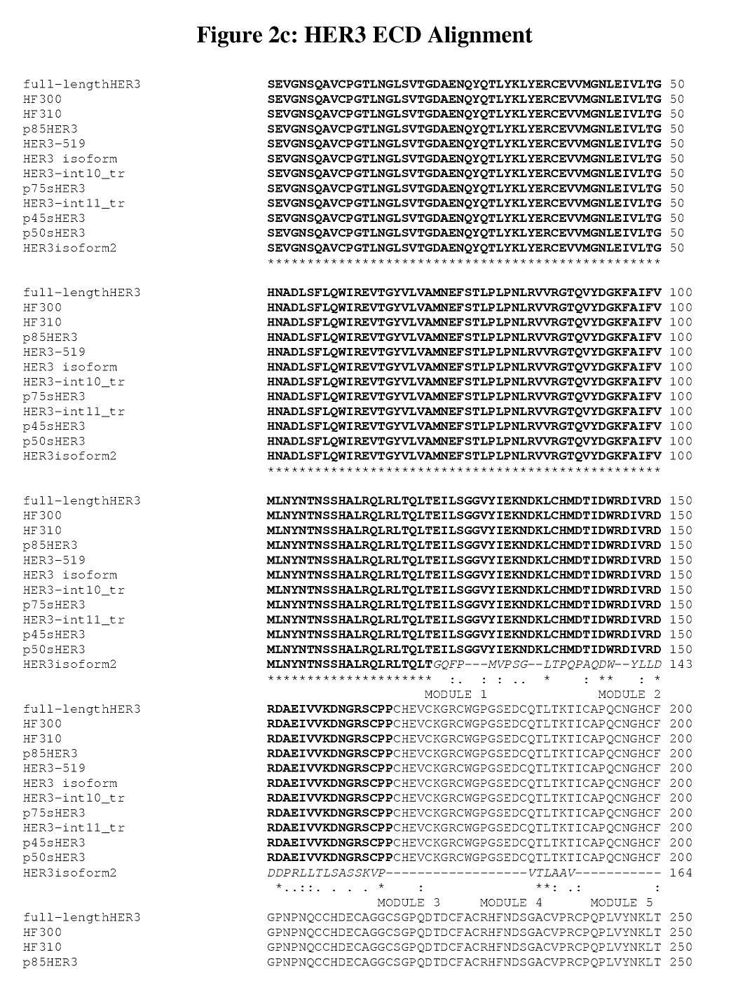

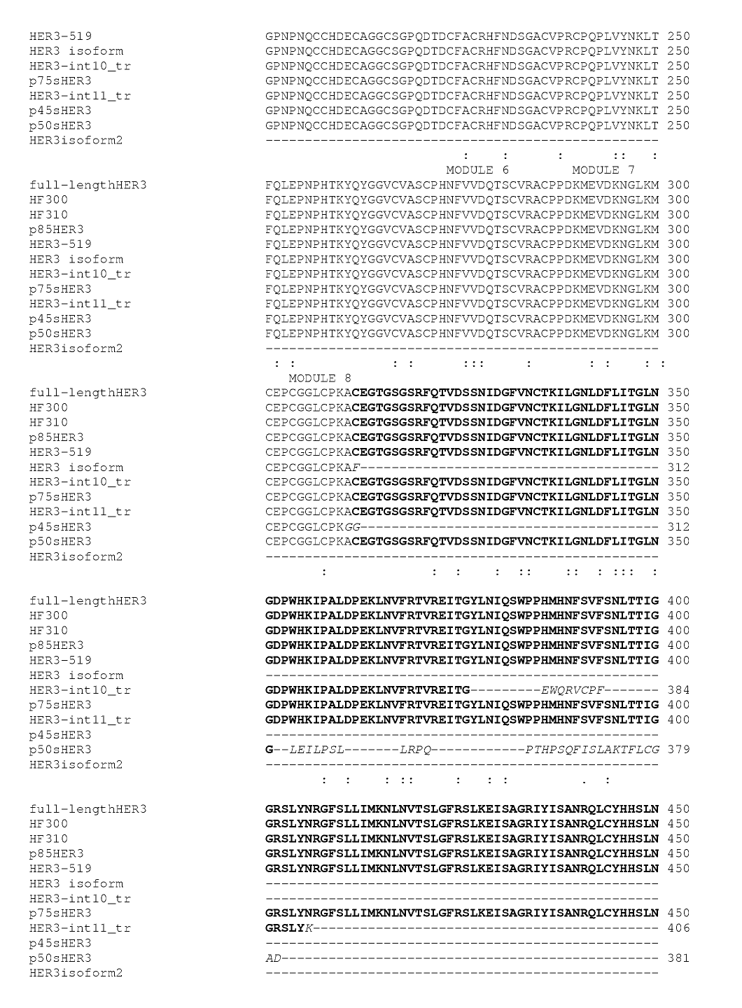

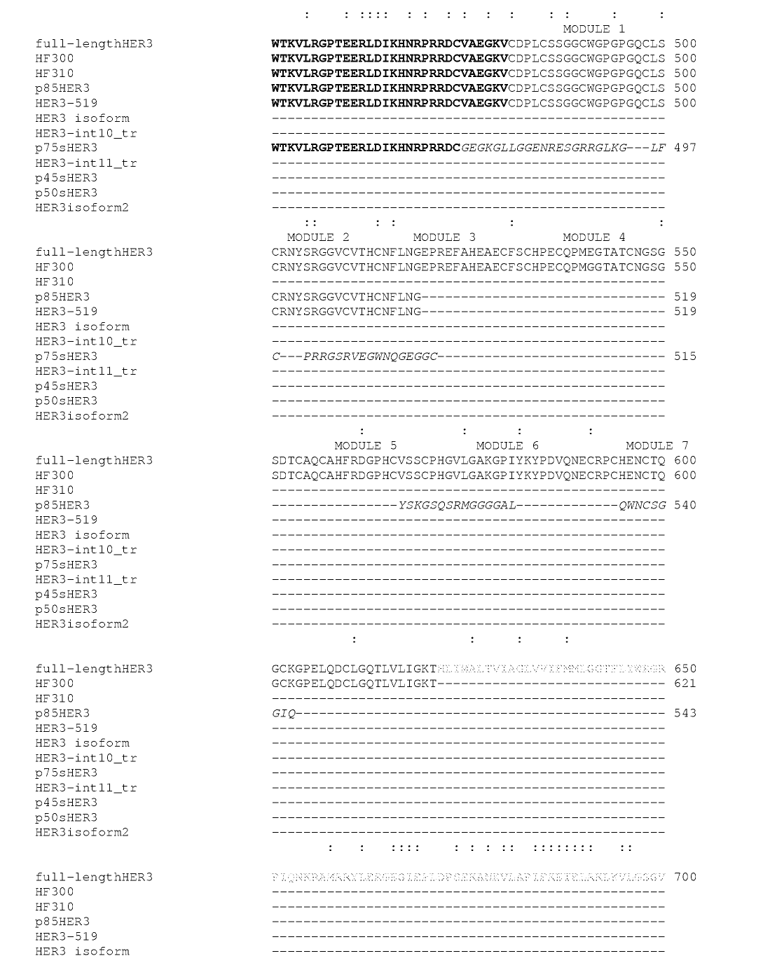

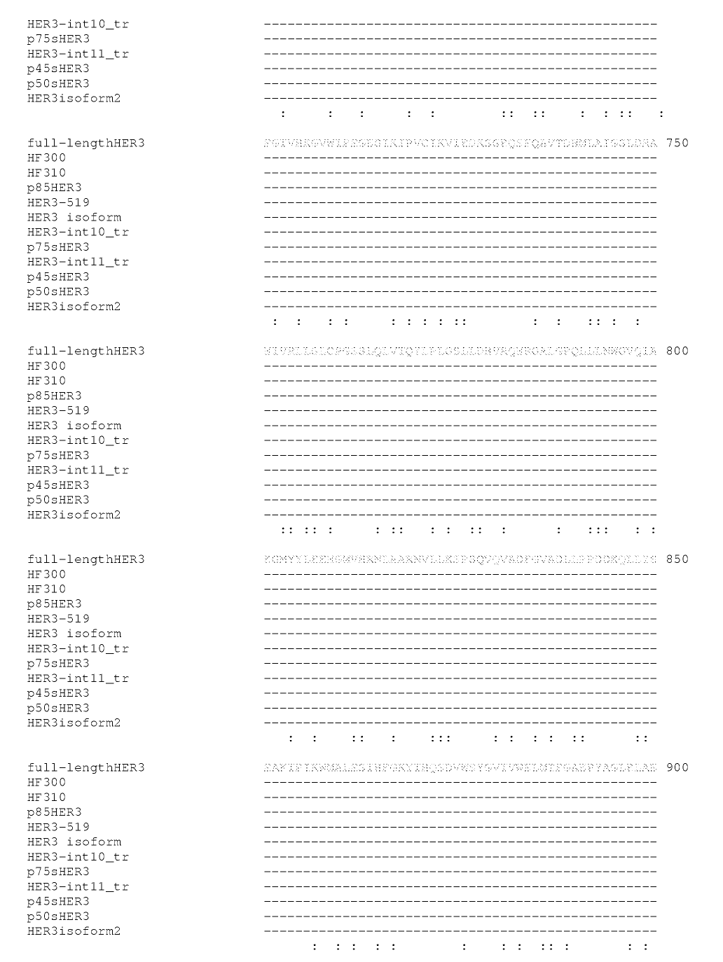

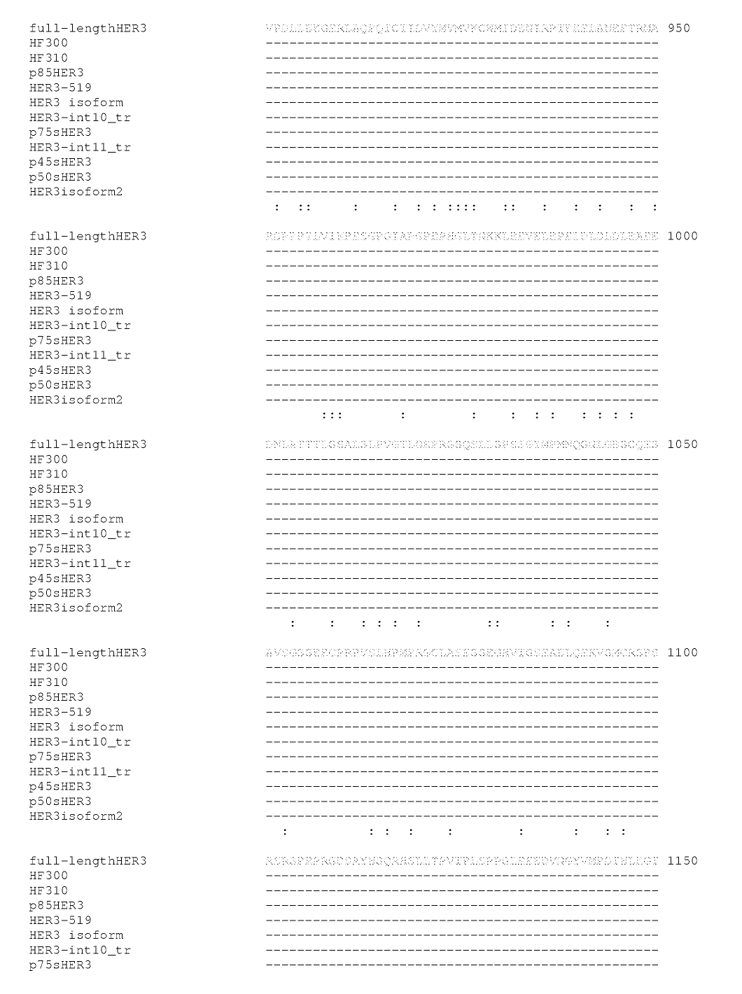

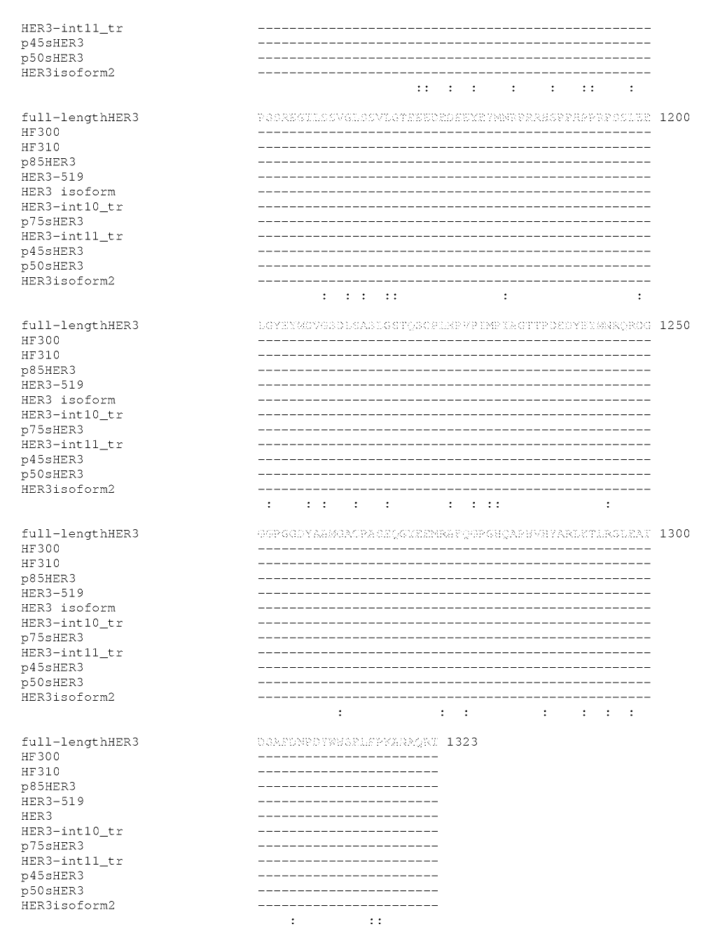

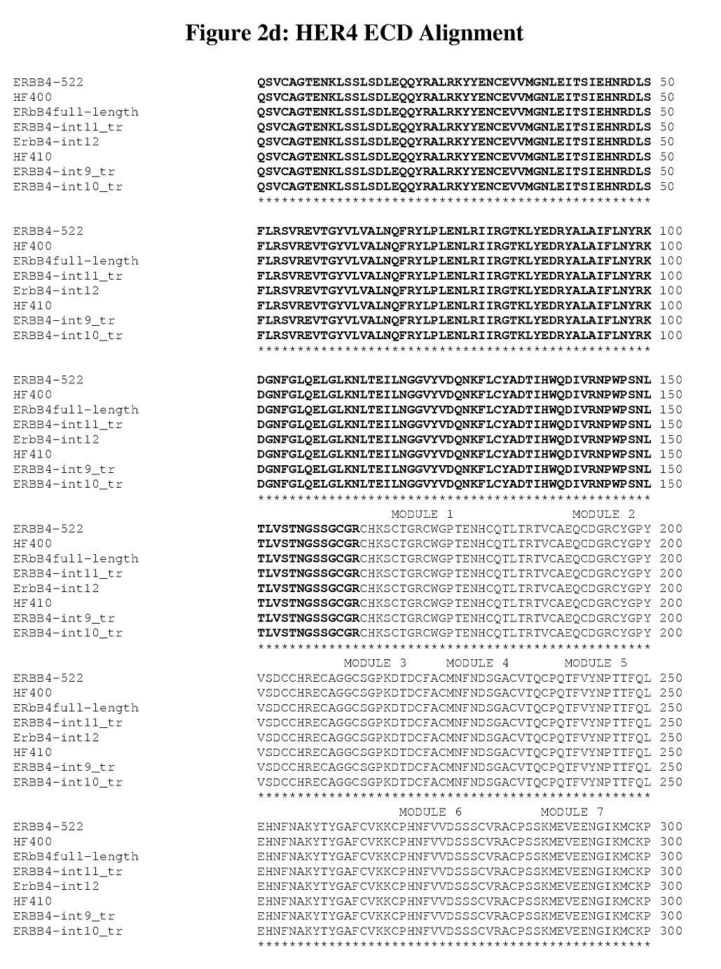

Sequences of exemplary human HER family receptors are set forth in SEQ ID NOS: 2 (HER1), 4 (HER2), 6 (HER3), and 8 (HER4) and are encoded by a sequence of nucleotides set forth in SEQ ID NOS: 1, 3, 5, and 7, respectively. Typically, encoded HER polypeptides undergo posttranslational processing to yield a mature polypeptide lacking a signal sequence. Amino acid sequences of mature full-length polypeptides are depicted and described in FIGS. 2(A)-(D) and the respective figure legend. For purposes herein, numbering of amino acids in describing exemplary HER family receptors, ECD portions thereof, or ECD isoforms are with respect to the numbering of the mature polypeptide, unless specified otherwise. In addition, the amino acid positions used to describe domain organizations are for illustrative purposes and are not meant to limit the scope of the embodiments provided. It is understood that polypeptides and the description of domains thereof are theoretically derived based on homology analysis and alignments with similar receptors. Thus, the exact locus can vary, and is not necessarily the same for each receptor.

As set forth in FIG. 1, each member of the HER family shares a common domain organization including an extracellular domain portion (ECD or ectodomain or extracellular domain) of about 620 amino acids, a transmembrane domain, and a cytoplasmic tyrosine kinase domain. The ECD portion exhibits four subdomains designated I (L1), II (S1), III (L2), and IV (S2). Sequence identity among the full-length HER family varies from 37% for HER1 (EGFR) and HER3 to 49% for HER1 and HER2, with varying degrees of sequence identity among each domain. For example, the tyrosine kinase domains have the highest sequence identity (about 59-81%), and the carboxy terminal domain as the lowest identity (about 12-31%). Within the ECD domain, subdomains I and III share approximately 37% sequence identity and domains II and IV are homologous and share about 17% sequence identity (Ferguson et al. (2003) Mol. Cell, 11:507-517).

Subdomains I and III are also referred to as L domains, and constitute the bilobal ligand binding site. The L domains each contain a single-stranded right-handed beta-helix of six turns that form a barrel-like structure capped off at each end by an α helix. Ligand binds between the L1 and L2 domains.

Subdomains II and IV are also referred to as S domains or cysteine rich (CR) domains (also called furin-like repeat domains), and constitute a cysteine rich region. The Cys rich region is composed of a succession of small disulfide-bonded modules, which form a rod-shaped structure. Two types of disulfide-bonded modules are seen in each domain: a C1 disulfide bond where a single disulfide bond constrains an intervening bow-like loop, and a C2 disulfide bond where two disulfide bonds link four successive cysteines in the pattern Cys1-Cys3 and Cys2-Cys4 to give a knot-like structure (Ferguson et al., (2003) Molecular Cell 11:507-517). Domain II contains three consecutive C2 modules followed by five C1 modules, while domain IV contains seven modules where the first two are C1 modules, followed by a C2 module, two C1 modules, and another C2 module.

In general, domains II and IV mediate both intramolecular and intermolecular contact of the HER structure. For example, intramolecular interactions occur between subdomains I and IV in a process referred to as “tethering”, where a β-loop projects from the fifth Cys rich module (see FIG. 1). This loop interacts with equivalent but smaller loops from module 5 and module 6 in domain IV. Interaction of domains II and IV is further stabilized by hydrogen bonds between the two regions, as well as by the contributions of carbohydrate. In addition, a side chain of an amino acid residue corresponding to Y246 in domain II of HER1 makes hydrogen bonds with the side chains of amino acid residues corresponding to D563 and K585 in domain IV. Corresponding amino acid residues in the ECD of mature HER family receptors important in mediating contacts between domains II/IV are set forth in Table 5. Interactions between domains II and IV are not present in HER2, in part due to the presence of non-conserved amino acid residues as compared to other HER family members (i.e. italicized residues in Table 5).

TABLE 5
Domain II/IV Contact Residues

| Residues | HER1 | HER2 | HER3 | HER4 |
|---|---|---|---|---|
| Domain II: | Tyr246 | Tyr 252 | Tyr246 | Tyr243 |
| | Tyr251 | Phe257 | Phe251 | Phe248 |
| | Gln252 | Glu258 | Gln252 | Gln249 |
| Domain IV: | Asp563 | Asp570 | Asp562 | Asp560 |
| | Gly564 | Pro571 | Gly563 | Gly561 |
| | His566 | Phe573 | His565 | Asn563 |
| | Lys585 | Lys593 | Lys583 | Lys581 |

Intermolecular interactions also occur and allow for receptor-receptor interactions that are necessary for homo- and heterodimerization characteristic of active HER receptors. In fact, the same loop in module 5 of domain II described above that mediates tethering also is responsible for dimerization. This loop is often termed the “dimerization arm”. The amino acid residue corresponding to Y246 also is important in facilitating intermolecular interactions required for dimerization.

HER family receptors further include a transmembrane (TM) domain (variably reported as beginning at residues 621, 622 or 626-644 or 647) and a cytoplasmic domain. The transmembrane domain spans the plasma membrane anchoring the receptor and generally includes hydrophobic residues. Typically, the residues that make up a transmembrane domain form an α-helix.

The juxtamembrane (JM) domain, which is the region located between the transmembrane and kinase domains, serves a variety of regulatory functions, such as for example, downregulation and ligand-dependent internalization events, basolateral sorting such as for example of EGFR in polarized cells, and association with proteins such as eps8 and calmodulin. In addition, the JM domain plays a role in receptor trafficking and is required (along with the transmembrane domain) for targeting EGFR to caveloae.

The tyrosine kinase domain is a conserved catalytic core common to receptor tyrosine kinases (RTKs) and is responsible for mediating transphosphorylation of carboxy-terminal tyrosine residues present in the carboxy-terminal domain. Activation of the tyrosine kinase domain occurs upon a conformational change induced upon binding of ligand to the receptor.

The carboxy-terminal (CT) domain contains tyrosine residues where phosphorylation modulates signal transduction. The tyrosine residues and nearby amino acids of each HER family member interact with a diverse second messengers to regulate specific biological and biochemical responses. For example, second messengers containing, for example, an SH2 (src homology-2) structure or a PTB domain recognize the phosphorylation “docking sites” and interact with the receptors to transmit the signal received at the receptor to either the cytoplasm or nucleus via interactions with other signaling components. There also are several serine/threonine residues where phosphorylation thereof affects receptor downregulation and endocytosis processes. Residues 984-996 in the C-terminus of EGFR (FIG. 1) serve as a binding site for actin and are involved in the formation of higher order receptor oligomers and/or receptor clustering after ligand activation of the kinase domain.

1. HER1 ECD Structure and Domain Organization

The domain organization of a full-length mature ECD and of various HER1 ECD isoforms is depicted in FIG. 2(A). The extracellular portion of HER1 includes residues 1-621 of a mature HER1 receptor and contains subdomains I (amino acid residues 1-165), II (amino acid residues 166-313), III (amino acid residues 314-481), and IV (amino acid residues 482-621). The I, II, and III domains of HER1 have structural and sequence homology to the first three domains of the type I insulin-like growth factor receptor (IGF-1R, see e.g., Garret et al., (2002) Cell, 110:763-773). Similar to IGF-1R, the L domains (i.e. domains I and III) have a structure of a six turn β helix capped at each end by a helix and a disulfide bond. As compared to IGF-1R, the HER1 sequence includes amino acid insertions that contribute to biochemical structures important for mediating ligand binding by HER1. Among these include a V-shaped excursion (residues 8-18), which sits over the large β sheet of domain I to form a major part of the ligand binding interface. In domain III, a corresponding region forms a loop (residues 316-326) that also is involved in ligand binding. A third insert region present in domain III (residues 351-369) is an extra loop in the second turn of domain III. This loop is the epitope for various antibodies that prevent ligand binding (i.e., LA22, LA58, and LA90, see e.g., Wu et al., (1989) J Biol Chem., 264:17469-17475). In addition, other loops in the fourth turn of the β helix solenoid are involved in ligand binding.

TGF-α, a ligand for HER1, interacts with the large β sheets of both the L domains I and III of one receptor molecule. Similarly, the ligand EGF also interacts with both domains I and III of HER1, although the interaction of EGF with domain III is considered to be the major binding site for EGF (Kim et al., (2002) FEBS, 269: 2323-2329). Cross-linking studies have determined that the N- and C-terminal portions of the EGF ligand interact with domains I and III, respectively, of the HER1 receptor. Amino acid G1y441 in domain III, corresponding to mature full-length HER1, is involved in mediating binding to EGF via interaction with Arg45 of human EGF. A 40 kDa fragment of HER1 of 202 amino acids (corresponding to amino acids 302-503 of a mature HER1 polypeptide) is sufficient to retain full ligand-binding capacity of HER1 to EGF. This 202 amino acid portion contains all of domain III, and only a few residues each of domain II and domain IV (Kohda et al., (1993) JBC 268: 1976).

Domain II of EGFR contains eight disulfide-bonded modules. Domain II interacts with both domains I and III. The contacts with domain III occurs via modules 6 and 7, while modules 7 and 8 have a degree of flexibility thereby functioning to create a hinge in the ligand-free form of the EGFR molecule. A large ordered loop is formed from module 5 of domain II and projects directly away from the ligand binding site. This loop corresponds to residues 240-260 (also described as residues 242-259) and contains an antiparallel β-ribbon. The loop (also called the dimerization arm) is important in mediating intramolecuar interactions as well as mediating receptor-receptor contacts. In the inactive or “tethered” conformation of HER1, the loop contributes to intramolecular interactions by inserting between similar loop structures in modules 5 and 6 corresponding to amino acids 561-569 and 572-585, respectively, of a mature full-length ECD (see FIG. 1). Amino acid residues that contribute to the domain II/IV interaction are set forth in Table 5 above.

Deletion of the domain II loop abolishes the ability of the HER1 ECD to dimerize, thus showing its importance in facilitating intermolecular interactions. Dimerization is mediated by projection of the loop out across domain II of a second HER molecule in a space between domain I, II, and III. For example, contact is made by residues 244-253 of the dimerization arm with residues 229-239, 262-278, and 282-288 on the concave face of domain II in a second HER molecule. Tyr246 in domain II makes hydrogen bonds with Gly264 and Cys283 residues in a second HER molecule, and the phenyl rings of Tyr246 also interacts with Ser262 and Ser282 of an adjacent molecule. Other amino acid contacts between domain II of an EGFR and another HER molecule include Tyr251 with Phe263, Gly264, Tyr275, and Arg285; Pro248 with Phe230 and Ala265; Met253 with Thr278; and Tyr251 with Arg285. In addition, Asn247 and Asn256 are important for maintaining the loop in the appropriate conformation. Most all of these residues are conserved among HER family members and function similarly between HER family receptors. Further, proline residues occur in the loop in HER family receptors at any one of positions 243, 248, 255, and 257, with HER3 containing three prolines. The proline residues stabilize the conformation of the loop further. For example, HER1 contains prolines at position 248 and 257.

In addition to the involvement of domain IV (modules 5 and 6) in tethering of an inactive HER1 molecule, at least part of module 1 of domain IV of HER1 also appears to be required to maintain the structural integrity of an active HER1 molecule. For example, as mentioned above, a 40 kDa proteolytic fragment of HER1 containing all of domain III and part of domains II and IV retains full-ligand binding ability. The portion of domain IV present in this molecule corresponds to amino acids 482-503, including all of module 1. The amino acid corresponding to Trp492 in a mature HER1 molecule plays a role in maintaining stability of the HER1 molecule by interacting with a hydrophobic pocket in domain III. A recombinant molecule of HER1 containing all of domains I, II, and III but lacking all of domain IV is unable to bind ligand (corresponding to amino acids 1-476 of a mature HER1, see e.g., Elleman et al., (2001) Biochemistry 40:8930-8939). Thus, at least all or a portion of module 1 of domain IV appears to be required for the ligand binding ability of HER1. The remainder of domain IV is expendable for ligand binding and signaling. For example, normal ligand binding and signaling properties of HER1 is present in a HER1 molecule missing residues 521-603 of a mature HER1 polypeptide.

2. HER2 ECD Structure and Domain Organization

The domain organization of a full-length mature HER2 ECD and various HER2 ECD isoforms is depicted in FIG. 2(B). The extracellular portion of HER2 includes residues 1-628 of a mature HER2 receptor and contains subdomains I (amino acid residues 1-172), II (amino acid residues 173-319), III (amino acid residues (320-488), and IV (amino acid residues 489-628). Despite having a similar domain organization, analysis of the crystal structure of HER2 has shown that HER2 does not possess the same intramolecular interactions that are characteristic of the “tethered”, inactive structure of the other HER family members. In other words, the loop in module 5 of domain II does not interact with residues of domain IV. Table 5 above depicts amino acids that mediate contacts between domains II/IV among HER family receptors, and sets forth those that are not conserved in HER2. For example, the Gly residue conserved at position 564, 563, and 561 of HER1, HER3, and HER4, respectively, is replaced by a proline in HER2. This proline residue sterically inhibits the interactions observed among the other HER family receptors (i.e. the Gly residue interacts with the corresponding HER3 amino acid Phe251). Consequently, due to sequence differences, HER2 does not exist in a “tethered”, inactive state, but constitutively exists in an active conformation, with its dimerization arm in domain II exposed.

The domain II dimerization arm, while having only 33-44% sequence homology among HER family receptors, is functionally highly conserved among all HER family receptors, including HER2. In HER2, this dimerization arm corresponds to amino acid residues 246-267 of mature HER2. Since HER2 is always present in an active, non-tethered conformation with its dimerization arm exposed, HER2 is the preferred heterodimerization partner for the other HER family members. HER2, however, does not form homodimers. The inability to form homodimers appears to be due to electrostatic repulsion, as the dimerization arm of HER2 and the pocket to which the dimerization arm makes contact in HER2 are both electronegative. The high electronegativity of HER2 can be accounted for by the greater number of acidic and basic residues in HER2 compared to the other HER family members. When HER2 is overexpressed in cells, however, it is able to homodimerize. The homodimerization observed upon overexpression involves a hydrophobic region in the carboxy terminal domain of HER2, particularly for ligand independent multimerization observed upon overexpression of the receptor (Garret et al. (2003) Mol. Cell, 11; 495-505).

In addition, unlike other HER family receptors, HER2 does not bind to ligand. One reason for the inability to bind ligand is the close proximity and relative orientation of the ligand binding domains I and III. In HER2, the opposing domains I and III make substantial direct contact with eachother. In this conformation, a ligand is unable to bind to any potential binding site because each binding site is occluded by the opposing ligand binding domain (Garret et al., (2003) Molecular Cell, 11:495-505). In addition, compared to other HER family members, HER2 contains sequence differences in the ligand binding interface of domains I and III that can inhibit ligand interaction. For example, Arg12 (corresponding to Thr15 in HER1, Ser18 in HER3, and Ser12 in HER4) and Pro14 (corresponding to Leu17 in HER1, Thr20 in HER3, and Leu14 in HER4) are different than the corresponding residues at the equivalent positions in the other HER family members. These residues are part of the v-shaped excursion which forms an extended β sheet with the ligand, and interfere with the ability of HER2 to bind ligand. Other sequence differences in domains I and III also account for the inability of HER2 to bind to ligand.

3. HER3 ECD Structure and Domain Organization

The domain organization of a full-length mature HER3 ECD and various HER3 ECD isoforms is depicted in FIG. 2(C). The extracellular portion of HER3 includes residues 1-621 of a mature HER3 receptor and contains subdomains I (amino acid residues 1-166), II (amino acid residues 167-311), III (amino acid residues (312-480), and IV (amino acid residues 481-621). Like other HER family receptors, the structure of domains I, II, and III of HER3 can be superimposed with IGF-1R, and exhibit many of the same structural features as other HER receptors. For example, domains I and III of HER3 exhibit the a β-helical structure, interrupted by extended repeats of disulfide-containing modules. A high degree of interdomain flexibility exists between domains II and III, not exhibited by IGF-1R. In addition, HER3 exhibits the characteristic β-hairpin loop or dimerization arm in domain II (corresponding to amino acids 242-259 of HER3). The β-hairpin loop provides for an intramolecular contact with conserved residues in domain IV resulting in a closed, or inactive HER3 structure. The residues important in this tethering interaction are set forth in Table 5 above, and include interaction of Y246 with D562 and K583, F251 with G563, and Q252 with H565. Upon binding of ligand, a conformational change reorients domains I and III exposing the dimerization arm from the tethered structure to allow for receptor dimerization.

Unlike other HER family receptors, HER3 does not have a functional kinase domain. Alterations of four amino acid residues in the kinase region that are otherwise conserved among all protein tyrosine kinases render the HER3 kinase dysfunctional. HER3, however, retains tyrosine residues in its carboxy terminal domain and is capable of inducing cellular signaling upon appropriate activation and transphosphorylation. Thus, homodimers of HER3 cannot support linear signaling. The preferential dimerization partner for HER3 is HER2.

4. HER4 ECD Structure and Domain Organization

The domain organization of a full-length mature HER4 ECD and various HER4 ECD isoforms is depicted in FIG. 2(D). The extracellular portion of HER4 includes residues 1-625 of a mature HER4 receptor and contains subdomains I (amino acid residues 1-163), II (amino acid residues 164-308), III (amino acid residues (309-477), and IV (amino acid residues 478-625). HER4 most closely resembles HER1 in that, like HER1, HER4 both is able to bind ligand and exhibit kinase activity. The domain organization, including the presence of the dimerization arm important for both tethering and dimerization is present in HER4. Table 5 above outlines the conserved residues in domain II and IV that lock the HER4 in an inactive state. The corresponding dimerization arm in HER1 corresponds to amino acid residues 237-258 of HER4. Of the ligand binding domains I and III, domain I is the principle domain responsible for the binding of the ligand neuregulin (NRG) to HER4. Domain I of HER4 recognizes N-terminal residues of NRG (Kim et al., (2002) Eur. J. Biochem 269:2323-2329).

The full-length HER4 receptor is expressed as four alternatively spliced isoforms. Two of the alternative spliced isoforms differ within the cytoplasmic tail (i.e. CYT-1 and CYT-2), and the other two differ within the juxtamembrane region (i.e. JM-a and JM-b). The result of the alternatively splicing is the generation of isoforms with different signaling capacities. For example, the CYT-1 isoform contains an additional exon that contains additional docking sites (i.e. SH2) for signaling molecules not present in the CYT-2 isoform. In addition, the JM isoforms differ in their sensitivity to proteinase cleavage, such as for example, by tumor necrosis factor-a converting enzyme (TACE).

5. HER Family Ligands, Ligand Specificity, and Ligand-Mediated Receptor Activation

Activity of members of the ErbB (HER) family of receptors requires ligand binding for dimerization, which leads to catalytic activity ultimately resulting in signal transduction. There are several HER-specific ligands that each belong to the EGF family of ligands (see e.g., Table 6). All EGF ligands have an EGF-like domain, which is a 45-55 amino acid motif with six cysteines that interact to form three loops covalently associated by disulfide bonds. This region is important for conferring binding specificity of the HER ligands. Additional structural motifs in EGF ligands include immunoglobulin-like domains, heparin-binding sites, and glycosylation sites. Generally, the ligands are initially expressed as membrane-anchored proteins that require proteolytic cleavage to achieve activity in solution and/or to bind to cell surface HER proteins. This requirement for cleavage acts to control ligand availability and receptor activation. Proteases involved in EGF ligand shedding include, for example, those from the metalloproteinase family including the disintegrin and metalloprotease (ADAM) family, and the matrix metalloproteinase (MMP) family. Activation of G-protein-coupled receptors (GPCRs) regulates the production of EGF ligands. In cancers, dysregulation of GPCR signaling and the prevalence of EGF ligands in tumors, is associated with the constitutive activation of HER receptors.

Table 6 lists ligands among the most well-known and characterized of these ligands, and their receptor specificity. The ligands are divided into three groups, based upon their receptor preference (outlined as Groups 1-3 in the Table below). None of the ligands bind to HER2, which heterodimerizes with each of the other family members. In the Table below, alternative names for the neuregulin (NRG) family of cytokines include Neu differentiation factors, NDFs, or heregulins (HRG). The Neuregulin/Heregulin family of ligands is structurally related growth factors derived from alternatively spliced NRG-1, NRG-2, NRG-3, or NRG-4 genes. For example, there are at least 14 soluble and transmembrane protein isoforms derived from the NRG-1 gene. Proteolytic processing of the extracellular domain of the transmembrane NRG-1 isoform releases soluble growth factors. HRG-1β is one of these and contains an Ig domain and an EGF-like domain that is necessary for direct binding to HER3 and HER4. A recombinant human HRG-1β containing only the EGF domain of heregulin β is sufficient to bind and activate HER receptors. Another isoform of the NRG-1 gene is HRG1-α. The binding affinity of HRGα is 100-fold weaker than HRGβ for HER3 and HER4 (Jones et al. (1999) FEBS Letters, 447: 227-231). There are at least two NRG-2 isoforms, called NRG2-α and NRG2-β. Both NRG2α and NRG2β are HER3 agonists and stimulate HER3 signaling. NRG2β also is an agonist of HER4, but NRG2α in not a potent stimulus of HER4 tyrosine phosphorylation or signaling. There are no other reported isoforms of NRG-3 and NRG-4.

TABLE 6
HER family ligands

| Ligand | HER1 | HER2 | HER3 | HER4 |
|---|---|---|---|---|
| 1. | | | | |
| Epidermal growth factor (EGF) | X | | | |
| Amphiregulin (AR) | X | | | |
| Transforming growth factor-α | X | | | |
| (TGF-α) | | | | |
| 2. | | | | |
| betacellulin (BTC) | X | | | X |
| heparin-binding EGF (HB-EGF) | X | | | X |
| epiregulin (EPR) | X | | | X |
| 3. | | | | |
| Neuregulin 1 (NRG-1) | | | X | X |
| Neuregulin 2 (NRG-2) | | | X | X |
| Neuregulin 3 (NRG-3) | | | | X |
| Neuregulin 4 (NRG-4) | | | | X |

Since there are well over 15 different EGF ligands that can bind to HER family members, control and regulation of HER family signaling is complex. Among factors that regulate this complex system of signaling include the tissue specific expression of the receptor ligands. For example, NRGs are expressed predominantly in parenchymal organs and in the embryonic central and peripheral nervous systems. In addition, although ligands typically are able to bind to monomeric receptors, they are unable to activate monomeric receptors. Instead, dimeric formation of receptors, and ultimately HER-mediated activation and signaling, is driven by the higher stability of a complex of two HER receptors and a ligand as compared to a monomeric receptor. Ligand binding to a monomeric receptor not only mediates a conformational change of a monomeric receptor to allow for receptor homo- or heterodimerization (see below), but ligands also stabilize the dimeric receptor once formed. Thus, for activation, various dimeric pairs depend on the concentration of receptors, as well as the concentration of ligand. Thus, activation of the HERs is controlled by the spatial and temporal expression of their ligands.

6. Dimerization Versus Tethering and Generation of Active Homo- and Heterodimers

The mechanisms governing the activation of HER family receptors rely upon ligand binding and the induction of a conformational change in the receptor. Typically, an equilibrium exists between the inactive and active forms of the HER receptors. At least in the case of HER1, approximately 95% are present on the cell surface in a tethered or inactive form; and only 5% are in the active form.

In the absence of bound ligand, in the monomeric receptor, the dimerization arm in domain II is buried in an intramolecular tether by interaction with subdomain IV within the same molecule, thereby autoinhibiting the receptor. Thus, normally, all HER receptors, except for HER2, are in an inactive or “tethered” conformation. The tethered conformation is a closed formation of the receptor that prevents interaction of the receptor with other HER family members. Normally, in this conformation the ligand binding domains I and III are held far apart. This is true for all HER family receptors, except for HER2. For HER2, even as a monomeric receptor, domains I and III are structurally close together and sterically inhibit the binding of ligand to this region. As a result, HER2 is unable to bind to ligand, and always has its dimerization arm exposed and ready to facilitate dimerization with another HER family receptor.

Ligand-induced dimerization of HER receptor molecules induces receptor activation and provides the normal downstream signaling mechanism of the HER family of receptors. Activating ligands interact with domains I and/or III, promoting a rearrangement in the ECD, resulting in opening of the tethered conformation and exposure of the dimerization arm. The bound ligand fixes the relative positions of domains I and III forcing them to rotate (approximately 130° for the case of HER1). This rearrangement breaks the intramolecular domain II/IV linkage, or tether, and frees up the dimerization arm so that it is able to participate in intermolecular interactions. This results in an “open” or active conformation of the receptor and renders the molecule competent to dimerize with other HER family members. HER2 is always in the open conformation, even as a monomer. Thus, even in the absence of ligand, HER2 is capable of dimerizing with another HER family member, although it does not dimerize with itself unless overexpressed. In the open configuration, the dimerization arm (see FIG. 1) protrudes out from domain II and is able to interact with a pocket at the base of the domain II dimerization loop in a second receptor via non-covalent interactions, such as homophilic and hydrophobic interactions, van der Waals interactions and hydrogen bonding, Mutations in the dimerization loop can lead to constitutive dimerization, which in the case of HER2 has been shown to induce cell transformation (Bazley et al. (2005) Endocrine-Related Cancer 12:S17-S27). There are contacts between the subdomain loop II and subdomains I and II. Higher order structures such as heterotetramers also can form (see, e.g., Jorissen et al. (2003) Exptl. Cell Res. 284:31-53).

The dimerization arm alone is not sufficient for dimerization. Additional interactions, including domain II/III interactions, stabilize receptor dimerization (see, e.g., Dawson et al., (2005) Mol. Cell. Biol. 25:7734-7742). As discussed above, while the dimerization arm is highly conserved among HER1, 2, 3 and 4, HER2 fails to form homodimers. For HER1, module 6 provides additional self-complementary interactions (including D279 and H280) for homodimerization. Module 7 is involved in HER2/HER3 heterodimerization. These residues are conserved among all four HER receptors. (see, e.g., Dawson et al., (2005) Mol. Cell. Biol. 25:7734-7742).

7. HER Family Receptor Activity

The HERs are expressed in various tissues of epithelial, mesenchymal and neuronal origin and regulate growth, survival, proliferation, and differentiation. Under normal physiological conditions, activation of the HERs is controlled by the spatial and temporal expression of their ligands, which are members of the EGF family of growth factors (see above). Ligand binding to HER receptors induces the formation of receptor homo- and heterodimers and activation of the intrinsic kinase domain, resulting in phosphorylation on specific tyrosine residues within the cytoplasmic tail. These phosphorylated residues serve as docking sites for a range of effector proteins, the recruitment of which leads to the activation of intracellular signaling pathways. For example, the phosphatidylinositol 3-kinase (P13K)-AKT pathway is stimulated by recruitment of the p85 adaptor subunit of P13K to the receptor. The mitogen-activated protein kinase (MAPK) pathway is activated by recruitment of growth-factor-receptor-bound protein 2 (GRB2) or SHC to the receptor.

Activation of each of the receptors differs from one another in several respects. For example, HER2 has no corresponding growth factor ligand, and HER3 has no well defined tyrosine kinase activity. These two receptors are generally co-dependent upon other members for their ability to signal, although HER2 is capable of potent signaling without a co-receptor or ligand when it is sufficiently overexpressed. In contrast, the HER3 homodimer is completely inactive due to the deficient kinase activity of the tyrosine kinase domain. Typically, HER heterodimers are more potent in signaling than are HER homodimers. This is because heterodimerization provides distinct cytoplasmic tails from two different receptors thereby providing additional phosphotyrosine residues and different patterns of phosphorylation for the recruitment of distinct effector molecules. Thus, HER heterodimerization is a mechanism by which signaling can be amplified and diversified. The HER2/HER3 heterodimer is the most potent receptor signaling pair. There are several reasons for the increased potency of the HER2/HER3 heterodimer. First, HER2 and HER3 are coupled to diverse signaling pathways including the mitogen-activated protein kinase (MAPK) pathway important in cell proliferation, and the phosphatidylinosition 3-kinase (PI3K)/Akt pathway which regulates cell survival and antiapoptotic signals. In addition, a HER2/HER3 heterodimer also has prolonged signaling due to efficient receptor recycling and inefficient downregulation of cell surface receptor expression.

Each of the HER receptors has been shown to have a role in diverse cellular processes including cell differentiation, cell proliferation, cell survivial, angiogenesis, and migration and invasion. HER receptors are essential mediators of cell proliferation and differentiation in the developing embryo and in adult tissues, but their inappropriate activation is associated with the development and severity of many cancers, including for example, breast, colon and prostate cancer, and other diseases. There are a number of mechanisms that affect the inappropriate activation of HER receptors associated with disease. Among these include, for example, gene amplification or transcriptional abnormalities leading to receptor overexpression, gene mutation, and autocrine stimulation resulting from the overproduction of HER ligands. Thus, targeting of HER receptors such as, for example, by pan-therapeutics provided herein, is a mechanism by which these processes can be modulated to treat diseases or conditions associated with inappropriate HER signaling. The following are among such activities and corresponding cellular processes mediated by HER receptor signaling. These processes, cell proliferation, cell survival, angiogenesis and cell migration and invasion are hallmarks of tumorigenesis. These processes also can be monitored in vitro, such as is described in Section G, to assess the feasibility of such therapeutics.

a. Cell Proliferation

HER receptor signaling plays a role in regulating proliferation through control of the cell cycle checkpoint. For example, HER2 overexpression dysregulates the G1-S transition and drives cell proliferation. Robust signaling induced by HER2 results in increased levels of the proteins c-Myc and cyclin D. Each of these proteins acts to sequester the protein p27, which is a cyclin kinase inhibitor. Cyclin E-CDK2 mediates cell cycle entry. Sequestration of p27 prevents its binding to cyclin E-CDK2 to inhibit its activity, and thus uncontrolled cell proliferation results. Inhibition of HER2 signaling results in a downregulation of the MAPK and P13K/AKT pathways, which decreases levels of c-Myc and cyclin D. This permits uncomplexed p27 to bind to and inactivate cyclin E-CDK2 to prevent continued cell proliferation.

b. Cell Survival

HER family receptors regulate cell survival by modulating effector proteins involved in the intrinsic pathway of apoptosis. For example, cell survival by HER signaling is mediated through the PI3K/AKT pathway, which targets substrates that inhibit the proapoptotic proteins BAD and caspases 9. In addition, target substrates phosphorylated by AKT also include transcription factors that inhibit the expression of several pro-apoptotic genes, such as for example, FAS ligand, as well as other transcription factors (i.e. NF-κB) that upregulate levels of pro-survivial proteins, such as for example, BCL-X_{L}.

c. Angiogenesis

HER signaling induces the expression of a variety of proangiogenic factors, such as for example, vascular endothelial growth factor (VEGF). For example, HER1 activation induces VEGF production. In addition, overexpression of HER2 is associated with increased VEGF production in colon, pancreatic, gastric, breast, renal cell, and non-small lung cell cancers. The angiogenic effects of VEGF is related to its role in the development of new blood vessels (i.e. angiogenesis) and in vascular maintenance or the survival of immature blood vessels, through its binding and activation of two related receptors expressed on endothelial cells (i.e., VEGFR-1 and VEGFR-2). Angiogenesis plays a role in tumorigenesis.

d. Migration and Invasion

Stimulation of HER signaling also mediates various aspects of cell motility and migration, which play important roles during embryonic development, wound healing, and in tumor growth and metastasis. Cell motility responses can be initiated by a broad spectrum of signaling pathways induced upon HER activation. For example, activation of the PLCγ-dependent pathway by HER1 is linked to HER1-induced cell migration, since inhibition of this enzyme blocks EGF-induced cell movement (Jorissen et al. (2003) Exp. Cell Res. 284:31-53). The mechanism of EGF-mediated cell migration has been linked to stimulation of actin cytoskeleton rearrangement due to PLC-γ-mediated release of actin-modifying proteins (i.e. gelsolin, profiling, cofilin, and CapG). MAPK also plays a role in HER-mediated cell motility, such as for example, by modulating integrin levels. Other signaling pathways or effector molecules involved in HER-mediated cell migration and motility include P13-K, and the downstream effector molecules Rac, involved in membrane ruffling and lamellipodia formation, and Rho, involved in cell rounding and cortical actin polymerization.

In addition, migration and invasion induced by HER signaling also has been linked to the increased expression of matrix metalloproteinases (MMP), which cleave constituents of the extracellular matrix. For example, stimulation of HER3 and HER4 by neuregulin is linked with invasion and the generation of proteolytic activity by tumor cells due to the induction of MMP-2 and MMP-9.

8. Other CSR ECDs

In addition to targeting HER family members, therapeutics provided herein also can be designed to target any other cell surface receptor (CSR), or their ligands, involved in a disease process, including but not limited to, oncogenesis, angiogenesis, or inflammatory diseases. In particular, the other ECD is from a receptor that participates in or is involved in development of resistance to therapeutics that target one receptor.

Typically, such a CSR is a receptor tyrosine kinase (RTK). Generally, such a therapeutic contains the ECD, or portion thereof, of the CSR sufficient to interact with ligand and/or to prevent receptor dimerization. Examples of RTKs include, but are not limited to, epidermal growth factor (EGF) receptors (as discussed above), platelet-derived growth factor (PDGF) receptors, fibroblast growth factor (FGF) receptors, insulin-like growth factor (IGF) receptors, nerve growth factor (NGF) receptors, vascular endothelial growth factor (VEGF) receptors, receptors to ephrin (termed Eph), hepatocyte growth factor (HGF) receptors (termed MET), TIE/Tie-1 or TEK/Tie-2 (the receptor for angiopoietin-1), discoidin domain receptors (DDR) and others, such as Tyro3/Ax1. Other CSRs for which an ECD portion can be used a therapeutic include, but are not limited to, a TNFR (i.e. TNFR1, TNFR2, CD27, 4-1BB, OX40, HVEM, LtβR, CD30, GITR, CD40, and others), or RAGE. Table 7 lists exemplary CSRs, and sets forth the amino acids which make up the ECD of the respective polypeptide. Exemplary sequences of RTKs and other CSRs and the encoded amino acids are set forth in any of SEQ ID NOS: 193-262.

TABLE 7
Exemplary Cell Surface Receptors, and ECD portions thereof

| | | | SEQ | | | SEQ |
|---|---|---|---|---|---|---|
| | | | ID | | | ID |
| Family | Member | nt ACC. # | NO: | prt ACC. # | ECD | NO: |
| PDGFR | CSF1R | NM_005211 | 193 | NP_005202 | 20-512 | 194 |
| | FLT3 | NM_004119 | 195 | NP_004110 | 27-543 | 196 |
| | KIT | NM_000222 | 197 | NP_000213 | 23-520 | 198 |
| | PDGFRA | NM_006206 | 199 | NP_006197 | 24-524 | 200 |
| | PDGFRB | NM_002609 | 201 | NP_002600 | 33-531 | 202 |
| DDR | DDR1 | NM_013993 | 203 | NP_054699 | 19-416 | 204 |
| | DDR2 | NM_006182 | 205 | NP_006173 | 22-399 | 206 |
| EPH | EPHA1 | NM-005232 | 207 | NP_005223 | 24-547 | 208 |
| | EPHA2 | NM-004431 | 209 | NP_004422 | 25-534 | 210 |
| | EPHA3 | NM-005233 | 211 | NP_005224 | 21-541 | 212 |
| | EPHA4 | NM_004438 | 213 | NP_004429 | 20-547 | 214 |
| | EPHA5 | L36644 | 215 | P54756 | 25-573 | 216 |
| | EPHA6 | AL133666 | 217 | CAB63775 | 23-549 | 218 |
| | EPHA7 | NM_004440 | 219 | NP_004431 | 25-556 | 220 |
| | EPHA8 | NM_020526 | 221 | NP_065387 | 31-542 | 222 |
| | EPHB1 | NM_004441 | 223 | NP_004432 | 18-540 | 224 |
| | EPHB2 | AF025304 | 225 | P29323 | 19-543 | 226 |
| | EPHB3 | NM_004443 | 227 | NP_004434 | 34-559 | 228 |
| | EPHB4 | NM_004444 | 229 | NP_004435 | 16-539 | 230 |
| | EPHB6 | NM_004445 | 231 | NP_004436 | 17-579 | 232 |
| FGFR | FGFR1 | M34641 | 233 | P11362 | 22-376 | 234 |
| | FGFR2 | NM_000141 | 235 | NP_000132 | 22-377 | 236 |
| | FGFR3 | NM_000142 | 237 | NP_000133 | 23-375 | 238 |
| | FGFR4 | NM_002011 | 239 | NP_002002 | 22-369 | 240 |
| MET | MET | NM_000245 | 241 | NP_000236 | 25-932 | 242 |
| | RON | NM_002447 | 243 | NP_002438 | 25-957 | 244 |
| TIE | TEK | NM_000459 | 245 | NP_000450 | 23-745 | 246 |
| | (Tie-2) | | | | | |
| | TIE | NM_005424 | 247 | NP_005415 | 22-759 | 248 |
| | (Tie-1) | | | | | |
| TNFR | TNFR1 | NM_001065 | 249 | NP_001056 | 22-211 | 250 |
| | TNFR2 | NM_001066 | 251 | NP_001057 | 23-257 | 252 |
| VEGFR | VEGFR1 | NM_002019 | 253 | NP_002010 | 27-758 | 254 |
| | VEGFR2 | NM_002253 | 255 | NP_002244 | 20-764 | 256 |
| | VEGFR3 | NM_002020 | 257 | NP_002011 | 25-775 | 258 |
| IGF-1R | IGF-1R | X04434 | 259 | P08069 | 31-935 | 260 |
| RAGE | RAGE | M91211 | 261 | Q15109 | 23-342 | 262 |

The ectodomains of RTKs, including growth factor receptors, are made up of a diverse group of modular domains, including, but not limited to, fibronectin type III, cysteine-rich, epidermal growth factor, and immunoglobulin (Ig)-like domains. For many RTKs, the Ig-like domain is responsible for ligand binding (see e.g., Wiesmann et al. (2000) J Mol. Med. 78:247-260). An Ig-like domain typically contains 80-110 residues that form two antiparallel β-sheets of three to five β-strands, with the β-sheets in some cases connected by a disulfide bond. Ig-like domains are grouped into four classes: the V (variable), I (intermediate), and C1 and C2 (constant), depending on the number of β-strands. For example, the domain of the C2 class contains the smallest number of β-strands containing 4 in the first β-sheet and four in the second β-sheet. Table 8 depicts exemplary RTK family members that contain Ig-like domains, and the ligands to which they bind.

TABLE 8

| Ectodomain Structure | Receptors | Ligands |
|---|---|---|
| 7 Ig-like domains | VEGFR1 | VEGF; PLGF |
| | VEGFR2 (KDR) | VEGF; VEGF-C |
| | VEGFR3 | VEGF-C |
| 5 Ig-like domains | PDGFRA, PDGFRB | PDGF-AA; PDGF-BB; |
| | | PDGF-AB |
| | CSF1R | SCF |
| | SCFR | SCF |
| | Flt-3 | Flt-3L |
| 3 Ig-like domains | FGFR1-FGFR4 | FGF1-FGF18 |
| 2 Ig-like, 2 Cys-rich, | Trk-A, TRK-B, | NGF; NT3; NT4/5; |
| 1 Leu-rich domain | TRK-C | BDNF |
| 2 Ig-like, 2 fibronectin | AXL. EYK, TYRO-3 | GAS6; Protein S |
| type III domains | | |
| 2 Ig-like. 3 fibronectin | Tie-1 | |
| type III, 3 EGF domains | Tie-2 (TEK) | Angiopoietin-1; |
| | | Angiopoietin-2 |
| 1 Ig-like, 1 Cys-rich | ROR1, ROR2 | |
| and 1 Kringle domain | | |

The following discussion is for exemplification. It is understood that an ECD or portion thereof that is required for ligand binding and/or dimerization can be combined in a heteromultimer, particularly with a HER ECD or portion thereof.

(a) VEGFR1 (Flt-1) and VEGFR2 (KDR)

VEGFR1 and VEGFR2 bind to VEGF and play a role in VEGF-induced angiogenic responses. VEGFR1 is required for endothelial cell morphogenesis, while VEGFR2 plays a role in mitogenesis. The ECD structure of both VEGFR1 and VEGFR2 contain seven Ig-like domains, and both receptors bind similarly to VEGF, although VEGFR1 also binds to the ligand PIGF. Thus, the differences in function between VEGFR1 and VEGFR2 appear to be in the intracellular tyrosine kinase sequence of the receptors and their different signal transduction properties. The related receptor VEGFR3 also contains seven Ig-like domains, but does not bind to VEGF. For the sequence of VEGFR1 depicted in SEQ ID NO:254, the first Ig-like domain corresponds to amino acids 32-123, the second Ig-like domain corresponds to amino acids 151-214, the third Ig-like domain corresponds to amino acids 230-327, the fourth Ig-like domain corresponds to amino acids 335-421, the fifth Ig-like domain corresponds to amino acids 428-553, the sixth Ig-like domain corresponds to amino acids 556-654, and the seventh Ig-like domain corresponds to amino acids 661-747. For the sequence of VEGFR2 depicted in SEQ ID NO:256, the first Ig-like domain corresponds to amino acids 46-110, the second Ig-like domain corresponds to amino acids 141-207, the third Ig-like domain corresponds to amino acids 224-320, the fourth Ig-like domain corresponds to amino acids 328-414, the fifth Ig-like domain corresponds to amino acids 421-548, the sixth Ig-like domain corresponds to amino acids 551-660, and the seventh Ig-like domain corresponds to amino acids 667-753.

For VEGFR1, the second Ig-like domain (domain 2) determines ligand binding and specificity, as deletion of this domain from the VEGFR1 ECD abolishes the receptor's ability to bind VEGF (Smyth et al. (1996) EMBO J. 15:4919-4927). Deletion of the other domains only reduces binding to VEGF, but does not abolish it. Domain 2 alone, however, is insufficient to bind VEGF. Domain 1 and 2, or domains 2 and 3 also showed no or minimal binding to VEGF. An ECD portion of VEGFR1 containing only domains 1, 2, and 3 has essentially identical affinity for VEGF as a full-length VEGFR1.

(b) FGFR1-FGFR4

The ECD of FGFRs contains three Ig-like domains. For example, for the sequence of FGFR2 depicted in SEQ ID NO:236, the first Ig-like domain corresponds to amino acids 39-125, the second Ig-like domain corresponds to amino acids 154-247, and the third Ig-like domain corresponds to amino acids 256-358. There are four FGFRs generated by alternative splicing. Individual FGFRs are activated by a subset of ligands (among at least 19 related FGF ligands), and alternative splicing in Ig domain III can dramatically change the specificity for certain ligands (Chellaiah et al. (1999) JBC, 274:34785-34794). Thus, the major ligand binding sites for FGF ligands are typically located within distinct Ig-like domains, most generally domain 2 and domain 3 (Cheon et al. (1994) PNAS, 91:989-993). For example, mutation of domain 3 in FGFR2 inhibits the binding of FGF2, without affecting the binding of FGF1 and FGF7. In addition, studies with chimeric FGFR molecules have determined that FGF1 binds to either domain 2 or domain 3; FGF2 preferentially recognizes the distal sequence of FGFR1 containing Ig domain 2 and 3; FGF8 recognizes sequences both N-terminal and C-terminal to Ig domain 2 or FGFR3; and FGF9 binding is dependent on sequences N-terminal to and including Ig domain 2 in FGFR3, with no requirement for domain 3 (Chellaiah et al. (1999) JBC, 274:34785-34794). For binding of FGF to FGFRs, the presence of heparin optimizes the ligand binding affinity.

(c) IGF-1R

Exemplary of RTK receptors is IGF-1R. The insulin receptor family contains homologous tyrosine kinase receptors, including insulin receptor (IR), insulin-like growth factor 1 receptor (IFG1R), and insulin receptor-related receptor. Both the IR and IGF-1R are synthesized as single polypeptide chains and are proteolytically cleaved to yield two distinct chains, termed α and β, linked by disulfide bonds. The α chain is the extracellular portion of the receptor and binds ligand, while the β chain has an extracellular region, a single transmembrane segment and an intracellular tyrosine kinase domain that mediates signal transduction upon binding of ligand. The extracellular portion of the IGF-1R has six structurally distinct domains. The first three are homologous to HER extracellular domains I-III, namely L1 (corresponding to amino acids 51-61 of SEQ ID NO:260), a cysteine-rich domain (corresponding to amino acids 175-333 of SEQ ID NO:260), and L2 (corresponding to amino acids 352-467 of SEQ ID NO:260). These three domains form the minimal ligand binding portion of the receptor and mediate low-affinity binding to insulin. C-terminal to the L2 domain are three extracellular fibronectin type 3 modules, one in the α chain (corresponding to amino acids 489-587 of SEQ ID NO:260), one in the α-β linking module (corresponding to amino acids 611-703 of SEQ ID NO:260), and a third in the β chain (corresponding to amino acids 831-926 of SEQ ID NO:260). The α and β chains form an αβ heterodimer and two heterodimers associate via disulfide bonding to form the intact (αβ)2 receptor. As with HER family receptors, ligand binding is required to activate the receptor and induce transphosphorylation of the cytoplasmic domain. Activation of IGF-1R is involved in cell growth, transformation, and apoptosis.

(d) RAGE and Other CSRs

Other CSR ECDs contemplated herein, include those from RAGE CSRS (see, copending U.S. application Ser. No. 11/429,090) and references cited therein for a description of RAGE CSRs and also for exemplary ECDs and CSR isoforms. Table 7 above also set forth the sequence of a full-length RAGE and the ECD portion thereof.

### D. Components of ECD Multimers and the Formation of ECD Multimers

ECD heteromultimers include at least two different ECDs, or portions thereof for binding to ligand and/or dimerization. In exemplary embodiments herein, at least one of the component ECDs is a HER ECD, generally at least one of a HER1, 3, or 4, or a portion thereof for ligand binding and/or dimerization. Generally, at least two of the ECDs are HERs, particular HER1 and HER3 or HER4. Other ECDs include ECDs from other CSRs, generally RTKs, particularly any associated with oncogenesis or angiogenesis or inflammatory diseases, and typically any associated with resistance to drugs targeted to a single cell surface receptor. ECD polypeptides also can be hybrid ECD molecules containing domains from two or more CSRs. The ECDs in the heteromultimers are linked, whereby multimers, at least heterodimers form.

Any linkage is contemplated that permits or results in interaction of the ECDs to form a heteromultimer, whereby the resulting multimeric molecule interacts with ligand for of one or all of the ECD cognate receptors and/or interacts with one or both of the cognate receptor(s) or other interacting receptor to inhibit dimerization. Such linkages can be any stable linkage based upon covalent and non-covalent interactions.

1. ECD Polypeptides

ECD polypepetides for use in the generation of ECD multimers provided herein can be all or part of an ECD of a CSR such as, for example, any RTK, or any ECD-containing portion thereof. Typically, unless the ECD is all or part of a HER2, the resulting ECD retains its ability to bind ligand. In addition, an ECD that is of the HER family, for example all or part of HER1, HER2, HER3, or HER4 typically also retains its ability to dimerize with a HER family receptor, including full-length HER family receptors. Thus, where a multimer partner is a HER ECD, the HER ECD polypeptide portion includes at least a sufficient portion of subdomain I and subdomain III to bind ligand, and a sufficient portion of subdomain II for dimerization. Generally, the HER ECD also contains at least part of module 1 of subdomain IV. The remainder of subdomain IV is optional.

(a) HER Family Full Length ECD

The ECD polypeptide contained within HER multimers provided herein can be a full-length ECD of a HER polypeptide. For HER polypeptides, the HER ECD contains domains I, II, III, and IV sufficient to enable binding of ligand and to mediate dimerization with a cognate or related HER family receptor. HER ECD polypeptide also include allelic or species variants, or other known variants within the ECD portion of a HER polypeptide so long as the resulting HER ECD polypeptide retains its ability to bind to ligand and/or to dimerize with a cognate receptor or related HER family receptor.

(i) HER1 ECD

A full-length HER1 ECD polypeptide can be used in the formation of ECD multimers provided herein. Such a full length HER1 ECD contains amino acid residues 1-621 of a mature HER1 receptor (HER1-621; HF100). The nucleotide sequence of the HF100 molecule is set forth in SEQ ID NO:11 and encodes a full length HER1 ECD polypeptide having a sequence of amino acids set forth in SEQ ID NO:12. A full-length HER1 ECD polypeptide includes any having one or more variations in amino acid sequence as compared to the exemplary HER1 ECD polypeptide set forth in SEQ ID NO:12. Exemplary of variations in a HER1 polypeptide are any variations corresponding to any allelic variants in a precursor HER1 polypeptide as set forth in SEQ ID NO:263. Exemplary variations in a HER1 full-length ECD polypeptide include any one or more variations corresponding to any one or more of R74Q, P242R, R497K, or C604S in SEQ ID NO:12.

(ii) HER2 ECD

ECD multimers provided herein also can contain a full-length HER2 ECD polypeptide containing amino acid residues 1-628 of a mature HER2 receptor (HER2-650; HF200). The nucleotide sequence of the HF200 molecule is set forth in SEQ ID NO:17 and encodes a full length HER2 ECD polypeptide having a sequence of amino acids set forth in SEQ ID NO:18. A full-length HER2 ECD polypeptide includes any having one or more variations in amino acid sequence as compared to the exemplary HER2 ECD polypeptide set forth in SEQ ID NO:18. Exemplary of variations in a HER2 polypeptide are any variations corresponding to any allelic variants in a precursor HER2 polypeptide as set forth in SEQ ID NO:264. Exemplary variations in a HER2 full-length ECD polypeptide include any one or more variations corresponding to any one or more of W430C in SEQ ID NO:18.

(iii) HER3 ECD

In another example, a full-length HER3 ECD polypeptide can be used in the formation of ECD multimers provided herein. Such a HER3 ECD polypeptide contains amino acid residues 1-621 of a mature HER3 receptor (HER3-621; HF300). The nucleotide sequence of the HF300 molecule is set forth in SEQ ID NO:25 and encodes a full length HER3 ECD polypeptide having a sequence of amino acids set forth in SEQ ID NO:26. A full-length HER3 ECD polypeptide includes any having one or more variations in amino acid sequence as compared to the exemplary HER3 ECD polypeptide set forth in SEQ ID NO:26. Exemplary of variations in a HER3 polypeptide are any variations corresponding to any allelic variants in a precursor HER3 polypeptide as set forth in SEQ ID NO:265. Exemplary variations in a HER3 full-length ECD polypeptide include any one or more variations corresponding to any one or more of G541E in SEQ ID NO:26.

(iv) HER4 ECD

ECD multimers provided herein also can contain a full-length HER4 ECD polypeptide containing amino acid residues 1-625 of a mature HER4 receptor (HER4-650; HF400). The nucleotide sequence of the HF400 molecule is set forth in SEQ ID NO:31 and encodes a full length HER4 ECD polypeptide having a sequence of amino acids set forth in SEQ ID NO:32. A full-length HER4 ECD polypeptide includes any having one or more variations in amino acid sequence as compared to the exemplary HER4 ECD polypeptide set forth in SEQ ID NO:32. Exemplary of variations in a HER4 polypeptide are any variations corresponding to any allelic variants in a precursor HER4 polypeptide as set forth in SEQ ID NO:266. Exemplary variations in a HER4 full-length ECD polypeptide include any one or more amino acid variations corresponding to the sequence of amino acids set forth in SEQ ID NO:32.

(b) HER Family Truncated ECD

The ECD polypeptide contained within HER multimers provided herein can be a truncated ECD of a HER polypeptide. For truncated HER polypeptides, the HER ECD typically contains a sufficient portion of domains I and III to bind ligand, and a sufficient portion of domain II to mediate receptor dimerization. Generally, truncated HER ECDs also contain at least a portion of module 1 of domain IV to, for example, stabilize the molecule. Any remaining portion of domain IV is optional. Additionally, a truncated ECD polypeptide also can include additional sequence not part of the HER ECD, so long as the additional sequence does not inhibit or interfere with the ligand binding and/or receptor dimerization of the HER ECD polypeptide. For example, truncated ECD polypeptides can include polypeptides generated by alternative splicing, such as, but not limited to, polypeptides that contain intron-encoded amino acids. Truncated HER ECD polypeptide also include allelic or species variants, or other known variants within the ECD portion of a truncated HER polypeptide so long as the resulting truncated HER ECD polypeptide retains its ability to bind to ligand and/or to dimerize with a cognate receptor or related HER family receptor.

(i) Truncated HER1 ECD

In one example a truncated HER1 ECD polypeptide that can be used in the ECD multimers provided herein contains amino acid residues 1-501 of a mature HER1 receptor (HER1-501; HF110). The nucleotide sequence of the HF110 molecule is set forth in SEQ ID NO:9 and encodes a truncated HER1 ECD polypeptide having a sequence of amino acids set forth in SEQ ID NO:10. HF110 contains all of domains I, II, and III of a cognate HER1 ECD, and all of module 1 of domain IV.

Also contemplated for use in ECD multimers are truncated HER1 ECD polypeptides generated from alternative splicing. Such isoforms include any known in the art, or described in related U.S. Patent Publication No. US 2005-0239088, or provided herein below as intron fusion proteins. One such exemplary truncated HER1 ECD polypeptide is EGFR isoform b (NP_{—}958439; SEQ ID NO:129) encoded by a sequence of nucleotides set forth in SEQ ID NO:128. This truncated HER1 ECD polypeptide is 628 amino acids, including a signal peptide corresponding to amino acid residues 1-24, and contains one additional amino acid at its C-terminal end not present in a cognate HER1 ECD. The mature form of the precursor truncated HER1 ECD polypeptide set forth in SEQ ID NO:129 (not including the signal sequence) is 604 amino acids in length as depicted in FIG. 2(A), and contains domains I, II, and III, and most all of domain IV up to and including most of module 7 of a cognate HER1 ECD. In an additional example, a truncated HER1 ECD polypeptide can include EGFR isoform d (NP_{—}958441; SEQ ID NO:131) encoded by a sequence of nucleotides set forth in SEQ ID NO:130. This truncated HER1 ECD polypeptide is 705 amino acids, including a signal peptide corresponding to amino acid residues 1-24, and contains 76 additional amino acids at its C-terminal end not present in a cognate HER1 ECD. The mature form of the precursor truncated HER1 ECD polypeptide set forth in SEQ ID NO:131 (not including the signal sequence) is 681 amino acids in length as depicted in FIG. 2(A), and contains domains I, II, and III, and most of domain IV including up to and most of module 7 of a cognate HER1 ECD.

A truncated HER1 ECD polypeptide includes any having one or more variations in amino acid sequence as compared to, for example, the exemplary truncated HER1 ECD polypeptide set forth in SEQ ID NO:10, 129, or 131. Exemplary of variations in a HER1 polypeptide are any variations corresponding to any allelic variants in a precursor HER1 polypeptide as set forth in SEQ ID NO:263. Exemplary variations in a truncated HER1 ECD polypeptide include any one or more variations corresponding to any one or more of R74Q, P242R, or R497K in SEQ ID NO:10. Exemplary variations also can include any one or more amino acid variations corresponding to R98Q, P266R, R521K, C628S or, V674I in a truncated HER1 polypeptide having a sequence of amino acids set forth in SEQ ID NO:129 or 131.

(ii) Truncated HER2 ECD

ECD multimers also can contain truncated HER2 ECD polypeptides. For example, a truncated HER2 ECD polypeptide containing amino acid residues 1-573 of a mature HER2 receptor (HER2-595; HF210) can be used in the formation of ECD multimers. The nucleotide sequence of the HF210 molecule is set forth in SEQ ID NO:15 and encodes a truncated HER2 ECD polypeptide having a sequence of amino acids set forth in SEQ ID NO:16. HF210 includes all of domains I, II, and III, and up to and including part of module 5 of domain IV of a cognate HER2 ECD. Also provided herein as a multimerization partner is a truncated HER2 ECD polypeptide containing amino acid residues 1-508 of a mature Her2 receptor (HER2-530; HF220). The nucleotide sequence of HF220 is set forth in SEQ ID NO: 13 and encodes a truncated HER2 ECD polypeptide having a sequence of amino acids set forth in SEQ ID NO:14. HF220 includes all of domains I, II, and III, and up to and including al of module 1 of domain IV of a cognate HER2 receptor.

Also contemplated for use in ECD multimers are truncated HER2 ECD polypeptides generated from alternative splicing. Such isoforms include any known in the art, or described in related U.S. Patent Publication No. US 2005-0239088, or provided herein below as intron fusion proteins. One such exemplary truncated HER2 ECD polypeptide is ErbB2.1e having a sequence of amino acids set forth in SEQ ID NO:137. This truncated HER2 ECD polypeptide is 633 amino acids, including a signal peptide corresponding to amino acid residues 1-22. The mature form of the precursor truncated HER2 ECD polypeptide set forth in SEQ ID NO:137 (not including the signal sequence) is 611 amino acids in length as depicted in FIG. 2(B), and contains domains I, II, and III, and most all of domain IV up to and including most of module 7 of a cognate HER2 ECD. In an additional example, a truncated HER2 ECD polypeptide is ErbB2.1d having a sequence of amino acids set forth in SEQ ID NO:136. This truncated HER2 ECD polypeptide is 680 amino acids, including a signal peptide corresponding to amino acid residues 1-24 that contains a two amino acid insert as compared to the signal peptide in a cognate HER2 set forth in SEQ ID NO:4. ErbB2.1d also contains 30 additional amino acids at its C-terminal end not present in a cognate HER2 ECD. The mature form of the precursor truncated HER2 ECD polypeptide set forth in SEQ ID NO:136 (not including the signal sequence) is 656 amino acids in length as depicted in FIG. 2(B), and contains domains I, II, and III, and most of domain IV including all of modules 1-7 of a cognate HER2 ECD.

A truncated HER2 ECD polypeptide includes any having one or more variations in amino acid sequence as compared to, for example, the exemplary truncated HER2 ECD polypeptide set forth in SEQ ID NO:14, 16, 136, and 137. Exemplary of variations in a HER2 polypeptide are any variations corresponding to any allelic variants in a precursor HER2 polypeptide as set forth in SEQ ID NO:264. Exemplary variations in a truncated HER2 ECD polypeptide include any one or more variations corresponding to W430C in SEQ ID NO:14 or 16. Exemplary variations also can include any one or more amino acid variations corresponding to W452C or W454C in a truncated HER2 polypeptide having a sequence of amino acids set forth in SEQ ID NO:137 or 136, respectively.

(iii) Truncated HER3 ECD

An ECD multimer also can contain a truncated HER3 ECD polypeptide containing amino acid residues 1-500 of a mature HER3 receptor (HER3-500; HF310). The nucleotide sequence of the HF310 molecule is set forth in SEQ ID NO:19 and encodes a truncated HER3 ECD polypeptide having a sequence of amino acids set forth in SEQ ID NO:20. HF310 includes all of domains I, II, and III, and up to and including part of module 1 of domain IV of a cognate HER3 ECD. In another example, an ECD multimer can contain a truncated HER3 ECD polypeptide containing amino acid residues 1-519 of a mature HER3 receptor (HER3-519). The nucleotide sequence of HER3-519 is set forth in SEQ ID NO: 23 and encodes a truncated HER3 ECD polypeptide having a sequence of amino acids set forth in SEQ ID NO:24. HER3-519 includes all of domains I, II, and III, and up to and including part of module 3 of domain IV of a cognate HER3 receptor.

Also contemplated for use in ECD multimers are truncated HER3 ECD polypeptides generated from alternative splicing. Such isoforms include any known in the art, or described in related U.S. Patent Publication No. US 2005-0239088, or provided herein below as intron fusion proteins. One such exemplary truncated HER3 ECD polypeptide is p85HER3 set forth in SEQ ID NO:22 and encoded by a sequence of nucleotides set forth in SEQ ID NO:21. This truncated HER3 ECD polypeptide is 562 amino acids, including a signal peptide corresponding to amino acid residues 1-19, and contains 24 additional amino acid at its C-terminal end not present in a cognate HER3 ECD. The mature form of the precursor truncated HER3 ECD polypeptide set forth in SEQ ID NO:22 (not including the signal sequence) is 543 amino acids in length as depicted in FIG. 2(C), and contains domains I, II, and III, and up to and including part of module 3 of domain IV of a cognate HER3 ECD.

A truncated HER3 ECD polypeptide includes any having one or more variations in amino acid sequence as compared to, for example the exemplary truncated HER3 ECD polypeptide set forth in SEQ ID NO:14, 16, 136, and 137. Exemplary of variations in a HER3 polypeptide are any variations corresponding to any allelic variants in a precursor HER3 polypeptide as set forth in SEQ ID NO:265.

(iv) Truncated HER4 ECD

Additionally, an ECD multimer can be formed containing a truncated HER4 ECD. One exemplary truncated HER4 ECD polypeptide contains amino acid residues 1-522 of a mature HER4 receptor (HER4-522). The nucleotide sequence of the HER4-522 molecule is set forth in SEQ ID NO:29 and encodes a truncated HER4 ECD polypeptide having a sequence of amino acids set forth in SEQ ID NO:30. HER4-522 includes all of domains I, II, and III, and up to and including module 1 of domain IV of a cognate HER3 ECD. Another exemplary truncated HER4 ECD polypeptide contains amino acid residues 1-460 of a mature HER4 receptor (HF410; HER4-485). The nucleotide sequence of HF410 is set forth in SEQ ID NO: 27 and encodes a truncated HER4 ECD polypeptide having a sequence of amino acids set forth in SEQ ID NO:28. HF410 includes all of domains I, II, and most of domain III of a cognate HER4 ECD.

Also contemplated for use in ECD multimers are truncated HER4 ECD polypeptides generated from alternative splicing. Such isoforms include any known in the art, or described in related U.S. Patent Publication No. US 2005-0239088, or provided herein below as intron fusion proteins. One such exemplary truncated HER4 ECD polypeptide is ErbB4_int12 set forth in SEQ ID NO:159 and encoded by a sequence of nucleotides set forth in SEQ ID NO:158. This truncated HER4 ECD polypeptide is 506 amino acids, including a signal peptide corresponding to amino acid residues 1-25, and contains 10 additional amino acid at its C-terminal end not present in a cognate HER4 ECD. The additional amino acids are encoded by a portion of intron 12 of the HER4 gene retained as an alternative splice product. The mature form of the precursor truncated HER4 ECD polypeptide set forth in SEQ ID NO:159 (not including the signal sequence) is 481 amino acids in length as depicted in FIG. 2(D), and contains domains I, II, and most of domain III of a cognate HER4 ECD.

A truncated HER4 ECD polypeptide includes any having one or more variations in amino acid sequence as compared to, for example the exemplary truncated HER4 ECD polypeptides set forth in SEQ ID NO:28, 30, and 159. Exemplary of variations in a HER3 polypeptide are any variations corresponding to any allelic variants in a precursor HER4 polypeptide as set forth in SEQ ID NO:266.

(c) Hybrid ECD

Provided herein are hybrid ECDs or portion thereof that contain subdomains from two or more HER receptors. Generally, a hybrid ECD contains all or a sufficient portion of domains I or III of one or more HER receptors to bind to ligand, and all or a sufficient portion of domain II to mediate receptor dimerization from the same or another HER ECD. Thus, a hybrid ECD molecule can contain portions of all HER family ECDs, generally a portion of three HER family ECDs and at least a portion of two HER family ECDs. Typically such ECDs include subdomain II from HER2 and subdomains I and III, which can be from the same or different receptor, from ErbB1, 3 or 4. Each subdomain portion is selected such that the resulting ECD dimerizes and binds to at least one, and can bind to two or more (different), ligands. Hence, the combinations of domains are selected such that it binds to at least one ligand, and can bind to two ligands, and also includes a sufficient portion of subdomain II for dimerization. Exemplary of such hybrids is a monomeric hybrid ECD that contains subdomain I from HER3 or HER4, subdomain II from HER2 and subdomain III from HER1. For example, provided is a hybrid ECD that contains subdomain I from ErbB3, subdomain II from ErbB2 and subdomain III from ErbB 1. HRG will bind to HER3 or HER4 (subdomain I), and EGF will interact primarily with subdomain III of HER1 (see, e.g., Singer et al., (2001) J. Biol. Chem. 276:44266-44274; Kim et al. (2002) Eur. J. Biochem. 269:2323-2329). Hence, the hybrid binds to at least two ligands (see, e.g., Singer et al., (2001) J. Biol. Chem. 276:44266-44274). Furthermore, upon addition of a multimerization domain and formation of chimeric multimers, the resulting chimeric molecule can interact with at least two differ HER receptors and at least two different ligands.

(d) Other CSR or RTK ECDs, or Portions Thereof

Other ECD polypeptides, including any ECD portion, or fragment thereof of a CSR or other RTK sufficient to bind ligand, can be used in the formation of an ECD multimer provided herein. Typically, such CSR ECDs, or portions thereof, are ECDs of any CSR involved in an etiology of a disease and/or an ECD of a CSR involved in resistance to drugs targeted to a single cell surface receptor. Exemplary CSR or RTK receptors are set forth in Table 7, which also denotes the respective ECD portion of each respective receptor. Thus, any full-length ECD as set forth in Table 7 is contemplated for use as a multimerization partner herein. Portions or fragments of a full-length ECD of any of the CSRs depicted in Table 7 also are contemplated for use as a multimerization partner, so long as the portion or fragment retains its ability to bind ligand and/or dimerized with a cognate receptor. For example, a portion or fragment of a VEGFR ECD, such as a VEGFR1, contains at least a sufficient portion of Ig-domains 1, 2, and 3 to bind to ligand. In another example, a portion or fragment of a FGFR ECD, such as any of FGFR1-4, contains at least a sufficient portion of Ig-domains 2 and 3 to bind ligand. In an additional example, a portion or fragment of an IGF-1R ECD contains at least a sufficient portion of the L1 domain, the cysteine-rich domain, and the L2 domain to bind to ligand and/or mediate receptor dimerization.

(e) Alternatively Spliced Polypeptide Isoforms

Other ECD polypeptides for use in the formation of ECD multimers provided herein include any isoform containing an ECD portion of a CSR, or fragment thereof, and optionally additional amino acids that do not align with domain sequence of a cognate receptor. Such ECD polypeptides include, for example, alternatively spliced CSRs or other RTKs. Typically, an ECD-containing polypeptide isoform binds ligand and/or dimerizes with a cell surface receptor. Alternatively spliced isoforms include those generated, for example, by exon extension, exon insertion, exon deletion, exon truncation, or intron retension. Such alternatively spliced isoforms are known in the art (see for e.g., U.S. Pat, No. 6,414,130; published U.S. Patent Application Nos. US2005/0239088, US2004/0022785A1, US20050123538; published International Patent Application Nos. WO0044403, WO0161356, and WO0214470) and set forth in any one of SEQ ID NOS: 22, 129, 131, 133, 135, 136, 137, 138, 139, 143, 144, 149, 150, 151, 301-399, and 408-413. For example, alternatively spliced isoforms include isoforms of HER1 including, but not limited to, any set forth in SEQ ID NO: 129, 131, or 133; isoforms of HER2 including, but not limited to herstatin or variants thereof set forth in any of SEQ ID NOS: 135 or 385-399 or other alternatively spliced isoforms, including but not limited to any set forth in SEQ ID NO: 136-139, or 408-413; isoforms of HER3 including, but not limited to, any set forth in SEQ ID NOS: 22, 143, 144, 149, 150, or 151.

Alternatively spliced isoforms also can include other isoforms of a HER1 gene. The HER1 gene (SEQ ID NO:400) is composed of 28 exons interrupted by 27 introns. In the exemplary genomic sequence of HER1 provided herein as SEQ ID NO:400, exon 1 includes nucleotides 1-254, including the 5′-untranslated region. The start codon begins at nucleotide position 167. Intron 1 includes nucleotides 255-614; exon 2 includes nucleotides 615-766; intron 2 includes nucleotides 767-1126; exon 3 includes nucleotides 1127-1310; intron 3 includes nucleotides 1311-1670; exon 4 includes nucleotides 1671-1805; intron 4 includes nucleotides 1806-2165; exon 5 includes nucleotides 2166-2234; intron 5 includes nucleotides 2235-2594; exon 6 includes nucleotides 2595-2713; intron 6 includes nucleotides 2714-3073; exon 7 includes nucleotides 3074-3215; intron 7 includes nucleotides 3216-3575; exon 8 includes nucleotides 3576-3692; intron 8 includes nucleotides 3693-4052; exon 9 includes nucleotides 4043-4179; intron 9 includes nucleotides 4180-4539; exon 10 includes nucleotides 4540-4613; intron 10 includes nucleotides 4614-4973; exon 11 includes nucleotides 4974-5063; intron 11 includes nucleotides 5064-5423; exon 12 includes nucleotides 5424-5623; intron 12 includes nucleotides 5624-5983; exon 13 includes nucleotides 5984-6116; intron 13 includes nucleotides 6117-6476; exon 14 includes nucleotides 6477-6567; intron 14 includes nucleotides 6568-6927; exon 15 includes nucleotides 6928-7085; intron 15 includes nucleotides 7086-7445; exon 16 includes nucleotides 7446-7484; intron 16 includes nucleotides 7485-7844; exon 17 includes nucleotides 7845-7988; intron 17 includes nucleotides 7987-8346; exon 18 includes nucleotides 8347-8469; intron 18 includes nucleotides 8470-8829; exon 19 includes nucleotides 8830-8295; intron 19 includes nucleotides 8929-9288; exon 20 includes nucleotides 9289-9474; intron 20 includes nucleotides 9475-9834; exon 21 includes nucleotides 9835-9990; intron 21 includes nucleotides 9991-10350; exon 22 includes nucleotides 10351-10426; intron 22 includes nucleotides 10427-10786; exon 23 includes nucleotides 10787-10933; intron 23 includes nucleotides 10934-11293; exon 24 includes nucleotides 11294-11391; intron 24 includes nucleotides 11392-11751; exon 25 includes nucleotides 11752-11919; intron 26 includes nucleotides 11920-12279; exon 26 includes nucleotides 12280-12327; intron 26 includes nucleotides 12328-12687; exon 27 includes nucleotides 12688-12796; intron 27 includes nucleotides 12797-13156; and exon 28 includes nucleotides 13157-15233. The stop codon in exon 28 begins at nucleotide position 13516, and the remainder of exon 28 includes the 3′-untranslated region. Following RNA splicing and the removal of the introns, the primary transcript of HER1 contains exons 1-28 and encodes a polypeptide of 1210 amino acids (SEQ ID NO:2). Alternative spliced isoforms of the HER1 gene are described and set forth in Example 10, and include isoform with a retained intron sequence. A sequence of such an exemplary HER1 isoforms is set forth in SEQ ID NO:126, and encodes a polypeptide having an amino acid sequence set forth in SEQ ID NO:127.

Alternatively spliced isoforms also can include other isoforms of a HER2 gene. The HER2 gene (SEQ ID NO:401) is composed of 27 exons interrupted by 26 introns. In the exemplary genomic sequence of HER provided herein as SEQ ID NO:401, exon 1 includes nucleotides 181-349, including the 5′-untranslated region. The start codon begins at nucleotide position 277. Intron 1 includes nucleotides 350-709; exon 2 includes nucleotides 710-861; intron 2 includes nucleotides 862-1221; exon 3 includes nucleotides 1222-1435; intron 3 includes nucleotides 1436-1795; exon 4 includes nucleotides 1796-1930; intron 4 includes nucleotides 1931-2290; exon 5 includes nucleotides 2291-2359; intron 5 includes nucleotides 2360-2719; exon 6 includes nucleotides 2720-2835; intron 6 includes nucleotides 2836-3195; exon 7 includes nucleotides 3196-3337; intron 7 includes nucleotides 3338-3697; exon 8 includes nucleotides 3698-3817; intron 8 includes nucleotides 3818-4177; exon 9 includes nucleotides 4178-4304; intron 9 includes nucleotides 4305-4664; exon 10 includes nucleotides 4665-4738; intron 10 includes nucleotides 4739-5098; exon 11 includes nucleotides 5099-5189; intron 11 includes nucleotides 5190-5549; exon 12 includes nucleotides 5550-5749; intron 12 includes nucleotides 5750-6109; exon 13 includes nucleotides 6110-6242; intron 13 includes nucleotides 6243-6602; exon 14 includes nucleotides 6603-6696; intron 14 includes nucleotides 6694-7053; exon 15 includes nucleotides 7054-7214; intron 15 includes nucleotides 7215-7574; exon 16 includes nucleotides 7575-7622; intron 16 includes nucleotides 7623-7982; exon 17 includes nucleotides 7983-8121; intron 17 includes nucleotides 8122-8481; exon 18 includes nucleotides 8482-8604; intron 18 includes nucleotides 8605-8964; exon 19 includes nucleotides 8695-9067; intron 19 includes nucleotides 9068-9427; exon 20 includes nucleotides 9428-9610; intron 20 includes nucleotides 9611-9970; exon 21 includes nucleotides 9971-10126; intron 21 includes nucleotides 10127-10486; exon 22 includes nucleotides 10487-10562; intron 22 includes nucleotides 10563-10922; exon 23 includes nucleotides 10923-11069; intron 23 includes nucleotides 11070-11429; exon 24 includes nucleotides 11430-11527; intron 24 includes nucleotides 11528-11887; exon 25 includes nucleotides 11888-12076; intron 26 includes nucleotides 12077-12436; exon 26 includes nucleotides 12437-12689; intron 26 includes nucleotides 12690-13049 and exon 27 includes nucleotides 13050-14018. The stop codon in exon 27 begins at nucleotide position 13403, and the remainder of exon 27 includes the 3′-untranslated region. Following RNA splicing and the removal of the introns, the primary transcript of HER2 contains exons 1-27 and encodes a polypeptide of 1255 amino acids (SEQ ID NO:4). Alternative spliced isoforms of the HER2 gene are described in set forth in Example 10, and include those with a retained intron sequence. A sequence of such an exemplary HER2 isoforms is set forth in SEQ ID NO:140, and encodes a polypeptide having an amino acid sequence set forth in SEQ ID NO:141.

Alternatively spliced isoforms also can include other isoforms of a HER3 gene. The HER3 gene (SEQ ID NO:402) is composed of 28 exons interrupted by 27 introns. In the exemplary genomic sequence of HER3 provided herein as SEQ ID NO:402, exon 1 includes nucleotides 181-460, including the 5′-untranslated region. The start codon begins at nucleotide position 379. Intron 1 includes nucleotides 461-820; exon 2 includes nucleotides 821-972; intron 2 includes nucleotides 973-1332; exon 3 includes nucleotides 1333-1519; intron 3 includes nucleotides 1520-1879; exon 4 includes nucleotides 1880-2005; intron 4 includes nucleotides 2006-2365; exon 5 includes nucleotides 2366-2431; intron 5 includes nucleotides 2432-2791; exon 6 includes nucleotides 2792-2910; intron 6 includes nucleotides 2911-3270; exon 7 includes nucleotides 3237-3412; intron 7 includes nucleotides 3413-3772; exon 8 includes nucleotides 3773-3886; intron 8 includes nucleotides 3887-4246; exon 9 includes nucleotides 4247-4367; intron 9 includes nucleotides 4368-4727; exon 10 includes nucleotides 4728-4801; intron 10 includes nucleotides 4802-5161; exon 11 includes nucleotides 5162-5252; intron 11 includes nucleotides 5253-5612; exon 12 includes nucleotides 5613-5818; intron 12 includes nucleotides 5819-6178; exon 13 includes nucleotides 6179-6311; intron 13 includes nucleotides 6312-6671; exon 14 includes nucleotides 6672-6762; intron 14 includes nucleotides 6763-7122; exon 15 includes nucleotides 7123-7277; intron 15 includes nucleotides 7278-7637; exon 16 includes nucleotides 7638-7691; intron 16 includes nucleotides 7692-8051; exon 17 includes nucleotides 8052-8193; intron 17 includes nucleotides 8194-8553; exon 18 includes nucleotides 8554-8673; intron 18 includes nucleotides 8674-9033; exon 19 includes nucleotides 9034-9132; intron 19 includes nucleotides 9133-9492; exon 20 includes nucleotides 9493-9678; intron 20 includes nucleotides 9679-10038; exon 21 includes nucleotides 10039-10194; intron 21 includes nucleotides 10195-10554; exon 22 includes nucleotides 10555-10630; intron 22 includes nucleotides 10631-10990; exon 23 includes nucleotides 10991-11137; intron 23 includes nucleotides 11138-11497; exon 24 includes nucleotides 11498-11595; intron 24 includes nucleotides 11596-11955; exon 25 includes nucleotides 11956-12147; intron 26 includes nucleotides 12148-12507; exon 26 includes nucleotides 12508-12579; intron 26 includes nucleotides 12580-12939; exon 27 includes nucleotides 12940-13240; intron 27 includes nucleotides 13241-13600; and exon 28 includes nucleotides 13601-14875. The stop codon in exon 28 begins at nucleotide position 14125, and the remainder of exon 28 includes the 3′-untranslated region. Following RNA splicing and the removal of the introns, the primary transcript of ErbB3 contains exons 1-28 and encodes a polypeptide of 1342 amino acids (SEQ ID NO:6). Alternative spliced isoforms of the HER3 gene are described in set forth in Example 10, and include those with a retained intron sequence. Sequence of such exemplary HER3 isoforms are set forth in SEQ ID NO:145 and 147, and encodes a polypeptide having an amino acid sequence set forth in SEQ ID NO:146 and 148, respectively.

Alternatively spliced isoforms also can include other isoforms of a HER4 gene. The HER4 gene (SEQ ID NO:403) is composed of 28 exons interrupted by 27 introns. In the exemplary genomic sequence of HER4 provided herein as SEQ ID NO:403, exon 1 includes nucleotides 181-295, including the 5′-untranslated region. The start codon begins at nucleotide position 215. Intron 1 includes nucleotides 296-655; exon 2 includes nucleotides 656-807; intron 2 includes nucleotides 808-1167; exon 3 includes nucleotides 1168-1354; intron 3 includes nucleotides 1355-1714; exon 4 includes nucleotides 1715-1849; intron 4 includes nucleotides 1850-2209; exon 5 includes nucleotides 2210-2275; intron 5 includes nucleotides 2276-2635; exon 6 includes nucleotides 2636-2754; intron 6 includes nucleotides 2755-3114; exon 7 includes nucleotides 3115-3256; intron 7 includes nucleotides 3257-3616; exon 8 includes nucleotides 3617-3730; intron 8 includes nucleotides 3731-4090; exon 9 includes nucleotides 4091-4217; intron 9 includes nucleotides 4218-4577; exon 10 includes nucleotides 4578-4651; intron 10 includes nucleotides 4652-5011; exon 11 includes nucleotides 5012-5102; intron 11 includes nucleotides 5103-5462; exon 12 includes nucleotides 5463-5662; intron 12 includes nucleotides 5663-6022; exon 13 includes nucleotides 6023-6155; intron 13 includes nucleotides 6156-6515; exon 14 includes nucleotides 6516-6609; intron 14 includes nucleotides 6610-6969; exon 15 includes nucleotides 6970-7124; intron 15 includes nucleotides 7125-7484; exon 16 includes nucleotides 7485-7559; intron 16 includes nucleotides 7560-7919; exon 17 includes nucleotides 7920-8052; intron 17 includes nucleotides 8053-8412; exon 18 includes nucleotides 8413-8535; intron 18 includes nucleotides 8536-8895; exon 19 includes nucleotides 8896-8994; intron 19 includes nucleotides 8995-9354; exon 20 includes nucleotides 9355-9540; intron 20 includes nucleotides 9541-9900; exon 21 includes nucleotides 9901-10056; intron 21 includes nucleotides 10057-10416; exon 22 includes nucleotides 10417-10492; intron 22 includes nucleotides 10493-10852; exon 23 includes nucleotides 10853-10999; intron 23 includes nucleotides 11000-11359; exon 24 includes nucleotides 11360-11457; intron 24 includes nucleotides 11458-11817; exon 25 includes nucleotides 11818-11988; intron 26 includes nucleotides 11989-12348; exon 26 includes nucleotides 12349-12396; intron 26 includes nucleotides 12397-12756; exon 27 includes nucleotides 12757-13054; intron 27 includes nucleotides 13055-13414; and exon 28 includes nucleotides 13415-15385. The stop codon in exon 28 begins at nucleotide position 13858, and the remainder of exon 28 includes the 3′-untranslated region. Following RNA splicing and the removal of the introns, the primary transcript of HER4 contains exons 1-28 and encodes a polypeptide of 1308 amino acids (SEQ ID NO:8). Alternative spliced isoforms of the HER4 gene are described in set forth in Example 10, and include those with a retained intron sequence. Sequence of such exemplary HER4 isoforms are set forth in SEQ ID NO:152, 154, 156, or 158, and encodes a polypeptide having an amino acid sequence set forth in SEQ ID NO:153, 155, 157, or 159, respectively.

Alternatively spliced isoforms also can include an isoform of a IGF-1R gene. The IGF1-R gene (SEQ ID NO:404) is composed of 21 exons interrupted by 20 introns. In the exemplary genomic sequence of IGF1-R provided herein as SEQ ID NO:404, exon 1 includes nucleotides 181-306, including the 5′-untranslated region. The start codon begins at nucleotide position 213. Intron 1 includes nucleotides 307-666; exon 2 includes nucleotides 667-1212; intron 2 includes nucleotides 1213-1572; exon 3 includes nucleotides 1573-1884; intron 3 includes nucleotides 1885-2255; exon 4 includes nucleotides 2256-2394; intron 4 includes nucleotides 2395-2754; exon 5 includes nucleotides 2755-2899; intron 5 includes nucleotides 2990-3259; exon 6 includes nucleotides 3260-3474; intron 6 includes nucleotides 3475-3834; exon 7 includes nucleotides 3835-3961; intron 7 includes nucleotides 3962-4321; exon 8 includes nucleotides 4322-4560; intron 8 includes nucleotides 4561-4920; exon 9 includes nucleotides 4921-5088; intron 9 includes nucleotides 5089-5448; exon 10 includes nucleotides 5449-5653; intron 10 includes nucleotides 5654-6013; exon 11 includes nucleotides 6014-6297; intron 11 includes nucleotides 6298-6657; exon 12 includes nucleotides 6658-6794; intron 12 includes nucleotides 6795-7154; exon 13 includes nucleotides 7155-7314; intron 13 includes nucleotides 7315-7674; exon 14 includes nucleotides 7675-7777; intron 14 includes nucleotides 7778-8137; exon 15 includes nucleotides 8138-8208; intron 15 includes nucleotides 8209-8568; exon 16 includes nucleotides 8569-8798; intron 16 includes nucleotides 8799-9158; exon 17 includes nucleotides 9159-9269; intron 17 includes nucleotides9270-9629; exon 18 includes nucleotides 9630-9789; intron 18 includes nucleotides 9790-10149; exon 19 includes nucleotides 10150-10279; intron 19 includes nucleotides 10280-10639; exon 20 includes nucleotides 10640-10774; intron 20 includes nucleotides 10775-11134 and exon 21 includes nucleotides 11135-12356. The stop codon in exon 21 begins at nucleotide position 11514, and the remainder of exon 21 includes the 3′-untranslated region. Following RNA splicing and the removal of the introns, the primary transcript of IGF1-R contains exons 1-21 and encodes a polypeptide of 1367 amino acids (SEQ ID NO:290). Alternative spliced isoforms of the IGF1-R gene are described in set forth in Example 11, and include those with a retained intron sequence. Sequence of such exemplary IGF1-R isoforms are set forth in SEQ ID NOS:297 or 299, and encodes a polypeptide having an amino acid sequence set forth in SEQ ID NOS:298 or 300, respectively.

The alternative spliced isoforms of HER1, HER2, HER3, HER4, and IGF1-R provided herein and set forth in SEQ ID NOS:127, 141, 146, 148, 153, 155, 157, 159, 298, or 300 can be used in the formation of an ECD multimer provided herein. Alternatively, the isoforms can be used alone or in combination with any other isoform, for the treatment of any diseases mediated by their cognate receptor. Exemplary of such diseases are any angiogenic, tumorgenic, or inflammatory disease, in particular cancers, such as are described herein and known to one of skill in the art.

2. Formation of ECD Multimers

ECD multimers, including HER ECD multimers, can be covalently-linked, non-covalently-linked, or chemically linked multimers of receptor ECDs, to form dimers, trimers, or higher multimers. In some instances, multimers can be formed by dimerization of two or more ECD polypeptides. Multimerization between two ECD polypeptides can be spontaneous, or can occur due to forced linkage of two or more polypeptides. In one example, multimers can be linked by disulfide bonds formed between cysteine residues on different ECD polypeptides. In another example, multimers can include an ECD polypeptide joined via covalent or non-covalent interactions to peptide moieties fused to the soluble polypeptide. Such peptides can be peptide linkers (spacers), or peptides that have the property of promoting multimerization. In an additional example, multimers can be formed between two polypeptides through chemical linkage, such as for example, by using heterobifunctional linkers.

a. Peptide Linkers

Peptide linkers can be used to produce polypeptide multimers, such as for example a multimer where one multimerization partner is all or a part of an ECD of a HER family receptor. In one example, peptide linkers can be fused to the C-terminal end of a first polypeptide and the N-terminal end of a second polypeptide. This structure can be repeated multiples times such that at least one, preferably 2, 3, 4, or more soluble polypeptides are linked to one another via peptide linkers at their respective termini. For example, a multimer polypeptide can have a sequence Z₁-X-Z₂, where Z₁ and Z₂ are each a sequence of all or part of an ECD of a cell surface polypeptide and where X is a sequence of a peptide linker. In some instances, Z₁ and/or Z₂ is a all or part of an ECD of a HER family receptor. In another example, Z₁ and Z₂ are the same or they are different. In another example, the polypeptide has a sequence of Z₁-X-Z₂(-X-Z)ₙ, where “n” is any integer, i.e. generally 1 or 2.

Typically, the peptide linker is of sufficient length to allow a soluble ECD polypeptide to form bonds with an adjacent soluble ECD polypeptide. Examples of peptide linkers include -Gly-Gly-, GGGGG (SEQ ID NO:273), GGGGS or (GGGGS)n (SEQ ID NO:174), SSSSG or (SSSSG)n (SEQ ID NO:187), GKSSGSGSESKS (SEQ ID NO:175), GGSTSGSGKSSEGKG (SEQ ID NO: 176), GSTSGSGKSSSEGSGSTKG (SEQ ID NO: 177), GSTSGSGKPGSGEGSTKG (SEQ ID NO: 178), EGKSSGSGSESKEF (SEQ ID NO: 179), or AlaAlaProAla or (AlaAlaProAla)n (SEQ ID NO:188), where n is 1 to 6, such as 1, 2, 3, or 4. Exemplary linkers include:

(1) Gly4Ser with NcoI ends SEQ ID NO. 189

CCATGGGCGG CGGCGGCTCT GCCATGG

(2) (Gly4Ser)2 with NcoI ends SEQ ID NO. 190

CCATGGGCGG CGGCGGCTCT GGCGGCGGCG GCTCTGCCAT GG

(3) (Ser4Gly)4 with NcoI ends SEQ ID NO. 191

CCATGGCCTC GTCGTCGTCG GGCTCGTCGT CGTCGGGCTC
GTCGTCGTCG GGCTCGTCGT CGTCGGGCGC CATGG

(4) (Ser4Gly)2 with NcoI ends SEQ ID NO. 192

CCATGGCCTC GTCGTCGTCG GGCTCGTCGT CGTCGGGCGC CATGG

Linking moieties are described, for example, in Huston et al. (1988) PNAS 85:5879-5883, Whitlow et al. (1993) Protein Engineering 6:989-995, and Newton et al., (1996) Biochemistry 35:545-553. Other suitable peptide linkers include any of those described in U.S. Pat. Nos. 4,751,180 or 4,935,233, which are hereby incorporated by reference. A polynucleotide encoding a desired peptide linker can be inserted between, and in the same reading frame as a polynucleotide encoding a soluble ECD polypeptide, using any suitable conventional technique. In one example, a fusion polypeptide has from two to four soluble ECD polypeptides, including one that is all or part of a HER ECD polypeptide, separated by peptide linkers.

b. Heterobifunctional Linking Agents

Linkage of an ECD polypeptide to another ECD polypeptide to create a heteromultimeric fusion polypeptide can be direct or indirect. For example, linkage of two or more ECD polypeptide can be achieved by chemical linkage or facilitated by heterobifunctional linkers, such as any known in the art or provided herein.

Numerous heterobifunctional cross-linking reagents that are used to form covalent bonds between amino groups and thiol groups and to introduce thiol groups into proteins, are known to those of skill in this art (see, e.g., the PIERCE CATALOG, ImmunoTechnology Catalog & Handbook, 1992-1993, which describes the preparation of and use of such reagents and provides a commercial source for such reagents; see, also, e.g., Cumber et al. (1992) Bioconjugate Chem. 3:397-401; Thorpe et al. (1987) Cancer Res. 47:5924-5931; Gordon et al. (1987) Proc. Natl. Acad Sci. 84:308-312; Walden et al. (1986) J. Mol. Cell Immunol. 2:191-197; Carlsson et al. (1978) Biochem. J. 173: 723-737; Mahan et al. 91987) Anal. Biochem. 162:163-170; Wawryznaczak et al. (1992) Br. J. Cancer 66:361-366; Fattom et al. (1992) Infection & Immun. 60:584-589). These reagents can be used to form covalent bonds between the N-terminal portion of an ECD polypeptide and C-terminus portion of another ECD polypeptide or between each of those portions and a linker. These reagents include, but are not limited to: N-succinimidyl-3-(2-pyridyldithio)propionate (SPDP; disulfide linker); sulfosuccinimidyl 6-[3-2-pyridyldithio)propion [symbol] amido] [symbol] hexanoate (sulfo-LC-SPDP); succinimidyloxycarbonyl-α-methyl benzyl thiosulfate (SMBT, hindered disulfate linker); succinimidyl 6-[3-(2-pyridyldithio)propionami [symbol] do] [symbol] hexanoate (LC-SPDP); sulfosuccinimidyl 4-(N-maleimidomethyl)cyclohexane-1-carboxylate (sulfo-SMCC); succinimi [symbol] dyl 3-(2-pyridyldithio)butyrate (SPDB; hindered disulfide bond linker); sulfosuccinimidyl 2-(7-azido-4-methylcoumarin-3-acetamide) ethyl-1,3′-dithiopropionate (SAED); sulfo-succinimidyl 7-azido-4-methylcoumarin-3-acetate (SAMCA); sulfosuccinimidyl-6-[alpha-methyl-alpha-(2-pyridyldithio)toluamido]-hexanoate (sulfo-LC-SMPT); 1,4-di-[3′-(2′-pyridyldithio)propion-amido]butane (DPDPB); 4-succinimidyloxycarbonyl-α-methyl-α-(2-pyridylthio)toluene (SMPT, hindered disulfate linker);sulfosuccinimidyl-6-[α-methyl-α-(2-pyrimiyldi-thio)toluamido]hexanoate (sulfo-LC-SMPT); m-maleimidobenzoyl-N-hydroxy-succinimide ester (MBS); m-maleimidobenzoyl-N-hydroxysulfo-succinimide ester (sulfo-MBS); N-succinimidyl(4-iodoacetyl)aminobenzoate (SIAB; thioether linker); sulfosuccinimidyl-(4-iodoacetyl)amino benzoate (sulfo-SIAB); succinimidyl-4-(p-maleimi-dophenyl)butyrate (SMPB); sulfosuccinimidyl4-(p-maleimido-phenyl)buty-rate (sulfo-SMPB); azidobenzoyl hydrazide (ABH). These linkers, for example, can be used in combination with peptide linkers, such as those that increase flexibility or solubility or that provide for or eliminate steric hindrance. Any other linkers known to those of skill in the art for linking a polypeptide molecule to another molecule can be employed. General properties are such that the resulting molecule is biocompatible (for administration to animals, including humans) and such that the resulting molecule is a heteromultimeric molecule that modulates the activity of a cell surface molecule, such as a HER, or other cell surface molecule or receptor.

c. Polypeptide Multimerization Domains

Interaction of two or more polypeptides can be facilitated by their linkage, either directly or indirectly, to any moiety or other polypeptide that are themselves able to interact to form a stable structure. For example, separate encoded polypeptide chains can be joined by multimerization, whereby multimerization of the polypeptides is mediated by a multimerization domain. Typically, the multimerization domain provides for the formation of a stable protein-protein interaction between a first chimeric polypeptide and a second chimeric polypeptide. Chimeric polypeptides include, for example, linkage (directly or indirectly) of a nucleic acid encoding an ECD portion of a polypeptide with a nucleic acid encoding a multimerization domain. Typically, at least one multimerization partner is a nucleic acid encoding all of part of a HER ECD linked directly or indirectly to a multimerization domain. Homo- or heteromultimeric polypeptides can be generated from co-expression of separate chimeric polypeptides. The first and second chimeric polypeptides can be the same or different.

Generally, a multimerization domain includes any capable of forming a stable protein-protein interaction. The multimerization domains can interact via an immunoglobulin sequence, leucine zipper, a hydrophobic region, a hydrophilic region, or a free thiol which forms an intermolecular disulfide bond between the chimeric molecules of a homo- or heteromultimer. In addition, a multimerization domain can include an amino acid sequence comprising a protuberance complementary to an amino acid sequence comprising a hole, such as is described, for example, in U.S. patent application Ser. No. 08/399,106. Such a multimerization region can be engineered such that steric interactions not only promote stable interaction, but further promote the formation of heterodimers over homodimers from a mixture of chimeric monomers. Generally, protuberances are constructed by replacing small amino acid side chains from the interface of the first polypeptide with larger side chains (e.g., tyrosine or typtophan). Compensatory cavities of identical or similar size to the protuberances are optionally created on the interface of the second polypeptide by replacing large amino acid side chains with smaller ones (e.g., alanine or threonine).

An ECD chimeric polypeptide, such as for example any provided herein, can be joined anywhere, but typically via its N- or C-terminus, to the N- or C-terminus of a multimerization domain to form a chimeric polypeptide The linkage can be direct or indirect via a linker. Also, the chimeric polypeptide can be a fusion protein or can be formed by chemical linkage, such as through covalent or non-covalent interactions. For example, when preparing a chimeric polypeptide containing a multimerization domain, nucleic acid encoding all or part of an ECD of a polypeptide can be operably linked to nucleic acid encoding the multimerization domain sequence, directly or indirectly or optionally via a linker domain. Typically, the construct encodes a chimeric protein where the C-terminus of the ECD polypeptide is joined to the N-terminus of the multimerization domain. In some instances, a construct can encode a chimeric protein where the N-terminus of the ECD polypeptide is joined to the N- or C-terminus of the multimerization domain.

A polypeptide multimer contains two chimeric proteins created by linking, directly or indirectly, two of the same or different ECD polypeptides directly or indirectly to a multimerization domain. In some examples, where the multimerization domain is a polypeptide, a gene fusion encoding the ECD-multimerization domain chimeric polypeptide is inserted into an appropriate expression vector. The resulting ECD-multimerization domain chimeric proteins can be expressed in host cells transformed with the recombinant expression vector, and allowed to assemble into multimers, where the multimerization domains interact to form multivalent polypeptides. Chemical linkage of multimerization domains to ECD polypeptides can be effected using heterobifunctional linkers as discussed above.

The resulting chimeric polypeptides, and multimers formed therefrom, can be purified by any suitable method such as is described in detail in Section F below, such as, for example, by affinity chromatography over Protein A or Protein G columns. Where two nucleic acid molecules encoding different ECD chimeric polypeptides are transformed into cells, formation of homo- and heterodimers will occur. Conditions for expression can be adjusted so that heterodimer formation is favored over homodimer formation.

i. Immunoglobulin Domain

Multimerization domains include those comprising a free thiol moiety capable of reacting to form an intermolecular disulfide bond with a multimerization domain of an additional amino acid sequence. For example, a multimerization domain can include a portion of an immunoglobulin molecule, such as from IgG1, IgG2, IgG3, IgG4, IgA, IgD, IgM, and IgE. Generally, such a portion is an immunoglobulin constant region (Fc). Preparations of fusion proteins containing soluble ECD polypeptides fused to various portions of antibody-derived polypeptides (including the Fc domain) has been described, see e.g., Ashkenazi et al. (1991) PNAS 88: 10535; Byrn et al. (1990) Nature, 344:677; and Hollenbaugh and Aruffo, (1992) “Construction of Immnoglobulin Fusion Proteins,” in Current Protocols in Immunology, Suppl. 4, pp. 10.19.1-10.19.11.

Antibodies bind to specific antigens and contain two identical heavy chains and two identical light chains covalently linked by disulfide bonds. Both the heavy and light chains contain variable regions, which bind the antigen, and constant (C) regions. In each chain, one domain (V) has a variable amino acid sequence depending on the antibody specificity of the molecule. The other domain (C) has a rather constant sequence common among molecules of the same class. The domains are numbered in sequence from the amino-terminal end. For example, the IgG light chain is composed of two immunoglobulin domains linked from N- to C-terminus in the order V_{L}-C_{L}, referring to the light chain variable domain and the light chain constant domain, respectively. The IgG heavy chain is composed of four immunoglobulin domains linked from the N- to C-terminus in the order V_{H}—C_{H}1-C_{H}2-C_{H}3, referring to the variable heavy domain, contain heavy domain 1, constant heavy domain 2, and constant heavy domain 3. The resulting antibody molecule is a four chain molecule where each heavy chain is linked to a light chain by a disulfide bond, and the two heavy chains are linked to each other by disulfide bonds. Linkage of the heavy chains is mediated by a flexible region of the heavy chain, known as the hinge region. Fragments of antibody molecules can be generated, such as for example, by enzymatic cleavage. For example, upon protease cleavage by papain, a dimer of the heavy chain constant regions, the Fc domain, is cleaved from the two Fab regions (i.e. the portions containing the variable regions).

In humans, there are five antibody isotypes classified based on their heavy chains denoted as delta (δ), gamma (γ), mu (μ) and alpha (α) and epsilon (ε), giving rise to the IgD, IgG, IgM, IgA, and IgE classes of antibodies, respectively. The IgA and IgG classes contain the subclasses IgA1, IgA2, IgG1, IgG2, IgG3, and IgG4. Sequence differences between immunoglobulin heavy chains cause the various isotypes to differ in, for example, the number of C domains, the presence of a hinge region, and the number and location of interchain disulfide bonds. For example, IgM and IgE heavy chains contain an extra C domain (C4), that replaces the hinge region. The Fc regions of IgG, IgD, and IgA pair with each other through their Cγ3, Cδ3, and Cα3 domains, whereas the Fc regions of IgM and IgE dimerize through their Cμ4 and Cε4 domains. IgM and IgA form multimeric structures with ten and four antigen-binding sites, respectively.

ECD immunoglobulin chimeric polypeptides provided herein include a full-length immunoglobulin polypeptide. Alternatively, the immunoglobulin polypeptide is less than full length, i.e. containing a heavy chain, light chain, Fab, Fab2, Fv, or Fc. In one example, the ECD immunoglobulin chimeric polypeptides are assembled as monomers or hetero-or homo-multimers, and particularly as dimer or tetramers. Chains or basic units of varying structures can be utilized to assemble the monomers and hetero- and homo-multimers. For example, an ECD polypeptide can be fused to all or part of an immunoglobulin molecule, including all or part of C_{H}, C_{L}, V_{H}, or V_{L} domain of an immunoglobulin molecule (see. e.g., U.S. Pat. No. 5,116,964). Chimeric ECD polypeptides can be readily produced and secreted by mammalian cells transformed with the appropriate nucleic acid molecule. The secreted forms include those where the ECD polypeptide is present in heavy chain dimers; light chain monomers or dimers; and heavy and light chain heterotetramers where the ECD polypeptide is fused to one or more light or heavy chains, including heterotetramers where up to and including all four variable regions analogues are substituted. In some examples, one or more than one nucleic acid fusion molecule can be transformed into host cells to produce a multimer where the ECD portions of the multimer are the same or different. In some examples, a non-ECD polypeptide light-heavy chain variable-like domain is present, thereby producing a heterobifunctional antibody. In some examples, a chimeric polypeptide can be made fused to part of an immunoglobulin molecule lacking hinge disulfides, in which non-covalent or covalent interactions of the two ECDs polypeptide portions associate the molecule into a homo- or heterodimer.

(a) Fc Domain

Typically, the immunoglobulin portion of an ECD chimeric protein includes the heavy chain of an immunoglobulin polypeptide, most usually the constant domains of the heavy chain. Exemplary sequences of heavy chain constant regions for human IgG sub-types are set forth in SEQ ID NOS:163 (IgG1), SEQ ID NO:164 (IgG2), SEQ ID NO: 165 (IgG3), and SEQ ID NO: 166 (IgG4). For example, for the exemplary heavy chain constant region set forth in SEQ ID NO:163, the CH1 domain corresponds to amino acids 1-98, the hinge region corresponds to amino acids 99-110, the CH2 domain corresponds to amino acids 111-223, and the CH3 domain corresponds to amino acids 224-330.

In one example, an immunoglobulin polypeptide chimeric protein can include the Fc region of an immunoglobulin polypeptide. Typically, such a fusion retains at least a functionally active hinge, C_{H}2 and C_{H}3 domains of the constant region of an immunoglobulin heavy chain. For example, a full-length Fc sequence of IgG1 includes amino acids 99-330 of the sequence set forth in SEQ ID NO:163. An exemplary Fc sequence for hIgG1 is set forth in SEQ ID NO: 167, and contains almost all of the hinge sequence corresponding to amino acids 100-110 of SEQ ID NO:163, and the complete sequence for the CH2 and CH3 domain as set forth in SEQ ID NO:163. Another exemplary Fc polypeptide is set forth in PCT application WO 93/10151, and is a single chain polypeptide extending from the N-terminal hinge region to the native C-terminus of the Fc region of a human IgG1 antibody (SEQ ID NO:168). The precise site at which the linkage is made is not critical: particular sites are well known and can be selected in order to optimize the biological activity, secretion, or binding characteristics of the ECD polypeptide. For example, other exemplary Fc polypeptide sequences begin at amino acid C109 or P113 of the sequence set forth in SEQ ID NO: 163 (see e.g., US 2006/0024298).

In addition to hIgG1 Fc, other Fc regions also can be included in the ECD chimeric polypeptides provided herein. For example, where effector functions mediated by Fc/FcγR interactions are to be minimized, fusion with IgG isotypes that poorly recruit complement or effector cells, such as for example, the Fc of IgG2 or IgG4, is contemplated. Additionally, the Fc fusions can contain immunoglobulin sequences that are substantially encoded by immunoglobulin genes belonging to any of the antibody classes, including, but not limited to IgG (including human subclasses IgG1, IgG2, IgG3, or IgG4), IgA (including human subclasses IgA1 and IgA2), IgD, IgE, and IgM classes of antibodies. Further, linkers can be used to covalently link Fc to another polypeptie to generate an Fc chimera.

Modified Fc domains also are contemplated herein for use in chimeras with ECD polypeptides, see e.g. U.S. Patent Publication No. US 2006/0024298; and International Patent Publication No. WO 2005/063816 for exemplary modifications. In some examples, the Fc region is such that it has altered (i.e. more or less) effector function than the effector function of an Fc region of a wild-type immunoglobulin heavy chain. The Fc regions of an antibody interacts with a number of Fc receptors, and ligands, imparting an array of important functional capabilities referred to as effector functions. Fc effector functions include, for example, Fc receptor binding, complement fixation, and T cell depleting activity (see e.g., U.S. Pat. No. 6,136,310). Methods of assaying T cell depleting activity, Fc effector function, and antibody stability are known in the art. For example, the Fc region of an IgG molecule interacts with the FcγRs. These receptors are expressed in a variety of immune cells, including for example, monocytes, macrophages, neutrophils, dendritic cells, eosinophils, mast cells, platelets, B cells, large granular lymphocytes, Langerhans' cells, natural killer (NK) cells, and γδT cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. The ability to mediate cytotoxic and phagocytic effector functions is a potential mechanism by which antibodies destroy targeted cells. Recognition of and lysis of bound antibody on target cells by cytotoxic cells that express FcγRs is referred to as antibody dependent cell-mediated cytotoxicity (ADCC). Other Fc receptors for various antibody isotypes include FcεRs (IgE), FcαRs (IgA), and FcμRs (IgM).

Thus, a modified Fc domain can have altered affinity, including but not limited to, increased or low or no affinity for the Fc receptor. For example, the different IgG subclasses have different affinities for the FcγRs, with IgG1 and IgG3 typically binding substantially better to the receptors than IgG2 and IgG4. In addition, different FcγRs mediate different effector functions. FcγR1, FcγRIIa/c, and FcγRIIIa are positive regulators of immune complex triggered activation, characterized by having an intracellular domain that has an immunoreceptor tyrosine-based activation motif (ITAM). FcγRIIb, however, has an immunoreceptor tyrosine-based inhibition motif (ITIM) and is therefore inhibitory. Thus, altering the affinity of an Fc region for a receptor can modulate the effector functions induced by the Fc domain.

In one example, an Fc region is used that is modified for optimized binding to certain FcγRs to better mediate effector functions, such as for example, ADCC. Such modified Fc regions can contain modifications corresponding to any one or more of G20S5, G20A, S23D, S23E, S23N, S23Q, S23T, K30H, K30Y, D33Y, R39Y, E42Y, T44H, V48I, S51E, H52D, E56Y, E56I, E56H, K58E, G65D, E67L, E67H, S82A, S82D, S88T, S108G, S108I, K110T, K110E, K110D, A111D, A114Y, A114L, A114I, I116D, I116E, I116N, I116Q, E117Y, E117A, K118T, K118F, K118A, and P180L of the exemplary Fc sequence set forth in SEQ ID NO:167, or combinations thereof. A modified Fc containing these mutations can have enhanced binding to an FcR such as, for example, the activating receptor FcγIIIa and/or can have reduced binding to the inhibitory receptor FcγRIIb (see e.g., US 2006/0024298). Fc regions modified to have increased binding to FcRs can be more effective in facilitating the destruction of cancer cells in patients, even when linked with an ECD polypeptide. There are a number of possible mechanisms by which antibodies destroy tumor cells, including anti-proliferation via blockage of need growth pathways, intracellular signaling leading to apopotosis, enhanced down-regulation and/or turnover of receptors, ADCC, and via promotion of the adaptive immune response.

In another example, a variety of Fc mutants with substitutions to reduce or ablate binding with FcγRs also are known. Such muteins are useful in instances where there is a need for reduced or eliminated effector function mediated by Fc. This is often the case where antagonism, but not killing of the cells bearing a target antigen is desired. Exemplary of such an Fc is an Fc mutein described in U.S. Pat. No. 5,457,035 and set forth in SEQ ID NO:169. The amino acid sequence of this mutein is identical to the Fc sequence presented in SEQ ID NO:168, except that amino acid 19 has been changed from Leu to Ala, amino acid 20 has been changed from Leu to Glu, and amino acid 22 has been changed from Gly to Ala. Similar mutations can be made in any Fc sequence such as, for example, the exemplary Fc sequence set forth in SEQ ID NO:167. This mutein exhibits reduced affinity for Fc receptors.

In some instances, an ECD polypeptide Fc chimeric protein provided herein can be modified to enhance binding to the complement protein C1q. In addition to interacting with FcRs, Fc also interact with the complement protein C1q to mediate complement dependent cytotoxicity (CDC). C1q forms a complex with the serine proteases C1r and C1s to form the C1 complex. C1q is capable of binding six antibodies, although binding to two IgGs is sufficient to activate the complement cascade. Similar to Fc interaction with FcRs, different IgG subclasses have different affinity for C1q, with IgG1 and IgG3 typically binding substantially better than IgG2 and IgG4. Thus, a modified Fc having increased binding to C1q mediates enhanced CDC, which is a possible mechanism by which antibodies promote tumor cell destruction. Exemplary modifications in an Fc region that increase binding to C1q include, but are not limited to, amino acid modifications corresponding to K110W, K110Y, and E117S in SEQ ID NO:167.

In an additional example, an Fc region can be utilized that is modified in its binding to FcRn, thereby improving the pharmacokinetics of an ECD-Fc chimeric polypeptide. FcRn is the neonatal FcR, the binding of which recycles endocytosed antibody from the endosomes back to the bloodstream. This process, coupled with preclusion of kidney filtration due to the large size of the full length molecule, results in favorable antibody serum half-lives ranging from one to three weeks. Binding of Fc to FcRn also plays a role in antibody transport. Exemplary modifications in an Fc protein for enhanced binding to FcRn include modifications of amino acids corresponding to T34Q, T34E, M212L, and M212F in SEQ ID NO:267.

Typically, a polypeptide multimer is a dimer of two chimeric proteins created by linking, directly or indirectly, two of the same or different ECD polypeptide to an Fc polypeptide. In some examples, a gene fusion encoding the ECD-Fc chimeric protein is inserted into an appropriate expression vector. The resulting ECD-Fc chimeric proteins can be expressed in host cells transformed with the recombinant expression vector, and allowed to assemble much like antibody molecules, where interchain disulfide bonds form between the Fc moieties to yield divalent ECD polypeptides. Typically, a host cell and expression system is a mammalian expression system to allow for glycosylation of the amino acid corresponding to N81 in SEQ ID NO:167. Glycosylation at this position is important for stabilizing the Fc proteins. Other host cells also can be used where glycosylation at this position is not a consideration.

The resulting chimeric polypeptides containing Fc moieties, and multimers formed therefrom, can be easily purified by affinity chromatography over Protein A or Protein G columns. Where two nucleic acids encoding different ECD chimeric polypeptides are transformed into cells, the formation of heterodimers must be biochemically achieved since ECD chimeric molecules carrying the Fc-domain will be expressed as disulfide-linked homodimers as well. Thus, homodimers can be reduced under conditions that favor the disruption of inter-chain disulfides, but do no effect intra-chain disulfides. Typically, chimeric monomers with different extracellular portions are mixed in equimolar amounts and oxidized to form a mixture of homo- and heterodimers. The components of this mixture are separated by chromatographic techniques. Alternatively, the formation of this type of heterodimer can be biased by genetically engineering and expressing ECD fusion molecules that contain an ECD polypeptide, followed by the Fc-domain of hIgG, followed by either c-jun or the c-fos leucine zippers (see below). Since the leucine zippers form predominantly heterodimers, they can be used to drive the formation of the heterodimers when desired. ECD chimeric polypeptides containing Fc regions also can be engineered to include a tag with metal chelates or other epitope. The tagged domain can be used for rapid purification by metal-chelate chromatography, and/or by antibodies, to allow for detection of western blots, immunoprecipitation, or activity depletion/blocking in bioassays.

(b). Protuberances-Into-Cavity (i.e. Knobs and Holes)

In one aspect, an ECD multimer is engineered to contain an interface between a first chimeric polypeptide and a second chimeric polypeptide to facilitate hetero-oligomerization over homo-oligomerization. Typically, a multimerization domain of one or both of the first and second ECD chimeric polypeptide is a modified antibody fragment such that the interface of the antibody molecule is modified to facilitate and/or promote heterodimerization. In some cases, the antibody molecule is a modified Fc region. Thus, modifications include introduction of a protuberance into a first Fc polypeptide and a cavity into a second Fc polypeptide such that the protuberance is positionable in the cavity to promote complexing of the first and second Fc-containing chimeric ECD polypeptides.

Typically, stable interaction of a first chimeric polypeptide and a second chimeric polypeptide is via interface interactions of the same or different multimerization domain that contains a sufficient portion of a CH3 domain of an immunoglobulin constant domain. Various structural and functional data suggest that antibody heavy chain association is directed by the CH3 domain. For example, X-ray crystallography has demonstrated that the intermolecular association between human IgG1 heavy chains in the Fc region includes extensive protein/protein interaction between CH3 domain whereas the glycosylated CH2 domains interact via their carbohydrate (Deisenhofer et al. (1981) Biochem. 20: 2361). In addition, there are two inter-heavy chain disulfide bonds which are efficiently formed during antibody expression in mammalian cells unless the heavy chain is truncated to remove the CH2 and CH3 domains (King et al. (1992) Biochem. J. 281:317). Thus, heavy chain assembly appears to promote disulfide bond formation rather than vice versa. Engineering of the interface of the CH3 domain promotes formation of heteromultimers of different heavy chains and hinders the assembly of corresponding homomultimers (see e.g., U.S. Pat. No. 5,731,168; International Patent Application WO 98/50431 and WO 2005/063816; Ridgway et al. (1996) Protein Engineering, 9:617-621).

Thus, an ECD multimer provided herein can be formed between an interface of a first and second chimeric ECD polypeptide where the multimerization domain of the first polypeptide contains at least a sufficient portion of a CH3 interface of an Fc domain that has been modified to contain a protuberance and the multimerization domain of the second polypeptide contains at least a sufficient portion of a CH3 interface of an Fc domain that has been modified to contain a cavity. All or a sufficient portion of a modified CH3 interface can be from an IgG, IgA, IgD, IgE, or IgM immunoglobulin. Interface residues targeted for modification in the CH3 domain of various immunoglobulin molecules are set forth in U.S. Pat. No. 5,731,168. Generally, the multimerization domain is all or a sufficient portion of a CH3 domain derived from an IgG antibody, such as for example, IgG1.

Amino acids targeted for replacement and/or modification to create protuberances or cavities in a polypeptide are typically interface amino acids that interact or contact with one or more amino acids in the interface of a second polypeptide. A first polypeptide that is modified to contain protuberance amino acids include replacement of a native or original amino acid with an amino acid that has at least one side chain which projects from the interface of the first polypeptide and is therefore positionable in a compensatory cavity in an adjacent interface of a second polypeptide. Most often, the replacement amino acid is one which has a larger side chain volume than the original amino acid residue. One of skill in the art knows how to determine and/or assess the properties of amino acid residues to identify those that are ideal replacement amino acids to create a protuberance. Generally, the replacement residues for the formation of a protuberance are naturally occurring amino acid residues and include, for example, arginine (R), phenylalanine (F), tyrosine (Y), or tyrptophan (W). In some examples, the original residue identified for replacement is an amino acid residue that has a small side chain such as, for example, alanine, asparagines, aspartic acid, glycine, serine, threonine, or valine.

A second polypeptide that is modified to contain a cavity is one that includes replacement of a native or original amino acid with an amino acid that has at least one side chain that is recessed from the interface of the second polypeptide and thus is able to accommodate a corresponding protuberance from the interface of a first polypeptide. Most often, the replacement amino acid is one which has a smaller side chain volume than the original amino acid residue. One of skill in the art knows how to determine and/or assess the properties of amino acid residues to identify those that are ideal replacement residues for the formation of a cavity. Generally, the replacement residues for the formation of a cavity are naturally occurring amino acids and include, for example, alanine (A), serine (S), threonine (T) and valine (V). In some examples, the original amino acid identified for replacement is an amino acid that has a large side chain such as, for example, tyrosine, arginine, phenylalanine, or typtophan.

The CH3 interface of human IgG1, for example, involves sixteen residues on each domain located on four anti-parallel β-strands which buries 1090 Å2 from each surface (see e.g., Deisenhofer et al. (1981) Biochemistry, 20:2361-2370; Miller et al., (1990) J Mol. Biol., 216, 965-973; Ridgway et al., (1996) Prot. Engin., 9: 617-621; U.S. Pat. No. 5,731,168). Modifications of a CH3 domain to create protuberances or cavities are described, for example, in U.S. Pat. No. 5,731,168; International Patent Applications WO98/50431 and WO 2005/063816; and Ridgway et al., (1996) Prot. Engin., 9: 617-621. For example, modifications in a CH3 domain to create protuberances or cavities can be replacement of any amino acid corresponding to the interface amino acid Q230, V231, Y232, T233, L234, V246, S247, L248, T249, C250, L251, V252, K253, G254, F255, Y256, K275, T276, T277, P278, V279, L280, D281, G285, S286, F287, F288, L289, Y290, S291, K292, L293, T294, and V295 of the sequence set forth in SEQ ID NO:163. In some examples, modifications of a CH3 domain to create protuberances or cavities are typically targeted to residues located on the two central anti-parallel β-strands. The aim is to minimize the risk that the protuberances which are created can be accommodated by protruding into the surrounding solvent rather than being accommodated by a compensatory cavity in the partner CH3 domain. Exemplary of such modifications include, for example, replacement of any amino acid corresponding to the interface amino acid T249, L251, P278, F288, Y290, and K292. Exemplary of amino acid pairs for modification in a CH3 domain interface to create protuberances/cavity interactions include modification of T249 and Y290; and F288 and T277. For example, modifications can include T249Y and Y290T; T249W and Y290A; F288A and T277W; F288W and T277S; and Y290T and T249Y.

In some example, more than one interface interaction can be made. For example, modifications also include, for example, two or more modifications in a first polypeptide to create a protuberance and two or more medications in a second polypeptide to create a cavity. Exemplary of such modifications include, for example, modification of T249Y and F288A in a first polypeptide and modification of T277W and Y290T in a second polypeptide; modification of T277W and F288W in a first polypeptide and modification of T277S and Y290A in a second polypeptide; or modification of F288A and Y290A in a first polypeptide and T249W and T277S in a second polypeptide.

As with other multimerization domains described herein, including all or part of any immunoglobulin molecule or variant thereof, such as an Fc domain or variant thereof, an Fc variant containing CH3 protuberance/cavity modifications can be joined to an ECD polypeptide anywhere, but typically via its N- or C-terminus, to the N- or C-terminus of a first and/or second ECD polypeptide to form a chimeric polypeptide. The linkage can be direct or indirect via a linker. Also, the chimeric polypeptide can be a fusion protein or can be formed by chemical linkage, such as through covalent or non-covalent interactions. Typically, a knob and hole molecule is generated by co-expression of a first ECD polypeptide linked to an Fc variant containing CH3 protuberance modification(s) with a second ECD polypeptide linked to an Fc variant containing CH3 cavitity modification(s).

ii. Leucine Zipper

Another method of preparing ECD polypeptide multimers involves use of a leucine zipper domain. Leucine zippers are peptides that promote multimerization of the proteins in which they are found. Typically, leucine zipper is a term used to refer to a repetitive heptad motif containing four to five leucine residues present as a conserved domain in several proteins. Leucine zippers fold as short, parallel coiled coils, and are believed to be responsible for oligomerization of the proteins of which they form a domain. Leucine zippers were originally identified in several DNA-binding proteins (see e.g., Landschulz et al. (1988) Science 240:1759), and have since been found in a variety of proteins. Among the known leucine zippers are naturally occurring peptides and derivatives thereof that dimerize or trimerize. Recombinant chimeric proteins containing an ECD polypeptide linked, directly or indirectly, to a leucine zipper peptide can be expressed in suitable host cells, and the ECD polypeptide multimer that forms can be recovered from the culture supernatant.

Leucine zipper domains fold as short, parallel coiled coils (O'Shea et al. (1991) Science, 254:539). The general architecture of the parallel coiled coil has been characterized, with a “knobs-into-holes” packing, first proposed by Crick in 1953 (Acta Crystallogr., 6:689). The dimer formed by a leucine zipper domain is stabilized by the heptad repeat, designated (abcdefg)n (see e.g., McLachlan and Stewart (1978) J. Mol. Biol. 98:293), in which residues a and d are generally hydrophobic residues, with d being a leucine, which lines up on the same face of a helix. Oppositely-charged residues commonly occur at positions g and e. Thus, in a parallel coiled coil formed from two helical leucine zipper domains, the “knobs” formed by the hydrophobic side chains of the first helix are packed into the “holes” formed between the side chains of the second helix.

The leucine residues at position d contribute large hydrophobic stabilization energies, and are important for dimer formation (Krystek et al. (1991) Int. J. Peptide Res. 38:229). Hydrophobic stabilization energy provides the main driving force for the formation of coiled coils from helical monomers. Electrostatic interactions also contribute to the stoichiometry and geometry of coiled coils.

(a). Fos and Jun

Two nuclear transforming proteins, fos and jun, exhibit leucine zipper domains, as does the gene product of the murine proto-oncogene, c-myc. The leucine zipper domain is necessary for biological activity (DNA binding) in these proteins. The products of the nuclear oncogenes fos and jun contain leucine zipper domains that preferentially form a heterodimer (O'Shea et al. (1989) Science, 245:646; Turner and Tijian (1989) Science, 243:1689). For example, the leucine zipper domains of the human transcription factors c-jun and c-fos have been shown to form stable heterodimers with a 1:1 stoichiometry (see e.g., Busch and Sassone-Corsi (1990) Trends Genetics, 6:36-40; Gentz et al., (1989) Science, 243:1695-1699). Although jun-jun homodimers also have been shown to form, they are about 1000-fold less stable than jun-fos heterodimers.

Thus, typically an ECD polypeptide multimer provided herein is generated using a jun-fos combination. Generally, the leucine zipper domain of either c-jun or c-fos is fused in frame at the C-terminus of an ECD of a polypeptide by genetically engineering fusion genes. Exemplary amino acid sequences of c-jun and c-fos leucine zippers are set forth in SEQ ID NOS:170 and 171, respectively. In some instances, a sequence of a leucine zipper can be modified, such as by the addition of a cysteine residue to allow formation of disulfide bonds, or the addition of a tyrosine residue at the C-terminus to facilitate measurement of peptide concentration. Such exemplary sequences of encoded amino acids of a modified c-jun and c-fos leucine zipper are set forth in SEQ ID NOS: 172 and 173, respectively. In addition, the linkage of an ECD polypeptide with a leucine zipper can be direct or can employ a flexible linker domain, such as for example a hinge region of IgG, or other polypeptide linkers of small amino acids such as glycine, serine, threonine, or alanine at various lengths and combinations. In some instances, separation of a leucine zipper from the C-terminus of an encoded polypeptide can be effected by fusion with a sequence encoding a protease cleavage sites, such as for example, a thrombin cleavage site. Additionally, the chimeric proteins can be tagged, such as for example, by a 6×His tag, to allow rapid purification by metal chelate chromatography and/or by epitopes to which antibodies are available, such as for example a myc tag, to allow for detection on western blots, immunoprecipitation, or activity depletion/blocking bioassays.

(b). GCN4

A leucine zipper domain also is found in a nuclear protein that functions as a transcriptional activator of a family of genes involved in the General Control of Nitrogen (GCN4) metabolism in S. cerevisiae. The protein is able to dimerize and bind promoter sequences containing the recognition sequence for GCN4, thereby activating transcription in times of nitrogen deprivation. An exemplary sequence of a GCN4 leucine zipper capable of forming a dimeric complex is set forth in SEQ ID NO: 180.

Amino acid substitutions in the a and d residues of a synthetic peptide representing the GCN4 leucine zipper domain (i.e. amino acid substibutions in the sequence set forth as SEQ ID NO:180), have been found to change the oligomerization properties of the leucine zipper domain. For example, when all residues at position a are changed to isoleucine, the leucine zipper still forms a parallel dimer. When, in addition to this change, all leucine residues at position d also are changed to isoleucine, the resultant peptide spontaneously forms a trimeric parallel coiled coil in solution. An exemplary sequence of such a GNC4 leucine zipper domain capable of forming a trimer is set forth in SEQ ID NO:181. Substituting all amino acids at position d with isoleucine and at postion a with leucine results in a peptide that tetramerizes. Such an exemplary sequence of a leucine zipper domain of GCN4 capable of forming tetramers is set forth in SEQ ID NO:182. Peptides containing these substitutions are still referred to as leucine zipper domains since the mechanism of oligomer formation is believed to be the same as that for traditional leucine zipper domains such as the GCN4 described above and set forth in SEQ ID NO:180.

iii. Other Multimerization Domains

Other multimerization domains are known to those of skill in the art and are any that facilitate the protein-protein interaction of two or more polypeptides that are separately generated and expressed as ECD fusions. Examples of other multimerization domains that can be used to provide protein-protein interactions between two chimeric polypeptides include, but are not limited to, the barnase-barstar module (see e.g., Deyev et al., (2003) Nat. Biotechnol. 21:1486-1492); selection of particular protein domains (see e.g., Terskikh et al., (1997) PNAS 94: 1663-1668 and Muller et al., (1998) FEBS Lett. 422:259-264); selection of particular peptide motifs (see e.g., de Kruif et al., (1996) J. Biol. Chem. 271:7630-7634 and Muller et al., (1998) FEBS Lett. 432: 45-49); and the use of disulfide bridges for enhanced stability (de Kruif et al., (1996) J. Biol. Chem. 271:7630-7634 and Schmiedl et al., (2000) Protein Eng. 13:725-734). Exemplary of another type of multimerization domain is one where multimerization is facilitated by protein-protein interactions between different subunit polypeptides, such as is described below for PKA/AKAP interaction.

(a). R/PKA-AD/AKAP

Heteromultimeric ECD polypeptides also can be generated utilizing protein-protein interactions between the regulatory (R) subunit of cAMP-dependent protein kinase (PKA) and the anchoring domains (AD) of A kinase anchor proteins (AKAPs, see e.g., Rossi et al., (2006) PNAS 103:6841-6846). Two types of R subunits (RI and RII) are found in PKA, each with an α and β isoform. The R subunits exist as dimers, and for RII, the dimerization domain resides in the 44 amino-terminal residues (see e.g., SEQ ID NO: 183). AKAPs, via the interaction of their AD domain, interact with the R subunit of PKA to regulate its activity. AKAPs bind only to dimeric R subunits. For example, for human RIIα, the AD binds to a hydrophobic surface formed from the 23 amino-terminal residues. An exemplary sequence of AD is AD1 set forth in SEQ ID NO:184, which is a 17 amino acid residue sequence derived from AKAP-IS, a synthetic peptide optimized for RII-selective binding. Thus, a heteromultimeric ECD polypeptide can be generated by linking (directly or indirectly) a nucleic acid encoding an ECD polypeptide, such as a HER ECD polypeptide, with a nucleic acid encoding an R subunit sequence (i.e. SEQ ID NO:183). This results in a homodimeric molecule, due to the spontaneous formation of a dimer effected by the R subunit. In tandem, another ECD polypeptide fusion can be generated by linking a nucleic acid encoding another ECD polypeptide to a nucleic acid sequence encoding an AD sequence. Upon co-expression of the two components, such as following co-transfection of the ECD chimeric components in host cells, the dimeric R subunit provides a docking site for binding to the AD sequence, resulting in a heteromultimeric molecule. This binding event can be further stabilized by covalent linkages, such as for example, disulfide bonds. In some examples, a flexible linker residue can be fused between the nucleic acid encoding the ECD polypeptide and the multimerization domain. In another example, fusion of a nucleic acid encoding an ECD polypeptide can be to a nucleic acid encoding an R subunit containing a cysteine residue incorporated adjacent to the amino-terminal end of the R subunit to facilitate covalent linkage (see e.g., SEQ ID NO:185). Similarly, fusion of a nucleic acid encoding a partner ECD polypeptide can be to a nucleic acid encoding an AD subunit also containing incorporation of cysteine residues to both the amino- and carboxyl-terminal ends of AD (see e.g., SEQ ID NO:186).

3. Chimeric ECD Polypeptides

Chimeric ECD polypeptides are prepared as described herein for use in the formation of ECD multimers. Chimeric ECD polypeptides typically contain all or part of an ECD of a CSR linked directly or indirectly to a multimerization domain. Exemplary multimerization domains are any described herein including, but not limited to, an immunoglobulin sequence (i.e. a constant region (Fc)), a leucine zipper, compatible protein-protein interaction domains, a coiled-coil motif, a helix loop motif, a complementary hydrophobic regions, complementary hydrophilic regions, a proturberance-into-cavity and a compensatory cavity of identical or similar size, and any others sufficient to form stable multimers. To allow for the formation of multimeric molecules, multimerization domains are the same or complementary between a first chimeric polypeptide and a second chimeric polypeptide. Monomers of separate chimeric ECD polypeptides, once expressed, are stably associated via the multimerization domain to form multimeric ECD polypeptides.

Any ECD portion of a CSR can be used as a multimer partner. For example, any of the ECDs described above, or those set forth in any of SEQ ID NOS:10, 12, 14, 16, 18, 20, 22, 24, 26, 28, 30, 32, 34, 127, 129, 131, 133, 135, 136, 137, 138, 139, 141, 143, 144, 146, 148, 149, 150, 151, 153, 155, 157, 159, 298, 200, or 301-399 or any ECD portion of a CSR, including an ECD of a FGFR, a VEGFR, IGF1-R and splice variants thereof, such as ECD portions of any CSR described in Table 7 and set forth in any of SEQ ID NOS: 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, or 262 can be used to generate chimeric ECD polypeptides, where all or part of the ECD polypeptide is linked to a multimerization domain. Typically, at least one, but sometimes both, of the ECD portions is all or a portion of a HER family receptor sufficient to bind ligand and/or dimerize (i.e. all or part of a HER1, HER2, HER3, or HER4 molecule) linked to a multimerization domain. Examples of ECD, or portions thereof, of HER family receptors for use as multimerization partners are described herein above and are set forth in any of SEQ ID NOS: 10, 12, 14, 16, 18, 20, 22, 24, 26, 28, 30, 32, 129, 131, 136, 137, and 159. In some examples, at least one of the multimer partners is all or part of the ECD of a HER1 receptor. For example, exemplary of multimeric HER ECD polypeptides is a multimer formed between the ECD, or portion thereof, of HER1/HER3 or HER1/HER4. Additionally, a chimeric ECD polypeptide for use in the formation of an ECD multimer can include hybrid ECD polypeptides linked to a multimerization domain.

In one example, ECD chimeric polypeptides include linkage, directly or indirectly, of an ECD polypeptide with a sequence from an immunoglobulin molecule. In one example, the multimerizing component is an immunoglobulin-derived domain from human IgG, IgM, IgD, IgM, or IgA, or comparable immunoglobulin domains from other animals including, but not limited to mice. In other examples, the multimerizing component is selected from any of the Fc domain of IgG, the heavy chain of IgG, and the light chain of IgG. Typically, the Fc domain of IgG is used, and can be selected from an IgG isotype including IgG1, IgG2, IgG3, and IgG4, as well as any allotype within each isotype group. In most instances, the Fc domain is of IgG1, or a derivative thereof which can be modified for specifically desired properties as described herein. The Fc portion most often contains at least part of the hinge region, and the

CH2 and CH3 domains of an immunoglobulin heavy chain. An exemplary Fc sequence for use as a multimerizing component is set forth in SEQ ID NO:167, but others are known, for example, depending upon the length of the hinge portion used in the Fc sequence. Typically, fusion of an ECD polypeptide is by direct linkage with the Fc sequence, but also can be by indirect linkage such as through peptide linkers or chemical linkers including heterobifunctional crosslinking agents. Generally, the N-terminal ECD, or portion thereof, of a CSR including any HER family receptor, is fused at the C-terminus to the Fc portion of human IgG1, and a linker peptide and/or an epitope tag if necessary.

a. Exemplary Chimeric HER ECD Polypeptides

Chimeric polypeptides included for use in the formation of ECD multimers provided herein include any containing a full-length ECD, or truncated portions thereof, of HER1 and an Fc multimerizing component, and optionally an epitope tag such as a c-myc or His tag for the purification and/or detection of the HER1 ECD chimeric polypeptide. Exemplary HER1-Fc chimeric polypeptides are set forth in SEQ ID NOS: 38 and 40, and encoded by a sequence of nucleotides set forth in SEQ ID NOS: 37 and 39, respectively. For example, the exemplary HER1-Fc chimeric polypeptide set forth as SEQ ID NO:38 (HF110-Fc; HER1-501/Fc; HFD110) contains the truncated ECD sequence of HER1 set forth in SEQ ID NO:10 (corresponding to amino acids 1-501 of SEQ ID NO:38), operatively linked at the N-terminus to a sequence containing a XhoI restriction linker (corresponding to amino acids 502-503), a peptide linker sequence (corresponding to amino acids 504-508), and a sequence for an Fc multimerizing component (corresponding to amino acids 509-739). In another example, the exemplary HER1-Fc chimeric polypeptide set forth as SEQ ID NO:40 (HF100-Fc; HER1-621/Fc; HFD100) contains a full-length ECD sequence of HER1 set forth in SEQ ID NO:12 (corresponding to amino acids 1-621 of SEQ ID NO:40), a peptide linker sequence (corresponding to amino acids 622-626), and a sequence for an Fc multimerizing component (corresponding to amino acids 627-857. In addition, HER1-Fc molecules, including for example the exemplary HF110-Fc and HF100-Fc molecules, can optionally contain an epitope tag. For example, the exemplary HF110-Fc molecule set forth in SEQ ID NO:38 also can optionally include a myc epitope tag set (corresponding to amino acids 740-749 of SEQ ID NO:38). In another example, the HF100-Fc molecule set forth in SEQ ID NO:40, also can optionally include a His epitope tag or other tag (i.e. HFD100T). An exemplary HFD100T molecule is set forth in SEQ ID NO:406 an contains a full-length ECD sequence of HER1 (corresponding to amino acids 1-621 of SEQ ID NO:406), operatively linked at the N-terminus to a sequence containing an XbaI linker (corresponding to amino acids 622-623), a peptide linker sequence (corresponding to amino acids 624-627), a sequence for an Fc multimerizing component (corresponding to amino acids 628-858), a sequence containing an AgeI linker (corresponding to amino acids 859-860), and a sequence for a 6×His tag (corresponding to amino acids 861-866 of SEQ ID NO:406).

Chimeric polypeptides included for use in the formation of ECD multimers provided herein include any containing a full-length ECD, or truncated portions thereof, of HER2 and an Fc multimerizing component, and optionally an epitope tag such as a c-myc tag or His tag for the purification and/or detection of the HER2 ECD chimeric polypeptide. An exemplary HER2-Fc chimeric polypeptides is set forth in SEQ ID NOS: 42, and encoded by a sequence of nucleotides set forth in SEQ ID NO:41. The exemplary HER2-Fc chimeric polypeptide set forth as SEQ ID NO:40 (HF200-Fc; HER2-650/Fc; HFD200) contains the full-length ECD sequence of HER2 set forth in SEQ ID NO:18 (corresponding to amino acids 1-628 of SEQ ID NO:42), operatively linked at the N-terminus to a sequence containing a peptide linker sequence (corresponding to amino acids 629-633), and a sequence for an Fc multimerizing component (corresponding to amino acids 634-864). In addition, HER2-Fc molecules, including for example the exemplary HF200-Fc molecule, can optionally contain an epitope tag.

Chimeric polypeptides included for use in the formation of ECD multimers provided herein include any containing a full-length ECD, or truncated portions thereof, of HER3 and an Fc multimerizing component, and optionally an epitope tag such as a c-myc tag or His for the purification and/or detection of the HER3 ECD chimeric polypeptide. An exemplary HER3-Fc chimeric polypeptide is set forth in SEQ ID NOS: 44 and 46, and encoded by a sequence of nucleotides set forth in SEQ ID NOS: 43 and 45, respectively. For example, the exemplary HER3-Fc chimeric polypeptide set forth in SEQ ID NO:44 (HF310-Fc; HER3-500/Fc; HFD310) contains the truncated ECD sequence of HER3 set forth in SEQ ID NO:20 (corresponding to amino acids 1-500 of SEQ ID NO:44), operatively linked at the N-terminus to a sequence containing a peptide linker sequence (corresponding to amino acids 501-505), and a sequence for an Fc multimerizing component (corresponding to amino acids 506-736). In another example, the exemplary HER3-Fc chimeric polypeptide set forth in SEQ ID NO:46 (HF300-Fc; HER3-621/Fc; HFD300) contains the full-length ECD sequence of HER3 set forth in SEQ ID NO:26 (corresponding to amino acids 1-621 of SEQ ID NO:46), operatively linked at the N-terminus to a sequence containing a peptide linker sequence (corresponding to amino acids 622-626), and a sequence for an Fc multimerizing component (corresponding to amino acids 627-857). In addition, HER3-Fc molecules, including for example the exemplary HF310-Fc and HF300-Fc molecules, can optionally contain an epitope tag.

Chimeric polypeptides included for use in the formation of ECD multimers provided herein include any containing a full-length ECD, or truncated portions thereof, of HER4 and an Fc multimerizing component, and optionally an epitope tag such as a c-myc or His tag for the purification and/or detection of the HER4 ECD chimeric polypeptide. An exemplary HER4-Fc chimeric polypeptides is set forth in SEQ ID NO: 48, and encoded by a sequence of nucleotides set forth in SEQ ID NO:47. The exemplary HER4-Fc chimeric polypeptide set forth as SEQ ID NO:48 (HF400-Fc; HER4-650/Fc; HFD400) contains the full-length ECD sequence of HER4 set forth in SEQ ID NO:32 (corresponding to amino acids 1-625 of SEQ ID NO:48), operatively linked at the N-terminus to a sequence containing a peptide linker sequence (corresponding to amino acids 626-630), and a sequence for an Fc multimerizing component (corresponding to amino acids 631-861). In addition, HER4-Fc molecules, including for example the exemplary HF400-Fc molecule, can optionally contain an epitope tag.

E. ECD Multimers

ECD multimers provided herein contain at least two ECD polypeptides that are stably associated via interactions of their respective multimerization domains. The ECD multimers can be homo-multimers, but most often are heteromultimers where the ECD polypeptide components of the multimer are different. ECD heteromultimers are pan-receptor therapeutics, including pan-HER therapeutics. ECD multimers target several epitopes on HER family members. Thus, the resulting ECD multimeric molecule modulates, typically inhibits, the activity of two or more cognate or interacting CSRs. Modulation can be via interation with one or more ligands and/or via dimerization with a full-length cognate receptor or other interacting CSR. Thus, the multimeric ECD polypeptide bind to one or more ligands, generally two or more ligands, of each of the respective ECD polypeptide and/or dimerize with a cognate receptor or interacting receptor on the cell surface. Thus, the resultant ECD polypeptide multimers are useful as antagonists of cognate CSRs. Such antagonists are useful in treating disease resulting from ligand binding and/or activation of the cognate receptor.

HER family receptors are most often in an inactive form, with only up to 5% of the HER molecules on the transmembrane in an active configuration. Normally, for full-length HER receptors, the mechanism governing the transition of inactive to active form is ligand binding. Ligand binding reorients the orientation of the receptor molecule forcing the dimerization arm to shift from a tethered conformation to a conformation that has the potential to dimerize with another HER molecule. Active forms of HER molecules can be mimicked by forcing dimerization of all or part of the extracellular domain of a HER molecule with a multimerization domain such as, but not limited to, an Fc fragment. Thus, the fusion of a HER ECD with a multimerization domain forces the HER molecule to adopt a ligand-independent activated conformation (i.e. untethered), similar to the constitutively activated HER2 molecule. For example, where the multimerization domain is an Fc molecule, expression of a chimeric polypeptide can be produced as a homodimer where dimerization is forced between two expressed monomeric polypeptides via interactions of the Fc domain. In some instances, such a homodimer can result in improved properties of the ECD polypeptide as compared to a monomeric form of the ECD. In one example, linkage of all or part of a HER ECD with a Fc multimerization domain can create a high affinity receptor complex capable of high ligand binding affinity where the monomeric form of the ECD is unable to bind ligand. For example, as described in Example 4, a monomeric ECD molecule containing the complete ECD of a mature HER1 receptor (i.e. amino acids 1-621) shows only minimal binding to EGF. When the ECD polypeptide is linked to an Fc multimerization domain the ability of the homodimeric HER1 ECD molecule to bind to EGF is greatly increased.

Utilization of this same mechanism for the stabilization of heteromultimers of CSR molecules is proposed for the creation of pan-receptor ECD multimers, including pan-HER ECD multimers, as broad based high affinity receptor therapeutics.

Thus, among the activities of a pan-receptor therapeutic is as a high affinity soluble receptor complex having affinity for more than one ligand. Thus, a pan-receptor multimer can be used as a ligand trap to sequester ligands, including growth factor ligands. The ligands that can be sequestered by the ECD multimer are those that are known to bind or interact with the polypeptide ECDs of the multimer. Where the components of the ECD multimer contain all or a part of one or more ECDs of a HER molecule sufficient to bind ligand, the ECD multimer potentially can sequester any one or more of the ligand combinations set forth in Table 6. For example, at least 10 different ligands can be targets if the multimer is a combination of HER1 and HER4. Alternatively, if the multimer is a combination of HER1 and HER3, any one or more ligands including EGF, amphiregulin, TGF-α, betacellulin, heparin-binding EGF, epiregulin, or neuregulin 1 or 2 (heregulin 1 or 2) can be sequestered by the multimeric molecule. Thus, in some cases where one of the ECD polypeptide components of the multimer is a HER molecule such as, for example, HER1, and the other is all of part of another CSR, the ECD multimer can interact with at least 7 ligands, six of which are ligands recognized by the ECD of HER1 and the remaining one or more ligands recognized by the partner ECD polypeptide. The additional ligand can be a growth factor or other ligand molecule involved in a disease process such as, but not limited to, a proliferative disease, angiogenic disease, or inflammatory disease. Exemplary of such ligands include VEGF, FGF, insulin, HGF, angiopoietin, and others. In an additional example, an ECD multimer that is created from a combination of one or more hybrid ECD polypeptides can be engineered such that it contains sufficient ligand binding portions for two, three, or up to four different CSRs and thus has the ability to sequester 2, 3, 4, 5, 6, 7, 8, 9, 10, 11, 12, 13, 14, 15, or more ligands from their respective full-length CSR.

Modulation of CSRs by ECD multimers provided herein also can be via direct interaction with a cognate or interacting transmembrane receptor. For example, activation of most all RTK receptors is via dimerization with a co-receptor to generate full-length homo- and heterodimeric receptors to allow for autophosphorylation of the catalyitic tail for effector recruitment and downstream signaling. For example, HER receptors dimerize in various combinations as one mechanism to amplify and diversify HER signaling. All combinations of full-length HER receptors have been observed, with HER2 as the most typical dimerization partner. Thus, any interference with the ability of CSRs, particularly RTKS including HERs, to dimerize would impair receptor-mediated signaling. Exemplary of molecules that can impair CSR dimerization are ECD multimers, particularly HER ECD multimers. The activated, high affinity, form of HER ECD multimers that result from fusion with a multimerization domain, for example fusion with an Fc protein, predicts a “back-to-back” conformation that, whether or not bound by ligand, presents the dimerization arm in domain II in a configuration for interaction with transmembrane receptors. Such an interaction could interfere with the ability of a full-length HER receptor to partner with another full-length HER receptor at the transmembrane, thereby inhibiting activation of the receptor. Similar interactions and inhibition is contemplated for other CSR ECDs, including other RTK ECD multimers, that interfere with dimerization of cognate receptors. Thus, in addition or instead of sequestering ligands, a pan-receptor multimer provided herein can dimerize with one or more receptors to inhibit their activity. As described below, activity of a transmembrane receptor can be assessed by assays including, but not limited to, phosphorylation or cell proliferation.

Typically, ECD multimers are dimers, but also can be trimers or higher order multimers depending, for example, on the multimerization domain chosen for multimer formation. For example, an Fc domain will result in a dimeric molecule. In addition, generally a multimerization domain that is a leucine zipper also will result in a dimeric ECD molecule, however, variant forms of leucine zipper such as, for example, a variant GCN4 can be used to create a trimeric or higher ordered multimer. Where higher ordered multimerization domains are desired, multimerization domains can be chosen accordingly. Those of skill in the art are familiar with the structural organizations of exemplary multimerization domain such as, for example, any provided herein.

a. Full-Length HER1 ECD and all or Part of an ECD of Another CSR

Provided herein is an ECD multimer that contains as a first polypeptide a full-length ECD of a HER1 linked to a multimerization domain, and as a second polypeptide all or part of an ECD of another CSR also linked to a multimerization domain. The multimerization domain of the first and second polypeptide can be the same or different, but where different the multimerization domains are complementary to allow for a stable protein-protein interaction between multimer components. Exemplary of a full-length HER1 ECD polypeptide is HF100, which includes amino acids 1-621 of a mature HER1 receptor such as set forth in SEQ ID NO:12, or allelic or species variants thereof. The ECD of a second polypeptide can be all or part of an ECD of any CSR, particularly any CSR involved in a disease process involving proliferation, angiogenesis, or inflammation, so long as the ECD polypeptide is not a full-length HER2 molecule. The ECD of a second polypeptide, however, can be part of the ECD of a HER2 molecule sufficient to dimerize with other HER molecules. Exemplary of truncated HER2 ECD polypeptides include the HF220 molecule set forth in SEQ ID NO:18 and the HF210 molecule set forth in SEQ ID NO: 16, and allelic variants thereof. In some instances, an ECD multimer containing the full-length HER1 molecule and the truncated HER2 molecule HF210 is preferred, as the presence of modules 2-5 in subdomain IV of the truncated HER2 molecule influences the dimerization ability of the truncated HER2 molecule, such as is described in Example 5.

An ECD multimer containing as a first polypeptide a full-length HER1 ECD, can have as its second polypeptide component all or part of an ECD of a HER3 or HER4 receptor. Particular of such an ECD multimer is one that has the capability of binding two or more ligands from among an EGF, amphiregulin, TGF-α, betacellulin, heparin-binding EGF, or epiregulin, and one or more neuregulin. Such a polypeptide also can dimerize with any one or more of the HER receptors. For example, an ECD multimer that is combined with all or part of a HER4 ECD polypeptide has the capacity to bind any of neregulins 1-4, including any isoforms thereof. Exemplary of such an ECD multimer is one where the first polypeptide of the multimer is a full-length HER1 ECD (i.e. HF100 set forth in SEQ ID NO:12, or allelic variants thereof) and the second polypeptide is a truncated HER4 polypeptide competent to bind ligand such as, but not limited to, the HF410 molecule set forth in SEQ ID NO:28, or allelic variants of. The HER4 portion of the ECD multimer also can be a full-length HER4 molecule containing the complete ECD portion of a mature HER4 receptor such as is set forth in SEQ ID NO:32 (i.e. HF400). In some examples, multimerization of a HER1 ECD and all or part of a HER4 ECD is mediated via a multimerization domain. For example, the exemplary chimeric polypeptides set forth in SEQ ID NO:40 (HF100-Fc, or an epitope tagged version such as is set forth in SEQ ID NO:406) and set forth in SEQ ID NO:48 (HF400-Fc) can be co-expressed to produce a multimeric molecule.

Typcially, however, a full length HER1 ECD polypeptide is combined in a multimer with all or part of a HER3 ECD polypeptide such that the resulting multimer has the capacity to bind any of neregulins 1 or 2, including any isoforms thereof and/or dimerize with any one or more HER receptors on the cell surface. Exemplary of such an ECD multimer is one where the first polypeptide is a full-length HER1 ECD and the second polypeptide of the multimer is all or a portion of a HER3 polypeptide. HER1 and HER3 are two of the most commonly overexpressed receptors. Thus, an ECD multimer of HER1 and HER3 has the ability to trap ligands binding to two of the most commonly overexpressed receptors, while sparing some ligands that bind to HER4 (i.e. neuregulin 3 and neuregulin 4), which has not been shown to have a broad activity in cancer (Barnes et al. (2005) Clin Cancer Res 11:2163-8; Srinivasan et al. (1998) J Pathol. 185:236-45).

In one example, an ECD multimer of a HER1 ECD and a HER3 ECD can include as a first polypeptide a full-length of a HER1 ECD, and as a second polypeptide a truncated HER3 ECD polypeptide, where each polypeptide is linked to a multimerization domain. As mentioned above, exemplary of a full-length HER1 molecule is the HF100 molecule (SEQ ID NO:12), or allelic variants thereof. Any truncated HER3 ECD polypeptide is contemplated so long as it retains its ability to bind any one or more of a neuregulin 1 or 2 isoforms and/or to dimerize. Exemplary of such truncated HER3 ECD polypeptides include HF310 set forth in SEQ ID NO:20, p85HER3 set forth in SEQ ID NO:22, or ErbB3-519 set forth in SEQ ID NO:24, or allelic variants thereof. For example, the exemplary chimeric polypeptides set forth in SEQ ID NO:40 (HF100-Fc, or an epitope tagged version thereof such as is set forth in SEQ ID NO:406) and set forth in SEQ ID NO:44 (HF310-Fc) can be co-expressed to produce a multimeric molecule.

In another example, an ECD multimer of a HER1 ECD and a HER3 ECD can include as a first polypeptide a full-length of a HER1 ECD, such as the HF100 molecule (SEQ ID NO:12), and as a second polypeptide a full-length HER3 ECD molecule, where each polypeptide is linked to a multimerization domain. An exemplary full-length HER3 ECD molecule includes amino acids 1-621 of a mature HER3 full-length receptor, such as set forth in SEQ ID NO:26 (HF300). A full-length ECD multimer of HER1/HER3 can be linked by interactions of their respective multimerization domains. The multimerization domain of the first full-length HER1 ECD polypeptide and second HER3 ECD polypeptide can be the same or different, but where different the multimerization domains are complementary to allow for a stable protein-protein interaction between multimer components. In one example, each of the first and second polypeptides are linked to an Fc fragments such as, but not limited to, an IgG1 Fc fragment. Exemplary of full-length HER1 and HER3 ECD chimeric polypeptides linked to an Fc fragment are set forth in SEQ ID NO:40 or SEQ ID NO:46, respectively. Thus, a HER1/HER3 ECD multimer can be formed upon co-expression of a nucleic acid sequence encoded a polypeptide having an amino acid sequence set forth in SEQ ID NO:40 (or an epitope tagged version thereof such as set forth in SEQ ID NO:406) and SEQ ID NO:46 (or an epitope tagged version thereof such as set forth in SEQ ID NO:407), or allelic variants thereof. In addition, if necessary, either or both of the sequences of the chimeric polypeptides set forth in SEQ ID NO:40 or SEQ ID NO:46 can contain the addition of an epitope tag such as a c-myc of His tag, which then can be incorporated into the resulting HER1/HER3 ECD multimer. For example, a multimer can be generated where one or both chimeric polypeptides has a sequence of amino acids set forth in SEQ ID NO:406 and/or SEQ ID NO:407.

Additionally, the second polypeptide that can be combined with a full-length HER1 ECD to form an ECD multimer can be a CSR ECD polypeptide of any length so long as the second ECD polypeptide retains its ability to bind to ligand and/or dimerize. Exemplary ECD polypeptides that can be combined in a multimer with a full-length HER1 ECD polypeptide include but are not limited to all of part of VEGFR1 or 2, FGFR1-4, IGF1-R, Tie-1, Tie-2, MET, PDGFRA or B, PDGFRB, Epha1-8, TNFR, RAGE, or any other CSR involved in a disease process characterized by proliferative, angiogenic, or inflammatory components. Exemplary sequences of full-length ECD polypeptides of exemplary CSRs are set forth in Table 7. Portions thereof sufficient to bind ligand are known in the art as described herein for some exemplified RTKs. If not known, the subdomains required for ligand binding can be empirically determined based on alignments with related receptors and/or by using recombinant DNA techniques in concert with ligand binding assays. Other CSRs, and ECD portions thereof, contemplated for use in a multimer with a full-length HER1 ECD polypeptide can be empirically determined based on the disease to be treated, and/or on the contribution of a CSR to resistance to drugs targeted to a single cell surface receptor. In addition, alternatively spliced isoforms of any CSR can be used in multimers with a full-length HER1 ECD polypeptide. Exemplary of these are isoforms of IGF-1R such as are described in Example 11, and set forth as SEQ ID NOS: 298-300. Other CSR isoforms that can be used in ECD multimers are set forth in any of SEQ ID NOS: 301-384.

b. Two or More Truncated ECD Components

Also provided herein is an ECD multimeric molecule formed between two or more truncated ECD portions of any CSR ECD, where at least one of the CSRs is a shortened HER molecule. Typically, at least one of the truncated ECD portion is sufficient to bind ligand and/or dimerize with a CSR, typically both, unless the truncated ECD polypeptide is derived from HER2 in which case the polypeptide portion must at least be competent to dimerize with another cell surface receptor. Such a molecule can act as a pan-receptor therapeutic by modulating, typically inhibiting, one or more of a HER receptor and/or another CSR. Modulation can be by sequestering ligand and/or by dimerizing with the CSR. In some examples, each of the first and second polypeptide components can be linked directly or indirectly via a multimerization domain. The multimerization domain of the first and second polypeptide can be the same or different, but where different the multimerization domains are complementary to allow for a stable protein-protein interaction between multimer components. The ECD multimer can be formed between two shortened HER polypeptides, typically truncated ECD polypeptides of different HER receptors that retain their ligand binding ability and/or dimerize. One of skill in the art can determine the portions of HER molecules to use in creating the ECD multimer, such that at least one, typically both, of the shortend HER polypeptides retain their ability to bind ligand and/or to dimerize. For example, generally a truncated HER1, 2, or 3 molecule contains a sufficient portion of subdomains I and III to bind ligand, a sufficient portion of subdomain II to dimerize, and at least module I of subdomain IV. A truncated HER2 molecule generally contains at least a sufficient portion of subdomains I, II, and III, and at least modules 2-5 of subdomain IV to dimerize.

Any combination of a truncated HER ECD is contemplated for use in a hybrid ECD multimer. For example, a truncated HER1 ECD polypeptide can be combined with a truncated HER2, HER3, or HER4 polypeptide; a truncated HER2 ECD polypeptide can be combined with a truncated HER3 or HER4 ECD polypeptide; and a truncated HER3 polypeptide can be combined with a truncated HER4 ECD polypeptide. Exemplary of truncated HER polypeptides include any described herein such as, for example, any set forth in SEQ ID NOS: 10, 14, 16, 20, 24, 28, 30, 34, alternative splice variants of a HER receptor, for example any set forth in SEQ ID NOS: 22, 127, 129, 131, 133, 135, 136, 137, 138, 139, 141, 143, 144, 146, 148, 149, 150, 151, 153, 155, 157, or 159, or any allelic or species variants thereof. In one example a herstatin molecule or variant thereof (such as set forth in any of SEQ ID NOS:135, or 385-399) can be combined with any other truncated ECD HER polypeptide. In one example, an ECD multimer can include as a first polypeptide part of a HER1 ECD, and as a second polypeptide part of a HER3 ECD polypeptide, where each polypeptide is linked to a multimerization domain. Exemplary of a truncated HER1 molecule is HF110 (SEQ ID NO:10), or allelic variants thereof. Exemplary of a truncated HER3 molecule is HF310 (SEQ ID NO:20), p85-HER3 (SEQ ID NO:22), or ErbB3-519 (SEQ ID NO:24, or allelic variants thereof. For example, the exemplary chimeric polypeptide set forth in SEQ ID NO:38 (HER1-501/Fc; HFD110, with or without a c-myc tag) and the chimeric polypeptide set forth in SEQ ID NO:44 (HER3-500/Fc; HFD310) can be coexpressed to produce a multimeric molecule that is a truncated HER1/HER3 ECD heteromultimer.

In other examples, an ECD multimer provided herein can contain as a first polypeptide a truncated HER ECD polypeptide and as a second polypeptide another truncated CSR ECD polypeptide that is not of the HER family of receptors. As above, the truncated HER ECD polypeptide can be a portion of an ECD of a HER1, HER2, HER3, or HER4 receptor so long as at least one of the polypeptide components of the multimer, typically both, binds to ligand and/or dimerizes with a transmembrane receptor. Exemplary truncated HER family receptors include, but are not limited to, any set forth in any of SEQ ID NOS: 10, 14, 16, 20, 22, 24, 26, 28, 30, 34, 127, 129, 131, 133, 135, 136, 137, 138, 139, 141, 143, 144, 146, 148, 149, 150, 151, 153, 155, 157, 159, or 385-399, or any allelic or species variants thereof. A chimeric ECD polypeptide can include all or part of a ECD polypeptide of a another cell surface receptor linked to a multimerization domain. Any truncated ECD CSR combination is contemplated herein to form an ECD multimer with a shortened HER ECD polypeptide, and can be empirically determined based on the disease to be treated, the contribution of a respective CSR to that disease, the known ligands for the CSR, the contribution of a CSR to resistance to drugs targeted to a single cell surface receptor, and other factors. Exemplary of CSRs are described herein above and include, but are not limited to, IGF-R1, VEGFR (i.e. VEGFR1 or VEGFR2), FGFR (i.e. FGFR1, FGFR2, FGFR3, or FGFR4), TNFR, PDGFRA or PDGFRB, MET, Tie (Tie-1 or Tie-2), an Eph receptor, or a RAGE. Exemplary sequences of full-length ECD polypeptides of exemplary CSRs are set forth in Table 7. Portions thereof sufficient to bind ligand are known in the art such as is described herein for some exemplified RTKs. If not known, the subdomains required for ligand binding can be empirically determined based on alignments with related receptors and/or by using recombinant DNA techniques in concert with ligand binding assays. In addition, alternatively spliced isoforms of any CSR can be used in multimers. Exemplary of these are isoforms of IGF-1R such as are described in Example 11, and set forth as SEQ ID NOS: 298-300. Other CSR isoforms that can be used in ECD multimers are set forth in any of SEQ ID NOS: 301-384.

c. Hybrid ECD Multimers

Provided herein are ECD multimers where at least one or both of the chimeric ECD polypeptides of the multimer is a hybrid ECD molecule containing ligand binding domains and/or dimerization domains from part of the ECD portion of any two or more CSR linked to a multimerization domain. Such hybrid ECD molecules are described herein above. For example, one such hybrid ECD polypeptide contains subdomain II from HER2 and subdomains I and III, which can be from the same or different receptor, from HER1, 3 or 4. Other combinations of a hybrid ECD can be empirically determined based on the known subdomain activities of relevant CSRs. Typically, at least one of the subdomains of one of the ECD hybrids confers dimerization ability to the resulting ECD multimer. Two or more of the same or different hybrid ECD molecules can be linked together directly or indirectly. In one example, the hybrid ECD molecules can be linked via fusion of a first hybrid ECD polypeptide with a multimerization domain and fusion of a second hybrid ECD polypeptide with the same or complementary multimerization domain. Formation of a hybrid ECD multimer is accomplished following co-expression of the respective encoding nucleic acid for the first and second polypeptide.

Additionally, ECD multimers can be formed where only one of the polypeptides of the multimer is a hybrid ECD and the second polypeptide is all or part of any other CSR molecule, such as for example any full-length ECD polypeptide described above or any truncated ECD polypeptide described above. Typically, the other CSR ECD polypeptide is all or part of a HER family receptor, alternative spliced isoforms of HER family receptors, or allelic variants thereof. Other CSRs, other than HER family receptors, can be combined with a hybrid ECD and can be selected as appropriate depending on the disease to be treated and/or the association of the CSR to resistance to drugs targeted to a single cell surface receptor.

d. ECD Components that are the Same or Derived from the Same CSR

Also provided herein are homo- or hetero-multimers that modulate at least one, sometimes two or more CSRs, by sequestering ligand and/or by directly interacting with a cognate CSR or other interacting CSR. Such ECD multimers can be homomultimers, typically homodimers, of a first ECD polypeptide linked to a multimerization domain, and a second ECD polypeptide linked to a multimerization domain where the first and second polypeptide are the same. Alternatively, such ECD multimers can be heteromultimers, where each of the first and second ECD polypeptide are derived from the same cognate CSR, but are different. Typically, but not always, where the ECD components are the same or derived from the same receptor, the activity of only a single receptor will be targeted. For example, in some instances, an ECD multimer that has as a first polypeptide a full-length IGF1-R ECD (i.e. corresponding to amino acids 31-935 of SEQ ID NO:260) and as a second polypeptide the same polypeptide as the first, or a truncated or isoform thereof, is a candidate thereaputic for modulating the activity of at least a full-length IGF1-R. In another example, a homo- or hetero-multimer containing a herstatin and/or another HER2 ECD component is a candidate for modulating at least one, but typically two or more CSRs, such as by directly interacting with full-length HER1, HER3, or HER4 receptors on the cell surface.

### F. Methods of Producing Nucleic Acid Encoding Chimeric ECD polypeptide Fusions and Production of the Resulting ECD Multimers

Any suitable method for generating the chimeric polypeptides between ECDs, portions thereof, particularly portions sufficient for ligand binding and/or receptor dimerization, and also alternatively splice portions, and a multimerization domain can be used. Similarly, formation of multimers from the chimeric polypeptides, can be achieved by any method known to those of skill in the art. As noted, the multimers typically include and ECD from at least one HER family member, typically a HER1 or a HER3 or HER4, and a second HER family member and/or an ECD from a CSR, such as IGF1-R, a VEGFR, and FGFR or other receptor involved in tumorigenesis or inflammatory or other disease processes.

Exemplary methods for generating nucleic acid molecules encoding ECD chimeric polypeptides, including ECD polypeptides linked directly or indirectly, to a multimerization domain described herein, are provided. Such methods include in vitro synthesis methods for nucleic acid molecules such as PCR, synthetic gene construction and in vitro ligation of isolated and/or synthesized nucleic acid fragments. Nucleic acid molecules for CSR, including HER family receptors or other RTKs, can be isolated by cloning methods, including PCR of RNA and DNA isolated from cells and screening of nucleic acid molecule libraries by hybridization and/or expression screening methods.

ECD polypeptides, or portions thereof, can be generated from nucleic acid molecules encoding ECD polypeptides using in vitro and in vivo synthesis methods. ECD multimers, containing one or more chimeric ECD polypeptide such as, for example, ECD-Fc protein fusions or linkage of ECDs with any other multimerization domain, can be generated following expression in any organism suitable to produce the required amounts and forms of ECD polypeptide multimers needed for administration and treatment. Expression hosts include prokaryotic and eukaryotic organisms such as E. coli, yeast, plants, insect cells, mammalian cells, including human cell lines and transgenic animals. ECD polypeptides or ECD polypeptide multimers also can be isolated from cells and organisms in which they are expressed, including cells and organisms in which ECD polypeptides are produced recombinantly and those in which isoforms are synthesized without recombinant means such as genomically-encoded isoforms produced by alternative splicing events.

1. Synthetic Genes and Polypeptides

Nucleic acid molecules encoding ECD polypeptides can be synthesized by methods known to one of skill in the art using synthetic gene synthesis. In such methods, a polypeptide sequence of an ECD is “back-translated” to generate one or more nucleic acid molecules encoding an ECD, or portion thereof. The back-translated nucleic acid molecule is then synthesized as one or more DNA fragments such as by using automated DNA synthesis technology. The fragments are then operatively linked to form a nucleic acid molecule encoding an ECD polypeptide. Chimeric ECD polypeptide can be generated by joining nucleic acid molecules encoding an ECD polypeptide with additional nucleic acid molecules such as any encoding a multimerization domain, or other nucleic acid encoding an epitope or fusion tags, regulatory sequences for regulating transcription and translation, vectors, and other polypeptide-encoding nucleic acid molecules. ECD-encoding nucleic acid molecules also can be operatively linked with other fusion tags or labels such as for tracking, including radiolabels, and fluorescent moieties.

The process of backtranslation uses the genetic code to obtain a nucleotide gene sequence for any polypeptide of interest, such as an ECD polypeptide. The genetic code is degenerate, 64 codons specify 20 amino acids and 3 stop codons. Such degeneracy permits flexibility in nucleic acid design and generation, allowing for example, the incorporation of restriction sites to facilitate the linking of nucleic acid fragments and/or the placement of unique identifier sequences within each synthesized fragment. Degeneracy of the genetic code also allows the design of nucleic acid molecules to avoid unwanted nucleotide sequences, including unwanted restriction sites, splicing donor or acceptor sites, or other nucleotide sequences potentially detrimental to efficient translation. Additionally, organisms sometimes favor particular codon usage and/or a defined ratio of GC to AT nucleotides. Thus, degeneracy of the genetic code permits design of nucleic acid molecules tailored for expression in particular organisms or groups of organisms. Additionally, nucleic acid molecules can be designed for different levels of expression based on optimizing (or non-optimizing) of the sequences. Back-translation is performed by selecting codons that encode a polypeptide. Such processes can be performed manually using a table of the genetic code and a polypeptide sequence. Alternatively, computer programs, including publicly available software can be used to generate back-translated nucleic acid sequences.

To synthesize a back-translated nucleic acid molecule, any method available in the art for nucleic acid synthesis can be used. For example, individual oligonucleotides corresponding to fragments of an ECD-encoding sequence of nucleotides are synthesized by standard automated methods and mixed together in an annealing or hybridization reaction. Such oligonucleotides are synthesized such that annealing results in the self-assembly of the gene from the oligonucleotides using overlapping single-stranded overhangs formed upon duplexing complementary sequences, generally about 100 nucleotides in length. Single nucleotide “nicks” in the duplex DNA are sealed using ligation, for example with bacteriophage T4 DNA ligase. Restriction endonuclease linker sequences can, for example, then be used to insert the synthetic gene into any one of a variety of recombinant DNA vectors suitable for protein expression. In another, similar method, a series of overlapping oligonucleotides are prepared by chemical oligonucleotide synthesis methods. Annealing of these oligonucleotides results in a gapped DNA structure. DNA synthesis catalyzed by enzymes such as DNA polymerase I can be used to fill in these gaps, and ligation is used to seal any nicks in the duplex structure. PCR and/or other DNA amplification techniques can be applied to amplify the formed linear DNA duplex.

Additional nucleotide sequences can be joined to an ECD-encoding nucleic acid molecule thereby generating an ECD fusion, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to an ECD-encoding nucleic acid molecule. Examples of such sequences include, but are not limited to, promoter sequences designed to facilitate intracellular protein expression, or precursor sequences designed to facilitate protein secretion. Other examples of nucleotide sequences that can be operatively linked to an ECD-encoding nucleic acid molecule include sequences that facilitate the purification and/or detection of a polypeptide. For example, a fusion tag such as an epitope tag or fluorescent moiety can be fused or linked to an isoform. Additional nucleotide sequences such as sequences specifying protein binding regions also can be linked to ECD-encoding nucleic acid molecules. Such regions include, but are not limited to, sequences to facilitate uptake of an ECD polypeptide into specific target cells, or otherwise enhance the pharmacokinetics of the synthetic gene.

ECD polypeptides also can be synthesized using automated synthetic polypeptide synthesis. Cloned and/or in silico-generated polypeptide sequences can be synthesized in fragments and then chemically linked. Alternatively, chimeric molecules can be synthesized as a single polypeptide. Such polypeptides then can be used in the assays and treatment administrations described herein.

2. Methods of Cloning and Isolating ECD Polypeptides

ECD-encoding nucleic acid molecules, including ECD fusion-encoding nucleic acid molecules, can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Nucleic acid molecules encoding ECD polypeptides also can be isolated using library screening. For example, a nucleic acid library representing expressed RNA transcripts as cDNAs can be screened by hybridization with nucleic acid molecules encoding ECD polypeptides or portions thereof. For example, a nucleic acid sequence encoding a portion of an ECD polypeptide, such as for example, a portion of module 1 of domain IV of a HER family ECD, can be used to screen for domain IV-containing molecules based on hybridization to homologous sequences.

Expression library screening can be used to isolate nucleic acid molecules encoding an ECD polypeptide. For example, an expression library can be screened with antibodies that recognize a specific ECD or a portion of an ECD. Antibodies can be obtained and/or prepared which specifically bind an ECD polypeptide or a region or peptide contained in an ECD. Antibodies which specifically bind an ECD can be used to screen an expression library containing nucleic acid molecules encoding an ECD, such as an ECD of a HER family receptor. Methods of preparing and isolating antibodies, including polyclonal and monoclonal antibodies and fragments therefrom are well known in the art. Methods of preparing and isolating recombinant and synthetic antibodies also are well known in the art. For example, such antibodies can be constructed using solid phase peptide synthesis or can be produced recombinantly, using nucleotide and amino acid sequence information of the antigen binding sites of antibodies that specifically bind a candidate polypeptide. Antibodies also can be obtained by screening combinatorial libraries containing of variable heavy chains and variable light chains, or of antigen-binding portions thereof. Methods of preparing, isolating and using polyclonal, monoclonal and non-natural antibodies are reviewed, for example, in Kontermann and Dubel, eds. (2001) “Antibody Engineering” Springer Verlag; Howard and Bethell, eds. (2001) “Basic Methods in Antibody Production and Characterization” CRC Press; and O'Brien and Aitkin, eds. (2001) “Antibody Phage Display” Humana Press. Such antibodies also can be used to screen for the presence of an ECD polypeptide, for example, to detect the expression of a ECD polypeptide in a cell, tissue or extract.

Methods for amplification of nucleic acids can be used to isolate nucleic acid molecules encoding an ECD polypeptide, include for example, polymerase chain reaction (PCR) methods. A nucleic acid containing material can be used as a starting material from which an ECD-encoding nucleic acid molecule can be isolated. For example, DNA and mRNA preparations, cell extracts, tissue extracts, fluid samples (e.g. blood, serum, saliva), samples from healthy and/or diseased subjects can be used in amplification methods. Nucleic acid libraries also can be used as a source of starting material. Primers can be designed to amplify an ECD molecule. For example, primers can be designed based on expressed sequences from which an ECD molecule is generated. Primers can be designed based on back-translation of an ECD amino acid sequence. Nucleic acid molecules generated by amplification can be sequenced and confirmed to encode an ECD.

3. Methods of Generating and Cloning ECD Polypeptide Chimeras

Chimeric proteins are polypeptides that comprise two or more regions derived from different, or heterologous, proteins or peptides. Chimeric proteins can contain several sequences, including a signal peptide sequence, one or more sequences for an ECD of a CSR such as a HER family receptor, or portion thereof, and any other heterologous sequence such as a linker sequence, a multimerization domain sequence (i.e. Fc domain, leucine zipper, or other multimer-forming sequence), and/or sequences for epitope tags or other moieties that facilitate protein purification. For example, an ECD polypeptide can be linked directly to another polypeptide (i.e. another ECD polypeptide or portion thereof and/or a multimerization domain) to form a fusion protein. Alternatively, the proteins can be separated by a distance sufficient to ensure that the protein forms proper secondary and tertiary structures. Suitable linker sequences (1) will adopt a flexible extended conformation, (2) will not exhibit a propensity for developing an ordered secondary structure which could interact with the functional domains of the fusion polypeptide, and (3) will have minimal hydrophobic or charged character which could promote interaction with the functional protein domains. Exemplary linker sequences are discussed above and generally include those containing Gly, Asn, or Ser, or other neutral amino acids including Thr or Ala. Generally, linkage of an ECD portion with a heterologous sequence is by recombinant DNA techniques as described above. Alternatively, the heterologous sequence can be covalently linked to the ECD portion by heterobifunctional crosslinking agents, such as any described herein.

Generally, an ECD fusion molecule encodes a chimeric polypeptide having all or part of an ECD of a CSR sufficient to bind ligand linked to a heterologous polypeptide that facilitates multimer formation, such as a multimerization domain.

Additionally, an ECD polypeptide also can be linked, directly or indirectly, to one or more other heterologous sequences. For example, an ECD chimeric polypeptide also can include fusion with a tag polypeptide, which provides an epitope to which an anti-tag antibody can selectively bind. Such epitope tagged forms of ECD polypeptide fusions are useful, as the presence of the presence thereof can be detected using a labeled antibody against the tag polypeptide. Also, provision of the epitope tag allows the ECD fusion polypeptide to be readily purified by affinity purification using an anti-tag antibody.

Chimeric proteins can be prepared using conventional techniques of enzyme cutting and ligation of fragments from desired sequences. For example, desired sequences can be synthesized using an oligonucleotide synthesizer, isolated from the DNA of a parent cell which produces the protein by appropriate restriction enzyme digestion, or obtained from a target source, such as a cell, tissue, vector, or other target source, by PCR of genomic DNA with appropriate primers. In one example, ECD chimeric sequences can be generated by successive rounds of ligating DNA target sequences, amplified by PCR, into a vector at engineered recombination site. For example, a nucleic acid sequence for one or more ECD polypeptides, fusion tag, and/or a multimerization domain sequence can be PCR amplified using primers that hybridize to opposite strands and flank the region of interest in a target DNA. Cells or tissues or other sources known to express a target DNA molecule, or a vector containing a sequence for a target DNA molecule, can be used as a starting product for PCR amplification events. The PCR amplified product can be subcloned into a vector for further recombinant manipulation of a sequence, such as to create a fusion with another nucleic acid sequence already contained within a vector, or for the expression of a target molecule.

PCR primers used in the PCR amplification also can be engineered to facilitate the operative linkage of nucleic acid sequences. For example, non-template complementary 5′ extension can be added to primers to allow for a variety of post-amplification manipulations of the PCR product without significant effect on the amplification itself. For example, these 5′ extensions can include restriction sites, promoter sequences, restriction enzyme linker sequences, a protease cleavage site sequence or sequences for epitope tags. In one example, for the purpose of creating a fusion sequence, sequences that can be incorporated into a primer include, for example, a sequence encoding a myc epitope tag or other small epitope tag, such that the amplified PCR product effectively contains a fusion of a nucleic acid sequence of interest with an epitope tag.

In another example, incorporation of restriction enzyme sites into a primer can facilitate subcloning of the amplification product into a vector that contains a compatible restriction site, such as by providing sticky ends for ligation of a nucleic acid sequence. Subcloning of multiple PCR amplified products into a single vector can be used as a strategy to operatively link or fuse different nucleic acid sequences. Examples of restriction enzyme sites that can be incorporated into a primer sequence can include, but are not limited to, an Xho I restriction site (CTCGAG, SEQ ID NO:267), an NheI restriction site (GCTAGC, SEQ ID NO:268), a Not I restriction site (GCGGCCGC, SEQ ID NO: 269), an EcoRI restriction site (GAATTC, SEQ ID NO:270), an AgeI site (ACCGGT, SEQ ID NO:271) or an Xba I restricition site (TCTAGA, SEQ ID NO:272). Other methods for subcloning of PCR products into vectors include blunt end cloning, TA cloning, ligation independent cloning, and in vivo cloning.

The creation of an effective restriction enzyme site into a primer requires the digestion of the PCR fragment with a compatible restriction enzyme to expose sticky ends, or for some restriction enzyme sites, blunt ends, for subsequent subcloning. There are several factors to consider in engineering a restriction enzyme site into a primer so that it retains its compatibility for a restriction enzyme. First, the addition of 2-6 extra bases upstream of an engineered restriction site in a PCR primer can greatly increase the efficiency of digestion of the amplification product. Other methods that can be used to improve digestion of a restriction enzyme site by a restriction enzyme include proteinase K treatment to remove any thermostable polymerase that can block the DNA, end-polishing with Klenow or T4 DNA polymerase, and/or the addition of spermidine. An alternative method for improving digestion efficiency of PCR products also can include concatamerization of the fragments after amplification. This is achieved by first treating the cleaned up PCR product with T4 polynucleotide kinase (if the primers have not already been phosphorylated). The ends may already be blunt if a proofreading thermostable polymerase such as Pfu was used or the amplified PCR product can be treated with T4 DNA polymerase to polish the ends if a non-proofreading enzyme such as Taq is used. The PCR products can be ligated with T4 DNA ligase. This effectively moves the restriction enzyme site away from the end of the fragments and allows for efficient digestion.

Prior to subcloning of a PCR product containing exposed restriction enzyme sites into a vector, such as for creating a fusion with a sequence of interest, it is sometimes necessary to resolve a digested PCR product from those that remain uncut. In such examples, the addition of fluorescent tags at the 5′ end of a primer can be added prior to PCR. This allows for identification of digested products since those that have been digested successfully will have lost the fluorescent label upon digestion.

In some instances, the use of amplified PCR products containing restriction sites for subsequent subcloning into a vector for the generation of a fusion sequence can result in the incorporation of restriction enzyme linker sequences in the fusion protein product. Generally such linker sequences are short and do not impair the function of a polypeptide so long as the sequences are operatively linked.

The nucleic acid molecule encoding an ECD chimeric polypeptide can be provided in the form of a vector which comprises the nucleic acid molecule. One example of such a vector is a plasmid. Many expression vectors are available and known to those of skill in the art and can be used for expression of an ECD polypeptide, including chimeric ECD polypeptide. The choice of expression vector can be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector. In addition, many expression vectors offer either an N-terminal or C-terminal epitope tag adjacent to the multiple cloning site so that any resulting protein expressed from the vector will have an epitope tag inserted in frame with the polypeptide sequence. An exemplary expression vector with an inserted epitope tag is the pcDNA/myc-His mammalian expression vector (Invitrogen, SEQ ID NO:161). Thus, for example, expression of an ECD polypeptide from this vector result in the expression of a polypeptide containing a C-terminal myc-His tag, where the myc-His tag has a sequence of amino acids set forth in SEQ ID NO:162. Thus, any ECD polypeptide, or portion thereof, can be expressed with a myc-His tag. Such exemplary polypeptides that contain a tag are described in the Examples and are designated with a “T”, for example, a HER1-621(T) molecule is a polypeptide containing the full-length of a HER1 ECD followed by a C-terminal myc-His tag. Exemplary sequences of ECD polypeptides provided herein containing an epitope tag sequence are set forth in SEQ ID NO:274 and 275. Any ECD polypeptide, or truncated portion thereof, can be generated by any method known to one of skill in the art that contains an epitope tag such as, but not limited to, a c-myc tag, a His tag, or a c-myc/His tag combination as set forth in SEQ ID NO:162.

4. Expression Systems

DNA encoding a chimeric polypeptide, such as any provided herein, is transfected into a host cell for expression. In some instances where ECD multimeric polypeptides are desired whereby multimerization is mediated by a multimerization domain, then the host cell is transformed with DNA encoding separate chimeric ECD molecules that will make the multimer, with the host cell optimally being selected to be capable of assembling the separate chains of the multimer in the desired fashion. Assembly of the separate monomer polypeptides is facilitated by interaction of each respective multimerization domain, which is the same or complementary between chimeric ECD polypeptides. Where HER family receptor ECDs, or portions thereof, are one or both ECD portions of the multimeric polypeptide, the multimerization domain is selected such that assembly of the monomers orients the dimerization arm of the HER molecule away from the partner multimer molecule. This orientation is referred to as “back-to-back” and ensures that the dimerization arm is accessible for dimerization with a cognate HER on the cell surface.

ECD polypeptides, including chimeric ECD polypeptides, can be expressed in any organism suitable to produce the required amounts and form of polypeptide needed for administration and treatment. Generally, any cell type that can be engineered to express heterologous DNA and has a secretory pathway is suitable. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

a. Prokaryotic Expression

Prokaryotes, especially E. coli, provide a system for producing large amounts of proteins such as ECD polypeptides and ECD polypeptide fusions provided herein. Other microbial strains may also be used, such as bacilli, for example Bacillus subtilis, various species of Pseudomonas, or other bacterial strains. Transformation of bacteria, including E. coli, is a simple and rapid technique well known to those of skill in the art. In such prokaryotic systems, plasmid vectors which contain replications sites and control sequences derived from a species compatible with the host are often used. For example, common vectors for E. coli include PBR322, pUC18, pBAD, and their derivatives. Commonly used prokaryotic control sequences, which contain promoters for transcription initiation, optionally with an operator, along with ribosome binding-site sequences, include such commonly used promoters as the beta-lactamase (penicillinase) and lactose (lac) promoter systems, the tryptophan (trp) promoter system, the arabinose promoter, and the lambda-derived P1 promoter and N-gene ribosome binding site. Any available promoter system compatible with prokaryotes, however, can be used. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

ECD polypeptides can be expressed in the cytoplasmic environment of E. coli. The cytoplasm is a reducing environment and for some molecules, this can result in the formation of insoluble inclusion bodies. Reducing agents such as dithiothreotol and β-mercaptoethanol and denaturants, such as guanidine-HC1 and urea can be used to resolubilize the proteins. An alternative approach is the expression of ECD polypeptides, including ECD polypeptide fusions, in the periplasmic space of bacteria which provides an oxidizing environment and chaperonin-like and disulfide isomerases and can lead to the production of soluble protein. In some examples, a precursor or signal sequence for use in bacteria including an OmpA, OmpF, Pe1B, or other precursor sequence, is fused to the protein to be expressed, such as by replacing an endogenous precursor sequence, which directs the protein to the periplasm. The leader peptide is then removed by signal peptidases inside the periplasm. Examples of periplasmic-targeting precursor or leader sequences include the pe1B leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast

Yeasts such as Saccharomyces cerevisae, Schizosaccharomyces pombe, Yarrowia lipolytica, Kluyveromyces lactis and Pichia pastoris are well known yeast expression hosts that can be used for production of ECD polypeptides. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoter. Other yeast promoters include promoters for synthesis of glycolytic enxymes, e.g., those for 3-phosphoglycerate kinase, or those from the enolase gene or the Leu2 gene obtained from Yep13. Expression vectors often include a selectable marker such as LEU2, TRP1, HIS3 and URA3 for selection and maintenance of the transformed DNA. An exemplary expression vector system for use in yeast is the POT1 vector systems (see e.g., U.S. Pat. No. 4,931,373), which allows transformed cells to be selected by growth in glucose-containing media. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase, or any other heterologous or homologous precursor sequence that promotes the secretion of a polypeptide in yeast. A protease cleavage site such as for example the Kex-2 protease, can be engineered to remove the fused sequences from the expressed polypeptides as they exit the secretion pathway. Yeast also are capable of glycosylation at Asn-X-Ser/Thr motifs.

c. Insect Cells

Insect cells, particularly using baculovirus expression, are useful for expressing polypeptides such as ECD polypeptides, including ECD polypeptide fusions. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. For example, a mammalian tissue plasminogen activator precursor sequence facilitates expression and secretion of proteins by insect cells. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian cells

Mammalian expression systems can be used to express ECD polypeptides, including ECD polypeptide fusions provided herein. Expression constructs can be transferred to mammalian cells by viral infection such as by using an adenovirus vector or by direct DNA transfer such as by conventional transfection methods involving liposomes, calcium phosphate, DEAE-dextran and by physical means such as electroporation and microinjection. Exemplary expression vectors include, fore example, pcDNA3.1/myc-His (Invitrogen, SEQ ID NO:161). Expression vectors for mammalian cells typically include an mRNA cap site, a TATA box, a translational initiation sequence (Kozak consensus sequence) and polyadenylation elements. Such vectors often include transcriptional promoter-enhancers for high-level expression, for example the SV40 promoter-enhancer, the human cytomegalovirus (CMV) promoter, such as the hCMV-MIE promoter-enhancer, and the long terminal repeat of Rous sarcoma virus (RSV), or other viral promoters such as those derived from polyoma, adenovirus II, bovine papillom virus or avian sarcoma viruses. Additional suitable mammalian promoters include β-actin promoter-enhancer and the human metallothionein II promoter. These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ζ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293T, 293S, 2B8, and HKB cells. Cell lines also are available adapted to serum-free media which facilitates purification of secreted proteins from the cell culture media. One such example is the serum free EBNA-1 cell line (Pham et al., (2003) Biotechnol. Bioeng. 84:332-42.)

e. Plants

Transgenic plant cells and plants can be used to express ECD polypeptides. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline syntase promoter, the ribose bisphosphate carboxylase promoter and the ubiquitin and UBQ3 promoters. Selecable markers such as hygromycin, phosphomannose isomerase and neomycin phosphoransferase are often used to facilitate selection and maintenance of transformed cells. Transformed plant cells can be maintained in culture as cells, aggregates (callus tissue) or regenerated into whole plants. Transgenic plant cells also can include algae engineered to produce CSR isoforms (see for example, Mayfield et al. (2003) PNAS 100:438-442). Because plants have different glycosylation patterns than mammalian cells, this can influence the choice of CSR isoforms produced in these hosts.

5. Methods of Transfection and Transformation

Transformation or transfection of host cells is accomplished using standard techniques suitable to the chosen host cells. Methods of transfection are known to one of skill in the art, for example, calcium phosphate and electroporation, as well as the use of commercially available cationic lipid reagents, such as Lipofectamine™, Lipofectamine™2000, or Lipofectin® (Invitrogen, Carlsbad Calif.), which facilitate transfection. Depending on the host cell used, transformation is performed using standard techniques appropriate to such cells. Calcium treatment, employing calcium chloride for example, or electroporation is generally used for prokaryotes or other cells that contain substantial cell-wall barriers. Infection with Agrobacterium tumefaciens is used for transformation of certain plant cells. For mammalian cells without such cell walls, calcium phosphate precipitation can be employed. General aspects of transformation are described for plant cells (see e.g., Shaw et al., (1983) Gene, 23:315,

WO89/05859), mammalian cells (see e.g., U.S. Pat. No. 4,399,216, Keown et al., Methods in Enzymolog., (1990) 185:527; Mansour et al., (1988) Nature 336:348), or yeast cells (see e.g. Val Solingen et al., (1977) J Bact (1977) 130:946, Hsiao et al., (1979) Proc. Natl. Acad. Sci., 76:3829). Other methods for introducing DNA into a host cell include, but are not limited to, nuclear microinjection, electroporation, bacterial protoplast fusion with intact cells, or using polycations such as polybrene or polyornithine.

6. Recovery and Purification of ECD Polypeptides, Chimeric Polypeptides, and the Resulting ECD Multimers

ECD polypeptides and chimeric ECD polypeptides, including ECD polypeptide multimers, can be isolated using various techniques well-known in the art. One skilled in the art can readily follow known methods for isolating polypeptides and proteins in order to obtain one of the isolated polypeptides or proteins provided herein. These include, but are not limited to, immunochromatography, HPLC, size-exclusion chromatography, and ion-exchange chromatography. Examples of ion-exchange chromatography include anion and cation exchange and include the use of DEAE Sepharose, DEAE Sephadex, CM Sepharose, SP Sepharose, or any other similar column known to one of skill in the art. Isolation of an ECD polypeptide or ECD multimer polypeptide from the cell culture media or from a lysed cell can be facilitated using antibodies directed against either an epitope tag in a chimeric ECD polypeptide or against the ECD polypeptide and then isolated via immunoprecipiation methods and separation via SDS-polyacrylamide gel electrophoresis (PAGE). Alternatively, an ECD polypeptide or chimeric ECD polypeptide including ECD multimers can be isolated via binding of a polypeptide-specific antibody to an ECD polypeptide and/or subsequent binding of the antibody to protein-A or protein-G sepharose columns, and elution of the protein from the column. The purification of an ECD polypeptide also can include an affinity column or bead immobilized with agents which will bind to the protein, followed by one or more column steps for elution of the protein from the binding agent. Examples of affinity agents include concanavalin A-agarose, heparin-toyopearl, or Cibacrom blue 3Ga Sepharose. A protein can also be purified by hydrophobic interaction chromatography using such resins as phenyl ether, butyl ether, or propyl ether.

In some examples, a chimeric ECD polypeptide can be purified using immunoaffinity chromatography. In such examples, an ECD polypeptide can be expressed as a fusion protein with an epitope tag such as described herein including, but not limited to, maltose binding protein (MBP), glutathione-S-transferase (GST) or thioredoxin (TRX), myc tag and/or a His tag. Kits for expression and purification of such fusion proteins are commercially available from New England BioLab (Beverly, Mass.), Pharmacia (Piscataway, N.J.), Invitrogen, and others. The protein also can be fused to a tag and subsequently purified by using a specific antibody directed to such an epitope. In some examples, an affinity column or bead immobilized with an epitope tag-binding agent can be used to purify an ECD polypeptide fusion. For example, binding agents can include glutathione for interaction with a GST epitope tag, immobilized metal-affinity agents such as Cu2+ or Ni2+ for interaction with a Poly-His tag, anti-epitope antibodies such as an anti-myc antibody, and/or any other agent that can be immobilized to a column or bead for purification of an chimeric ECD protein.

Where a purified homo- or heteromultimeric molecule is desired containing an Fc domain or a mixture thereof, the molecule can be recovered or purified using methods known to one of skill in the art and as detailed in the Examples. Where a host cell is co-expressed with nucleic acid encoding a first polypeptide containing an Fc domain, and nucleic acid encoding a second polypeptide also containing an Fc domain, the resulting expressed molecule will form as a homodimers of the first polypeptide, homodimers of the second polypeptide, and heterodimers of the first and second polypeptide, where each dimer is linked via interactions of the Fc multimerization domain. The combinations of the homo- and hetero-dimers can be recovered from the culture medium as a secreted polypeptide, although it also can be recovered from host cell lysate when directly produced without a signal sequence. If the homo- or heteromultimer is membrane bound, it can be released from the membrane using a suitable detergent solution (e.g., Triton-X 100).

Homo- or heterodimers having antibody constant domains or mixtures thereof can be conveniently purified from conditioned medium, away from other particulate cell debris or contaminating proteins, by a variety of methods including, but not limited to, hydroxylapatite chromatography, gel electrophoresis, dialysis, or affinity chromatography. Where the multimer has a CH3 domain, the Bakerbond ABX™ resin (J. T. Baker, Phillipsburg, N.J.) is useful for purification. Other techniques for protein purification such as fractionation on an ion-exchange column, ethanol precipitation, reverse phase HPLC, chromatography on silica, chromatography on heparin Sepharose, chromatography on an anion or cation exchange resin (such as a polyaspartic acid column), chromatofocusing, SDS-PAGE, and ammonium sulfate precipitation are also available depending on the polypeptide to be recovered.

In addition Protein A or Protein G can be used. The suitability of Protein A as an affinity ligand depends on the species and isotype of the immunoglobulin Fc domain that is used in the chimera. Protein A can be used to purify immunoadhesins that are based on human γ1, γ2, or γ4 heavy chains (Lindmark et al. (1983) J. Immunol. Meth. 62:1-13). Protein G is recommended for all mouse isotypes and for human γ3 (Guss et al. (1986) EMBO J. 5:1567-1575). The matrix to which the affinity ligand, such as Protein A or Protein G, or other affinity ligand capable of interacting with the multimeric molecule), is most often agarose, but other matrices are available. Mechanically stable matrices such as controlled pore glass or poly(styrenedivinyl)benzene allow for faster flow rates and shorter processing times than can be achieved with agarose. The conditions for binding an immunoadhesion to the protein A or G affinity column are dictated entirely by the characteristics of the Fc domain; that is, is species and isotype. Generally, when the proper ligand is chosen, efficient binding occurs directly from unconditioned culture fluid. The bound ECD-Fc containing molecule can be eluted at acidic pH (at or above 3.0), or in a neutral pH buffer containing a mildly chaotropic salt. Alternatively, or in addition, the bound molecule can be eluted with excess IgG. If necessary, the eluted molecules can be neuralized at basic pH. The resulting purified molecule contains purified (typically greater than 95%) homo- and heteromultimers.

Several factors can be used to enrich for the heteromultimeric molecule away from the homodimers including, but not limited to, the use of anti-epitope tags or receptor-specific antibodies that recognize only one chimeric polypeptide component of the multimeric molecule. For example, one of the chimeric polypeptides can be fused to an epitope tag (i.e. c-myc or His). Thus, following purification, such as for example using a Protein A affinity column or other initial purification method depending on the multimerization domain used, the purified molecule can be further enriched using a second affinity column or other matrix. For example, any binding agent can be immobilized to an affinity column or bead for the further purification of an ECD multimer. Exemplary of this is immobilization of metal affinity agents such as Ni2+ for nickel affinity methal chromatography column. Where only a first chimeric polypeptides is recognized by the second affinity column, homodimers containing the second chimeric polypeptide can be washed away leaving only homodimers of the first polypeptide and heterodimers of the first and second polypeptide. Further successive affinity steps can be used to purify the heteromultimer. Such further affinity steps include the immobilization on an affinity column or other matrix of an anti-receptor antibody or a ligand recognizing only the second chimeric polypeptide present in the heteromultimer but not the remaining homomultimer. For example, Example 3 describes the purification of a HER1/HER3 ECD multimer using an EGF affinity column as the final purification step followed by a preparative SEC column to remove any excess ligand. As a final enrichment method, similar affinity columns can be empirically designed using, for example, any binding agent, ligand, or anti-receptor antibody that recognizes one component of the ECD multimer, depending on the components of the ECD multimer.

Additionally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, also can be employed to provide a substantially homogeneous isolated recombinant protein.

Prior to purification, conditioned media containing the secreted ECD polypeptide, including chimeric ECD polypeptide and/or ECD multimers, can be clarified and/or concentrated. Clarification can be by centrifugation followed by filtration. Concentration can be by any method known to one of skill in the art, such as for example, using tangential flow membranes or using stirred cell system filters. Various molecular weight (MW) separation cut offs can be used for the concentration process. For example, a 10,000 MW separation cutoff can be used. The Examples detail various methods of purifying heteromultimers of HER1/HER3 (e.g., Rb200 and Rb200h) as well as purifying mixtures of homomultimers (HER1/HER1 and HER3/HER3) and heteromultimers (HER1/HER3). Accordingly, in one aspect, the invention provides for a composition comprising a mixture of heteromultimers and homomultimers wherein the heteromultimer comprises an ECD or portion thereof from HER1 and another ECD or portion thereof from HER3 and wherein the homomultimers comprise an ECD or portion thereof from HER1 or an ECD or portion thereof from HER3. The mixture can have the ratio of the three multimer components in any ratio. In some cases, the ratio of the three multimer components is dependent on the type of expression system that is used. In one embodiment, the ratio of the three multimer components are about equal to each other.

### G. Assays to Assess or Monitor ECD Multimer Activities

Generally, an ECD multimer modulates one or more biological activities of one or more, typically two or more, cognate CSR or other interacting CSR. In vitro and in vivo assays can be used to monitor a biological activity of an ECD multimer. Exemplary in vitro and in vivo assays are provided herein to assess the biological activity of an RTK ECD multimer, in particular a HER ECD multimer. Many of the assays are applicable to other CSRs ECD multimers. In addition, numerous assays for biological activities of CSRs are known to one of skill in the art, and any assay known to assess the activity of a particular CSR can be chosen depending on the ECD multimer to be tested. Assays to test for the effect of ECD multimers on RTK activity include, but are not limited to, kinase assays, homodimerization and heterodimerization assays, protein:protein interaction assays, structural assays, cell signaling assays and in vivo phenotyping assays. Assays also include the use of animal models, including disease models in which a biological activity can be observed and/or measured. Dose response curves of an ECD multimer in such assays can be used to assess modulation of biological activities and as well as to determine therapeutically effective amounts of an ECD multimer for administration. Exemplary assays are described below.

1. Kinase/Phosphorylation Assays

Kinase activity can be detected and/or measured directly and indirectly. For example, antibodies against phosphotyrosine can be used to detect phosphorylation of an RTK. For example, activation of tyrosine kinase activity of an RTK can be measured in the presence of a ligand for an RTK. Transphosphorylation can be detected by anti-phosphotyrosine antibodies. Transphosphorylation can be measured and/or detected in the presence and absence of an ECD multimer, thus measuring the ability of an ECD multimer to modulate the transphosphorylation of an RTK. Briefly, cells expressing an RTK can be exposed to an ECD multimer and treated with ligand. Cells are lysed and protein extracts (whole cell extracts or fractionated extracts) are loaded onto a polyacrylamide gel, separated by electrophoresis and transferred to membrane, such as used for western blotting. Immunoprecipitation with anti-RTK antibodies also can be used to fractionate and isolate RTK proteins before performing gel electrophoresis and western blotting. The membranes can be probed with anti-phosphotyrosine antibodies to detect phosphorylation as well as probed with anti-RTK antibodies to detect total RTK protein. Control cells, such as cells not expressing RTK isoform and cells not exposed to ligand can be subjected to the same procedures for comparison.

Tyrosine phosphorylation also can be measured directly, such as by mass spectroscopy. For example, the effect of an ECD multimer on the phosphorylation state of an RTK can be measured, such as by treating intact cells with various concentrations of an ECD multimer and measuring the effect on activation of an RTK. The RTK can be isolated by immunoprecipitation and trypsinized to produce peptide fragments for analysis by mass spectroscopy. Peptide mass spectroscopy is a well-established method for quantitatively determining the extent of tyrosine phosphorylation for proteins; phosphorylation of tyrosine increases the mass of the peptide ion containing the phosphotyrosine, and this peptide is readily separated from the non-phosphorylated peptide by mass spectroscopy.

For example, tyrosine-1139 and tyrosine-1248 are known to be autophosphorylated in the HER2 RTK. Trypsinized peptides can be empirically determined or predicted based on polypeptide sequence, for example by using ExPASy-PeptideMass program. The extent of phosphorylation of tyrosine-1139 and tyrosine-1248 can be determined from the mass spectroscopy data of peptides containing these tyrosines. Such assays can be used to assess the extent of auto-phosphorylation of an RTK and the ability of an ECD multimer to modulate transphosphorylate of an RTK.

2. Complexation/Dimerization

Complexation, such as dimerization of RTKs and ECD multimers can be detected and/or measured. For example, isolated polypeptides can be mixed together, subject to gel electrophoresis and western blotting. RTKs and/or ECD multimers also can be added to cells and cell extracts, such as whole cell or fractionated extracts, and can be subject to gel electrophoresis and western blotting. Antibodies recognizing the polypeptides can be used to detect the presence of monomers, dimers and other complexed forms. Alternatively, labeled RTKs and/or labeled ECD multimers can be detected in the assays. Such assays can be used to compare homodimerization of an RTK or heterodimerization of two or more RTKs in the presence and absence of an ECD multimer. Assays also can be performed to assess the ability of an ECD multimer to dimerize with an RTK. For example a HER ECD multimer can be assessed for its ability to heterodimerize with HER1, HER2, HER3, and HER4. Additionally, an ECD multimer can be assessed for its ability to modulate the ability of an RTK to homo- or heterodimerize. For example, a HER ECD multimer can be assessed for its ability to modulate the heterodimerization of HER2 with HER1, HER3, or HER4, among other combinations.

In another example, molecular size exclusion analysis can be performed. Molecular size exclusion is performed with particular size exlusion columns, and eluted molecules compared to a set reference standard. Molecules can be administered alone or can be combined with another molecule. For example, any RTK polypeptide, chimeric polypeptide or ECD multimer can be administered to a size exclusion column. The elution volume can be determined and molecular weights calculated for each of the molecule, such as is described in Example 4. Alternativley, two or more polypeptides can be co-administered and the elution profile assessed to determine if the two or more polypeptides or molecules are capable of forming an oligomeric molecule.

3. Ligand Binding

Generally, RTKs bind one or more ligands. Ligand binding modulates the activity of the receptor and thus modulates, for example, signaling within a signal transduction pathway. Ligand binding to an ECD multimer and ligand binding of an RTK in the presence of an ECD multimer can be measured. For example, labeled ligand such as radiolabeled ligand can be added to purified or partially purified RTK in the presence and absence (control) of an ECD multimer. Immunoprecipitation and measurement of radioactivity can be used to quantify the amount of ligand bound to an RTK in the presence and absence of an ECD multimer. An ECD multimer also can be assessed for ligand binding such as by incubating an ECD multimer with labeled ligand and determining the amount of labeled ligand bound by an ECD multimer, for example, as compared to an amount bound by a wildtype or predominant form of a corresponding RTK.

4. Cell Proliferation Assays

A number of RTKs, for example VEGFR, HER family receptors, and other growth factor receptors are involved in cell proliferation. Effects of an ECD multimer on cell proliferation can be measured. Cells to be tested typically express the target RTK receptor. For example, ligand can be added to cells expressing an RTK. An ECD multimer can be added to such cells before, concurrently or after ligand addition and effects on cell proliferation measured. The level of proliferation of the cells can be assessed by labeling the cells with a dye such as Alamar Blue or Crystal Violet, or other similar dyes, followed by an optimal density measurement. MTT [3-(4,5-dimethylthiazol-2-y1)-2,5-diphenyltetrazolium bromide] also can be used to assess cell proliferation. The use of MTT as a proliferation reagent is based on the ability of a mitochondrial dehydrogenase enzyme from viable cells to cleave the tetrzolium rings of the pale yellow MTT and form a dark blue formazan crystals which accumulates in healthy cells as it is impermeable to cell membranes. Solubilization of cells by the addition of a detergent results in the release and solubilization of the crystals. The color, which is directly proportional to the number of viable, proliferating cells, can be quantified by spectrophotometric means. Thus, after incubation of selected cells with an ECD multimer in the presence or absence of ligand, MTT can be added to the cells, the cells can be solublized with detergent, and the absorbance read at 570 nm. Alternatively, cells can be pre-labeled with a radioactive label such as 3H-tritium, or other fluorescent label such as CFSE prior to proliferation experiments.

5. Cell Disease Model Assays

Cells from a disease or condition or which can be modulated to mimic a disease or condition can be used to measure/and or detect the effect of an ECD multimer. An ECD multimer is added or expressed in cells and a phenotype is measured or detected in comparison to cells not exposed to or not expressing an ECD multimer. Such assays can be used to measure effects including effects on cell proliferation, metastasis, inflammation, angiogenesis, pathogen infection and bone resorption.

For example, effects of an ECD multimer can be measured in angiogenesis. For example, tubule formation by endothelial cells such as human umbilical vein endothelial cells (HUVEC) in vitro can be used as an assay to measure angiogenesis and effects on angiogenesis. Addition of varying amounts of an ECD multimer to an in vitro angiogenesis assay is a method suitable for screening the effectiveness of an ECD multimer as a modulator of angiogenesis.

6. Animal Models

Animal models can be used to assess the effect of an ECD multimer. For example, the effects of an ECD multimer on cancer cell proliferation, migration and invasiveness can be measured. In one such assay, cancer cells such as ovarian cancer cells, after culturing in vitro, are trypsinized, suspended in a suitable buffer and injected into mice (e.g., into flanks and shoulders of model mice such as Balb/c nude mice). Mice are co-administered either before, concurrently, or after the administration of cancer cells to the mice by any suitable route of administration (i.e. subcutaneous, intravenous, intraperitoneal, and other routes). Tumor growth is monitored over time. Similar assays can be performed with other cell types and animal models, for example, murine lung carcinoma (LLC) cells and C57BL/6 mice and SCID mice. Tumor growth can be compared to mice not administered with an ECD multimer, or to mice who are deficient in the respective cognate receptor or interacting receptor of the ECD multimer.

In another example, effects of ECD multimers on ocular disorders can be assessed using assays such as a corneal micropocket assay. Briefly, mice are administered with an ECD multimer (or control) by injection 2-3 days before the assay. Subsequently, the mice are anesthetized, and pellets of a ligand such as VEGF or other growth factor ligand are implanted into the corneal micropocket of the eyes. Neovascularization is then measured, for example, 5 days following implantation. The effect of an ECD multimer on angiogenesis as compared to a control is then assessed.

Any animal models known in the art can be used to assess the effect of a ECD multimer such as a HER multimer, including transgenic mice, such as humanized transgenic mouse models such as atherosclerosis mice expressing DR and DQ major histocompatibility complex II molecules, which can be used as a model for example, for autoimmune diseases, including rheumatoid arthritis, celiac disease, multiple sclerosis, and insulin-dependent diabetes mellitus (Gregersen et al. (2004) Tissue Antigens 63(5):383-94), Apolipoprotein-E deficient mice (ApoE^{-/-}), which can be used as a model for atherosclerosis, IL-10 knockout mice, which can be used as a model, for example, for inflammatory bowel disease and Chrohn's disease (Scheinin et al. (2003) Clin. Exp. Immunol. 133(1):38-43), and Alzheimer's disease models such as transgenic mice overexpressing mutant amyloid precursor protein and mice expressing familial autosomal dominant-linked PS1. Animal models also include animals induced or treated to exhibit disease such as EAE induced animals used as a model for multiple sclerosis.

### H. Preparation, Formulation and Administration of ECD Multimers and ECD Multimer Compositions

ECD multimers and ECD multimer compositions, including HER ECD multimers and HER ECD multimer compositions, can be formulated for administration by any route known to those of skill in the art including intramuscular, intravenous, intradermal, intraperitoneal injection, subcutaneous, epidural, nasal, oral, rectal, topical, inhalational, buccal (e.g., sublingual), and transdermal administration or any route. ECD multimers can be administered by any convenient route, for example by infusion or bolus injection, by absorption through epithelial or mucocutaneous linings (e.g., oral mucosa, rectal and intestinal mucosa, etc.) and can be administered with other biologically active agents, either sequentially, intermittently or in the same composition. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Administration also can include controlled release systems including controlled release formulations and device controlled release, such as by means of a pump. The most suitable route in any given case will depend on the nature and severity of the disease or condition being treated and on the nature of the particular composition which is used.

Various delivery systems are known and can be used to administer ECD multimers, such as but not limited to, encapsulation in liposomes, microparticles, microcapsules, recombinant cells capable of expressing the compound, receptor mediated endocytosis, and delivery of nucleic acid molecules encoding ECD multimers such as retrovirus delivery systems.

Pharmaceutical compositions containing ECD multimers can be prepared. Generally, pharmaceutically acceptable compositions are prepared in view of approvals for a regulatory agency or otherwise prepared in accordance with generally recognized pharmacopoeia for use in animals and in humans. Pharmaceutical compositions can include carriers such as a diluent, adjuvant, excipient, or vehicle with which an ECD multimer is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents. These compositions can take the form of solutions, suspensions, emulsion, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. The formulation should suit the mode of administration.

Formulations are provided for administration to humans and animals in unit dosage forms, such as tablets, capsules, pills, powders, granules, sterile parenteral solutions or suspensions, and oral solutions or suspensions, and oil:water emulsions containing suitable quantities of the compounds or pharmaceutically acceptable derivatives thereof. Pharmaceutically therapeutically active compounds and derivatives thereof are typically formulated and administered in unit dosage forms or multiple dosage forms. Unit dose forms as used herein refer to physically discrete units suitable for human and animal subjects and packaged individually as is known in the art. Each unit dose contains a predetermined quantity of a therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging.

Dosage forms or compositions containing active ingredient in the range of 0.005% to 100% with the balance made up from non toxic carrier can be prepared. For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Pharmaceutical preparation also can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Formulations suitable for rectal administration can be provided as unit dose suppositories. These can be prepared by admixing the active compound with one or more conventional solid carriers, for example, cocoa butter, and then shaping the resulting mixture.

Formulations suitable for topical application to the skin or to the eye include ointments, creams, lotions, pastes, gels, sprays, aerosols and oils. Exemplary carriers include vaseline, lanoline, polyethylene glycols, alcohols, and combinations of two or more thereof. The topical formulations also can contain 0.05 to 15, 20, 25 percent by weight of thickeners selected from among hydroxypropyl methyl cellulose, methyl cellulose, polyvinylpyrrolidone, polyvinyl alcohol, poly(alkylene glycols), polyhydroxyalkyl, (meth)acrylates or poly(meth)acrylamides. A topical formulation is often applied by instillation or as an ointment into the conjunctival sac. It also can be used for irrigation or lubrication of the eye, facial sinuses, and external auditory meatus. It also can be injected into the anterior eye chamber and other places. A topical formulation in the liquid state can be also present in a hydrophilic three-dimensional polymer matrix in the form of a strip or contact lens, from which the active components are released.

For administration by inhalation, the compounds for use herein can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions of ECD multimers can be formulated for parenteral administration by injection, e.g., by bolus injection or continuous infusion. Formulations for injection can be presented in unit dosage form, e.g., in ampules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use.

Formulations suitable for transdermal administration can be presented as discrete patches adapted to remain in intimate contact with the epidermis of the recipient for a prolonged period of time. Such patches suitably contain the active compound as an optionally buffered aqueous solution of, for example, 0.1 to 0.2 M concentration with respect to the active compound. Formulations suitable for transdermal administration also can be delivered by iontophoresis (see, e.g., Pharmaceutical Research 3(6), 318 (1986)) and typically take the form of an optionally buffered aqueous solution of the active compound.

Pharmaceutical compositions also can be administered by controlled release means and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

In certain embodiments, liposomes and/or nanoparticles also can be employed with ECD multimer administration. Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one can operate at the same time.

Nanocapsules can generally entrap compounds in a stable and reproducible way. To avoid side effects due to intracellular polymeric overloading, such ultrafine particles (sized about 0.1 micometers in diameber) can be designed using polymers that can be degraded in vivo. Biodegradable polyalkyl-cyanoacrylate nanoparticles that meet these requirements are contemplated for use herein, and such particles can be easily made.

Administration methods can be employed to decrease the exposure of ECD multimers to degradative processes, such as proteolytic degradation and immunological intervention via antigenic and immunogenic responses. Examples of such methods include local administration at the site of treatment. ECD multimers also can be modified to modulate serum stability and half-life as well as reduce immunogenicity. Such modifications can be effected by any means known in the art and include addition of molecules to ECD multimers such as pegylation, and addition of carrier proteins such as serum albumin, and glycosylation (Raju et al. (2001) Biochemistry 40(3):8868-76; van Der Auwera et al. (2001) Am J Hematol. 66(4):245-51.). In addition, the Fc portion of those ECD multimers formed between the multimerization of Fc modulates serum stability and half-life.

Pegylation of therapeutics has been reported to increase resistance to proteolysis; increase plasma half-life, and decrease antigenicity and immunogencity. Examples of pegylation methodologies are known in the art (see for example, Lu and Felix, Int. J. Peptide Protein Res., 43: 127-138, 1994; Lu and Felix, Peptide Res., 6: 142-6, 1993; Felix et al., Int. J. Peptide Res., 46 : 253-64, 1995; Benhar et al., J. Biol. Chem., 269: 13398-404, 1994; Brumeanu et al., J Immunol., 154: 3088-95, 1995; see also, Caliceti et al. (2003) Adv. Drug Deliv. Rev. 55(10):1261-77 and Molineux (2003) Pharmacotherapy 23 (8 Pt 2):3S-8S). Pegylation also can be used in the delivery of nucleic acid molecules in vivo. For example, pegylation of adenovirus can increase stability and gene transfer (see, e.g., Cheng et al. (2003) Pharm. Res. 20(9): 1444-51).

Desirable blood levels can be maintained by a continuous infusion of the active agent as ascertained by plasma levels. It should be noted that the attending physician would know how to and when to terminate, interrupt or adjust therapy to lower dosage due to toxicity, or bone marrow, liver or kidney dysfunctions. Conversely, the attending physician would also know how to and when to adjust treatment to higher levels if the clinical response is not adequate (precluding toxic side effects), administered, for example, by oral, pulmonary, parental (intramuscular, intraperitoneal, intravenous (IV) or subcutaneous injection), inhalation (via a fine powder formulation), transdermal, nasal, vaginal, rectal, or sublingual routes of administration and can be formulated in dosage forms appropriate for each route of administration (see, e.g., International PCT application Nos. WO 93/25221 and WO 94/17784; and European Patent Application 613,683).

An ECD multimer is included in the pharmaceutically acceptable carrier in an amount sufficient to exert a therapeutically useful effect in the absence of undesirable side effects on the patient treated. Therapeutically effective concentration can be determined empirically by testing the compounds in known in vitro and in vivo systems, such as the assays provided herein.

The concentration of an ECD multimer in the composition will depend on absorption, inactivation and excretion rates of the complex, the physicochemical characteristics of the complex, the dosage schedule, and amount administered as well as other factors known to those of skill in the art.

The amount of an ECD multimer to be administered for the treatment of a disease or condition, for example cancer, autoimmune disease and infection can be determined by standard clinical techniques. In addition, in vitro assays and animal models can be employed to help identify optimal dosage ranges. The precise dosage, which can be determined empirically, can depend on the route of administration and the seriousness of the disease. Suitable dosage ranges for administration can range from about 0.01 pg/kg body weight to 1 mg/kg body weight and more typically 0.05 mg/kg to 200 mg/kg ECD multimer: patient weight.

An ECD multimer can be administered at once, or can be divided into a number of smaller doses to be administered at intervals of time. ECD multimers can be administered in one or more doses over the course of a treatment time for example over several hours, days, weeks, or months. In some cases, continuous administration is useful. It is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of compositions and combinations containing them.

### I. Exemplary Methods of Treatment with ECD Multimers

Provided herein are methods of treatment with ECD multimers and mixtures of ECD multimers for diseases and conditions. ECD multimers, including HER ECD multimers, can be used in the treatment of a variety of diseases and conditions involving CSRs, including RTKs and in particular the HER family of proteins, including those described herein. CSR signaling is involved in the etiology of a variety of diseases and disorders, and any such disease or disorder thereof is contemplated for treatment by an ECD multimer provided herein. Treatments using the ECD multimers provided herein, include, but are not limited to treatment of angiogenesis-related diseases and conditions including ocular diseases, atherosclerosis, cancer and vascular injuries, neurodegenerative diseases, including Alzheimer's disease, inflammatory diseases and conditions, including atherosclerosis, diseases and conditions associated with cell proliferation including cancers, and smooth muscle cell-associated conditions, and various autoimmune diseases. Exemplary treatments and preclinical studies are described for treatments and therapies of RTK-mediated, particularly HER-mediated, diseases and disorders by ECD multimers. Exemplary treatments of other CSR-mediated diseases and disorders such as, but not limited to, RAGE-mediated diseases and disorders are also described. Such descriptions are meant to be exemplary only and are not limited to a particular ECD multimer. Treatment can be effected by administering by suitable route formulations of the molecule, which can be provided in compositions as polypeptides and can be linked to targeting agents, for targeted delivery or encapsulated in delivery vehicles, such as liposomes, or delivered as naked nucleic acids or in vectors. The particular treatment and dosage can be determined by one of skill in the art. Considerations in assessing treatment include, the disease to be treated, the severity and course of the disease, whether the molecule is administered for preventive or therapeutic purposes, previous therapy, the patient's clinical history and response to therapy, and the discretion of the attending physician.

1. HER-Mediated Diseases or Disorders

HER (ErbB)-related diseases or HER receptor-mediated disease are any diseases, conditions or disorders in which a HER receptor and/or ligand is implicated in some aspect of the etiology, pathology or development thereof. In particular, involvement includes, for example, expression or overexpression or activity of a HER receptor family member or ligand. Diseases, include, but are not limited to proliferative diseases, including cancers, such as, but not limited to, pancreatic, gastric, head and neck, cervical, lung, colorectal, endometrial, prostate, esophageal, ovarian, uterine, glioma, bladder or breast cancer. Other conditions, include those involving cell proliferation and/or migration, including those involving pathological inflammatory responses, non-malignant hyperproliferative diseases, such as ocular conditions, skin conditions, conditions resulting from smooth muscle cell proliferation and/or migration, such as stenoses, including restenosis, atheroscelerosis, muscle thickening of the bladder, heart or other muscles, endometriosis, or rheumatoid arthritis. Other diseases that can be treated with a HER ECD multimer provided herein include any disease or disorder mediated by a HER family receptor or its ligands including, but not limited to, aggressiveness, growth retardation, schizophrenia, shock, parkinson's disease, Alzheimer's disease, cardiomyopathy congestive, pre-eclampsia, nervous system disease, and heart failure. Exemplary of such diseases or treatments are set forth below.

a. Cancer

As discussed, HER family receptors are frequently expressed in a variety or human carcinomas, and their expression has been associated with the pathogenesis of many cancers. For example, hyperactivation or dysregulation of HER signaling can lead to aberrant cell activation, including cell proliferation, angiogenesis, and migration and invasion, associated with tumorigenesis. Several mechanisms can account for the dysregulation of HER family receptor signaling that occurs in cancer, including, but not limited to, overproduction of ligands, overproduction of receptors, or constitutive activation of receptors. Because of their roles in cancers and other diseases, HER receptors are therapeutic targets. Co-expression of HER family members, however, often results in lack of response to such therapies, or in development of resistance through compensatory upregulation of alternative HER family members. Thus, HER ECD multimers provided herein can be used as an alternative treatment for cancer, particularly in cancers characterized or associated by co-expression of two or more cell surface receptors.

ECD multimers containing all or a part of a HER1, HER2, HER3, or HER4 ECD can be used in treatment of cancers. In one aspect, the invention provides for methods for treating various types of cancer, inflammatory diseases, angiogenic diseases or hyperproliferative diseases by administering a therapeutically effective amount of a pharmaceutical composition comprising a mixture of heteromultimers and homomultimers wherein the heteromultimer comprises an ECD or portion thereof from HER1 and another ECD or portion thereof from HER3 and wherein the homomultimers comprise an ECD or portion thereof from HER1 or an ECD or portion thereof from HER3. In some cases, the cancer is pancreatic, gastric, head and neck, cervical, lung, colorectal, endometrial, prostate, esophageal, ovarian, uterine, glioma, bladder, renal or breast cancer. In other cases, the disease being treated is a proliferative disease. Non-limiting examples of such proliferative disease include proliferation and/or migration of smooth muscle cells, or is a disease of the anterior eye, or is a diabetic retinopathy, or psoriasis. In other cases, the disease being treated is restenosis, ophthalmic disorders, stenosis, atherosclerosis, hypertension from thickening of blood vessels, bladder diseases, and obstructive airway diseases.

Examples of cancer to be treated herein include, but are not limited to, carcinoma, lymphoma, blastoma, sarcoma, and leukemia or lymphoid malignancies. Additional examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, renal cell cancer, esophageal cancer, glioma, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Combination therapies can be used with HER ECD multimers including anti-hormonal compounds, cardioprotectants, anti-cancer agents such as chemotherapeutics and growth inhibitory agents, and any other such as is described herein.

Cancers treatable with HER ECD multimers are generally cancers expressing at least one HER receptor, typically more than one HER receptor. Such cancers can be identified by any means known in the art for detecting HER expression. For example, HER2 expression can be assessed using a diagnostic/prognostic assay available which includes HERCEPTEST® (Dako). Paraffin embedded tissue sections from a tumor biopsy are subjected to the IHC assay and accorded a HER2 protein staining intensity criteria. Tumors accorded with less than a threshold score can be characterized as not overexpressing HER2, whereas those tumors with greater than or equal to a threshold score can be characterized as overexpressing HER2. In one example of treatment, HER2-overexpressing tumors are assessed as candidates for treatment with a HER ECD multimer, such as any HER ECD multimer provided herein.

b. Angiogenesis

Angiogenesis is a process involving the regulated formation of new blood vessels from existing ones, often that feed tumors and promote cancer metastasis. The production of VEGF is an essential factor for angiogenesis and the migration of cancer cells. A number of factors induce VEGF expression including EGF and TGF-α signaling through HER family receptors. In fact, both HER1 and HER2 are cancer-associated genes implicated in angiogenesis (Yance et al. (2006) Int. Can. Ther., 5: 9-29). HER family receptors also are differentially expressed on endothelial cells. For example, on normal endothelial cells, HER2, HER3, and HER4 are expressed, but on tumor-derived endothelial cells HER1, HER2, and HER4 are expressed (Amin et al. (2006) Cancer Res. 66:2173-80). Thus, as compared to normal cells, tumor-derived endothelial cells have a loss of HER3 expresssion and a gain of HER1 expression, consistent with the responsiveness of endothelial cells to EGF in the production of VEGF and the promotion of angiogenesis.

Targeting of HER family receptors, such as by ECD multimers provided herein, can be used as a treatment of angiogenesis. In vitro or in vivo assays can be used to assess the effects of ECD multimers on angiogenesis. For example, human breast cancer-derived MDA-MB-231 cells, which secrete the angiogenic factor VEGF, can be tested to determine if ECD multimers can antagonize the production of angiogenic factors. In addition, the activity of angiogenic factors produced in the supernatant of these cells, or in the presence of recombinant angiogenic factors in the presence or absence of ECD multimers, can be tested by assaying for the proliferation of human unbilical vein endothelial cells (HUVECs). HUVECs that are [3H]-thymidine incorporation into proliferating HUVECs can be compared to determine if proliferation is reduced in the presence of ECD multimers.

c. Neuregulin-Associated Diseases

The Neuregulins (NRGs) are a complex set of ligands (NRGs 1-4) encoded by four different genes. Some of these molecules are thought to be active in a transmembrane precursor form, such as free ligand (composed of the NRG extracellular domain). The transmembrane and free forms of NRG exert their biological effect through the HER1-4 receptors. These ligands have roles in neuromuscular synapse development, neuron-glial interactions, and cell interactions regulating heart development and function. Therapeutics derived from the extracellular domains of HERs1-4, such as monomeric, homodimeric, and heterodimeric molecules that contain the ligand binding domains of the HER family, can be used for treatment of diseases, such as neurological or neuromuscular diseases, which are associated with, e.g., caused by or aggravated by, exposure to at least one NRG. In one embodiment, the disease is associated with NRG1, including type I, II, and III of NRG1, which all bind to HER3 and HER4. Examples of NRG-associated diseases which may be treated by HER ECD therapeutics as described herein include, but are not limited to, Alzheimer's disease and schizophrenia.

An example of a neurological disease in which NRG is dysregulated may be Alzheimer's disease. Chaudhury et al. (2003) J Neuropathol Exp Neurol 62:42-54. Chaudhury et al. examined the expression and distribution of NRG1 and the erbB kinases in the hippocampus from cognitively normal aging humans, Alzheimer's disease patients, and double transgenic mice that express the Alzheimer's disease phenotype. The expression of both NRG-1 and erbB4 is specifically associated with reactive cellular elements within neuritic plaques, suggesting autocrine and/or paracrine interactions. HER ECD multimers as described herein can be used to treat Alzheimer's disease and related conditions. A variety of mouse models are available for human Alzheimer's disease including transgenic mice overexpressing mutant amyloid precursor protein and mice expressing familial autosomal dominant-linked PS1 and mice expressing both proteins (PS1 M146L/APPK670N:M671L). Alzheimer's models are treated such as by injection of HER ECD multimers. Plaque development can be assessed such as by observation of neuritic plaques in the hippocampus, entorhinal cortex, and cerebral cortex. using staining and antibody immunoreactivity assays.

Schizophrenia remains a serious and largely unresolvable disease of the nervous system. An estimated 1% of the world's population is afflicted with the severe behavioral, emotional, and cognitive impairments characteristic of the disease. Currently, it is considered a syndrome with a dearth of molecular markers to aid in diagnosis. Evidence for an association between NRG and schizophrenia was first presented by Stefannson et al. (2002) Am J Hum Genet 71:877-892. More recent data have suggested that increased levels of NRG1 transcrips are present in prefrontal cortex and peripheral leukocytes of patients with schizophrenia. Hashimoto et al. (2004) Mol Psychiatry 9:299-307; Petryshen et al. (2005) Mol Psychiatry 10:366-74. The connection between NRG1 and schizophrenia may be related to NRG1 reversal of long term potentiation of certain neural synapses. Kwon et al. (2005) J Neurosci 25:9378-83. HER ECD multimers as described herein can be used to treat schizophrenia.

d. Smooth Muscle Proliferative-Related Diseases and Conditions

HER ECD multimers can be utilized for the treatment of a variety of diseases and conditions involving smooth muscle cell proliferation in a mammal, such as a human. An example is treatment of cardiac diseases involving proliferation of vascular smooth muscle cells (VSMC) and leading to intimal hyperplasia such as vascular stenosis, restenosis resulting from angioplasty or surgery or stent implants, atherosclerosis and hypertension. In such conditions, an interplay of various cells and cytokines released act in autocrine, paracrine or juxtacrine manner, which result in migration of VSMCs from their normal location in media to the damaged intima. The migrated VSMCs proliferate excessively and lead to thickening of intima, which results in stenosis or occlusion of blood vessels. The problem is compounded by platelet aggregation and deposition at the site of lesion. α-thrombin, a multifunctional serine protease, is concentrated at site of vascular injury and stimulates VSMCs proliferation. Following activation of this receptor, VSMCs produce and secrete various autocrine growth factors, including PDGF-AA, HB-EGF and TGF. EGFRs are involved in signal transduction cascades that ultimately result in migration and proliferation of fibroblasts and VSMCs, as well as stimulation of VSMCs to secrete various factors that are mitogenic for endothelial cells and induction of chemotactic response in endothelial cells. Treatment with HER ECD multimers can be used to modulate such signaling and responses.

HER ECD multimers, such as HER ECD heteromultimers containing all or part of the ECD of one or both of HER2 and HER3 can be used to treat conditions where HERs such as HER2 and HER3 modulate bladder SMCs, such as bladder wall thickening that occurs in response to obstructive syndromes affecting the lower urinary tract. HER ECD multimers can be used in controlling proliferation of bladder smooth muscle cells, and consequently in the prevention or treatment of urinary obstructive syndromes.

HER ECD multimers can be used to treat obstructive airway diseases with underlying pathology involving smooth muscle cell proliferation. One example is asthma which manifests in airway inflammation and bronchoconstriction. EGF has been shown to stimulate proliferation of human airway SMCs and can be a factor involved in the pathological proliferation of airway SMCs in obstructive airway diseases. HER ECD multimers can be used to modulate effects and responses to EGF by HER1.

2. RTK-Mediated Diseases or Disorders

a. Angiogenesis-Related Ocular Conditions

ECD multimers including, but not limited to, those containing one or more ECD of a VEGFR, PDGFR, TIE/TEK, FGF, EGFR, and EphA, or portion thereof, can be used in treatment of angiogenesis related ocular diseases and conditions, including ocular diseases involving neovascularization. Ocular neovascular disease is characterized by invasion of new blood vessels into the structures of the eye, such as the retina or cornea. It is the most common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage also is associated with diabetic retinopathy, retinopathy of prematurity, corneal graft rejection, neovascular glaucoma and retrolental fibroplasia. Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Karposi sarcoma, Mooren ulcer, Terrien's marginal degeneration, marginal keratolysis, rheumatoid arthritis, systemic lupus, polyarteritis, trauma, Wegener's sarcoidosis, Scleritis, Stevens Johnson disease, pemphigoid radial keratotomy, and corneal graph rejection. Diseases associated with retinal/choroidal neovascularization include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargart's disease, pars planitis, chronic retinal detachment, hyperviscosity syndromes, toxoplasmosis, trauma and post-laser complications. Other diseases include, but are not limited to, diseases associated with rubeosis (neovascularization of the angle) and diseases caused by the abnormal proliferation of fibrovascular or fibrous tissue including all forms of proliferative vitreoretinopathy.

ECD multimer therapeutic effects on angiogenesis such as in treatment of ocular diseases can be assessed in animal models, for example in cornea implants, such as described herein. For example, modulation of angiogenesis such as mediated by an RTK can be assessed in a nude mouse model such as epidermoid A431 tumors in nude mice and VEGF-or PIGF-transduced rat C6 gliomas implanted in nude mice. ECD multimers can be injected as protein locally or systemically, Tumors can be compared between control treated and ECD multimer treated models to observe phenotypes of tumor inhibition including poorly vascularized and pale tumors, necrosis, reduced proliferation and increased tumor-cell apoptosis.

Examples of ocular disorders that can be treated with an ECD heteromultimer containing all or part of a TIE/TEK ECD are eye diseases characterized by ocular neovascularization including, but not limited to, diabetic retinopathy (a major complication of diabetes), retinopathy of prematurity (this devastating eye condition, that frequently leads to chronic vision problems and carries a high risk of blindness, is a severe complication during the care of premature infants), neovascular glaucoma, retinoblastoma, retrolental fibroplasia, rubeosis, uveitis, macular degeneration, and corneal graft neovascularization. Other eye inflammatory diseases, ocular tumors, and diseases associated with choroidal or iris neovascularization also can be treated with TIE/TEK ECD multimers.

ECD heteromultimers containing all or part of a PDGFR ECD also can be used in the treatment of proliferative vitreoretinopathy. Rabbit conjunctival fibroblasts (RCFs) can be injected into the vitreous part of an eye. For example, in a rabbit animal model, approximately 1×10⁵ RCFs are injected by gas vitreomy. Administration of an ECD multimer locally or systemically can be injected on the same day. Effects on proliferative vitreoretinopathy can be observed, for example, 2-4 weeks following surgery, such as attenuation of the disease symptoms.

ECD heteromultimers containing all or part of an EphA ECD can be used to treat diseases or conditions with misregulated and/or inappropriate angiogenesis, such as in eye diseases. For example, an EphA ECD multimer can be assessed in an animal model such as a mouse corneal model for effects on ephrinA-1 induced angiogenesis. Hydron pellets containing ephrinA-1 alone or with an ECD multimer are implanted in mouse cornea. Visual observations are taken on days following implantation to observe ECD multimer inhibition or reduction of angiogenesis.

b. Angiogenesis-Related Atherosclerosis

RTK ECD multimers, for example ECD heteromultimers containing one or both of all or part of an ECD of a VEGFR1 (Flt-1) or TIE/TEK, can be used to treat angiogenesis conditions related to atherosclerosis such as neovascularization of atherosclerosis plaques. Plaques formed within the lumen of blood vessels have been shown to have angiogenic stimulatory activity. VEGF expression in human coronary atherosclerotic lesions is associated with the progression of human coronary atherosclerosis.

Animal models can be used to assess ECD multimers in treatment of atherosclerosis. Apolipoprotein-E deficient mice (ApoE^{-/-}) are prone to atherosclerosis. Such mice are treated by injecting an ECD multimer, for example a VEGFR ECD multimer, over a time course such as for 5 weeks starting at 5, 10 and 20 weeks of age. Lesions at the aortic root are assessed between control ApoE^{-/-} mice and isoform-treated ApoE^{-/-} mice to observe reduction of atherosclerotic lesions in isoform-treated mice.

c. Additional Angiogenesis-Related Treatments

RTK ECD multimers, such as ECD heteromultimers containing all or part of a VEGFR ECD, or all or part of an EphA ECD also can be used to treat angiogenic and inflammatory-related conditions such as proliferation of synoviocytes, infiltration of inflammatory cells, cartilage destruction and pannus formation, such as are present in rheumatoid arthritis (RA). An autoimmune model of collagen type-II induced arthritis, such as polyarticular arthritis induced in mice, can be used as a model for human RA. Mice treated with an ECD multimer, such as by local injection of protein, can be observed for reduction of arthritic symptoms including paw swelling, erythema and ankylosis. Reduction in synovial angiogenesis and synovial inflammation also can be observed. Angiogenesis plays a key role in the formation and maintainance of the pannus in RA. ECD multimers can be used alone and in combination with other isoforms and other treatments to modulate angiogenesis. For example, angiogenesis inhibitors can be used in combination with ECD multimers to treat RA. Exemplary angiogenesis inhibitors include, but are not limited to, angiostatin, antangiogenic antithrombin III, canstatin, cartilage derived inhibitor, fibronectin fragement, IL-12, vasculostatin and others known in the art (see for example, Paleolog (2002) Arthritis Research Therapy 4 (supp 3) S81-S90)

Other angiogenesis-related conditions amenable to treatment with ECD multimers, including for example VEGFR ECD multimers, include hemangioma. One of the most frequent angiogenic diseases of childhood is the hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Many cases of hemangiomas exist that cannot be treated or are difficult to treat with therapeutics currently in use.

ECD multimers, such as VEGFR ECD multimers, can be employed in the treatment of such diseases and conditions where angiogenesis is responsible for damage such as in Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding and sometimes with pulmonary or hepatic arteriovenous fistula. Diseases and disorders characterized by undesirable vascular permeability also can be treated by ECD multimers. These include edema associated with brain tumors, ascites associated with malignancies, Meigs' syndrome, lung inflammation, nephrotic syndrome, pericardial effusion and pleural effusion.

Angiogenesis also is involved in normal physiological processes such as reproduction and wound healing. Angiogenesis is an important step in ovulation and also in implantation of the blastula after fertilization. Modulation of angiogenesis by ECD multimers, such as ECD heteromultimers containing all or part of a VEGFR ECD can be used to induce amenorrhea, to block ovulation or to prevent implantation by the blastula. ECD multimers also can be used in surgical procedures. For example, in wound healing, excessive repair or fibroplasia can be a detrimental side effect of surgical procedures and can be caused or exacerbated by angiogenesis. Adhesions are a frequent complication of surgery and lead to problems such as small bowel obstruction.

RTK ECD multimers useful in treatment of angiogenesis-related diseases and conditions also can be used in combination therapies such as with anti-angiogenesis drugs, molecules which interact with other signaling molecules in RTK-related pathways, including modulation of VEGFR ligands or other growth factor ligand. For example, the known anti-rheumatic drug, bucillamine (BUC), was shown to include within its mechanism of action the inhibition of VEGF production by synovial cells. Anti-rheumatic effects of BUC are mediated by suppression of angiogenesis and synovial proliferation in the arthritic synovium through the inhibition of VEGF production by synovial cells. Combination therapy of such drugs with EGF multimers can allow multiple mechanisms and sites of action for treatment.

d. Cancers

RTK isoforms such as isoforms of TIE/TEK, VEGFR, MET and FGFR can be used in treatment of cancers. RTK isoforms including, but not limited to, VEGFR isoforms such as Flt1 isoforms, FGFR isoforms such as FGFR4 isoforms, and EphA1 isoforms can be used to treat cancer. Examples of cancer to be treated herein include, but are not limited to, carcinoma, lymphoma, blastoma, sarcoma, and leukemia or lymphoid malignancies. Additional examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

For example, ECD heteromultimers containing all or part of a TIE/TEK ECD can be used in the treatment of cancers such as by modulating tumor-related angiogenesis. Vascularization is involved in regulating cancer growth and spread. For example, inhibition of angiogenesis and neovascularization inhibits solid tumor growth and expansion. Tie/Tek receptors such as Tie2 have been shown to influence vascular development in normal and cancerous tissues. TIE/TEK ECD multimers can be used as an inhibitor of tumor angiogenesis. Effects on angiogenesis can be monitored in an animal model such as by treating rat cornea with TIE/TEK ECD multimer formulated as conditioned media in hydron pellets surgically implanted into a micropocket of a rat cornea or as purified protein (e.g. 100 μg/dose) administered to the window chamber. For example, rat models such as F344 rats with avascular corneas can be used in combination with tumor-cell conditioned media or by implanting a fragment of a tumor into the window chamber of an eye to induce angiogenesis. Corneas can be examined histologically to detect inhibition of angiogenesis induced by tumor-cell conditioned media. TIE/TEK ECD multimers also can be used to treat malignant and metastatic conditions such as solid tumors, including primary and metastatic sarcomas and carcinomas.

ECD heteromultimers containing all or part of a FGFR4 ECD can be used to treat cancers, for example pituitary tumors. Animal models can be used to mimic progression of human pituitary tumor progress. For example, an N-terminally shortened form of FGFR, ptd-FGFR4, expressed in transgenic mice recapitulates pituitary tumorigenesis (Ezzat et al. (2002) J. Clin. Invest. 109:69-78), including pituitary adenoma formation in the absence of prolonged and massive hyperplasia. FGFR4 ECD multimers can be administered to ptd-FGFR4 mice and the pituitary architecture and course of tumor progression compared with control mice.

3. Other CSR-Mediated Diseases or Disorders

Also provided herein are treatment of a disease with an ECD heteromultimers containing at least as one of the components a non-RTK CSR such as, but not limited to, a TNFR or a RAGE. For example, an ECD multimer containing at least all or part of an ECD of a RAGE can be used to treat diabetes-related diseases and conditions including periodontal, autoimmune, vascular, and tubulointerstitial diseases. Treatments using RAGE ECD multimers also include treatment of ocular disease including macular degeneration, cardiovascular disease, neurodegenerative disease including Alzheimer's disease, inflammatory diseases and conditions including rhematoid arthritis, and diseases and conditions associated with cell proliferation including cancers. In another example, an ECD multimer containing at least all or part of an ECD of a TNFR family of receptor can be used to treat rheumatoid arthritis, Chrohn's disease, autoimmune disease, rheumatic diseases, inflammatory bowel disease, Alzheimer's disease, and other diseases particularly inflammatory diseases.

4. Selection of the ECD Polypeptide Components of an ECD Multimer

Determination of the components of an ECD multimer is a consideration when determining what ECD multimer molecule to use in treating a selected disease. Several factors can be empirically determined to rationally design an ECD heteromultimer for the treatment of a disease or disorder. First, the disease to be treated should be identified. Typically, such a disease is one which exhibits resistance to a single receptor-targeted therapy, for example, due to overexpression of multiple CSRs, including RTKS and in particular HERs, that contribute to the etiology of the disease. Second, one or more CSRs or ligands of a CSR involved in the etiology of the disease can be identified. Such CSRs or ligands can be a target of the designed ECD multimer such that the ECD multimer is designed to modulate, typically inhibit the activity of the CSRs or ligands thereof. Thus, an ECD multimer would contain as a component all or part of the ECD of the targeted CSR sufficient to dimerize with the CSR, and/or all or part of an ECD sufficient to bind to the targeted CSR ligand. One of skill in the art knows or could identify CSRs, including RTKs or HER family receptors and/or their ligands that are involved in the etiology of the selected diseases. For example, the contribution of CSR to some exemplary diseases and disorders are described above. Third, the components of the ECD sufficient to bind ligand and/or to dimerize with a cognate or interacting CSR can be determined. Such portions of exemplary ECD molecules are described herein, or are known or can be rationally determined by one of skill in the art, such as for example, based on alignments with related receptors and/or by using recombinant DNA techniques in concert with ligand binding assays. All or a portion of an ECD of at least least two or more identified target CSR can be linked directly or indirectly to form multimers, such as for example by their separate linkage to a multimerization domain. In some instances the multimers can be dimers or higher ordered multimers, depending on the method used to link the separate components. The resultant ECD multimer is then a candidate therapeutic for treating the selected disease.

For example, HER receptors, such as for example HER1, are involved in a variety of cancers, including but not limited to, those where HER1 is overexpressed (i.e. colorectal, head and neck, prostate, pancreatic, liver, lung, renal cell, breast, esophageal, ovarian, cervix/uterus, glioma, bladder and others). Thus, an ECD multimer can be designed that has as a component all or part of a HER1 ECD to target HER1 signaling as a mechanism of treating cancer. In the design of the heteromultimer, another CSR molecule that also is involved in the selected disease can be identified and used as the second polypeptide component of the heteromultimer. For example, other HER receptors and their ligands, are overexpressed or involved in a variety of cancers. For example, like HER1, HER3 is overexpressed in breast, colorectal, pancreatic, liver, and esophageal cancers. Thus, a candidate ECD thereapeutic for the treatment of a variety of cancers would be one that is a heteromultimer of all or part of the ECD of HER1 and all or part of the ECD of HER2. In a second example, a selected disease could be angiogenesis. One of skill in the art knows that both VEGFR1 and RAGE are involved in the etiology of angiogeneisis. Thus, a heteromultimer can be designed as a candidate thereapeutic that contains all or part of the ECD of a VEGFR1 and all or part of the ECD of a RAGE.

5. Patient Selection

As mentioned previously, a variety of diseases and disorders are caused by the inappropriate activation of a CSR, particularly a HER family receptor due to, for example, overproduction of ligands, overproduction of receptors, or constitutive activation of receptors. Often, a patient's response to a drug or molecule, such as ECD multimers provided herein, can be predicated on the correlative expression of a CSR or ligand to which the drug or molecule is targeted. Thus, if desired, prior to treatment of a disease or disorder, a patient can be assayed for the expression of a ligand or CSR to select for those patients who are predicted to have an increased responsiveness to treatment by an ECD multimer provided herein. For example, if an ECD multimer therapeutic targets at least one of a HER1 receptor, patients can be tested for expression of HER1. In another example, if a disease to be treated is known to be mediated by a specific ligand, patients can be assayed for the expression of the ligand prior to treatment with an ECD multimer that targets that ligand. The expression of a ligand or a CSR in a patient sample (i.e. blood, serum, tumor, tissue, cell, or other source), can be compared to a control or normal sample to select for those patients that have elevated levels of a CSR or ligand. Such patient selection can ensure treatment of a sub-population of those patients most predicted to respond to a given therapeutic.

In one aspect, expression of a CSR can be assessed in a patient. In one example, expression can be determined in a diagnostic or prognostic assay by evaluating increased levels of the CSR protein present on the surface of a tissue or cell (e.g., via an immunohistochemistry assay; IHC). Alternatively, or additionally, levels of CSR-encoding nucleic acid in the cells can be assessed, e.g., via fluorescent in situ hybridization (FISH; see WO 98/45479), southern blotting, or polymerase chain reaction (PCR), such as real-time quantitative PCR (RT-PCR). In addition, overexpression of a CSR can be assessed by measuring shed antigen (e.g. a soluble CSR) in a biological fluid such as serum (see e.g., U.S. Pat. No. 4,933,294; WO91/05264; U.S. Pat. No. 5,401,638; Sias et al. (1990) J. Immunol. Methods, 132:73-80). In another assay, cells can be isolated from a patient and exposed to a CSR-specific antibody which is optionally labeled with a detectable label, e.g., a radioactive isotope or fluorescent label, and binding of the antibody to cells can be assayed. In another example, the cells of a patient can be exposed to an antibody in vivo and binding of the antibody can be evaluated by, for example external scanning for radioactivity or by analyzing a biopsy taken from a patient previously exposed to the antibody. Any other assay known to one of skill in the art can be used to determine the levels of a CSR in a patient, such as but not limited to, immunoblot, an enzyme linked immunosorbent assay (ELISA), and others. In some cases, selection of patients having increased expression of phosphorylated forms of the receptor can be used to particularly identify those subset of patients with elevated levels of activated receptor. A variety of assays are known in the art to detect phosphorylation of CSRs including, but not limited to, immunoblots or ELISAs using, for example, anti-phosphotyrosine antibodies or anti-phospho specific CSR antibodies.

In some cases, levels of a CSR ligand can be determined as an indicator of patient selection. For example, levels of a ligand in a tissue or tumor of a patient can be determined using immunohistochemistry (IHC, see e.g., Scher et al. (1995) Clin. Cancer Research, 1:545-550). Alternatively, or additionally, levels of a ligand, in a sample, tissue, tumor, or other source can be determined according to any known procedure for detecting protein or encoding nucleic acid. Exemplary of this is ELISA, PCR including RT-PCR, flow cytometry, FISH, southern blotting, and others. Additionally, as above, CSR ligands can be evaluated using an in vivo diagnostic assay, e.g., by administering a molecule (such as an antibody) which binds the molecule to be detected and is tagged with a detectable label (i.e. a radioactive label) and externally scanning the patient for localization of the label. For example, a HER family receptor ligand such as TGF-α, EGF, or amphiregulin can be assayed for in a patient sample, such as in serum, using standard ELISA methods (i.e. commercially available ELISA kits such as from R&D systems), or by immunohistochemistry and tissue microarray in sections of formalin-fixed primary tumors (see e.g., Ishikawa et al. (2005) Cancer Res. 65:9176). In another example, RT-PCR can be used to assess ligand expression in patient cell samples, such as in tumor cells (Mahtouk et al. (2005) Oncogene, 24:3512-3524), or in the blood, bone marrow, or lymph nodes (such as in mononuclear cells isolated therefrom) of a patient.

6. Combination Therapies

ECD multimers such as RTK ECD multimers, including HER ECD multimers, can be used in combination with each other and as mixtures thereof with other existing drugs and therapeutics to treat diseases and conditions, with a therapeutic effect that is either additive or synergistic. For example, as described herein a number of ECD multimers can be used to treat angiogenesis-related conditions and diseases and/or control tumor proliferation. Such treatments can be performed in conjunction with anti-angiogenic and/or anti-tumorigenic drugs and/or therapeutics. Examples of anti-angiogenic and antitumorigenic drugs and therapies useful for combination therapies include tyrosine kinase inhibitors and molecules capable of modulating tyrosine kinase signal transduction can be used in combination therapies including, but not limited to, 4-aminopyrrolo[2,3-d]pyrimidines (see for example, U.S. Pat. No. 5,639,757), and quinazoline compounds and compositions (e.g., U.S. Pat. No. 5,792,771. Other compounds useful in combination therapies include steroids such as the angiostatic 4,9(11)-steroids and C21-oxygenated steroids, angiostatin, endostatin, vasculostatin, canstatin and maspin, angiopoietins, bacterial polysaccharide CM101 and the antibody LM609 (U.S. Pat. No. 5,753,230), thrombospondin (TSP-1), platelet factor 4 (PF4), interferons, metalloproteinase inhibitors, pharmacological agents including AGM-1470/TNP-470, thalidomide, and carboxyamidotriazole (CAI), cortisone such as in the presence of heparin or heparin fragments, anti-Invasive Factor, retinoic acids and paclitaxel (U.S. Pat. No. 5,716,981; incorporated herein by reference), shark cartilage extract, anionic polyamide or polyurea oligomers, oxindole derivatives, estradiol derivatives and thiazolopyrimidine derivatives.

Treatment of cancers including treatment of cancers overexpressing HER can include combination therapy with anti-cancer agents such as anti-HER antibodies, small molecule tyrosine kinase inhibitiors, antisense oligonucleotides, HER/ligand-directed vaccines, or immunoconjugates (i.e. antibodies coupled to radioactive isotope or cytotoxin). Exemplary of such anti-cancer agents include Gefitinib, Tykerb, Panitumumab, Erlotinib, Cetuximab, Trastuzimab, Imatinib, a platinum complex or a nucleoside analog. Other anticancer agents, include radiation therapy or a chemotherapeutic agent and/or growth inhibitory agent, including coadministration of cocktails of different chemotherapeutic agents. Examples of cytotoxic agents or chemotherapeutic agents include, for example, taxanes (such as paclitaxel and doxetaxel) and anthracycline antibiotics, doxorubicin/adriamycine, carminomycin, daunorubicin, aminiopterin, methotrexate, methopterin, dichloro-methotrexate, mitomycin C, porfiromycin, 5-fluorouracil, 6-mercaptopurine, cytosine arabinoside, podophyllotoxin, or podophyllotosin derivatives such as etpoposide or etoposide phosphate, melphalan, vinblastine, vincristine, leurosidine, vindesine, leurosidne, maytansinol, epothilone A or B, taxotere, taxol, and the like. Other such therapeutic agents include extramustine, cisplatin, combretastatin and analogs, and cyclophosphamide. Preparation and dosing schedules for such chemotherapeutic agents can be used according to manufacturers' instructions or as determined empirically by the skilled practitioner. Preparation and dosing schedules for such chemotherapy also are described in Chemotherapy Service Ed., M. C. Perry, Williams & Wilkins, Baltimore, Md. (1992).

Additional compounds can be used in combination therapy with ECD multimers. Anti-hormonal compounds can be used in combination therapies, such as with ECD multimers. Examples of such compounds include an anti-estrogen compound such as tamoxifen; an anti-progesterone such as onapristone and an anti-androgen such as flutamide, in dosages known for such molecules. It also can be beneficial to coadminister a cardioprotectant (to prevent or reduce myocardial dysfunction that can be associated with therapy) or one or more cytokines. In addition to the above therapeutic regimes, the patient can be subjected to surgical removal of cancer cells and/or radiation therapy.

Combination therapy can increase the effectiveness of treatments and in some cases, create synergistic effects such that the combination is more effective than the additive effect of the treatments separately. For example, combination therapy with a chemotherapeutic agent, e.g., a tyrosine kinase inhibitor, and an ECD multimer as described herein, may exhibit a synergistic inhibition of growth of tumor cells, i.e., a growth inhibition effect that is greater than the additive combination of the two agents administered separately.

Adjuvants and other immune modulators can be used in combination with ECD multimers in treating cancers, for example to increase immune response to tumor cells. Examples of adjuvants include, but are not limited to, bacterial DNA, nucleic acid fraction of attenuated mycobacterial cells (BCG; Bacillus-Calmette-Guerin), synthetic oligonucleotides from the BCG genome, and synthetic oligonucleotides containing CpG motifs (CpG ODN; Wooldridge et al. (1997) Blood 89:2994-2998), levamisole, aluminum hydroxide (alum), BCG, Incomplete Freud's Adjuvant (IFA), QS-21 (a plant derived immunostimulant), keyhole limpet hemocyanin (KLH), and dinitrophenyl (DNP). Examples of immune modulators include but are not limited to, cytokines such as interleukins (e.g., IL-2, IL-3, IL-4, IL-5, IL-6, IL-7, IL-9, IL-10, IL-11, IL-12, IL-13, IL-15, IL-16, IL-17, IL-18, IL-1α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and MIF, interferon, cytokines such as IL-2 and IL-12; and chemotherapy agents such as methotrexate and chlorambucil.

The Examples show that the use various forms of heteromultimers and mixtures of heteromultimers and homomultimers in addition to existing therapeutics provide syngergistic results.

### J. Methods for Identifying, Screening and Creating Pan-HER Therapeutics

In addition to ECD multimers provided herein, other candidate pan-HER therapeutics can be identified. Provided herein are methods to identify pan-HER therapeutics, and screening assays therefor. The methods are designed to identify molecules that target ECD subdomains to interfere with ligand binding and/or receptor dimerization and/or tethering by identifying molecules, such as small molecules and polypeptides, that interact with regions on more than one HER receptor family member that are involved in these activities. Such therapeutics can simultaneously target several members of the HER family who do not have multiple coexpression of HER receptors.

1. Targets for Pan-HER Therapeutics

To design such pan-HER therapeutic molecules, similar epitopes or conserved regions that are identified as having involvement in particular activites are identified. For example, regions involved in tethering are identified to screen for candidate molecules that stabilize or promote tethering; regions involved in ligand binding are identified to screen for candidates that interfere with ligand insteraction with two or more HER family members, and regions involved in dimerization are identified.

The regions were and are identified based on the crystal structure data for the receptor family. For example, the design of antagonist therapeutics target aspects of the receptor that determine whether the receptor is in an inactive or active conformation, in order to preferentially target the activated receptor forms which make up about 5% of the HER family receptors on the cell surface. Examples of such structural components predicted by the crystal structure includes, for example, structural components that hold the receptors in a tethered or inactive state, structural components that facilitate dimerization, and or structural components that facilitate ligand binding. Each of these are described below as a potential target for the design of a pan-HER therapeutic.

For example, regions in subdomains II (D II) and IV (D IV) are involved in tethering and in receptor dimerization. Conserved regions can be identified to screen for candidate compounds that inhibit dimerization of more than one HER family member and/or that stabilize tethers or cross-link domains to stabilize the tethered coformation. Such identified polypetides from several HER family members are exemplified in the Examples.

For this approach, homologous polypeptide sequences within each of the targeted structural regions were identified among each of the HER receptors (HER1, HER2, HER3, and HER4). In some examples, homologous regions in the IGF1-R, and other cell surface receptors, also can be aligned to identify potential target sequences. Typically, targeted sequences are derived by using amino acid sequences in one or more HER receptor (typically HER1 and/or HER3) and modeling from the crystal structure, followed by alignment of the identified sequences with other HER family receptors, and picking the most conserved sequences. Corresponding sequences in other HER receptors also are identified. Binding proteins to these targeted sequences can be identified such as, for example, using phage display. The binding proteins can be enriched to identify those that bind to one or more of these regions and 1) inhibit ligand binding, 2) inhibit association of receptors as dimers or heterodimers, and/or 3) inhibit the untethering reaction (i.e. activation of the HER molecule). In some instances, the affinity of the identified peptides can be increased by crosslinking of two or more peptides (i.e. creating peptide heterodimers) such that the crosslinked peptides bind to two regions of the same receptor molecule and prevent it from unfolding. The crosslinked peptides can be ones that recognize distinct epitopes in the same domain, or they can be ones that recognize distinct epitopes in different domains. For example, due to the proximity of domains II and IV in the tethered conformation of a HER receptors, a peptide that recognizes an epitope in domain II can be crosslinked to a peptide that recognizes an epitope in the domain IV tethering region to inhibit the untethering of the tethered conformation.

In one example, pan-HER therapeutic antagonists are designed to lock the receptor in an autoinhibited configuration by preventing dimerization. Thus, regions in domain II and/or regions in domain IV can be targeted. For example, regions in domain II in the dimerization arm, or regions surrounding the dimerization arm, can be targeted to prevent dimerization and association of HER family receptors. In another example, regions in domain IV can be targeted to prevent association of the dimerization arm with homologous regions in domain IV that occurs when the receptors are in a tethered confirmation. Thus, antagonists, such as peptides identified by phage display, or other molecules, such as antibody or other small molecule therapeutics can be identified that bind to distinct sites, for example on domain II of a single receptor, and thereby sterically inhibit its ability to dimerize. Targeted epitope regions that are conserved among HER family members based on alignment with HER3 in either of domain II or domain IV can be used as immunogens to generate antibodies to these regions, or can be used as target substrates to enrich for peptide binders to these sites using, for example, phage display technology. Example 8 describes the identification of exemplary homologous targeted epitope, which also are set forth in any of SEQ ID NOS:62-93 (domain II epitopes) or in any of SEQ ID NOS: 94-125 (domain IV epitopes). In addition, Example 5 describes an exemplary region in HER2 involved in dimerization (set forth in SEQ ID NO:405). Thus, for example, phage display can be used to identify peptides that bind to distinct sites in domain II and/or domain IV homologous regions that can separately bind to regions in domain II and or domain IV to hold the receptor in an autoinhibited configuration by inhibiting dimerization. Higher affinity peptide binders, can be made by generating peptide heterodimers such as is described herein below. An advantage of this approach is that it targets the untethered form of the receptor, which accounts for only about 5% of HER receptors on the cell surface. Thus, the resulting therapeutic will target only a subpopulation of those receptors that are actively signaling, instead of the 95% of receptors on the cell surface that are tethered and inactive. This will increase the effective targeting of the receptor and reduce the dose of drug needed since the total number of targets is decreased by about 15 to 20-fold.

In another example, similar homologous regions on domain II and domain IV can be targeted to generate pan-HER therapeutic antagonists that stabilize the tethered confirmation of a HER receptor. Such therapeutics would target the inactive form of the HER receptors (i.e. about 95% of HER cell surface receptors), and prevent their ability to adopt an active conformation. The feasibility of this approach is supported by the crystal structure data, which demonstrates an intimate interaction between domain II and IV in the untethered or inactive form of HER receptors. The crystal structure of the ECD of HER1 and HER3 suggests that, before ligand stimulation, the receptors are held on the cell surface in an autoinhibited or tethered configuration. In this configuration, intramolecular-specific contacts between the dimerization arm in domain II and a homologous region in domain IV constrain the relative orientation of the two regions responsible for ligand binding (i.e. domains I and III) so they cannot both contact the ligand simultaneously. These structure features suggest that the ligand-dependent HER receptor activation can be prevented if the receptors can be locked in the autoinhibited, tethered configuration. The proximity of domain II and IV sequences predicts that the sequences can be cross-linked because of their close proximity. Thus, the same epitope regions in domains II and IV as described above and in Example 8, and set forth in any of SEQ ID NOS:62-93 (domain II epitopes) and in any of SEQ ID NOS:94-125 (domain IV epitopes), can be targeted. For this approach, peptide binders that are identified, such as for example by phage display methodologies, are selected that target homologous regions in both of domain II and domain IV of HER family receptors. If two peptides, one that binds domain II and the other that binds domain IV are heterodimerized, such as using methods described herein, the peptides can cross-link interdomain regions (e.g. stabilize the domain II and IV interaction) in tethered, inactive HER family members. Thus, the resultant antagonist molecule binds to the tethered form of the receptors, and “locks” the tethered form in place, thereby preventing formation of the high affinity, untethered, form of the receptor.

In an additional example, the ligand binding regions in domain I and III can be targeted by pan-HER therapeutics identified by methods described herein. As above, homologous targeted regions that participate in ligand binding can be identified between HER family receptors. For example, regions of HER1 that participate in ligand binding can be determined by the crystal structure of HER1 in complex with TGF-alpha (Garrett et al. (2002) Cell, 110: 763-773). The crystal structure can be retrieved from PDB protein data bank with 1D, 1MOX. Homologous regions in other HER family receptors can be determined by multiple alignment of HER1, HER2, HER3, and HER4. Example 7 describes regions identified by such an alignment, and aligned sequences are set forth in any of SEQ ID NOS:54-61. These sequences can be targeted by, for example, combinatorial peptide libraries, phage display technology, or by the multiclonal approach (see e.g., Haurum and Bregenholt (2005) IDrugs, 8:404-409). A pan-HER therapeutic identified by such approaches would be expected to inhibit binding of diverse ligands to multiple HER receptors, by blocking sites, such as through steric inhibition, in domains I and/or III. Such a therapeutic would target inactive HER receptors, and inhibit their ability to adopt an active conformation, which occurs only after binding of ligand.

2. Screening Methods to Identify Pan-HER Therapeutics

Provided herein are methods to identify pan-HER therapeutics that target more than one HER family receptor. Collections of molecules are screened. Such collections, include, for example, small organic compounds and other biomolecules including peptides, saccharides, fatty acids, steroids, purines, pyrimidines, derivatives, structural analogs, or combinations thereof. In one example, the collections are screened against the identified polypeptides that are conserved among the receptor family and that participate in a particular activity.

The identified polypeptides also can be screened by any of a variety of methods for screening libraries of molecules to identify those that interact with the identified polypeptides. For example, candidate pan-HER therapeutics can be identified by phage display-derived peptides. Such peptides will be enriched to identify those that bind to the sequence elements conserved among the HER receptor family as discussed above (i.e. any one or more of the peptide epitopes set forth in any of SEQ ID NOS: 54-125, or 405.

a. Phage Display

Phage display technology, which is well established, involves producing libraries or peptides displayed on the phage. These can contain, for example, as many as 10¹⁰ different peptides, thus surpassing many combinatorial small-molecule libraries. The interaction of peptides (often 7-20 amino acids or more) with protein targets can be highly specific, sometimes more so than small molecules. Peptides can be modified to enhance their therapeutic efficacy. For example, brief serum residence and rapid renal filtration can be reduced by PEGylation or fusion with other serum proteins such as albumin. PEGylation not only increases serum residence but also can reduce immunogenicity. In addition, the affinity of peptides for protein targets can be improved by linking two or more synergistic, nonoverlapping peptides to form high affinity heterodimer binders.

The phage display and other such methods can be used in different ways. First, the polypeptides identified here can be screened against a library of displayed polypeptides to identify those polypeptides in the libraries that can be candidate pan-HER therapetuues. Alternatively, the peptides indentified herein, can be displayed and sreened against libraries of small molecules and other polyeptides to identify pan-HER therapeutic candidates.

i. Peptide Libraries

Peptide libraries produced and screened in methods provided herein are useful in providing new ligands for HER family receptors and in producing pan-HER therapeutics. Peptide libraries can be designed and panned according to methods described in detail herein, and methods generally available to those in the art (see e.g., U.S. Pat. No. 5,723,286 and U.S. Patent Application No. US20040023887). In one aspect, commercially available phage display libraries can be used (e.g., RAPIDLIB® or GRABLIB®, DGI BioTechnologies, Inc., Edison, N.J.; C7C Disulfide Constrained Peptide Library or 7-aa and 12-aa linear libraries, New England Biolabs). In another aspect, an oligonucleotide library can be prepared according to methods known in the art, and inserted into an appropriate vector for peptide expression. For example, vectors encoding a bacteriophage structural protein, preferably an accessible phage protein, such as a bacteriophage coat protein, can be used. Although one skilled in the art will appreciate that a variety of bacteriophage can be employed, typically the vector is, or is derived from, a filamentous bacteriophage, such as, for example, f1, fd, Pf1, M13, and others. In particular, the fd-tet vector has been extensively described in the literature (see, e.g., Zacher et al., (1980) Gene 9:127-140; Smith et al., (1985), Science 228:1315-1317; Parmley and Smith (1988) Gene, 73:305-318).

The phage vector is chosen to contain or is constructed to contain a cloning site located in the 5′ region of the gene encoding the bacteriophage structural protein, so that the peptide is accessible to receptors in an affinity enrichment procedure as described herein below. The structural phage protein is generally a coat protein. An example of an appropriate coat protein is pill. A suitable vector can allow oriented cloning of the oligonucleotide sequences that encode the peptide so that the peptide is expressed at or within a distance of about 100 amino acid residues of the N-terminus of the mature coat protein. The coat protein is typically expressed as a preprotein, having a leader sequence.

Typically, the oligonucleotide library is inserted so that the N-terminus of the processed bacteriophage outer protein is the first residue of the peptide, i.e., between the 3′-terminus of the sequence encoding the leader protein and the 5′-terminus of the sequence encoding the mature protein or a portion of the 5′ terminus. The library is constructed by cloning an oligonucleotide which contains the variable region of library members (and any spacers, as discussed below) into the selected cloning site. Using known recombinant DNA techniques (see generally, Sambrook et al., (1989) Molecular Cloning, A Laboratory Manual, 2d ed., Cold Spring Harbor Laboratory Press, Cold Spring Harbor, N.Y.) an oligonucleotide can be constructed which 1) removes unwanted restriction sites and adds desired ones; 2) reconstructs the correct protions of any sequences which have been removed (such as a correct signal peptidase site, for example), 3) inserts the spacer residues, if any; and/or 4) corrects the translation frame, if necessary, to produce active, infective phage.

The central portion of the oligonucleotide will generally contain one or more HER family receptor epitope binding sequences and, optionally, spacer sequences. The sequences are ultimately expressed as peptides (with or without spacers) fused to or in the N-terminus of the mature coat protein on the outer, accessible surface of the assembled bacteriophage particles. The size of the library will vary according to the number of variable codons, and hence the size of the peptides, which are desired. Generally the library will be at least about 10⁶ members, usually at least 10⁷, and typically 10⁸ or more members. To generate the collection of oligonucleotides which forms a series of codons encoding a random collection of amino acids and which is ultimately cloned into the vector, a codon motif is used, such as (NNK)ₓ, where N may be A, C, G, or T (nominally equimolar), K is G or T (nominally equimolar), and x is typically up to about 5, 6, 7, 8, or more, thereby producing libraries of penta-, hexa-, hepta-, and octa-peptides or larger. The third position may also be G or C, designated “S”. Thus, NNK or NNS 1) code for all the amino acids; 2) code for only one stop codon; and 3) reduce the range of codon bias from 6:1 to 3:1.

It should be understood that, with longer peptides, the size of the library that is generated can become a constraint in the cloning process. The expression of peptides from randomly generated mixtures of oligonucleotides in appropriate recombinant vectors is known in the art (see, e.g., Oliphant et al., Gene 44:177-183). For example, the codon motif (NNK)₆ produces 32 codons, one for each of 12 amino acids, two for each of five amino acids, three for each-of three amino acids and one (amber) stop codon. Although this motif produces a codon distribution as equitable as available with standard methods of oligonucleotide synthesis, it results in a bias against peptides containing one-codon residues. In particular, a complete collection of hexacodons contains one sequence encoding each peptide made up of only one-codon amino acids, but contains 729 (36) sequences encoding each peptide with only three-codon amino acids.

An alternative approach to minimize the bias against one-codon residues involves the synthesis of 20 activated trinucleotides, each representing the codon for one of the 20 genetically encoded amino acids. These are synthesized by conventional means, removed from the support while maintaining the base and 5-OH-protecting groups, and activated by the addition of 3′O-phosphoramidite (and phosphate protection with b-cyanoethyl groups) by the method used for the activation of mononucleosides (see, generally, McBride and Caruthers, 1983, Tetrahedron Letters 22:245). Degenerate oligocodons are prepared using these trimers as building blocks. The trimers are mixed at the desired molar ratios and installed in the synthesizer. The ratios will usually be approximately equimolar, but can be a controlled unequal ratio to obtain the over- to under-representation of certain amino acids coded for by the degenerate oligonucleotide collection. The condensation of the trimers to form the oligocodons is done essentially as described for conventional synthesis employing activated mononucleosides as building blocks (see, e.g., Atkinson and Smith, 1984, Oligonucleotide Synthesis, M. J. Gait, Ed., p. 35-82). This procedure generates a population of oligonucleotides for cloning that is capable of encoding an equal distribution (or a controlled unequal distribution) of the possible peptide sequences. Advantageously, this approach can be employed in generating longer peptide sequences, since the range of bias produced by the (NNK)₆ motif increases by three-fold with each additional amino acid residue.

When the codon motif is (NNK)ₓ, as defined above, and when x equals 8, there are 2.6×10¹⁰ possible octa-peptides. A library containing most of the octa-peptides can be difficult to produce. Thus, a sampling of the octa-peptides can be accomplished by constructing a subset library using up to about 10% of the possible sequences, which subset of recombinant bacteriophage particles is then screened. If desired, to extend the diversity of a subset library, the recovered phage subset may be subjected to mutagenesis and then subjected to subsequent rounds of screening. This mutagenesis step can be accomplished in two general ways: the variable region of the recovered phage can be mutagenized, or additional variable amino acids can be added to the regions adjoining the initial variable sequences.

To diversify around active peptides (i.e., binders) found in early rounds of panning, the positive phage can be sequenced to determine the identity of the active peptides. Oligonucleotides can then be synthesized based on these peptide sequences. The syntheses are done with a low level of all bases incorporated at each step to produce slight variations of the primary oligonucleotide sequences. This mixture of (slightly) degenerate oligonucleotides can then be cloned into the affinity phage by methods known to those in the art. This method produces systematic, controlled variations of the starting peptide sequences as part of a secondary library. It requires, however, that individual positive phage be sequenced before mutagenesis, and thus is useful for expanding the diversity of small numbers of recovered phage.

An alternate approach to diversify the selected phage allows the mutagenesis of a pool, or subset, of recovered phage. In accordance with this approach, phage recovered from panning are pooled and single stranded DNA is isolated. The DNA is mutagenized by treatment with, e.g., nitrous acid, formic acid, or hydrazine. These treatments produce a variety of damage to the DNA. The damaged DNA is then copied with reverse transcriptase, which misincorporates bases when it encounters a site of damage. The segment containing the sequence encoding the receptor-binding peptide is then isolated by cutting with restriction nuclease(s) specific for sites flanking the peptide coding sequence. This mutagenized segment is then recloned into undamaged vector DNA, the DNA is transformed into cells, and a secondary library is generated according to known methods. General mutagenesis methods are known in the art (see e.g., Myers et al., 1985, Nucl. Acids Res. 13:3131-3145; Myers et al., 1985, Science 229:242-246; Myers, 1989, Current Protocols in Molecular Biology Vol. I, 8.3.1-8.3.6, F. Ausubel et al., eds, J. Wiley and Sons, New York).

In another general approach, the addition of amino acids to a peptide or peptides found to be active, can be carried out using various methods. In one, the sequences of peptides selected in early panning are determined individually and new oligonucleotides, incorporating the determined sequence and an adjoining degenerate sequence, are synthesized. These are then cloned to produce a secondary library. Alternatively, methods can be used to add a second HER binding sequence to a pool of peptide-bearing phage. In accordance with one method, a restriction site is installed next to the first HER binding sequence. Preferably, the enzyme should cut outside of its recognition sequence. The recognition site can be placed several bases from the first binding sequence. To insert a second HER binding sequence, the pool of phage DNA is digested and blunt-ended by filling in the overhang with Klenow fragment. Double-stranded, blunt-ended, degenerately synthesized oligonucleotides are then ligated into this site to produce a second binding sequence juxtaposed to the first binding sequence. This secondary library is then amplified and screened as before.

While in some instances it is appropriate to synthesize longer peptides to bind certain receptors, in other cases it is desirable to provide peptides having two or more HER binding sequences separated by spacer (e.g., linker) residues. For example, the binding sequences can be separated by spacers that allow the regions of the peptides to be presented to the receptor in different ways. The distance between binding regions can be as little as 1 residue, or at least 2-20 residues, or up to at least 100 residues. Preferred spacers are 3, 6, 9, 12, 15, or 18 residues in length. For probing large binding sites or tandem binding sites (e.g., epitopes on domain II and epitopes on domain IV), the binding regions can be separated by a spacer of residues of up to 20 to 30 amino acids. The number of spacer residues when present will typically be at least 2 residues, and often will be less than 20 residues.

The oligonucleotide library can have binding sequences which are separated by spacers (e.g., linkers), and thus can be represented by the formula: (NNK)_{y}-(abc)ₙ-(NNK)_{z} where N and K are as defined previously (note that S as defined previously may be substituted for K), and y+z is equal to about 5, 6, 7, 8, or more, a, b and c represent the same or different nucleotides comprising a codon encoding spacer amino acids, n is up to about 3, 6, 9, or 12 amino acids, or more. The spacer residues may be somewhat flexible, comprising oligo-glycine, or oligo-glycine-glycine-serine, for example, to provide the diversity domains of the library with the ability to interact with sites in a large binding site relatively unconstrained by attachment to the phage protein. Rigid spacers, such as, e.g., oligo-proline, can also be inserted separately or in combination with other spacers, including glycine spacers. It may be desired to have the HER binding sequences close to one another and use a spacer to orient the binding sequences with respect to each other, such as by employing a turn between the two sequences, as might be provided by a spacer of the sequence glycine-proline-glycine, for example. To add stability to such a turn, it may be desirable or necessary to add cysteine residues at either or both ends of each variable region. The cysteine residues would then form disulfide bridges to hold the variable regions together in a loop, and in this fashion can also serve to mimic a cyclic peptide. Of course, those skilled in the art will appreciate that various other types of covalent linkages for cyclization can also be used.

Spacer residues as described above can also be situated on either or both ends of the HER binding sequences. For instance, a cyclic peptide can be designed without an intervening spacer, by having a cysteine residue on both ends of the peptide. As described above, flexible spacers, e.g., oligo-glycine, can facilitate interaction of the peptide with the selected receptors. Alternatively, rigid spacers can allow the peptide to be presented as if on the end of a rigid arm, where the number of residues, e.g., proline residues, determines not only the length of the arm but also the direction for the arm in which the peptide is oriented. Hydrophilic spacers, made up of charged and/or uncharged hydrophilic amino acids, (e.g., Thr, His, Asn, Gln, Arg, Glu, Asp, Met, Lys), or hydrophobic spacers of hydrophobic amino acids (e.g., Phe, Leu, Ile, Gly, Val, Ala) can be used to present the peptides to receptor binding sites with a variety of local environments.

Notably, some peptides, because of their size and/or sequence, may cause severe defects in the infectivity of their carrier phage. This causes a loss of phage from the population during reinfection and amplification following each cycle of panning. To minimize problems associated with defective infectivity, DNA prepared from the eluted phage can be transformed into appropriate host cells, such as, e.g., E. coli, preferably by electroporation (see, e.g., Dower et al., Nucl. Acids Res. 16:6127-6145), or well-known chemical means. The cells are cultivated for a period of time sufficient for marker expression, and selection is applied as typically done for DNA transformation. The colonies are amplified, and phage harvested for affinity enrichment in accordance with established methods. Phage identified in the affinity enrichment can be re-amplified by infection into the host cells. The successful transformants are selected by growth in an appropriate antibiotic(s), e.g., tetracycline or ampicillin. This can be done on solid or in liquid growth medium.

For growth on solid medium, the cells are grown at a high density (about 10⁸ to 10⁹ transformants per m²) on a large surface of, for example, L-agar containing the selective antibiotic to form essentially a confluent lawn. The cells and extruded phage are scraped from the surface and phage are prepared for the first round of panning (see, e.g., Parmley and Smith, 1988, Gene 73:305-318). For growth in liquid culture, cells can be grown in L-broth and antibiotic through about 10 or more doublings. The phage are harvested by standard procedures (see Sambrook et al., 1989, Molecular Cloning, 2^{nd} ed.). Growth in liquid culture can be more convenient because of the size of the libraries, while growth on solid media can provide less chance of bias during the amplification process.

For affinity enrichment of desired clones, generally about 10³ to 10⁴ library equivalents (a library equivalent is one of each recombinant; 10⁴ equivalents of a library of 10⁹ members is 10⁹×10⁴=10¹³ phage), but typically at least 10²library equivalents, up to about 10⁵ to 10⁶, are incubated with a receptor (or portion thereof) to which the desired peptide is sought. The receptor is in one of several forms appropriate for affinity enrichment schemes. In one example the receptor is immobilized on a surface or particle, and the library of phage bearing peptides is then panned on the immobilized receptor generally according to procedures known in the art. For example, the receptor can be expressed on the cell surface of a monolayer of cells (such as due to transfection, or utilizing a cell that naturally expresses the appropriate receptor). Additionally, the ECD portion of a HER molecule can be linked to an Fc domain and selection can be performed against a HER-Fc complex immobilized to protein A agarose. In such an example, a phage display library can be depleted against an irrelevant Fc fusion protein-protein A (or G) agarose complex. In an alternate scheme, a receptor is attached to a recognizable ligand (which can be attached via a tether). A specific example of such a ligand is biotin. The receptor, so modified, is incubated with the library of phage and binding occurs with both reactants in solution. The resulting complexes are then bound to streptavidin or avidin through the biotin moiety. The streptavidin can be immobilized on a surface such as a plastic plate or on particles, in which case the complexes (phage/peptide/receptor/biotin/streptavidin) are physically retained; or the streptavidin can be labeled, with a fluorophor, for example, to tag the active phage/peptide for detection and/or isolation by sorting procedures, e.g., on a fluorescence-activated cell sorter.

Enrichment of binding phage can be facilitated by subsequent pannings against more specified targets, for example, epitope regions identified in any of subdomains I-IV. Thus, for example, positive phage clones can be screened further against individual synthetic peptides, depending on the targeted subdomain of the HER molecule, such as for example any one or more set forth in any of SEQ ID NOS: 54-61 (subdomains I and III), any of SEQ ID NOS: 62-93 (subdomain II), and/or any of SEQ ID NOS: 94-125, or 405 (subdomain IV). The phage can be enriched against individual peptides set forth in any of SEQ ID NOS:54-125, or 405. Such an enrichment will allow for the determination of the phage binding sites on a HER family receptor. To identify those molecules that are pan-HER therapeutics subsequent screenings also can be performed on other HER family receptors, i.e. HER-Fc-protein A agarose complexes or a monolayer of cells expressing other HER receptors, to identify those molecules that bind to more than one HER family receptor.

At each step, phage that associate with a HER family receptor via non-specific interactions are removed by washing. The degree and stringency of washing required will be determined for each receptor/peptide of interest. A certain degree of control can be exerted over the binding characteristics of the peptides recovered by adjusting the conditions of the binding incubation and the subsequent washing. The temperature, pH, ionic strength, divalent cation concentration, and the volume and duration of the washing will select for peptides within particular ranges of affinity for the receptor. Selection based on slow dissociation rate, which is usually predictive of high affinity, is the most practical route. This can be done either by continued incubation in the presence of a saturating amount of free ligand, or by increasing the volume, number, and length of the washes. In each case, the rebinding of dissociated peptide-phage is prevented, and with increasing time, peptide-phage of higher and higher affinity are recovered. Additional modifications of the binding and washing procedures can be applied to find peptides that bind receptors under special conditions. Once a peptide sequence that imparts some affinity and specificity for the receptor molecule is known, the diversity around this binding motif can be embellished. For instance, variable peptide regions can be placed on one or both ends of the identified sequence. The known sequence can be identified from the literature, or can be derived from early rounds of panning.

ii. Multimeric Polypeptides (Heterodimeric Peptides)

Multimeric polypeptides (ligands) can be prepared by covalently linking amino acid sequences of two or more identified binding peptides, such as identified using phage display technology. Depending on the purpose intended for the multivalent ligand, polypeptides that bind to the same or different domain sites on a HER molecule can be combined to form a single molecule. Where the multivalent ligand is constructed to bind to the same or corresponding site on different receptors, or different subdomains of a receptor, the amino acid sequences of the peptide ligand for binding to the receptors can be the same or different, provided that if different amino acid sequences are used, they both bind to the same site. Other cell surface-specific polypeptides similarly can be prepared.

Multivalent polypeptides can be prepared by either expressing amino acid sequences which bind to the individual sites separately and then covalently linking them together, or by expressing the multivalent ligand as a single amino acid sequence which contains within it the combination of specific amino acid sequences for binding. Combining amino acid polypeptides that bind to distinct sites within a subdomain or between subdomains can be used to produce molecules that are higher affinity peptide ligands or that are capable of crosslinking together different subdomains on a HER receptor.

Whether produced by recombinant gene expression or by conventional linkage technology, the various polypeptides can be coupled through linkers of various length. Where linked sequences are expressed recombinantly, and based on an average amino acid length of about 4 angstroms, the linkers for connecting the two amino acid sequences typically range form about 3 to about 12 amino acids. The degree of flexibility of the linker between the amino acid sequences can be modulated by the choice of amino acids used to construct the linker. The combination of glycine and serine is useful for producing a flexible, relatively unrestrictive linker. A more rigid linker can be constructed using amino acids with more complex side chains within the linkage sequence.

In one example, preparation of multimeric constructs includes one or more binding peptides. For example, peptides identified by phage display as binding to a target are biotinylated and complexed with avidin, streptavidin, ore neutravidin to form tetrameric constructs. These tetrameric constructs are then incubated with a target, or portion thereof, such as, for example, a cell that expresses the desired HER target and cells that do not, and binding of the tetrameric construct is detected Binding can be detected using any method of detection known in the art. For example, to detect binding the avidin, streptavidin, or neutravidin can be conjugated to a detectable marker (e.g., a radioactive label, a fluorescent label, or an enzymatic label that undergoes a color change, such as HRP (horse radish peroxidase), TMB (tetramethyl benzidine), or alkaline phosphatase). The multimeric complexes optionally can be screened in the presence of serum. Thus, the assay can also be used to rapidly evaluate the effect of serum on the binding of peptides to the target.

The biotinylated peptides are preferably complexed with neutravidin-HRP. Neutravidin exhibits lower non-specific binding to molecules than the other alternatives due to the absence of lectin binding carbohydrate moieties and cell adhesion receptor-binding RYD domain in neutravidin (see e.g., Hiller et al. (1987) Biochem J. 248: 167-171; Alon et al. (1990) Biochem. Biophys. Res. Commum., 170:236-41).

The use of biotin/avidin complexes allows for relatively easy preparation of tetrameric constructs containing one to four different binding peptides. In addition, the affinity and avidity of a targeting construct can be increased by including two or more targeting moeieties that bind to different epitopes on the same target. The screening assays described herein can be useful in identifying combinations of binding polypeptides that have increased affinity and/or crosslink distinct subdomains (i.e. to stabilize the tethered conformation) when included in such multimeric constructs.

b. Computer-Aided Optimization

Another method that can be used for identifying pharmacologically active pan-HER therapeutic molecules is to use computer-aided optimization techniques to sort through the possible mutations that result in higher affinity binding to the ligand(s). The Examples provide guidance on how such computer-aided optimization techniques can be used. For examples, HER1, HER2, HER3 or HER4 with enhanced binding to ligands may be generated this way and used as components to make heteromultimers, homomultimers and mixtures of both.

c. Exemplary Screening Assays

Also provided herein are screening assays to identify pharmacologically active pan-HER therapeutic molecules. Pan cell surface-specific molecules similarly can be identified using known assays for particular cell surface receptor activities.

Pan-therapeutic molecules include, for example, 1) peptides such as soluble peptides, including Ig-tailed fusion peptides and members of random peptide libraries (see, e.g., Lam et al., 1991, Nature 354:82-84; Houghten et al., 1991, Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang et al., 1993, Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules. Exemplary molecules are peptide ligands identified from phage display methodologies, such as is described herein above.

Test molecules also can encompass numerous chemical classes, though typically they are organic molecules, preferably small organic compounds having a molecular weight of more than 50 and less than about 2,500 daltons. Such molecules can comprise functional groups necessary for structural interaction with proteins, particularly hydrogen bonding, and typically include at least an amine, carbonyl, hydroxyl or carboxyl group, preferably at least two of the functional chemical groups. Molecules often comprise cyclical carbon or heterocyclic structures and/or aromatic or polyaromatic structures substituted with one or more of the above functional groups. Molecules can be obtained from a wide variety of sources including libraries of synthetic or natural compounds. Synthetic compound libraries are commercially available from, for example, Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich Chemical Company, Inc. (Milwaukee, Wis.). Natural compound libraries comprising bacterial, fungal, plant or animal extracts are available from, for example, Pan Laboratories (Bothell, Wash.). In addition, numerous means are available for random and directed synthesis of a wide variety of organic compounds and biomolecules, including expression of randomized oligonucleotides.

Alternatively, libraries of natural compounds in the form of bacterial, fungal, plant and animal extracts can be readily produced. Methods for the synthesis of molecular libraries are readily available (see, e.g., DeWitt et al., 1993, Proc. Natl. Acad. Sci. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carell et al., 1994, Angew. Chem. Int. Ed. Engl. 33:2059; Carell et al., 1994, Angew. Chem. Int. Ed. Engl. 33:2061; and in Gallop et al., 1994, J. Med. Chem. 37:1233). In addition, natural or synthetic compound libraries and compounds can be readily modified through conventional chemical, physical and biochemical means (see, e.g., Blondelle et al., 1996, Trends in Biotech. 14:60), and can be used to produce combinatorial libraries. In another approach, previously identified pharmacological agents can be subjected to directed or random chemical modifications, such as acylation, alkylation, esterification, amidification, and the analogs can be screened for HER-modulating activity.

Numerous methods for producing combinatorial libraries are known in the art, including those involving biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide or peptide libraries, while the other four approaches are applicable to polypeptide, peptide, non-peptide oligomer, or small molecule libraries of compounds (K. S. Lam, 1997, Anticancer Drug Des. 12:145).

Libraries can be screened in solution by methods generally known in the art for determining whether ligands competitively bind at a common binding site. Such methods can include screening libraries in solution (e.g., Houghten, 1992, Biotechniques 13:412-421), or on beads (Lam, 1991, Nature 354:82-84), chips (Fodor, 1993, Nature 364:555-556), bacteria or spores (Ladner U.S. Pat. No. 5,223,409), plasmids (Cull et al., 1992, Proc. Natl. Acad. Sci. USA 89:1865-1869), or on phage (Scott and Smith, 1990, Science 249:386-390; Devlin, 1990, Science 249:404-406; Cwirla et al., 1990, Proc. Nat. Acad. Sci. USA 97:6378-6382; Felici, 1991, J. Mol. Biol. 222:301-310; Ladner, supra). Any one of the libraries, including any test molecules thereof, can be contacted with all or a portion of a HER molecule, such as any portion of a HER epitope region identified in subdomain I, II, III, or IV and set forth in any of SEQ ID NOS:54-125, and interaction of the test molecule with a HER ECD, or portion thereof, can be assessed. Candidate pan-HER therapeutics can be identified that display interaction with at least one or more of the epitope regions. Such pan-HER therapeutics also will display interaction with at least one or more full-length HER molecule, or ECD portion thereof, typically at least two, or at least three HER molecules.

Where the screening assay is a binding assay, all or a portion of a HER, or all or a portion of a HER ECD thereof such as any one of the peptide epitopes set forth in SEQ ID NOS:54-125 and 405, or a test molecule, can be joined to a label, where the label can directly or indirectly provide a detectable signal. Various labels include radioisotopes, fluorescent molecules, chemiluminescent molecules, enzymes, specific binding molecules, particles, e.g., magnetic particles, and the like. Specific binding molecules include pairs, such as biotin and streptavidin, digoxin and antidigoxin, and others. For the specific binding members, the complementary member would normally be labeled with a molecule that provides for detection, in accordance with known procedures. A variety of other reagents can be included in the screening assay. These include reagents like salts, neutral proteins, e.g. , albumin, detergents, and others, which are used to facilitate optimal protein-protein binding and/or reduce non-specific or background interactions. Reagents that improve the efficiency of the assay, such as protease inhibitors, nuclease inhibitors, or anti-microbial agents, can be used. The components are added in any order that produces the requisite binding. Incubations are performed at any temperature that facilitates optimal activity, typically between 4° and 40° C. Incubation periods are selected for optimum activity, but can also be optimized to facilitate rapid high-throughput screening. Normally, between 0.1 and 1 h will be sufficient. In general, a plurality of assay mixtures is run in parallel with different test agent concentrations to obtain a differential response to these concentrations. Typically, one of these concentrations serves as a negative control, i.e., at zero concentration or below the level of detection.

In one example, phage display libraries can be screened for ligands that bind to HER receptor molecules, or portions thereof, as described above. Details of the construction and analyses of these libraries, as well as the basic procedures for biopanning and selection of binders, have been published (see, e.g., WO 96/04557; Mandecki et al., 1997, Display Technologies—Novel Targets and Strategies, P. Guttry (ed), International Business Communications, Inc. Southborogh, Mass., pp. 231-254; Ravera et al., 1998, Oncogene 16:1993-1999; Scott and Smith, 1990, Science 249:386-390); Grihalde et al., 1995, Gene 166:187-195; Chen et al., 1996, Proc. Natl. Acad. Sci. USA 93:1997-2001; Kay et al., 1993, Gene 128:59-65; Carcamo et al., 1998, Proc. Natl. Acad. Sci. USA 95:11146-11151; Hoogenboom, 1997, Trends Biotechnol. 15:62-70; Rader and Barbas, 1997, Curr. Opin. Biotechnol. 8:503-508; all of which are incorporated herein by reference).

The designing of mimetics to a known pharmaceutically active compound is a known approach to the development of pharmaceuticals based on a “lead” compound. This might be desirable where the active compound is difficult or expensive to synthesize or where it is unsuitable for a particular method of administration, e.g., peptides are generally unsuitable active agents for oral compositions as they tend to be quickly degraded by proteases in the alimentary canal. Mimetic design, synthesis, and testing are generally used to avoid large-scale screening of molecules for a target property.

There are several steps commonly taken in the design of a mimetic from a compound having a given target property. First, the particular parts of the compound that are critical and/or important in determining the target property are determined. In the case of a peptide, this can be done by systematically varying the amino acid residues in the peptide (e.g., by substituting each residue in turn). These parts or residues constituting the active region of the compound are known as its “pharmacophore”.

Once the pharmacophore has been found, its structure is modeled according to its physical properties (e.g., stereochemistry, bonding, size, and/or charge), using data from a range of sources (e.g., spectroscopic techniques, X-ray diffraction data, and NMR). Computational analysis, similarity mapping (which models the charge and/or volume of a pharmacophore, rather than the bonding between atoms), and other techniques can be used in this modeling process. In a variant of this approach, the three dimensional structure of the ligand and its binding partner are modeled. This can be especially useful where the ligand and/or binding partner change conformation on binding, allowing the model to take account of this in the design of the mimetic. A template molecule is then selected, and chemical groups that mimic the pharmacophore can be grafted onto the template. The template molecule and the chemical groups grafted on to it can conveniently be selected so that the mimetic is easy to synthesize, is will be pharmacologically acceptable, does not degrade in vivo, and retains the biological activity of the lead compound. The mimetics found are then screened to ascertain the extent they exhibit the target property, or to what extent they inhibit it. Further optimization or modification can then be carried out to arrive at one or more final mimetics for in vivo or clinical testing.

Pan-HER therapeutics identified in the methods described above can be tested for their ability to functionally modulate one or more HER activity. Such activities are known to those of skill in the art and are described herein above in Section G. Exemplary of such assays include ligand binding, cell proliferation, cell phosphorylation, and complexation/dimerization. Thus, any candidate pan-HER therapeutic identified herein as a candidate based on high affinity binding to a HER molecule or portion thereof, can be tested in further screening assays to determine if the candidate therapeutic possesses pan-HER therapeutic properties, i.e. inhibitory properties against HER activation. For example, a pan-HER therapeutic that targets the dimerization arm in domain II optimally would inhibit the ability of a HER molecule to dimerize with itself or with other HER family molecules. Similarly, in the absence of dimerization such a candidate therapeutic also would be expected to inhibit the ability of a HER molecule to induce cell phosphorylation or cell proliferation when stimulated with the appropriate ligand. In another example, a pan-HER therapeutic that acts to stabilize the tether by, for example, crosslinking domains II and IV, would inhibit the ability of a HER molecule to transition to an activated state. Thus, such a candidate pan-HER therapeutic could be tested for its ability to modulate, typically inhibit, dimerization, or cell activation as assessed by cell proliferation of cell phosphorylation stimulated in the presence of ligand. In an additional example, a candidate pan-HER therapeutic could be tested for its ability to inhibit ligand binding by assaying for binding to any one or more HER family of ligands, including but not limited to EGF, amphiregulin, TGF-alpha, or any one of the neuregulins (i.e. HRGβ). Identified pan-HER therapeutics will modulate, typically inhibit, one or more of the above HER-mediated activities for at least two HER receptors.

### K. Examples

The following examples are included for illustrative purposes only and are not intended to limit the scope of the invention.

### Example 1

### Cloning of HER Extracellular Domains

Various HER derivatives containing all or part of the extracellular domain of a HER molecule were cloned and expressed.

### A. Cloning HER ECD Derivatives

HER1-621 (SEQ ID NO:12) was cloned as follows: the extracellular domain (amino acids 1-621 of the amino acid sequence of the full-length HER1 receptor (obtained from Gail Clinton; SEQ ID NO: 2) was PCR amplified and subcloned into pcDNA3.1 Myc-His vector (Invitrogen; see also SEQ ID No. 161 for sequence of a pcDNA3.1 Myc-His) via KpnI-Xho1 restriction sites to generate pcDNA/HER1-621-myc-His vector.

HER3-621 (SEQ ID NO:26) was cloned as follows: the extracellular domain (amino acids 1-621 of the amino acid sequence of the full length HER3 receptor (see, SEQ ID NO:6) was PCR amplified and subcloned into pcDNA 3.1 Myc-His vector via KpnI-XbaI restriction sites to generate a vector designated pcDNA/HER3-621-myc-His vector.

Additional ECD derivatives were cloned. Their designations and respective encoding nucleic acid and encoded amino sequence identifiers are set forth in the following Table:

TABLE 9
HER ECD derivatives

| | | | | | | SEQ | |
|---|---|---|---|---|---|---|---|
| HER | | | | | | ID NO | |
| Family | Name | Synonym | Type | AA to 501 | Novel AA | nt. | Aa |
| EGFR | HF110 | HER1-501 | 501 | 0 | | 9 | 10 |
| (HER1) | HF100 | HER1-621 | 501(+) | 120 | | 11 | 12 |
| HER2 | HF220 | HER2-530 | 501 | 0 | | 13 | 14 |
| | HF210 | HER2-595 | 501(+) | 65 | | 15 | 16 |
| | HF200 | HER2-650 | 501(+) | 120 | | 17 | 18 |
| HER3 | HF310 | HER3-500 | 501 | 0 | | 19 | 20 |
| | | P85HER3 | 501(+) | 18 | 25 | 21 | 22 |
| | | HER3-519 | 501(+) | 19 | | 23 | 24 |
| | HF300 | HER3-621 | 501(+) | 121 | | 25 | 26 |
| HER4 | HF410 | HER4-485 | 501(−) | −37 | | 27 | 28 |
| | | HER4-522 | 501 | 0 | | 29 | 30 |
| | HF400 | HER4-650 | 501(+) | 128 | | 31 | 32 |
| ERRP | HF120 | ERRP | 501(−) | −77 | 30 | 33 | 34 |
| | | tPA-ERRP | 501(−) | −77 | 30 | 35 | 36 |

FIGS. 2(A)-2(D) set forth alignments of each of these cloned isoforms with their respective cognate receptors.

### B. Protein Expression and Secretion

To express the HER ECD derivatives in human cells, human embryonic kidney 293T cells were seeded at 2×10⁶ cells/well in a 6-well plate and maintained in Dulbecco's modified Eagle's medium (DMEM) and 10% fetal bovine serum (Invitrogen). Cells were transfected using LipofectAMINE 2000 (Invitrogen) according to the manufacturer's instructions. On the day of transfection, 5 μg plasmid DNA was mixed with 15 μl of LipofectAMINE 2000 in 0.5 ml of serum-free DMEM. The mixture was incubated for 20 minutes at room temperature before it was added to the cells. Cells were incubated at 37° C. in a CO₂ incubator for 48 hours. To study the protein secretion of the HER ECD derivatives, the conditioned medium was collected 48 hours. Conditioned medium was analyzed by separation on SDS-polyacrylamide gels followed by immunoblotting using an anti-His antibody (Qiagen). Antibodies were diluted 1:5000.

Culture medium from cultured human cells was assessed for secretion of each of the HER ECD derivatives. Comparisons of the secretion of the HER ECD derivatives are set forth in Table 10 below.

TABLE 10
Protein Secretion of HER ECD derivatives

| HER Family | Name | Synonym | Secretion |
|---|---|---|---|
| EGFR (HER1) | HF110 | HER1-501 | +++ |
| | HF100 | HER1-621 | ++++ |
| HER2 | HF220 | HER2-530 | ++++ |
| | HF210 | HER2-595 | ++++ |
| | HF200 | HER2-650 | +++ |
| HER3 | HF310 | HER3-500 | ++ |
| | | P85HER3 | + |
| | | HER3-519 | + |
| | HF300 | HER3-621 | ++ |
| HER4 | HF410 | HER4-485 | − |
| | | HER4-522 | ++ |
| | HF400 | HER4-650 | +++ |
| ERRP | HF120 | ERRP | − |

### Example 2

### HER-Fc Fusion Preparation and Protein Expression

A. Cloning of the Fc Fragment of human IgG1

The Fc fragment of human IgG1 (set forth in SEQ ID NO:167, and corresponding to amino acids Pro100 to Lys330 of the sequence of amino acids set forth in SEQ ID NO:163) was PCR amplified from a single strand cDNA pool using the forward and reverse primer pair:

| | |
|---|---|
| 5′ CCC AAA TCT TGT GAC AAA ACT ACT | (SEQ ID NO: 49) |
| C 3′ | |
| 5′ TTT ACC CGG GGA CAG GGA G 3′ | (SEQ ID NO: 50) |

The PCR fragment was gel purified and subcloned into the pDrive cloning vector (Qiagen PCR cloning kit, Qiagen, Valencai Calif., SEQ ID NO:160) to generate pDrive/IgG1Fc.

### B. Fusion of Fc to HER Extracellular Domains

HER1-621/Fc (SEQ ID NO:40) was cloned as follows: the pcDNA/ HER1-621-myc-His vector was restriction digested with XhoI and AgeI. The cut plasmid was purified using Qiagen gel purification kit (Qiagen). The human IgG1 Fc fragment was PCR amplified from the pDrive/IgG1Fc vector using the following primers:

5′ ATTA CTCGAG GGA CGA ATG GAC CCC AAA TCT TGT GAC
AAA ACT C 3′
(containing an XhoI site, SEQ ID NO: 51)
5′ ACTT ACCGGT TTT ACC CGG GGA CAG GGA G 3′
(containing an AgeI site, SEQ ID NO: 52)

The PCR amplified Fc fragment was digested with XhoI and AgeI and ligated into the digested pcDNA/HER1-621-myc-His vector.

HER3-621/Fc (SEQ ID NO:46) was cloned as follows: the pcDNA/HER3-621-myc-His vector was restriction digested with XbaI and Age1. The cut plasmid was purified using Qiagen gel purification kit. The human IgG1 Fc fragment was PCR amplified from pDrive/IgG1 Fc by primers:

5′ ATTA TCTAGA GGA CGA ATG GAC CCC AAA TCT TGT GAC
AAA ACT C
(containing an XbaI site, SEQ ID NO: 53)
5′ ACTT ACCGGT TTT ACC CGG GGA CAG GGA G 3′
(containing an AgeI site, SEQ ID NO: 52)

The PCR amplified Fc fragment was digested with XbaI and AgeI and ligated into the digested pcDNA/HER3-621-myc-His vector.

The other fusion constructs were similarly prepared. All of the resulting fusion constructs were verified by DNA sequencing. Exemplary Fc fusion protein constructs are set forth below in the following Table:

TABLE 11

SEQ
ID NO

| HER Family | Name | Synonym | Nt. | aa |
|---|---|---|---|---|
| EGFR (HER1) | HF110-Fc-myc | HER1-501/Fc | 37 | 38 |
| | HF100-Fc | HER1-621/Fc | 39 | 40 |
| HER2 | HF200-Fc | HER2-650/Fc | 41 | 42 |
| HER3 | HF310-Fc | HER3-500/Fc | 43 | 44 |
| | HF300-Fc | HER3-621/Fc | 45 | 46 |
| HER4 | HF400-Fc | HER4-650/Fc | 47 | 48 |

### C. Protein Expression and Secretion

To generate HER-Fc chimeric proteins, a HER ECD Fc fusion construct (HER1-621/Fc; HER3-621/Fc; HER2-650/Fc; HER4-650/Fc) was individually transfected into 293T cells using lipofectamine 2000 (Invitrogen), as described in Example 1. Conditioned medium was collected 48 hours after transfection. Equal amounts of conditioned medium (20 μl) were separated on a denaturing protein gel. Western blots were probed with anti-His (Qiagen) or anti-Fc (Sigma) antibody to check the protein expression and secretion. Comparisons of the secretion of the HER ECD derivatives are depicted in Table 12 below.

TABLE 12
Protein Secretion of HER Fc fusion proteins

| Name | Molecule | Secretion |
|---|---|---|
| HER1-Fc | HER1-621/Fc | +++ |
| HER2-Fc | HER2-650/Fc | ++ |
| HER3-Fc | HER3-621/Fc | ++ |
| HER4-Fc | HER4-650/Fc | ++ |

To generate multimers of HER1 and HER3, the HER Fc fusion constructs (HER1-621/Fc and HER3-621/Fc) each were co-transfected into 293T cells using Lipofectamine 2000 (Invitrogen) in accord with the manufacturer's instructions. Conditioned medium from each transfection was collected 48 hours after transfection. Equal amounts of conditioned medium (20 μl) were separated on a denaturing protein gel. Western blots were probed with anti-His (Qiagen) or anti-Fc (Signma) antibody to check the protein expression and secretion.

To express the heterodimer of RB200h (also called HFD 100/300H (full length HER 1 ECD linked to full length HER 3 ECD via Fc domain)), the constructs of Her1 and Her3 were cotransfected in a ratio of 1:3 (Her1:Her3). The media is replaced with DMEM+1% FBS (low IgG) after 5 hours of TT. First conditioned media were collected 4 days post TT, followed by feeding and a second collection.

Suspension cell protein expression was also done using CHO cells and HEK 293T cells that were previously adapted to serum free media (FreeStyle 293). The HEK 293T cells were seeded in WaveBioReactors at 1×10⁶ cells/ml with Freestyle 293 media (Invitrogen). The next day HER ECD constructs (HER1-621/Fc and HER3-621/Fc) were TT into 293T cells using 25 kD linear PEI (Polysciences):DNA at a ratio of 1:2. To express the heterodimer of RB200h, the constructs of Her1 and Her3 were cotransfected in a ratio of 1:3 (Her1:Her3). After 5 hours of TT, the media volume is doubled. The viable cells and the protein production were monitored daily. Conditioned media were collected 6 days post TT.

### Example 3

### Purification of HER (HF) Derivatives and HER-Fc (HFD) Molecules

All HF molecules with a “T” suffix contain a C-terminal 6-histidine tail for metal affinity purification. All of these molecules were purified using Ni-affinity metal chromatography followed by preparative size-exclusion chromatography (SEC). First, conditioned medium (CM) containing a secreted HF molecule was clarified by centrifugation (30 min, 10K rpm) and then filtered (0.3 micron). Clarified CM was then concentrated 4× using a Pall tangential flow concentrator (Pall Corporation, Ann Arbor, Mich.) to bring the final CM volume to approximately 400 ml.

The CM was brought to 50 mM NaPO₄ (pH 8.0) and 350 mM NaCl by the addition of 10× Ni-NTA loading buffer. The solution was then loaded at a flow rate of 0.6 ml/min onto a 1.5 ml nickle affinity metal chromatography column (Ni-NTA Agarose, Qiagen, Germany) pre-equilibrated with Buffer A (Buffer A: 50 mM NaPO4 (pH 8), 350 mM NaCl). After loading the column was washed with Buffer A until the absorbence at 280 nm indicated no unbound protein remained. The HF molecule was then eluted by an isocratic gradient of Buffer A+150 mM imidazole. Peak fractions containing the HF molecule were pooled and concentrated to 1 ml then loaded onto a preparative SEC column (Superose 12 10/300 GL, Amersham Biosciences, Sweden). The peak fractions containing HF monomer were identified by immunoblotting with a horseradish peroxidase conjugated mouse anti-His6-Tag antibody (HyTest Ltd., Turku, Finland). HF molecule amino acid sequencing was carried out to confirm each molecule.

The HFD100/HFD300T heterodimer is an Fc fusion of HFD100 and HFD300T. Transient transfection to produce this molecule also produces the homodimers designated HFD100 and HFD300T. The HFD300T homodimer and the HFD100/HFD300T heterodimer were purified by Ni-NTA affinity chromatography (Ni-NTA Agarose, Qiagen, Germany) as HF300T contains a C-terminal 6-Histidine tag. Conditioned medium (CM) was clarified and concentrated as described above. The resulting CM was loaded onto a 1.5 ml ProteinA column (nProteinA Sepharose 4 Fast Flow, Amersham Biosciences, Sweden) and eluted with ImmunoPure IgG Elution Buffer (Pierce, Rockford, Ill.). Upon elution the fractions were neutralized by the addition of 50 μl 1M tris-HCl buffer (pH 8.0). Fractions containing protein were pooled and the solution brought to 50 mM NaPO₄ (pH 8.0) and 350 mM NaCl. This pool was loaded onto a 1.5 ml nickle affinity metal chromatography column (Ni-NTA Agarose, Qiagen, Germany) pre-equilibrated with Buffer A. The flow-through containing HFD100 homodimer was collected. After washing with Buffer A, HFD300T homodimer and HFD100/HFD300T heterodimer proteins were eluted with an isocratic gradient of Buffer A+150 mM imidazole.

A 10 ml EGF affinity column was produced by covalently linking 10 mg of EGF (R&D Systems, Minneapolis, Minn.) to a sepharose solid support using 3 ml of CNBr-activiated Sepharose 4 Fast Flow beads (Amersham Biosciences, Finland). Peak fractions from the Ni-NTA eluate were pooled and immediately chromatographed on the EGF affinity column. A peak corresponding to HFD300T was collected in the flow-through. The HFD100/HFD300T heterodimer was eluted with IgG elution buffer, and the fractions containing protein were pooled and chromatographed using a preparative SEC column (Superose 12 10/300 GL, Amersham Biosciences, Sweden). This step removes any EGF which eluted during the EGF affinity column step. Fractions containing purified HFD100/300T were neutralized with 50 μl 1M tris (pH 8.0), buffer exchanged into PBS and concentrated with a 30 kD-cutoff Amicon spin filtration column (Millipore, Billerica, Mass.).

RB600 was purified by taking conditioned media from transfected cells in Example 2 and clarifying by centrifugation at 12,000×g for 15 min at 4° C., followed by filtration through a 3 μm Versapore 3000T filter (Pall Corporation, East Hills, N.Y.). The clarified conditioned media was concentrated 10-fold by ultrafiltration through a 30 kDa cutoff Ultrasette Screen Channel tangential flow filtration device (Pall Corporation, East Hills, N.Y.) and applied to a MabSelect SuRe affinity column (GE Healthcare Biosciences AB, Sweden). The column was washed extensively with PBS containing 0.1% (v/v) TX-114 and eluted with an IgG elution buffer (Pierce Biotechnology Inc., Rockville, Ill.). The eluted fractions were immediately neutralized with 1M Tris-HCL to pH 8.0.

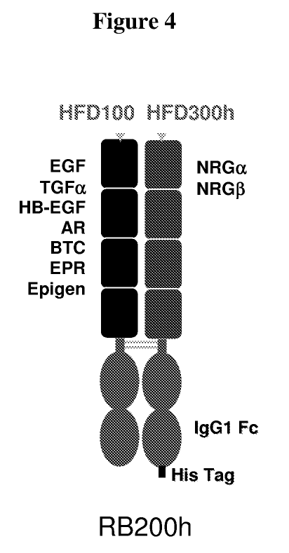

At this stage, Fc-containing proteins eluting from the MabSelect SuRe affinity column consisted of RB200h heterodimer as well as HFD100 and HFD300h homodimers. This mixture of RB200h heterodimer as well as HFD100 and HFD300h homodimers is called called RB600. This homodimer/heterodimer mixture was used directly as a mixture after dialyzing against PBS (RB600) or was used as the starting material for the further purification of RB200h (full length HER 1 ECD linked to full length HER 3 ECD via Fc domain; also called HFD1000/HFD300H). The structure of RB200h is shown in FIG. 4.

### Purity Analysis

Analytical Reversed-phase HPLC was used to determine protein purity. Reversed-phase HPLC of proteins was performed using an analytical C4 column (150×46 mm; 5 mm; 100 A) from Kromasil attached to an AKTA: Purifier System (GE-Healthcare). Buffer A consisted to 0.1% TFA (v/v) in water and Buffer B contained 25% 2-propanol; 75% Acetonitrile; 0.1% TFA (v/v). Typically, 50-100 mg of protein were loaded and a linear gradient of 5-95% buffer B was used to elute samples (flow rate=0.5 mL/min; gradient=6%/min.).

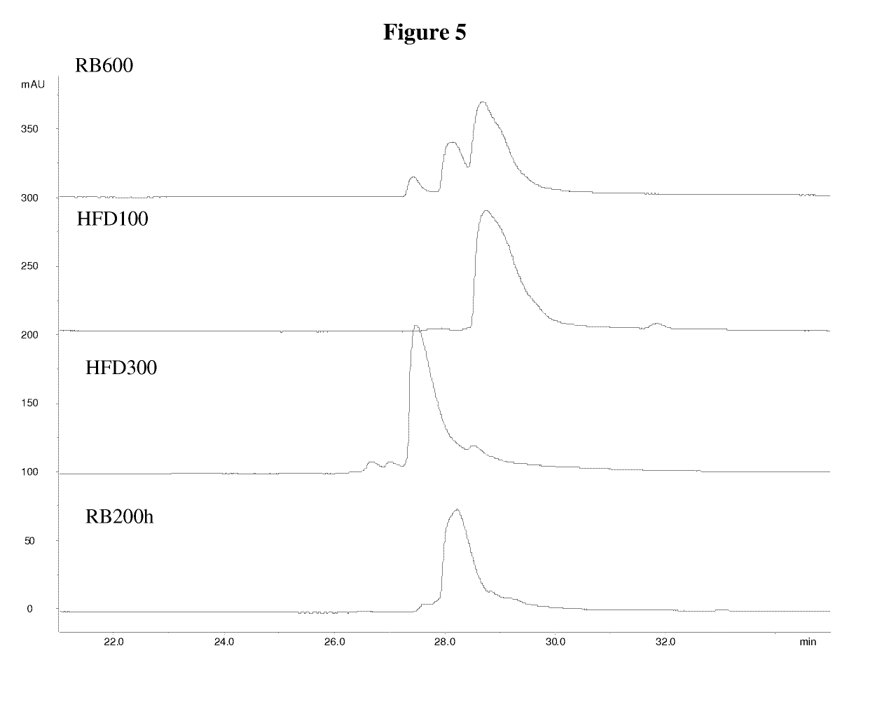

Under conditions in this system, the homodimer containing the 2 erbB3 chains elutes first followed by the heterodimer (RB200h) then the erbBl homodimer. Peak assignmenst were performed using two approaches. First, standards purified from singly transfected cells—coding for only one polypeptide chain—were used to identify the homodimer peaks (see FIG. 5). Second, fractions from each peak were submitted for N-terminal sequencing (Stanford: PAN facility) to verify initial assignments (data not shown).

The purification scheme employed combination of Protein-A, Ni-Sepharose and EGFR-Affybody columns. The purified RB200h was judged as >90% pure by SDS PAGE and reversed phased HPLC. As shown by the analytical reversed-phase HPLC chromatogram, the RB200h (full length HER 1 ECD linked to full length HER 3 ECD via Fc domain) is pure, with no more than 10% combined contamination with HFD 100 and HFD 300 (FIG. 5).

### Example 4

### Binding of HER ECD or HER-Fc to Ligand

### A. Binding of HER ECD Derivatives to Epidermal Growth Factor (EGF)

The extracellular domains of HER1 (HER1), HER2, HER3, and HER4 were fused to human Fc (see Examples 1 and 2) to produce chimeric polypeptides. HER ECD (HER-T) or HER-Fc was obtained from conditioned medium from cells transfected with the relevant vector (see Example 1 and 2 above). Supernatants were collected from 293T cells transiently transfected with the relevant cDNA constructs. Binding of radiolabeled EGF (Amersham) to supernatants containing HER1-621/Fc, HER2-650/Fc, HER3-621/Fc, HER4-650/Fc, HER1-501/Fc, HER1-621(T), HER1-501(T) was determined as follows: Binding was performed by mixing 20 μl of supernatant and 5 nM of ¹²⁵I-EGF with or without 1000× excess of cold EGF in Hepes buffer pH 7.5 at room temperature for 2 hours. BS³, a chemical crosslinker (Pierce) was added at the end of the binding assay to cross-link the bound molecules. Samples were separated on an SDS-PAGE gel and exposed to a film for detection. Estimated ¹²⁵I binding to HER molecules was normalized to the equal molar concentration. The results show that ¹²⁵I-EGF bound only to HER1 derivatives, and no binding of ¹²⁵I-EGF was detected to HER2-650/Fc (HFD200), HER3-650/Fc (HFD300), or to HER4-650/Fc (HDD400). Binding of ¹²⁵I-EGF to HER1-621/Fc (HFD100) was completely competed with excess cold EGF.

Western blotting with an anti-HER1 antibody (R&D Systems), followed by densitometry was used to estimate relative HER1-derivative levels and then to normalize ligand binding to each protein. The results show that HER1-621/Fc (HFD100) has greater binding affinity for ¹²⁵I-EGF than the HER1-501/Fc (HFD110) and HER1-501 (HF110), and much greater binding affinity than the non-Fc full length HER1 ECD (HER1-621; HF100). It is shown below that the Fc fusions form dimers upon expression. Thus, these ligand binding results show that the fusion/dimerization mediated by the Fc portion restores the high affinity binding of the full-length ECD of HER1 that exceeds that of the HER1-501 monomer molecule.

Additional experiments demonstrate that HFD100 (HER-621/Fc) and HFD110 (HER1-501/Fc) exhibit substantially increased binding to ¹²⁵I-EGF ligand compared to HF100, whereas HF110 exhibited no detectable binding to ¹²⁵I-EGF. Furthermore, data demonstrate that the HER1/HER3 (HFD100/HFD300) heterodimer bound to ¹²⁵I-EGF substantially more than HF100 and HF110, but less than the HFD100 or HFD110 homodimers, as expected.

### B. Binding of HER ECD Derivatives to Heregulin (HRG)

The binding of HER ECD derivatives to heregulin was performed using a similar assay as described for binding to EGF described in part A above. Briefly, supernatants were collected from 293T cells transiently transfected with cDNA constructs encoding HF300 (HER3-621), HF310 (HER3-501), HFD300 (HER3-621/Fc), HFD310 (HER3-501/Fc); and a purified HFD110/HFD310 heteromultimer (a construct of HF110 and HF310 linked via the Fc fragment of IgG1). Binding was performed by mixing increasing amounts of supernatants (from 2.5 μl-20 μl of supernatant) with 5 nM of ¹²⁵I-HRG in a total volume of 20 μl of Hepes buffer (pH 7.5) at room temperature for 2 hours. 1 mM of the BS₃ crosslinker was added at the end of the binding assay to cross-link the bound molecules. Binding reactions were separated on an SDS-PAGE gel. The protein gel was dried and exposed to a film for 2 and 6 hours.

The results show that all derivatives tested bound HRG to some extent, although at varying levels. For all derivatives tested, binding was dose-dependent with the greatest binding observed at 20 μl of supernatant. A parallel Western blot with an anti-HER3 antibody (R&D Systems), followed by densitometry was used to estimate relative HER3-derivative levels and then to normalize ligand binding to each protein based on equal number of binding sites, which are the equivalent to anti-HER3 binding sites. After such a normalization, the results showed that HRG displayed the lowest binding to the HF300 molecule, with only about 10% of the binding as compared to the other derivatives tested. Each of HF310, HFD300, HFD310, and HFD110/HFD310 showed equivalent binding to HRG following normalization.

### C. Comparative Analysis of Binding of HER Derivatives to Epidermal Growth Factor (EGF) and Heregulin (HRGβ)

The specificity of the various HER derivatives was compared by testing them for their binding to ¹²⁵I-EGF, a natural ligand for HER1, and ¹²⁵I-HRG, a natural ligand for HER3 and HER4. Binding of radiolabeled EGF to HER1-621/Fc, HER2-650/Fc, HER3-621/Fc, HER4-650/Fc was determined as described above. Binding of ¹²⁵I radiolabeled HRG to HER1-621/Fc, HER2-650/Fc, HER3-621/Fc, HER4-650/Fc was determined using the same conditions as described for binding of ¹²⁵I-EGF. Western blots were probed with anti-His antibody to compare protein levels. The results show that radiolabeled EGF binds only to HER1-621/Fc and not to the other molecules tested. Radiolabeled HRG binds only to HER3-621/Fc and HER4-650/Fc molecules.

Conditioned medium from cells co-transfected with HER1-621/Fc and HER3-621/Fc (see Example 2) or HER1-501/Fc and HER3-501/Fc was tested for binding to ¹²⁵I-EGF and ¹²⁵I-HRG. The data show that cells co-transfected with HER1-621/Fc:HER3-621/Fc produce protein that binds to radiolabeled EGF and to HRG.

Western blots were probed with anti-HER1 and anti-HER3 (R&D Systems) to compare protein levels. The binding of radiolabeled ligand was proportional to the amount of protein expressed by the co-transfected cells, which includes HER1/HER1 homodimers, HER1/HER3 heterodimers, and HER3/HER3 homodimers.

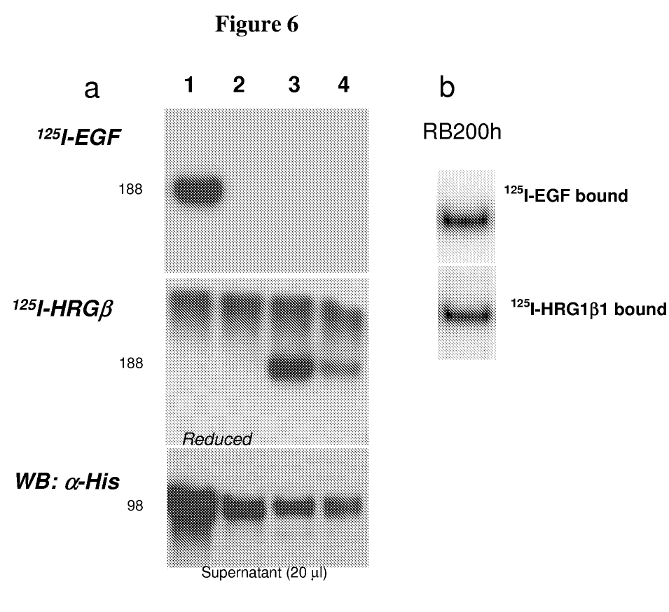

HER1-621/Fc homodimer (termed HFD100) bound ¹²⁵-I-EGF, whereas HER3-621/Fc homodimer (HFD300) and HER4-625/Fc (HFD400) bound ¹²⁵I-HRG1β1 (FIG. 6a). The HER2-628/Fc (HFD200) did not show any detectable ¹²⁵I-EGF or ¹²⁵I-HRG1β1 binding (FIG. 6a). The data show that HFD100, HFD200, HFD300, and HFD 400 retain their specificity for EGF and HRG1b1 (FIG. 6a): Lane 1: HFD100=HER1-621/Fc, Lane 2: HFD200=HER2-628/Fc, Lane 3: HFD300=HER3-621/Fc, and Lane 4:HFD400=HER4-625/Fc. In parallel studies crosslinking of these ligands could be competed by their respective unlabeled ligands, suggesting that the binding is specific.

A chimeric construct of HER1-621/Fc and HER3-621/Fc (termed RB200h) was made in order to create a pan-HER ligand binding Hermodulin. This molecule (RB200h) was tested for its ability to bind HER1 or HER3 ligands by crosslinking studies using ¹²⁵I-EGF or ¹²⁵I-HRG1β1. The data show that RB200h binds both EGF and HRG1β1 (FIG. 6b). These findings revealed that HER1 and HER3 in the chimeric Hermodulin (RB200h) retain their ability to bind their respective ligand and suggest RB200h as a candidate pan-HER ligand binder.

### Example 5

### Formation of Dimeric and Oligomeric Structures of HER Extracellular Domains and HER/Fc Molecules

In an activated form, HER molecules present their dimerization arm in an orientation to facilitate formation of dimerization with other cell surface receptors. Linkage of HER derivatives to the Fc domain predicts a “back-to-back” confirmation that would mimic an activated receptor. To demonstrate that HER derivatives and/or HER/Fc chimeric polypeptides form multimers, molecular size exclusion analysis was performed on the HER family extracellular domain polypeptides. This methodology permits simplified analysis of the ability of receptor ectodomains to associate as either homodimers or heterodimers. To perform molecular size exlusion analysis, eluted molecules were compared to reference standards. Table 13 below shows the molecular mass standards used and their elution volume. Smaller volumes elute in the retained volume of the column, while larger molecules elute in smaller volumes according to their increasing molecular mass.

TABLE 13

| Standard | Mol. Wt. | Elution Vol (ml) |
|---|---|---|
| Vitamin B- | 1350 | 11.80 |
| Myoglobin | 17,000 | 10.37 |
| Ovalbumin | 44,000 | 8.96 |
| Gamma globulin | 158,000 | 8.04 |
| Thyroglobulin | 670,000 | 6.94 |
| Aggregate | 2,631,657 | 6.05 |

Molecular size exclusion analysis was performed using a A TSK3000 size exclusion column (Tosoh Bioscience, Montgomeryville, Pa.) equilibrated with PBS at a flow rate of 0.7 ml/min. Gel filtration standards (BioRad, Hercules, Calif.) were used to calibrate the column. Their elution volumes and molecular weights were plotted. Elution volumes were determined for each unknown by injection of 30 μg of each molecule in PBS and their apparent molecular weights calculated. Flow was maintained over the column between injections. Molecular weights were determined using a Standard curve for molecular weight standards. Table 14 summarizes the results:

TABLE 14
Size exclusion analysis of HER ECD derivatives

| | | | Calculated | Apparent M. | M. Wt. |
|---|---|---|---|---|---|
| HER | | | M. Wt. | Wt. | (ap):M. |
| family | Name | Synonym | (M. Wt.) | (M. Wt. (ap)) | Wt. |
| HER1 | HF110T | HER1-501 | 60,000 | 112,170 | 1.87 |
| | HFD100T | HER1- | 180,000 | 970,003 | 5.39 |
| | | 621/Fc | | | |
| HER2 | HF210T | HER2-595 | 67,000 | 162,069 | 2.42 |
| | HF220T | HER2-530 | 60,000 | 81,676 | 1.36 |
| HER3 | HFD300T | HER3- | 180,000 | 843,627 | 4.69 |
| | | 621/Fc | | | |
| | HF300T | HER3-621 | 72,000 | 186,347 | 2.66 |
| | HF310T | HER3-500 | 63,000 | 110,755 | 1.76 |
| HER4 | HF410T | HER4-485 | 60,000 | 122,590 | 1.98 |

The data show that several of the extracellular domains of the HER family form multimeric structures. The compounds can trap ligand, and form “mock” dimers to prevent dimerization of transmembrane receptor and to thereby bind to and interfere with the activity of the transmembrane protein.

HER1-501 exhibited an apparent molecular mass 112,170 daltons, which is greater than its predicted mass of 60,000 daltons; HER2-595 exhibited an apparent molecular mass of 162,000 daltons versus a predicted mass of 67,000 daltons. HER2-530 (HF220T), which is missing a segment of the HER2 extracellular domain (CSQFLRGQECVEECRVLQGLPREYVNARHCLPCHPECQPQNGSVTCFGPE ADQCVACAHYKDPPF, corresponding to amino acids 508-573 in SEQ ID NO:16) spanning modules 2-5 in domain IV compared to HER2-595 (HF210T), does not form dimeric structures. This latter result indicates that this missing segment (or a portion of segment) is important for dimerization. The differences in the sequences of the two polypeptides are underlined below and made BOLD. The shaded sequences are the tags employed and are the same for both molecules. Since the tags are common to both molecules, they do not play a role in the observed effects on dimerization.

210 with affinity tag

(SEQ ID NO: 274)

TQVCTGTD MKLRLPASPE THLDMLRHLY QGCQVVQGNL
ELTYLPTNAS LSFLQDIQEV QGYVLIAHNQ VRQVPLQRLR
IVRGTQLFED NYALAVLDNG DPLNNTTPVT GASPGGLREL
QLRSLTEILK GGVLIQRNPQ LCYQDTILWK DIFHKNNQLA
LTLIDTNRSR ACHPCSPMCK GSRCWGESSE DCQSLTRTVC
AGGCARCKGP LPTDCCHEQC AAGCTGPKHS DCLACLHFNH
SGICELHCPA LVTYNTDTFE SMPNPEGRYT FGASCVTACP
YNYLSTDVGS CTLVCPLHNQ EVTAEDGTQR CEKCSKPCAR
VCYGLGMEHL REVRAVTSAN IQEFAGCKKI FGSLAFLPES
FDGDPASNTA PLQPEQLQVF ETLEEITGYL YISAWPDSLP
DLSVFQNLQV IRGRILHNGA YSLTLQGLGI SWLGLRSLRE
LGSGLALIHH NTHLCFVHTV PWDQLFRNPH QALLHTANRP
EDECVGEGLA CHQLCARGHC WGPGPTQCVN CSQFLRGQEC
VEECRVLQGL PREYVNARHC LPCHPECQPQ NGSVTCFGPE
ADQCVACAHY KDPPFLESRG PFEQKLISEE DLNMHTGHHH
HHH
220 with affinity tag

(SEQ ID NO: 275)

TQVCTGTD MKLRLPASPE THLDMLRHLY QGCQVVQGNL
ELTYLPTNAS LSFLQDIQEV QGYVLIAHNQ VRQVPLQRLR
IVRGTQLFED NYALAVLDNG DPLNNTTPVT GASPGGLREL
QLRSLTEILK GGVLIQRNPQ LCYQDTILWK DIFHKNNQLA
LTLIDTNRSR ACHPCSPMCK GSRCWGESSE DCQSLTRTVC
AGGCARCKGP LPTDCCHEQC AAGCTGPKHS DCLACLHFNH
SGICELHCPA LVTYNTDTFE SMPNPEGRYT FGASCVTACP
YNYLSTDVGS CTLVCPLHNQ EVTAEDGTQR CEKCSKPCAR
VCYGLGMEHL REVRAVTSAN IQEFAGCKKI FGSLAFLPES
FDGDPASNTA PLQPEQLQVF ETLEEITGYL YISAWPDSLP
DLSVFQNLQV IRGRILHNGA YSLTLQGLGI SWLGLRSLRE
LGSGLALIHH NTHLCFVHTV PWDQLFRNPH QALLHTANRP
EDECVGEGLA CHQLCARGHC WGPGPTQCVN LESRGPFEQK
LISEEDLNMH TGHHHHHH

The data also show that HER-Fc proteins also form high order oligomers. HER1-621/Fc and HER3-621/Fc each have predicted molecular weights of 180,000 daltons, and observed molecular weights by size exclusion chromatography of greater than 970,000 and 843,000 daltons, respectively. Because these assays were performed in the absence of ligand, this result further demonstrates that ligand is not needed in order to create a dimerized (or higher order) structure.

### Example 6

### HER Receptor Proliferation and Phosphorylation: Inhibition by HER Derivatives

### A. HER Expression Profiles in Cell Lines

HER expression level was analyzed by Fluorescence Activated Cell Sorting (FACS) to identify the receptors and relative amounts thereof on the surface of various cells lines. Selected cells were contacted with receptor-specific antibodies and the intensity of fluorescence upon binding cells with receptor-specific antibodies was assessed.

Cells were lifted from tissue culture plate with 5 nM EDTA and resuspended in PBS containing 1% of BSA (PBS.BSA). Cells in suspension were incubated with monoclonal antibodies against each of HER1, 2, 3 and 4 in respective tubes, for 1 hr at 4° C. After the first antibody incubation, cells were washed with cold PBS.BSA once. The second antibodies, against mouse or human IgG (depending upon the origin of the first antibodies) tagged with a fluorescent dye PE (Jackson), then were added. The cells were incubated for 30 min at 4° C. and washed twice with PBS.BSA wash. Cells were fixed by adding Cytofix (BD-554655) and kept in dark at 4° C. FACSs was performed using a Cell Sorter apparatus (BD FACSCalibur Flow Cytometer). 10,000 cells of each cell line were analyzed. The Mean Fluorescence Intensity (MFI) of each HER receptor in each cell lines were measure by MFI with BD CellQuest Pro Software. Scoring: ++++>1000 MFI, +++100-1000 MFI, ++50-100 MFI, +<50 MFI but have signal above background.

Table 15 presents the resulting expression profiles of the HER family of receptors in various cells lines.

TABLE 15
HER Expression Profiles in Cell Lines

| | | HER Expression | | | |
|---|---|---|---|---|---|
| | Cell lines | HER1 | HER2 | HER3 | HER4 |
| Tumor cell lines | A431 | +++ | + | | |
| | SK-BR3 | ++ | +++ | ++ | |
| | SK-OV3 | ++ | +++ | | |
| | MCF7 | | + | ++ | + |
| | MCF7/HER2 | | ++++ | ++ | |
| | ME180 | +++ | ++ | | |
| Non-tumor | PNT 1A | ++ | + | | |
| | HEK293 | + | ++ | | |

### B. Cell Proliferation Assay

Cell lines MCF7, ZR75-1, ME180 were purchased from ATCC and kept in 10% of FBS DMEM. Cells were seeded at 2000 per well in 96-well plate in 1% FBS supplemented DMEM. After 2-3 hr of seeding, increasing concentrations of candidate HER ECD derivatives were added to the culture in the present of ligands (EGF or HRGβ). Cells were incubated at 37° C. for about 72 hr. Cells relative density were measured by Alamar Blue method. Alamar Blue (Sigma) was prepared in PBS at concentration of 4 uM, added to the microplate at 1/10 volume of culture medium (final concentration 0.4 uM) and plates returned to the incubator. Fluorescence was read at Ex.=530 nm/Em.=590 nm after 2-4 hours at 37° C.

### Results

Cell Proliferation Data: The HFD100/300 preparation was a pool of HFD100/100, HFD300/300 and HFD100/300 molecules in unknown proportions. Nevertheless, the data evidence the ability of the hybrid material to perform inhibition. HFD 100/300 inhibited ME180 proliferation stimulated by HRGβ (5 nm). The data indicated greater than 80% inhibition at about 3 nM HFD100/300 as well as against EGF-stimulated HER1. HF310T inhibited MCF7 proliferation stimulated by HRGβ (about 95% at 1 μm).

### C. ELISA-based HER Receptor Phosphorylation Assay

Phosphorylation of HER receptors was assessed in an ELISA-based HER Receptor phosphorylation assay. Various cells (A431, MCF7, SK-BR3, SK-OV3, MCF7/HER2) were serum-starved in serum free medium for about 24 hr. Cells were then treated with increasing concentrations of candidate HER ECD derivatives (see below) for 30 min at 37° C., ligands (EGF, 3 nM and/or HRGβ, 5 nM were then added for 10 min incubation. After treatment, cells were washed with PBS once and lysed with 100 μl of 1× Cell Lysis Buffer (Cell Signaling) with addition of protease and phosphtase inhibitors (Protease Inhibitor Cocktail Set and Phosphatase Inhibitor Set, Calbiochem).

Cells were lysed on ice for 15 min and cell lysate were applied to a 96-well plate pre-coated with the respective receptor-specific capture antibodies (antibodies were purchased from R & D System) by manufacture recommended concentrations (0.4 to 4 ug/ml) and condition (in PBS, room temperature, overnight). Cell lysates were incubated with the capture Ab plate for 3 hrs at room temperature. Plates were washed 3× with PBST buffer. Anti-phosphotyrosine antibody clone 4G10 HRP-conjugated (Upstate) were diluted at 1:1000 in 1% of BSA.PBS and added to the plates, 100 μl/well for 1 hr to detect specifically HER receptors phosphorylated on tyrosine. After 3× PBST wash, the plates were developed by adding 100 μl of substrate solution (TMB, Sigma) and stopped by 50 μl of SDS stop solution. The optical density was determined by microplate reader at 650 nm (Molecular Devices, VERSAmax).

i. HER1-501

The ability of HER1-501 to inhibit phosphorylation of HER1 and HER2 was tested in A431 cells and MCF7 cells. Increasing concentrations of HER1-501, up to a maximum concentration of 600 nM, was added to cells in the presence of EGF. As expected, no phosphorylation of HER1 in MCF7 cells was observed. In contrast, HER1 dose-dependently inhibited the phosphorylation of HER1 in A431 cells, with an IC₅₀ of 98 nM. The maximal inhibition of HER1 phosphorylation achieved at 600 nM HER1-501 was about 60% compared to the absence of the protein. HER1-501 also dose-dependently inhibited the phosphorylation of HER2 in MCF7 and A431 cells with an IC₅₀ of 18 nM and 42 nM, respectively. The maximal inhibition of HER2 phosphorylation, in both cell lines tested, achieved at 600 nM HER1-501 was about 50% compared to the absence of the protein.

ii. HER2-595 and HER2-530

The ability of HER2-595 and HER2-530 to inhibit phosphorylation of HER2 and HER3 was tested in MCF7/HER2 cells. Increasing concentrations of HER2-595 or HER2-530 (0, 7.4 nM, 22.2 nM, 66.7 nM, 200 nM, and 600 nM) was added to cells in the presence of HRG. The data show that HER2-595 and HER2-530 dose-dependently inhibited the phosphorylation of HER2 and HER3; HER2-595 was more potent. The maximal inhibition of HER2 and HER3 phosphorylation achieved by 600 nM HER2-595 in MCF7/HER2 cells was about 55% compared to the absence of the protein, whereas the maximal inhibition achieved by 600 nM HER2-530 was about 35% compared to the absence of the protein.

iii. HER3-621 and HER3-500

The ability of HER3-621 and HER3-500 to inhibit phosphorylation of HER3 was tested in MCF7 cells. Increasing concentrations of HER3-621 and HER3-500, up to a maximum concentration of 600 nM, was added to cells in the presence of HRG. The data show that HER3-621 and HER3-500 dose-dependently inhibited the phosphorylation of HER3, although HER3-500 was more potent. The IC50 of HER3-500 was 39 nM, and the IC50 of HER3-621 was 48 nM. The maximal inhibition of HER3 phosphorylation in MCF7 cells achieved by 600 nM HER3-500 was about 78% compared to the absence of the protein, and the maximal inhibition achieved by 600 nM HER3-621 was about 38% compared to the absence of the protein.

The ability of HER3-621 and HER3-500 to inhibit phosphorylation of HER1 and HER3 was tested in SK-BR3 cells. Increasing concentrations of HER3-621 and HER3-500, up to a maximum concentration of 600 nM, was added to the cells in the presence of HRG. Phosphorylation of HER1 was not observed in SK-BR3 cells stimulated by HER3-500. Similar to MCF7 cells, HER3-621 and HER3-500 dose-dependently inhibited the phosphorylation of HER3 in SK-BR3 cells, with HER3-500 being more potent. The maximal inhibition of HER3 phosphorylation in SK-BR3 cells achieved by 600 nM HER3-500 was about 75% compared to the absence of the protein, and the maximal inhibition achieved by 600 nM HER3-621 was about 55% compared to the absence of the protein.

iv. HER1-621/Fc

The ability of HER1-621/Fc to inhibit phosphorylation of HER1 was tested in A431 cells. Increasing concentrations of HER1-621/Fc (from 0.8 nM to 600 nM) was added to the cells in the presence of EGF. HER1-621/Fc dose-dependently inhibited phosphorylation of HER1 in A431 cells, with an IC₅₀ of 8.8 nM. At 600 nM, HER1-621/Fc showed almost complete inhibition of HER1 phosphorylation, inhibiting phosphorylation by about 99% as compared to the absence of the protein.

v. HER3-621/Fc

The ability of HER3-621/Fc to inhibit phosphorylation of HER3 was tested in MCF7 cells. Increasing concentrations of HER3-621/Fc (from 0.8 nM to 600 nM) was added to the cells in the presence of HRG. HER3-621/Fc dose-dependently inhibited phosphorylation of HER3 in MCF7 cells. The maximal inhibition of HER3 phosphorylation in MCF7 cells achieved by 600 nM HER3-621/Fc was about 70% compared to the absence of the protein.

vi. HER1-621/Fc:HER3-621/Fc Chimera

The ability of HER1-621/Fc:HER3-621/Fc chimera to inhibit phosphorylation of HER1 was tested in A431 cells. Conditioned medium supernatant from cells co-transfected with HER1-621/Fc and Her3-621/Fc was serially diluted two-fold and added to cells in the presence of EGF. The recombinant protein in neat supernatant is about 2 μg/ml (about 10 nM). Supernatant from cells not transfected with the HER ECD/Fc proteins was used as a control. The results showed that the control supernatant showed little to no inhibition of HER1 phosphorylation, with only a small inhibition (less than 10%) observed by neat supernatant. In contrast, the supernatant containing the HER1-621/Fc:HER3-621/Fc chimera dose-dependently inhibited HER1 phosphorylation in A431 cells stimulated by EGF. The maximal inhibition of HER1 phosphorylation in A431 cells achieved by the neat supernatant containing the HER1-621/Fc:HER3-621/Fc chimeras was about 55% compared to the absence of protein.

### D. Inhibition of HER Receptor Proliferation and Phosphorylation by Purified HFD100/300 ECD Multimer

1. Phosphorylation

Phosphorylation of HER receptors was assessed by purified HFD100/300H as described in section C above. The ability of purified HFD100/300H (an ECD molecule containing HER1-621/Fc and HER3-621/Fc with a His epitope tag) to inhibit phosphorylation of HER1 and HER3 was tested in SK-BR3 cells. To assess effects of HER1 phosphorylation induced by EGF, increasing concentrations of HFD100/300H from 0.3 nM to 600 nM was added to cells in the presence of EGF. The results showed that the HFD100/300H molecule dose-dependently inhibited HER1 phosphorylation of SK-BR3 cells stimulated by EGF. The maximal inhibition of HER1 phosphorylation in SK-BR3 cells achieved at 600 nM of HFD100/300H was about 60% compared to the absence of protein. To assess effects of HER3 phosphorylation induced by HRGβ, increasing concentration of HFD100/300H from 0.3 nM to 600 nM was added to cells in the presence of HRGβ. The results showed that the HFD100/300H molecule dose-dependently inhibited HER3 phosphorylation of SK-BR3 cells stimulated by HRGI3 up to a concentration of about 67 nM where the level of inhibition reached a plateau. The maximal inhibition of HER3 phosphorylation of SK-BR3 cells achieved at concentrations ranging from 67 nM to 600 nM of HFD100/300H was about 65% compared to the absence of protein.

The effects of HFD100/300H on phosphorylation of HER1, HER2, and HER3 in SK-BR3 cells stimulated by either EGF or HRGβ was compared to 2C4 (also called petuzumab), which is a monoclonal antibody that targets the dimerization domain of HER2. The results show that HFD100/300H (600 nM) inhibited phosphorylation of HER1 (about 60%), HER2 (about 65%) and HER3 (about 55%) in SK-BR3 cells stimulated by ligand. The 2C4 monoclonal antibody inhibited phosphorylation of HER2 (about 35%), HER3 (about 65%), but showed no detectable inhibition of HER1 phosphorylation. Thus, as compared to the 2C4 antibody, HFD100/300H is a pan-HER inhibitor capable of inhibiting HER1, HER2, and HER3 phosphorylation.

2. Proliferation

The effects of purified HFD100/300H on proliferation of cells stimulated by HER ligands was assessed as described in part B above. The results show that purified HFD100/300 (purified by protein A) inhibited proliferation of HT-29 cells stimulated by either of EGF (3 nM) or HRG (5 nM) in a dose dependent manner. The maximal inhibition of proliferation achieved at about 200 nM of HFD100/300 was about 55% as compared to the absence of protein in the presence of both ligands tested. The effects of purified HFD100/300H (containing a His tag) on proliferation of ZR 75-1 cells stimulated by ligands also was tested. The results show that purified HFD100/300H inhibited proliferation of ZR-75-1 cells stimulated by HRG in a dose dependent manner with maximal inhibition of about 80% observed at about 600 nM. HFD100/300H also dose-dependently inhibited proliferation of ZR-75-1 cells stimulated by EGF up to about 1 nM where the inhibition observed plateaued up to a concentration of about 600 nM HFD100/300H. The maximal inhibition observed at about 1 nM of purified HFD100/300H was about 80% as compared to the absence of protein.

### E. Summary of the Inhibitory Effects of HER ECD Derivatives on HER Phosphorylation

A variety of the exemplary HER ECD molecules were tested for their ability to inhibit HER phosphorylation. A summary of the results is set forth in Table 16. Where no determination of inhibitory effects is indicated, the experiment was not performed. The results show that the HER1-621/Fc:HER3-621/Fc chimera is a Pan-HER candidate molecule.

TABLE 16
Summary of Inhibitory Effects of Candidate HER ECD Derivatives

| HER | | | Receptor Phosphorylation | | |
|---|---|---|---|---|---|
| family | Name | Synonym | HER1 | HER2 | HER3 |
| HER1 | HF110T | HER1-501 | Y | Y | N |
| | HFD100T | HER1-621/Fc | Y | | |
| ERRP | HF120T | ERRP | Y | Y | |
| HER2 | HF210T | HER2-595 | | Y | Y |
| | HF220T | HER2-530 | | Y | Y |
| HER3 | HF300T | HER3-621 | N | | Y |
| | HF310T | HER3-500 | N | | Y |
| | HFD300T | HER3-621/Fc | | | Y |
| HER1/3 | HFDH1/H3 | HER1- | Y | Y | Y |
| | (from CM) | 621/Fc:HER3- | | | |
| | | 621/Fc | | | |
| | HFDH1/H3 | HER1- | Y | Y | Y |
| | (purified) | 621/Fc:HER3- | | | |
| | | 621/Fc | | | |

### Example 7

### Identification of the Ligand Binding Surfaces of HER1, HER3, HER4, and the Analogous Sequences of HER2

The identification of the approximate ligand binding region for all four members of the HER family was determined. The regions were determined by the crystal structure of human EGFR (residues 1-501) in complex with TGF-alpha (PDB protein data bank with 1D, 1MOX, see e.g., Garrett et al. (2002) Cell, 110: 763-773) and the multiple alignment of HER1 (SEQ ID NO:2), HER2 (SEQ ID NO:4), HER3 (SEQ ID NO:6), and HER4 (SEQ ID NO:8) in their mature forms (i.e. lacking the signal peptide as compared to the reference SEQ ID NOS). The identification of amino acids in domain I (DI) and domain III (DIII) important for ligand binding are depicted in Table 17. The numbering is according to the mature form of the HER protein. These sequences of amino acids can be targeted to interfere with binding of ligand to the respective HER protein.

TABLE 17
Identification of HER amino acid sequences that confer ligand
binding

| | DI | | DIII | |
|---|---|---|---|---|
| HER Protein | aa residues | SEQ ID NO | aa residues | SEQ ID NO |
| HER1 | S11-N128 | 54 | L325-I467 | 55 |
| HER2 | D9-R136 | 56 | R333-T475 | 57 |
| HER3 | L14-K132 | 58 | Q322-K466 | 59 |
| HER4 | E8-Q126 | 60 | Q321-R463 | 61 |

### Example 8

### Identification of Target Polypeptides in Subdomain II (DII) and Subdomain IV (DIV) of HER Family Molecules

In this Example, contiguous regions from HER3, and HER1, HER2, and HER4, were identified for use as substrates for peptide-binding (for use in, for example, phage display) or as immunogens to create multiclonal antibodies, to identify molecules that could target the subdomain II (DII) or subdomain IV (DIV) of the HER family. Such molecule could serve as candidate pan-HER therapeutics to target dimerization domains and/or to target and stabilize tethering by interacting with DII and DIV sequences involved in tethering.

The sequences of DII or DIV among the HER family receptors were aligned. HER3 was the prototype for homology analysis, and peptides conserved by sequence were identified as DII or DIV targets. Table 18 below depicts the identified target peptides in DII, with the SEQ ID NO (#) indicated in the adjacent column. Table 19 below depicts the identified target peptides in DIV, with the SEQ ID NO (#) indicated in the adjacent column.

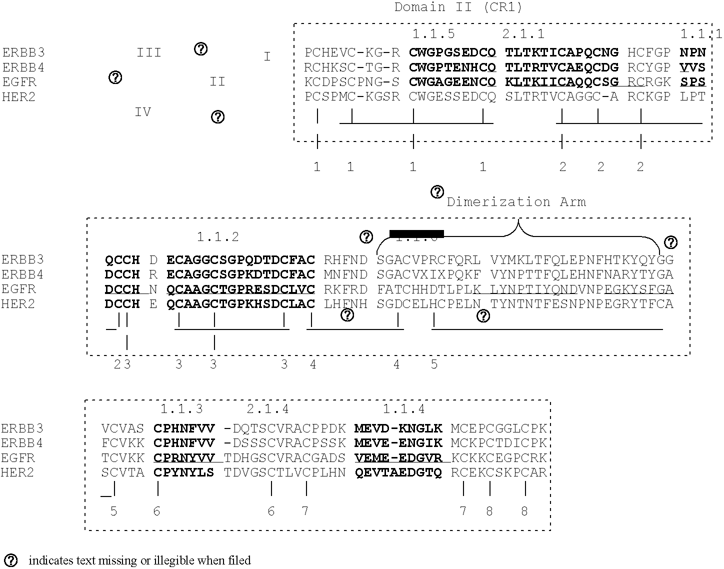

TABLE 18
Exemplary Target Polypeptides in Domain II (DII)

| | HER Family | | | | | | | |
|---|---|---|---|---|---|---|---|---|
| Pep. # | HER3 | # | HER4 | # | HER1 | # | HER2 | # |

| | | | | | | | | |
|---|---|---|---|---|---|---|---|---|
| 1.1.5 | CWGPGSEDCQ | 62 | CWGPTENNCQ | 64 | CWGAGEENCQ | 64 | CWGESSEDCQ | 65 |
| 2.1.1 | LTKTICAPQCNG | 66 | LTRTVCAEQCDG | 67 | LTKIICAQQCSG | 68 | LTRTVCAGGCA | 69 |
| 1.1.1 | NPNQCCH | 70 | YVSDCCH | 71 | SPSDCCH | 72 | LPTDCCH | 73 |
| 1.1.2 | ECAGGCSGPQDTDCFAC | 74 | ECAGGCSGPKDTDCFAC | 75 | QCAAGCTGPRESDCLVC | 76 | QCAAGCTGPKNSDCLAC | 77 |
| 1.1.6 | SGACVPRCPQPL | 78 | SGACVTQCPQTF | 79 | EATCKDTCPPLM | 80 | SGICELHCPALV | 81 |
| 1.1.3 | CPHNFVV | 82 | CPHNFVV | 83 | CPRNYVV | 84 | CPYNYLS | 85 |
| 2.1.4 | DQTSCVRACPPD | 86 | DSSSCVRACPSS | 87 | DHGSCVRACGAD | 88 | DHGSCVRACGAD | 89 |
| 1.1.4 | MEVDKNGLK | 90 | MEVEENGIK | 91 | YEMEEDGVR | 92 | QEVTAEDGTQ | 93 |

TABLE 19
Exemplary Target Polypeptides in Domain IV (DIV)

| | HER Family | | | | | | | |
|---|---|---|---|---|---|---|---|---|
| Pep. # | HER3 | # | HER4 | # | HER1 | # | HER2 | # |

| | | | | | | | | |
|---|---|---|---|---|---|---|---|---|
| 1.2.1 | LCSSGGCWGPGP | 94 | LCSSDGCWGPGP | 95 | LCSPEGCWGPEP | 96 | LCARGHCWGPGP | 97 |
| 1.2.5 | SCRNYSRGGV | 98 | SCRRFSRGRI | 99 | SCRNVSRGRE | 100 | NCSQFLRGQE | 101 |
| 1.2.2 | CNFLNGEPREF | 102 | CNLYDGEFREF | 103 | CNLLEGEPREF | 104 | CRVLQGLPREY | 105 |
| 1.2.6 | AHEAECF | 106 | ENGSICV | 107 | VENSECI | 108 | VNARHCL | 109 |
| 1.2.7 | TATCNGS | 110 | LLTCHGP | 111 | NITCTGR | 112 | SVTCFGP | 113 |
| 1.2.3 | GSTCAQCAHFRDGPHCV | 114 | GPDNCTKCSHFKDGPNCV | 115 | GPDNCIQCAHYIDGPHCV | 116 | EADQCVACAHYKDPPFCV | 117 |
| 2.2.1 | IYKYPDVQN | 118 | IFKYADPDR | 119 | VWKYADAGH | 120 | IWKFPDEEG | 121 |
| 1.2.4 | CRPCHENCTQGC | 122 | CHPCHPNCTQGC | 123 | CHLCHPNCTYGC | 124 | CQPCPINCTHSC | 125 |

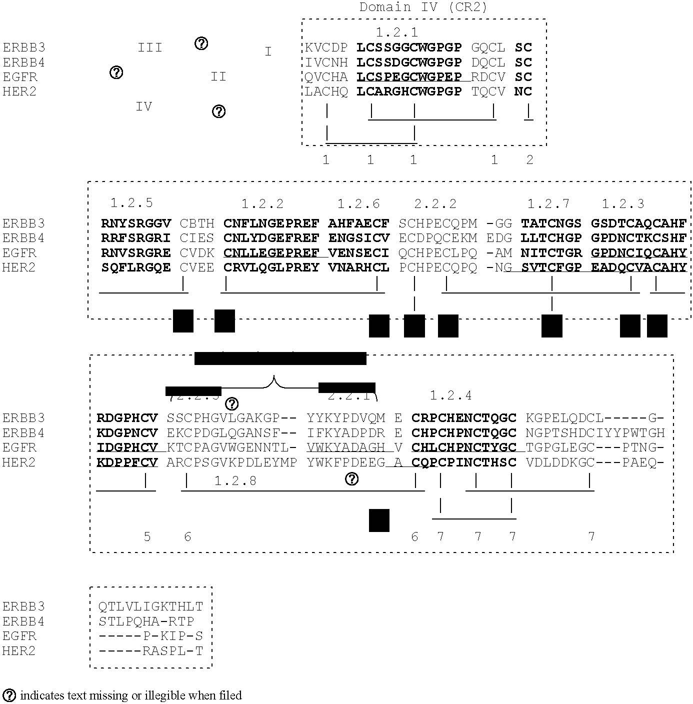

### Example 9

### Identification of Peptides by Phage Display that Bind Exposed, Conserved Residues in the HER Family

Phage display is exemplary of methods that can be used to screen for candidate therapeutics that interact with target polypeptides, such as those identified in Examples 7-8 and the identified target peptides set forth in any of SEQ ID NOS:54-125.

### A. Phage Library Selection

Phage display peptide libraries (constrained loop C7C library, and 7-aa and 12-aa linear libraries) were obtained from New England BioLabs. The phage display library was depleted against an irrelevant Fc fusion protein-protein A (or protein G) agarose complex. The depleted phage library was selected against human HER3-621/Fc-protein A agarose comples. The HER3-621/Fc which is the extracellular domain of HER3 fused with IgG1 Fc region was purchased from R&D systems, or prepared as described in Example 2. Phages were eluted with low pH buffer (or with synthetic peptide pools selected from sequence elements conserved in

HER3 domains, see Example 6 and 7 above). Four rounds of selection were performed, after which individual plaque was picked up at random and subjected to analysis by phage enzyme-linked immunosorbent assay (ELISA) and DNA sequencing following amplification in E. coli.

### B. Phage ELISA

To perform Phage ELISA, 96-well plates were coated with HER3-621/Fc; washed, and blocked with BSA/sucrose buffer. After blocking, individual phage culture medium are added to the wells and incubated for 2 hours at room temperature. Unbound phages are removed by repeated washing. Bound phages are detected using HRP conjugated M13 antibody (R&D Systems). Positive phage clones are screened further against individual synthetic peptides, which are selected from the HER3 extracellular domains conserved among ther HER receptor family members (see Example 6 and 7 above), to determine the possible phage binding sites on HER3. Similar phage binding can be carried out using monolayer cells expressing HER3.

### C. Identification of Peptides for Heterodimerization

Once positive phages are identified and binding peptides determined, avidin-biotin interaction was used to identify synergistic peptide pairs suitable for heterodimerization. The assay exploits the ability of a single avidin molecule to bind four different biotin molecules with high affinity and specificity. Briefly, biotinylated peptide and neutroavidin-HRP were mixed at a ration of 4:1. The mixture was incubated on a rotator at 4° C. for 60 minutes, followed by the addition of soft release avidin-sepharose to remove excess peptides. The soft release avidin sepharose was pelleted by centrifugation. The resulting supernatant was diluted to the desired concentration for HER3 binding assays.

### Example 10

### Method for Cloning other HER Isoforms

### A. Preparation of Messenger RNA

mRNA isolated from major human tissue types from healthy or diseased tissues or cell lines were purchased from Clontech (BD Biosciences, Clontech, Palo Alto, Calif.) and Stratagene (La Jolla, Calif.). Equal amounts of mRNA were pooled and used as templates for reverse transcription-based PCR amplification (RT-PCR).

B. cDNA Synthesis

mRNA was denatured at 70° C. in the presence of 40% DMSO for 10 min and quenched on ice. First-strand cDNA was synthesized with either 200 ng oligo(dT) or 20 ng random hexamers in a 20-μl reaction containing 10% DMSO, 50 mM Tris-HCl (pH 8.3), 75 mM KCl, 3 mM MgCl₂, 10 mM DTT, 2 mM each dNTP, 5 μg mRNA, and 200 units of StrataScript® reverse transcriptase (Stratagene, La Jolla, Calif.). After incubation at 37° C. for 1 h, the cDNA from both reactions were pooled and treated with 10 units of RNase H (Promega, Madison, Wis.).

### C. PCR Amplification

Forward and reverse primers for RT-PCR cloning were designed to clone splice variants of HER family members. Gene-specific PCR primers were selected using the Oligo 6.6 software (Molecular Biology Insights, Inc., Cascade, Colo.) and synthesized by Qiagen-Operon (Richmond, Calif.). The forward primers (F1, F2) were selected flanking the start codon. The reverse primers (R1) were selected from intron sequences of HER genes (Table 20) using the method described by Hiller et al. (Genome Biology (2005), 6: R58) (see Table 21). Each PCR reaction contained 10 ng of reverse-transcribed cDNA, 0.2 μM F1/R1 primer mix, 1 mM Mg(OAc)₂, 0.2 mM dNTP (Amersham, Piscataway, N.J.), 1× XL-Buffer, and 0.04 U/μl rTth DNA polymerase (Applied Biosystems) in a total volume of 70 μl. PCR conditions were 36 cycles of 94° C. for 45 sec, 60° C. for 1 min, and 68° C. for 2 min. The reaction was terminated with an elongation step of 68° C. for 20 min.

TABLE 20
LIST OF GENES FOR CLONING CSR Isoforms

| | | | | | SEQ | | | SEQ |
|---|---|---|---|---|---|---|---|---|
| | | Gene | | Catalytic | ID | | | ID |
| Family | Member | (SEQ ID NO.) | nt ACC. # | Domain | NO: | ORF | prt ACC.# | NO: |
| HER | EGFR | 400 | NM_005228 | 2380-3148 | 1 | 247-3879 | NP_005219 | 2 |
| | ERBB2 | 401 | NM_004448 | 2396-3164 | 3 | 239-4006 | NP_004439 | 4 |
| | ERBB3 | 402 | NM_001982 | 2318-3086 | 5 | 194-4222 | NP_001973 | 6 |
| | ERBB4 | 403 | NM_005235 | 2285-2953 | 7 | 34-3960 | NP_005226 | 8 |

TABLE 21
PRIMERS FOR PCR CLONING

| SEQ | | |
|---|---|---|
| ID NO | Primer Name | Sequence |
| 276 | EGFR-F1 | ATC GGG AGA GCC GGA GCG AG |
| 277 | EGFR-F2 | AGC AGC GAT GCG ACC CTC CG |
| 278 | EGFR-int11R1 | CCA GGC TTT GGC TGT GGT CA |
| 279 | HER2-F1 | ATG GGG CCG GAG CCG CAG T |
| 280 | HER2-F2 | GCA CCA TGG AGC TGG CGG C |
| 281 | HER2-int11R1 | ATC AGG CCC CCT CTT TCT CAG |
| 282 | HER3-F1 | TCC CTT CAC CCT CTG CGG A |
| 283 | HER3-F2 | GCG GAG TCA TGA GGG CGA A |
| 284 | HER3-int11R1 | CTG AAG ATG CCA TTT CCT CCA TAC |
| 285 | HER3-int10R1 | CAA TTT ATG CCA GTG GTT CAC CTA |
| 286 | HER4-F1 | ATT GTC AGC ACG GGA TCT GAG A |
| 287 | HER4-F2 | CTG AGA CTT CCA AAA AAT GAA GCC |
| 288 | HER4-int12R1 | AAT GGG AAA AAA TTT AAG TTT CTA |
| | | TGT T |

### D. Cloning and Sequencing of PCR Products

PCR products were electrophoresed on a 0.8% agarose gel, and DNA from detectable bands was stained with Gelstar (BioWhitaker Molecular Application, Walkersville, Md.). The DNA bands were extracted with the QiaQuick gel extraction kit (Qiagen, Valencia, Calif.), ligated into the pDrive UA-cloning vector (Qiagen), and transformed into DH10B cells. Recombinant plasmids were selected on LB agar plates containing 25 μg/ml kanamycin, 0.1 mM IPTG, and 60 μg/ml X-gal. For each transfection, 12 colonies were randomly picked and their cDNA insert sizes were determined by PCR with UA vector primers. Clones were then sequenced from both directions with M13 forward and reverse vector primers. All clones were sequenced entirely using custom primers for directed sequencing completion across gapped regions.

### E. Sequence Analysis

Computational analysis of alternative splicing was performed by alignment of each cDNA sequence to its respective genomic sequence using SIM4 (a computer program for analysis of splice variants). Only transcripts with canonical (e.g. GT-AG) donor-acceptor splicing sites were considered for analysis. Clones encoding HER isoforms were studied further (see below, Table 22).

### F. Exemplary HER Isoforms

Exemplary HER isoforms, prepared using the methods described herein, are set forth below in Table 22. Nucleic acid molecules encoding HER isoforms are provided and sequences thereof are set forth under the SEQ IDs noted in the Table. The amino acid sequences of exemplary HER isoform polypeptides are set forth under the noted of SEQ IDs.

TABLE 22
HER Isoforms

| | | | | | SEQ ID NOS |
|---|---|---|---|---|---|
| Gene | ID | Type | Length | Primers Used | (nt, aa) |
| EGFR | HER1-int11 | Intron fusion | 433 | EGFR-F1, EGFR-F2, | 126, 127 |
| | | | | EGFR-int11R1 | |
| ERBB2 | HER2-int11 | Intron fusion | 438 | HER2-F1, HER2-F2, | 140, 141 |
| | | | | HER2-int11R1 | |
| ERBB3 | HER3-int10 | Intron fusion | 403 | HER3-F1, HER3-F2, | 145, 146 |
| | | | | HER3-int11R1 | |
| ERBB3 | HER3-int11 | Intron fusion | 425 | HER3-F1, HER3-F2, | 147, 148 |
| | | | | HER3-int10R1 | |
| ERBB4 | ERBB4-int12_tr | Intron fusion | 506 | HER4-F1, HER4-F2, | 158, 159 |
| | | | | HER4-int12R1 | |
| ERBB4 | ERBB4_int11 | Intron fusion | 430 | | 156, 157 |
| ERBB4 | ERBB4_int10 | Intron Fusion | 421 | | 154, 155 |
| ERBB4 | ERBB4_int9 | Intron Fusion | 391 | | 152, 153 |

### Example 11

### Method for Cloning IGF1R Isoforms

### A. Preparation of Messenger RNA

mRNA isolated from major human tissue types from healthy or diseased tissues or cell lines were purchased from Clontech (BD Biosciences, Clontech, Palo Alto, Calif.) and Stratagene (La Jolla, Calif.). Equal amounts of mRNA were pooled and used as templates for reverse transcription-based PCR amplification (RT-PCR).

B. cDNA Synthesis

mRNA was denatured at 70° C. in the presence of 40% DMSO for 10 min and quenched on ice. First-strand cDNA was synthesized with either 200 ng oligo(dT) or 20 ng random hexamers in a 20-μl reaction containing 10% DMSO, 50 mM Tris-HCl (pH 8.3), 75 mM KCl, 3 mM MgCl₂, 10 mM DTT, 2 mM each dNTP, 5 μg mRNA, and 200 units of StrataScript® reverse transcriptase (Stratagene, La Jolla, Calif.). After incubation at 37° C. for 1 h, the cDNA from both reactions were pooled and treated with 10 units of RNase H (Promega, Madison, Wis.).

### C. PCR Amplification

Forward and reverse primers for RT-PCR cloning were designed to clone splice variants of IGF1R. Gene-specific PCR primers were selected using the Oligo 6.6 software (Molecular Biology Insights, Inc., Cascade, Colo.) and synthesized by Qiagen-Operon (Richmond, Calif.). The forward primers (F1, F2) were selected flanking the start codon. The reverse primers (R1) were selected from intron sequences of the IGFR1 genes (SEQ ID NO:404, Table 23) using the method described by Hiller et al. (Genome Biology (2005), 6: R58) (see Table 24). Each PCR reaction contained 10 ng of reverse-transcribed cDNA, 0.2 μM F1/R1 primer mix, 1 mM Mg(OAc)₂, 0.2 mM dNTP (Amersham, Piscataway, N.J.), 1× XL-Buffer, and 0.04 U/μl rTth DNA polymerase (Applied Biosystems) in a total volume of 70 μl. PCR conditions were 36 cycles of 94° C. for 45 sec, 60° C. for 1 min, and 68° C. for 2 min. The reaction was terminated with an elongation step of 68° C. for 20 min.

TABLE 23
LIST OF GENES FOR CLONING IGF1R Isoforms

| | Gene | | SEQ | | SEQ |
|---|---|---|---|---|---|
| | SEQ ID | | ID | | ID |
| Protein | NO: | nt ACC. # | NO: | prt ACC.# | NO: |
| IGF1R | 404 | X04434 | 289 | CAA28030 | 290 |

TABLE 24
PRIMERS FOR PCR CLONING

| | | | | | | |
|---|---|---|---|---|---|---|
| SEQ ID NO | Primer Name | Size | Sequence | Position | Tm | Length |

| | | | | | | |
|---|---|---|---|---|---|---|
| 291 | IGF1R_F1 | | TGA GAA AGG GAA TTT CAT CCC | 14 | 65 | 21 |
| 292 | IGF1R_F2 | | AGG AAT GAA GTC TGG CTC G | 42 | 66 | 20 |
| 293 | IGF1R_intron10R1 | 2280 | GGC TCC GTC TCA GTG GCT AC | 2358 | 66 | 20 |
| 294 | IGF1R_intron11R1 | 2496 | CTA GGT TGT GAG GAA GGT GGC | 2558 | 66 | 21 |
| 295 | IGF1R_intron12R1 | 2664 | AGG AGG TAA CCT GTG CAG TCA | 2724 | 64 | 21 |
| 296 | IGF1R_intron13R1 | 3039 | ATG TAA GCC AGG TTG AAA GCA | 3110 | 65 | 21 |

### D. Cloning and Sequencing of PCR Products

PCR products were electrophoresed on a 0.8% agarose gel, and DNA from detectable bands was stained with Gelstar (BioWhitaker Molecular Application, Walkersville, Md.). The DNA bands were extracted with the QiaQuick gel extraction kit (Qiagen, Valencia, Calif.), ligated into the pDrive UA-cloning vector (Qiagen), and transformed into DH10B cells. Recombinant plasmids were selected on LB agar plates containing 25 μg/ml kanamycin, 0.1 mM IPTG, and 60 μg/ml X-gal. For each transfection, 12 colonies were randomly picked and their cDNA insert sizes were determined by PCR with UA vector primers. Clones were then sequenced from both directions with M13 forward and reverse vector primers. All clones were sequenced entirely using custom primers for directed sequencing completion across gapped regions.

### E. Sequence Analysis

Computational analysis of alternative splicing was performed by alignment of each cDNA sequence to its respective genomic sequence using SIM4 (a computer program for analysis of splice variants). Only transcripts with canonical (e.g. GT-AG) donor-acceptor splicing sites were considered for analysis. Clones encoding IGF1R isoforms were studied further (see below, Table 25).

### F. Exemplary IGF1R Isoforms

Exemplary IGF1R isoforms, prepared using the methods described herein, are set forth below in Table 25. Nucleic acid molecules encoding IGF1R isoforms are provided and sequences thereof are set forth in any of SEQ ID NOS: 297 and 299. The amino acid sequences of exemplary HER isoform polypeptides are set forth in any of SEQ ID NOS: 298 and 300.

TABLE 25
IGF1R Isoforms

| | | | | Novel | | SEQ ID NOS |
|---|---|---|---|---|---|---|
| Gene | ID | Type | Length | length | Primers Used | (aa, nt) |
| IGF1R | SR024A03 | Intron | 759 | 25 | IGF1R_F1, | 297, 298 |
| | | fusion | | | IGF1R_F2; | |
| | | | | | IGF1R_intron10R1 | |
| IGF1R | SR024B04 | Intron | 831 | 3 | IGF1R_F1, | 299, 300 |
| | | fusion | | | IGF1R_F2; | |
| | | | | | IGF1R_intron11R1 | |

### Example 12

### Synergistic Inhibition of Tumor Cell Growth with HER 1 ECD/HER 3 ECD Heteromultimer and Tyrosine Kinase Inhibitors (TKI's)

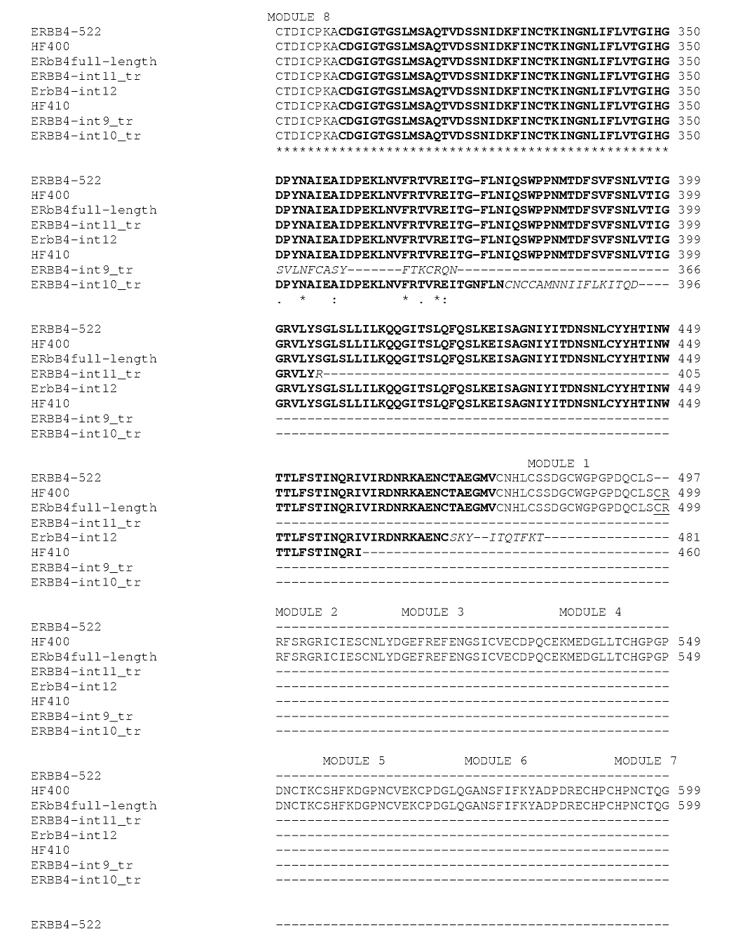

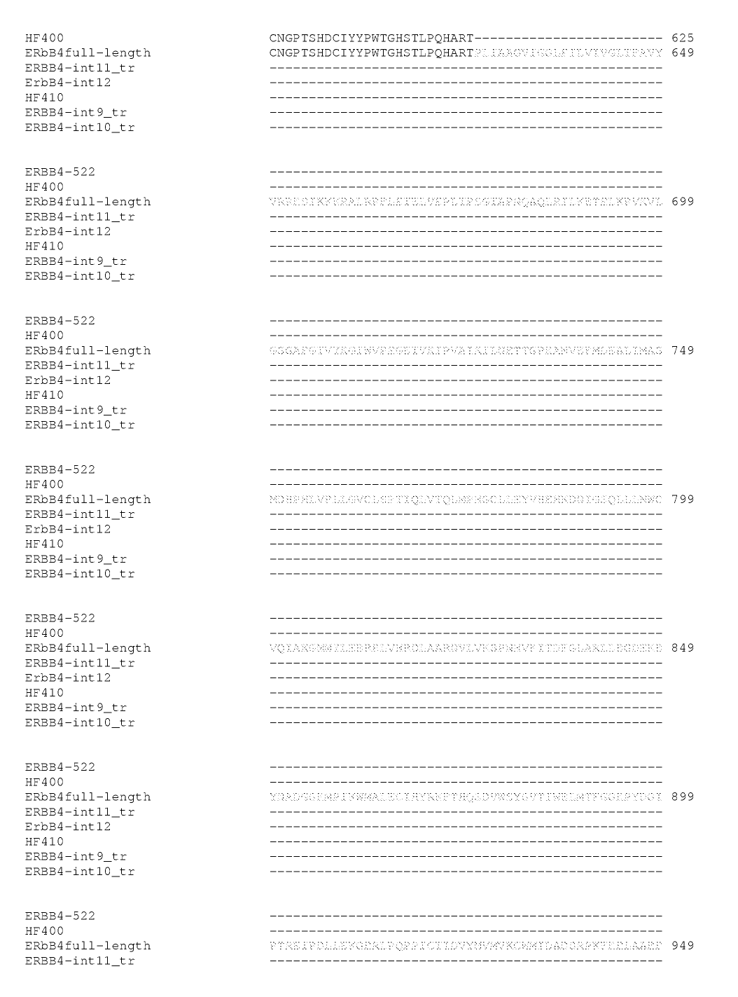

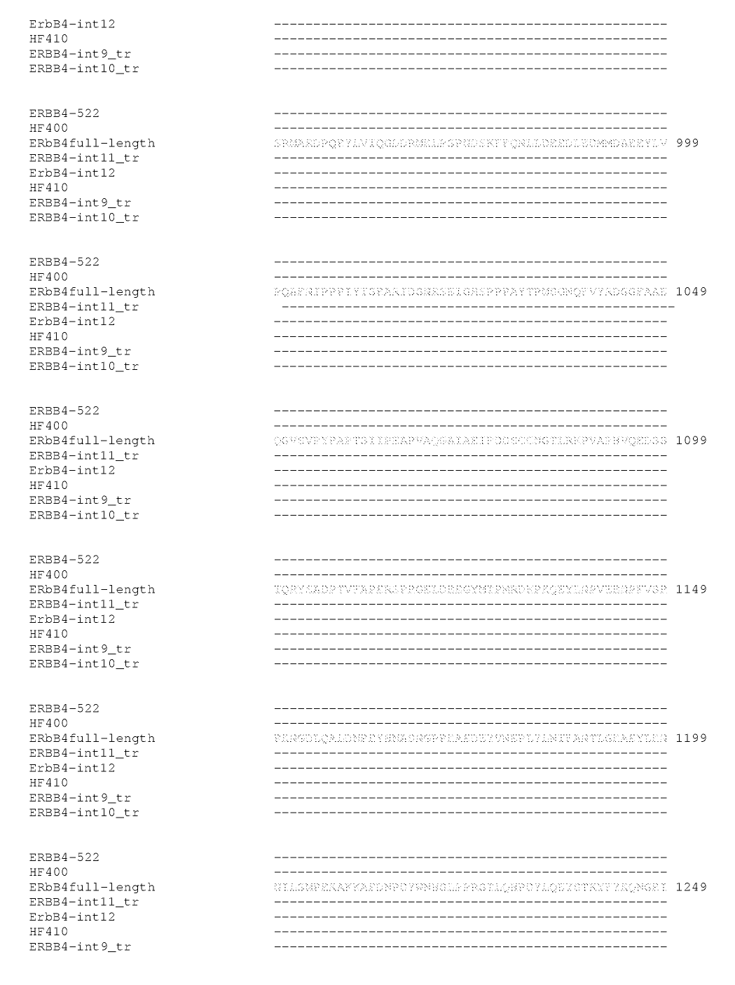

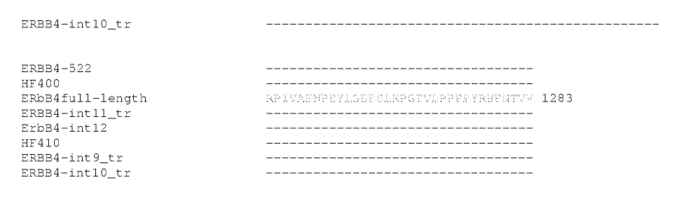

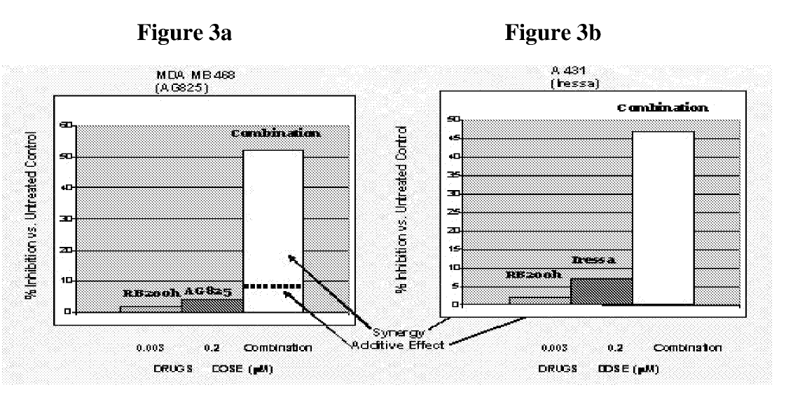

Exponentially growing tumor cells (purchased from the ATCC) were transferred to a 96-well microdilution plate at density of 1000 cells/well. (MDA MB 468 breast cancer cells were used for the experiment depicted in FIG. 3a, and A 431 squamous cell carcinoma cells were used for the experiment depicted in FIG. 3b.) Cells were allowed to attach for 24 h and test compounds were added to a final dilution of 1×: 1 uM for RB200h (full length HER 1 ECD linked to full length HER 3 ECD via Fc domain), and 50 uM of either AG-825 (an inhibitor of the HER2 associated tyrosine kinase; Osherov et al., 1993; FIG. 3A); or 50 uM of Gefitinib/Iressa (an inhibitor of the EGFR associated kinase; Herbst, 2002; FIG. 3b). Compounds were then applied simultaneously in duplicate and serial twofold dilutions were performed.

Following 72-h incubation, cells were washed with phosphate buffered saline (PBS) and stained with 0.5% crystal violet in methanol. Plates were then washed gently in water and allowed to dry overnight. Crystal violet bound to protein of attached cells was dissolved in Sorenson's buffer (0.025M sodium citrate, 0.025 M citric acid in 50% ethanol), 0.1 ml/well. Plates were analyzed in an ELISA plate reader at 540 nm wavelength. Fraction of surviving cells relative to control were plotted and analyzed (CalcuSyn; Biosoft, Cambridge, UK).

Results from the lowest concentrations tested are shown in FIGS. 3A and 3B. The dashed line across the columns labeled “Combination” is the result expected from an additive effect of the drugs tested (RB200h plus AG 825, FIG. 3A; RB200h plus Iressa, FIG. 3B). As shown in FIGS. 3A and 3B, the combination of the HER1 ECD/HER3 ECD heteromultimer (RB200h) with either tyrosine kinase inhibitor tested exhibited a synergistic growth inhibitory effect, much greater than the additive effect of the combination on growth.

This result is significant because it means toxicities associated with chemotherapeutics may be avoided by combination with RB200h. In particular, the life threatening toxicity of Iressa (see http://www.medseape.com/viewarticle/456223) approved for treatment of non-small cell lung cancer treatment, may be avoided. In addition, only about 30% of Asians and 10% of Caucasians express the mutation of the EGFR/HER1, which is required for Iressa/Gefitinib efficacy, and a similar situation may exist for other TKI's (http://en.wikipedia.org/wiki/Gefitinib). Mechanisms of resistance (other than retention of wild type amino acid sequence by the tumor associated EGFR tyrosine kinase) have also been described. Among these are acquisition of “second site” mutations (Pao et al., 2005), and overexpression of growth factors (Ishikawa et al., 2005). Thus, if the sensitivity can be increased and the toxicities associated with the TKI's can be avoided by combination with RB200h, or other receptor multimers, by synergistic enhancement of efficacy vs. toxicity. This will result in a dramatic increase in the number of patients who can be successfully treated for cancer or other diseases involving tyrosine kinases.

### Example 13

### Binding of EGF and NRG1β1 to RB200h by Biacore Surface Plasmon Resonance

In order to determine the affinity of growth factor ligands for RB200h (also called100/300h (an ECD molecule containing HER1-621/Fc and HER3-621/Fc with a HIS epitope tage)), binding studies by Biacore was done. Binding experiments were performed with the surface plasmon resonance-based biosensor instrument BIAcore 3000 (BIAcore AB, Uppsala, Sweden) at 25° C. For ligand immobilization, lyophilized human, carrier-free EGF and HRG (R&D Systems) were dissolved in HBP-ES buffer (20 mM HEPES, 150 mM NaCl, 3 mM EDTA, pH 7.5, BIAcore AB) and diluted to 0.2 mg/ml. RB200h in PBS was diluted to 0.2 mg/ml in the same buffer. Immobilization of these molecules to a BIAcore CM5 chip was carried out using NHS/EDC coupling. Either EGF or NRG1β1 was immobilized on the Biacore chips, followed by flow of RB200h solution. Once a target surface resonance of 10000 response units was reached, the surface was quenched with ethanolamine. A blank flow cell was prepared for all experiments.

Injections at different flow rates and at different analyte concentrations were done to confirm the absence of mass transfer effects. The final measurements shown in Table 26 were done in either duplicate or triplicate. Data evaluation was performed by global fitting using Scrubber (BioLogic software). The dissociation constant (Kd) of a ligand was determined from the ratio of rates of ligand dissociation to ligand association rates. Data from these studies revealed that the Hermodulin RB200h bound to EGF with a Kd of 24 nM whereas it bound NRG1β1 with a Kd of 56 nM (Table 26).

TABLE 26
Binding Affinity

| Molecule in solution | Molecule on surface | KD (nM) |
|---|---|---|
| RB200h | EGF | 24 |
| RB200h | NRG1β1 | 56 |

### Example 14

### Saturation Binding Studies of RB200h with Europium Labeled EGF or NRG1β1

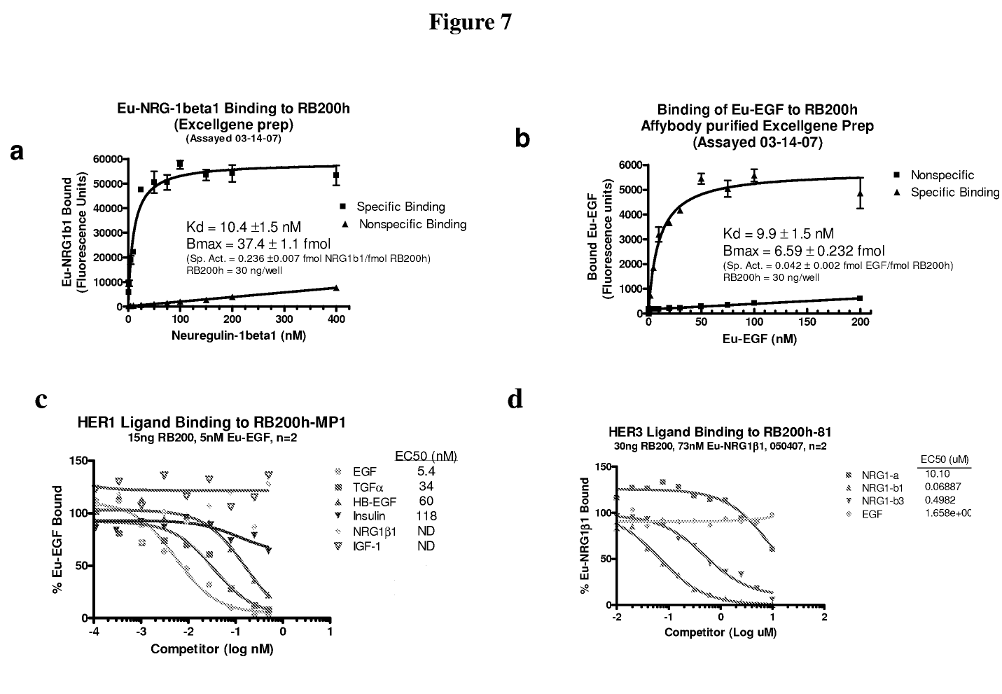

Because by Biacore method, binding of HER3 ligand (NRG1β1) to RB200h could only be determined when NRG1β1 was immobilized, binding studies of RB200h was done by another method, time resolved fluorescence assay (DELFIA). The ligand binding activities of hermodulins were determined by DELFIA method using europium tagged ligands, Eu-EGF, for HER1 ligand binding activity, or with Eu-NRG1β1 for HER3 ligand binding activity on anti-IgG Fc coated microtiter plates. RB200h was immobilized on anti-Fc coated 96-well plates and binding affinities of EGF or NRG1β1 were determined using a lanthanide (europium) tagged ligands (Eu-EGF or Eu-NRG1β1) over a wide range of concentrations as indicated in FIGS. 7a and b. The DELFIA 96-well yellow plates (PerkinElmer) were coated with anti-human IgG Fc antibody (Sigma) at 0.5 μg/well (100 μl/well volume) at 4° C. overnight. The plates were washed twice with PBS/0.05% Tween-20 and then blocked with PBS buffer containing 1% BSA, 5% sucrose and 0.01% sodium azide for 2 hrs at room temperature, approximately 22° C. (RT). After blocking, the buffer was aspirated, the plates were air-dried overnight at RT, sealed and then stored desiccated at 4° C. for up to one month. On the day of the assay, anti-IgG Fc coated plates were washed 3-times with DELFIA L*R Wash buffer (PerkinElmer), and the hermodulins were added at 10 or 20 ng/well in 50 μl/well volume in DELFIA Binding Buffer. After incubation at 30° C. for 2 hrs with gentle shaking, 50 μl of europium (Eu) labeled ligands at various concentrations indicated in the figures or below were added to the wells.

For saturation binding studies, replicate wells contained 100-fold excess unlabeled ligand together with Eu-tagged ligand for determining nonspecific binding. For routine assays of ligand binding activities of hermodulins, studies were done as above except that, a fixed saturating concentration of 30 nM Eu-EGF alone (for total binding) or in the presence of 5 uM unlabled EGF (for nonspecific binding) was used to quantify HER1 ligand binding capacity. Similarly, to quantify for HER3 ligand binding capacity, hermodulins were assayed with 100 nM Eu-NRG1β1 alone (for total binding) or in the presence of 10 μM unlabeled NRG1β1 (for nonspecific binding). Following ligand additions, incubations were performed at 30° C. for 2 hrs with gentle shaking. Then, the plates were set on ice, rapidly washed 3 times with ice-cold DELFIA wash buffer containing 0.02% Tween-20 (PerkinElmer) to remove unbound ligand. To quantify bound Eu-tagged ligands, 130 μl/well of DELFIA enhancement solution was added, the plate incubated at RT for 15 min, then read on a fluorescence plate reader (Envision, model 2100, PerkinElmer) under Eu time-resolved filter settings. The data were analyzed using GraphPad Prism for one-site or two-site binding curve fitting software to generate Kd and Bmax. For routine assays, specific binding activities of the hermodulins were expressed as fmol ligand bound per mg protein or per fmol hermodulin.

The Hermodulin RB200h bound either Eu-EGF or Eu-NRG1β1 in a saturable manner. The bindings of the Eu tagged ligands could be displaced by their respective unlabeled ligands EGF or NRG1β1, indicating that the binding is specific (FIGS. 7a and b). The Kd for Eu-EGF or NRG1β1 were approximately 10 nM. Additionally, NRG1β1 binds to immobilized RB200h with higher affinity (Kd˜10 nM) than observed via Biacore. Taken together, the data show that RB200h binds HER1 and HER3 ligands with high affinity.

### Example 15

### Hermodulin RB200h Inhibits EGF and Neuregulin-1beta Stimulated HER Family Protein Tyrosine Phosphorylations

The above examples demonstrated that the Hermodulin RB200h binds both EGF (HER1 ligand) and NRG1β1 (HER3 ligand). Studies were then done to determine whether RB200h would block ligand-induced stimulation of tyrosine phosphorylation of HER family proteins (wherein the ligand is either EGF or NRG1β1).

### Methods

Cell Lines and Tissue Culture

The human colorectal adenocarcinoma cell line HT-29, human lung carcinoma A549, gastric carcinomoa NCI-N87, mammary gland ductal carcinoma ZR-75-1, epidermoid carcinoma A431 and mammary gland adenocarcinoma cell line SK-BR-3, ACHN renal cancer cell line were purchased from the American Type Culture Collection (Manassa, Va.), whereas SUM149 cells were from Asterand. HT-29 and SK-BR-3 cells were cultured in McCoys 5a (Mediatech, Herndon, Va.) supplemented with 10% fetal bovine serum, NCI-N87 and ZR-75-1 cells were cultured in RPMI (Mediatech) supplemented with 10% fetal bovine serum, and A549 and A431 cells were cultured in DMEM (Mediatech) supplemented with 10% fetal bovine serum,. The SUM149 cells were cultured in Ham's F-12 medium supplemented with insulin (5 ug/ml), hydrocortisone (1 μg/ml), HEPES buffer (10 mM), and 5% fetal bovine serum. All cells were grown in incubators at 37° C., in a humidified atmosphere with 5% CO2 and 95% air. The cells were subcultured twice per week.

Phosphotyrosine ELISA for HER Family Proteins

A431, A549, HT-29, N87, SK-BR-3 and ZR-75-1 cells of cell lines were tested. A431 cells, have high levels of HER1 and low levels of HER2 and HER3. Cells were seeded in 96-well plates in growth medium at densities appropriate for their respective growth rates, typically 5,000-20,000 cells per well, and incubated overnight, followed by 24 hours of serum starvation. The quiescent cells were pretreated with 50 μl/well DMEM containing 0.1% BSA (Sigma, St. Louis, Mo.) and the serially diluted inhibitor (hermodulins or Herceptin, or Erbitux) added and cells incubated for 30 minutes at 37° C., 5% CO₂. The HER family protein phosphorylation was stimulated with growth factor (3 nM EGF or 1 nM NRG-β1) for 10 minutes at 37° C., 5% CO₂. After stimulation, the plates with cells were placed on ice, washed once with 200 μl/well ice-chilled PBS and lysed with 100 μl/well of ice-cold 1× Cell Lysis Buffer (Cell Signaling, Danvers, Mass.) containing phosphatase inhibitor cocktail set I and set II (EMD Biosciences, San Diego, Calif.) and protease inhibitor cocktail for general use (Sigma) for approximately 30 minutes on ice.

In initial studies, it was discovered that there was carryover of RB200h in lysates derived from cells treated with this hermodulin and this level of RB200h competed with HER1 binding to its capture antibody, but no significant competition by RB200h was observed for HER3 or HER2 binding to their respective capture antibodies described below. This competition by RB200h with HER1 for the HER1 capture antibody was eliminated by clarifying the lysates with Protein-A-Sepharose beads, which bound the Fc domain of RB200h, as described below. This was verified from experiments where RB200h at the highest concentration used in the study was spiked in the cell lysate containing HER1, HER2 and HER3 and then treated with Protein-A beads, followed by ELISA on the HER1 or HER2 or HER3 capture antibodies.

As described above, cell lysates from cells treated with RB200h were incubated with 20 μl/well of 50% proteinA-Sepharose bead slurry (Invitrogen, Carlsbad, Calif.), equilibrated in lysis buffer, overnight at 4° C. on a plate shaker, to clarify RB200h. The beads were then removed from the lysates by centrifugation and the supernatant, which was free of RB200h contamination, was used for phosphotyrosine ELISA. The HER1 or HER2 or HER3 capture antibody plates for ELISA were prepared as follows. The 96-well Immulon 4HXB microtiter plates (Thermo, Waltham, Mass.) were coated with the below described capture antibodies in PBS, 100 μl/well, for 2 hours at room temperature or overnight at 4° C. The following anti-HER extracellular domain capture antibodies were used. For HER1 detection, anti-human EGFR antibody (#AF231, 0.4 μs/ml) was the capture antibody; for HER2 detection, human anti-ErbB2 capture antibody (#DYC1768, 4 μg/ml) was used only for studies with RB200h (see below); for HER3 detection, human Erb3 DuoSet IC (#DYC1769, 4 μg/ml) was the capture antibody. We found that Herceptin competed with HER2 binding to the HER2 capture antibody mentioned above (DYC1768), but that Herceptin did not compete with cellular HER2 binding to the anti-ErbB2 capture antibody called AF1129 from R & D Systems. Thus, when Herceptin or C225 were used, HER2 detection was done in cell lysates captured on anti-human ErbB2 antibody (#AF1129, 2 μg/ml). All capture antibodies were from R&D Systems (Minneapolis, Minn.) diluted in PBS and blocked with 2% bovine serum albumin (Equitech, Kerrville, Tex.) and 0.05% Tween-20 (Fisher, Waltham, Mass.) in PBS. Cell lysate (75 ul) processed as above, was transferred to each well of the coated plates, incubated overnight at 4° C. with mixing, and then washed 4 times with PBS containing 0.05% Tween-20 (PBS-Tween). Tyrosine phosphorylation on HER proteins was detected with 100 ul/well of an anti-phosphotyrosine-HRP conjugate (R&D Systems), diluted according to the manufacturers instructions in PBS containing 2% BSA, and incubated for 2 hours at room temperature. The plates were washed 4 times with PBS-Tween, and then developed with 100 μl/well TMB substrate followed by 100 μl/well Stop Reagent for TMB (both from Sigma). Color development time was varied so that the optical densities of the developed plates were between 0.5 to 1.0. The optical density was determined by a VERSAmax microplate reader (Molecular Devices, Sunnyvale, Calif.) at 650 nm.

### Results

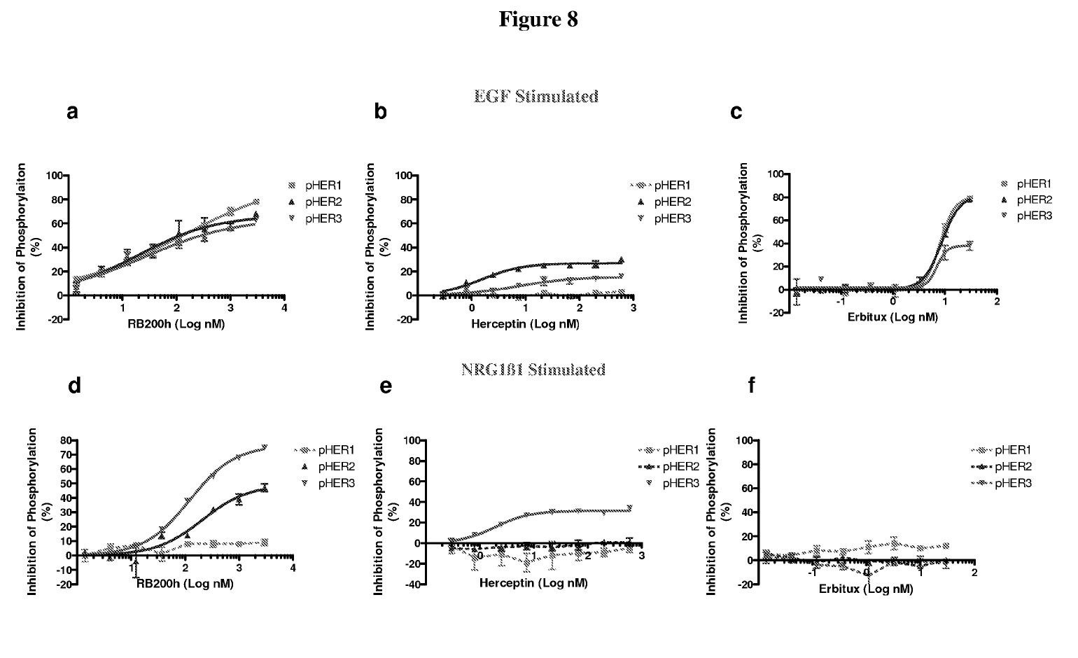

EGF treatment of A431 cells resulted in stimulation of tyrosine phosphorylation of all three HER proteins: HER1 the most stimulation (˜10-fold), followed by HER2 (4-fold) and then HER3 (2-fold). EGF stimulated phosphorylation of HER1 by 2- to 10-fold in all cell lines tested, but it stimulated HER2 phosphorylation by 1.6- to 4-fold only in A431, HT-29, SK-BR-3 and ZR-75-1 cells of cell lines tested, listed in Table 27. EGF-induced stimulation of HER3 phosphorylation by 2- to 3-fold only in A431 and SK-BR-3 cells of the cell lines tested (Table 27). When A431 cells were treated with increasing dose of RB200h, followed by stimulation with EGF, there was a dose-dependent inhibition of tyrosine phosphorylation of all three HER1, HER2 and HER3 proteins, compared with only EGF-stimulated cells, as determined by anti-phosphotyrosine ELISA. (FIG. 8a). The greatest response with RB200h, approximately 75% inhibition with an EC50 of 160 nM was observed for HER1 phosphorylation (FIG. 8a, and Tables 27 and 28). This inhibitory effect of RB200h on EGF-stimulated phosphorylation was observed in all cell lines tested, listed in Table II. However, of the other HER family directed biolgics such as Herceptin and C225 (Erbitux), only C225, which inhibits EGF binding to HER1, was as efficacious as RB200h (Tables 27 and 28). Herceptin did not inhibit EGF stimulated phosphorylation of HER proteins to any significant levels, these studies are discussed further below.

TABLE 27
Inhibition of HER family protein phosphorylation (PanHER Index)
by RB200h and other biologics.

| | RB200h | | | | | | C225 |
|---|---|---|---|---|---|---|---|
| Cell | Exp | | Herceptin | | C225 (1 nM) | | (30 nM) |
| line | #1 | Exp #2 | Exp #1 | Exp #2 | Exp #1 | Exp #2 | Exp #1 |
| | Cells Stimulated by 3 nM EGF | | | | | | |
| A431 | 65 | 68 | 21 | 16 | −4 | 0 | 65 |
| A549 | −12 | 26 | −1 | 1 | 33 | 32 | 29 |
| HT29 | 48 | 26 | −15 | −3 | 50 | 52 | 67 |
| N87 | 52 | 46 | 10 | 6 | 39 | 27 | 51 |
| SKBR3 | 58 | 61 | 17 | 19 | 42 | 47 | 63 |
| ZR751 | 18 | 23 | −38 | −15 | 28 | 23 | 28 |
| | Cells Stimulated by 1 nM NRG1b1 | | | | | | |
| A431 | 45 | 44 | 9 | 10 | 1 | 3 | 4 |
| A549 | −6 | 7 | −15 | −3 | 6 | 3 | 7 |
| HT29 | 47 | 53 | 23 | 4 | 17 | 11 | 20 |
| N87 | 40 | 40 | −2 | 2 | 8 | 5 | 19 |
| SKBR3 | 29 | 42 | −4 | −1 | 26 | 7 | 13 |
| ZR751 | 57 | 51 | 7 | 23 | 6 | 9 | 14 |

TABLE 28
Inhibition of EGF or NRG1β1 stimulated HER family protein tyrosine
phosphorylation by RB200h, Herceptin or Erbitux in tumor cells.

| | | RB200h EC50 | | | | | |
|---|---|---|---|---|---|---|---|
| | | (nM) | | Herceptin EC50 (nM) | | Erbitux EC50 (nM) | |
| Cell Line | HER prt | EGF | NRG | EGF | NRG | EGF | NRG |
| A431 | pHER1 | 160* | ND | ND | ND | 8.1 | ND |
| | pHER2 | 20 | 208 | 1.6 | ND | 8.7 | ND |
| | pHER3 | 26 | 121 | 7.4 | 2.0 | 7.0 | ND |
| A549 | pHER1 | 44 | ND | ND | ND | 0.30 | ND |
| | pHER2 | ND | ND | ND | ND | ND | ND |
| | pHER3 | ND | ND | ND | ND | ND | ND |
| HT29 | pHER1 | 20 | 550* | ND | ND | 0.22 | 0.10 |
| | pHER2 | ND | 110 | ND | ND | 0.24 | 0.20 |
| | pHER3 | ND | 180 | 25 | 1.1 | 470* | ND |
| N87 | pHER1 | 35 | 720* | ND | ND | 1.4 | 8.0 |
| | pHER2 | 19 | ND | ND | ND | 2.2 | 500* |
| | pHER3 | 3.7 | 320 | 4.4 | 3.1 | 0.32/ND | ND |
| SKBR3 | pHER1 | 450* | 350 | 5.7 | 1.9 | 0.27 | 1.2 |
| | pHER2 | 120 | ND | ND | 1.4 | 0.29 | ND |
| | pHER3 | 65 | 280 | 5.6 | ND | 0.14 | ND |
| ZR751 | pHER1 | 103 | 24 | ND | ND | 0.12 | ND |
| | pHER2 | 47 | 91 | 0.79 | 9.3 | 0.77 | ND |
| | pHER3 | ND | 96 | ND | 1.5 | ND | ND |

Besides stimulation of HER1 phosphorylation, EGF caused stimulation of HER2 (4-fold) and HER3 (3-fold) phosphorylations, suggesting that EGF induced HER1 heterodimerization with HER2 or HER3. This EGF-stimulated HER2 or HER3 phosphorylations were also inhibited to approximately 60% by RB200h (FIG. 8a). Because growth factors, such as EGF, induce heterodimerization of HER family receptor proteins and induce transphorylations of their respective partners, it is important to asses the inhibitory efficacy of a molecule on all three HER proteins stimulated by a ligand. This was done by expressing the inhibition of phosphorylation data as “panHER Index”.

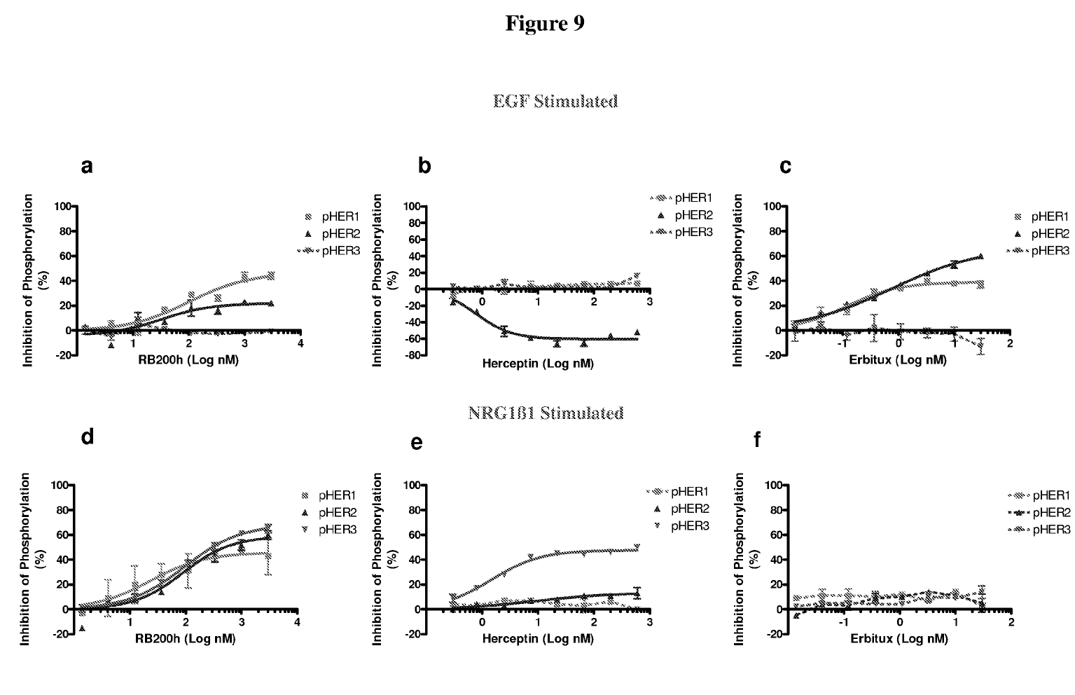

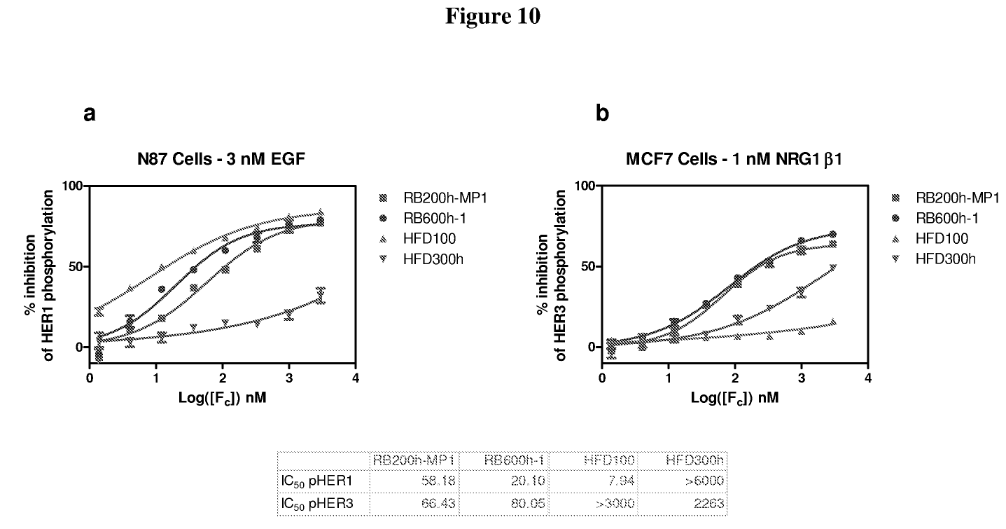

This measures the average % inhibition of HER family proteins and is derived as follows: panHER index equals (% inhibition of ligand stimulated phosphorylation of [HER130 HER2+HER3]/3) by a hermodulin or another agent. The panHER index for RB200h in A431 cells stimulated by EGF was 70%, indicating an effective blockade of EGF induced signaling of HER proteins (Table 27). In another tumor cell line ZR-75-1 breast cancer cells, which have low levels of HER1, but moderate levels of HER2 and HER3, RB200h inhibited EGF stimulated HER1 and HER2 phosphorylations by 40 and 20% respectively, with a pan HER index of ˜20% with an EC50 of 50 to 100 nM (FIG. 9a, Tables 27 and 28). In ZR-75-1 cells there was no significant increase in HER3 phosphorylation following EGF stimulation, consequently there was no effect on HER3 phosphorylation by RB200h in EGF treated cells.

NRG1β1 (HER3 ligand) treatment of A431 cells resulted in approximately 2- to 4-fold stimulation of HER3 phosphorylation. This level of stimulation of HER3 phosphorylation by NRG1β1 was seen in other cells except for ZR-75-1, where the NRG1β1 produced approximately 7-fold stimulation of HER3 phosphorylation. In most tumor cells studied, NRG1β1 stimulated phosphorylation of HER2, but HER1 phosphorylation was observed only in some tumor cell lines tested. In NRG1β1 stimulated A431 or ZR-75-1 cells, RB200h caused a dose-dependent inhibition of HER3 phosphorylation the most, and to a maximum inhibition of 60 to 80%, with an EC50 of ˜120 nM and panHER index of 45 to 60% (FIGS. 8d and 9d, Tables 27 and 28). NRG1β1 stimulation of A431 cells did not lead to any significant change in HER1 phosphorylation, hence no effect of RB200h on HER1 observed. On the other hand, NRG1β1 treatment of ZR-75-1 cells resulted in stimulation of all three HER1, HER2 and HER3 phosphorylation and these phosphorylations were inhibited by RB200h by 40 to 60%, with a panHER index of ˜50% and EC50 of 24 to 90 nM, depending on the HER protein (FIG. 9d, Tables 27 and 28). Similar studies with RB200h were conducted with other tumor cell lines and RB200h inhibited EGF or NRG1β1 stimulated phosphorylations, in a diverse range of tumor cells as well (Tables 27 and 28).

The effect of other biologics directed at HER family proteins, known to modulate HER family protein phosphorylation, namely, C225 or Erbitux (HER1 directed) and Herceptin (HER2 directed) on EGF or NRG1β1 stimulated phosphorylation was tested. In A431 and ZR-75-1 cells, C225 caused a dose-dependent inhibition of EGF-stimulated HER1 phosphorylation the most, with an EC50 of ˜8 nM and a maximum effect of 40 to 80% inhibition (FIGS. 8c and 9c, Tables 27 and 28). Similarly, C225 inhibited EGF stimulated HER1 phosphorylation in other cell lines tested with comparable efficacy as RB200h (Tables 27 and 28). In EGF stimulated A431 or ZR-75-1 cells, C225 also inhibited HER2 phosphorylation, but inhibited HER3 phosphorylation only in A431 cells, similar to effects of RB200h, but with lower efficacy towards HER3 compared with RB200h (FIG. 8c, Tables 27 and 28). However, unlike the effect of RB200h, C225, which binds HER1, did not inhibit NRG1β1 stimulated phosphorylation of HER family proteins in any of the cell lines tested (FIGS. 8c and 9c, and Tables 27 and 28).

Herceptin, directed at HER2, was tested for its ability to modulate HER family protein tyrosine phosphorylation. In EGF stimulated A431, Herceptin caused low levels (˜20%) inhibition of HER2 or HER3 phosphorylations only, whereas in NRG1β1 stimulated cells only HER3 phosphorylation was inhibited to a low, ˜30% inhibition (FIGS. 8b and e, Tables 27 and 28). However, in EGF stimulated ZR-75-1 cells, Herceptin did not inhibit HER family protein phosphorylation, but instead caused approximately 60% stimulation of HER2 phosphorylation (FIG. 9b). However, Herceptin in constrast to its effect on HER2, it inhibited HER3 phosphorylation by 50% following NRG1β1 stimulation of ZR-75-1 cells. Inhibition of HER3 tyrosine phosphorylation to low levels, 20 to 30%, by Herceptin, particularly in NRG1β1 stimulated cells, was consistenly observed in all cell lines (A431, A549, HT29, N87, SK-BR-3 and ZR-75-1 cells) tested. Of the afore-mentioned cell lines tested, only in A431 cells treatment with Herceptin resulted in a slight inhibition (˜20%) of HER2 phosphorylation. In all other cell lines, mentioned above, Herceptin treatment resulted in stimulation of HER2 tyrosine phosphorylation ranging from 10 to 60% stimulation compared with untreated cells.

Similar studies on inhibition of ligand stimulated phosphorylation by RB200h, Herceptin, and C225 was done in other cells lines as well. The data is summarized in Tables 27 and 28. By comparing the mean % inhibition (panHER Index) of HER family protein phosphorylation for RB200h, Herceptin and C225 for several cell lines, the hermodulin RB200h was most effective in inhibiting ligand induced phosphorylation of HER family proteins. While C225 was as efficacious RB200h in inhibiting EGF stimulated phosphorylation of HER family proteins, it was not efficacious in inhibiting NRG1β1 stimulated HER protein phosphorylation. With NRG1β1 stimulated cells, only RB200h and not Herceptin or C225 was effective in suppressing phosphorylation of all HER family proteins as judged by the panHER index. (Tables 27 and 28). The data show that the hermodulin RB200h, but not C225 or Herceptin blocks both EGF (HER1 ligand) or NRG1β1 (HER3 ligand) stimulated tyrosine phosphorylation of all three HER family, HER1, HER2, and HER3, phosphorylation. Taken together, the data show that RB200h is a ligand trap for HER1 and HER3 proteins and has a broad anti HER activity.

### Example 16

### Diverse Range of HER1 and HER3 Ligands Bind to RB200h

Studies were done to determine whether other HER1 ligands besides EGF or HER3 ligands besides NRG1β1 bound to RB200h. In these studies, binding ability of a ligand was tested by its ability to displace either Eu-EGF or Eu-NRG1β1 bound to RB200h. The experiment was conducted as described in Example 14.

As shown in FIGS. 7c and d, unlabeled EGF, HB-EGF, TGF-alpha inhibited Eu-EGF binding, indicating that these HER1 ligands bind to RB200h. In similar studies, NRG1-alpha, NRG1β3a, and NRG1β1, but not EGF inhibited Eu-NRG1β1 binding to RB200h, indicating that these neuregulins bind to RB200h (FIG. 7d). Moreover, growth factors such as insulin or insulin-like growth factor-1, which are unrelated to HER family ligands, did not compete for either Eu-EGF or Eu-NRG1β1 binding (FIGS. 7c and d), indicating that RB200h is specific for binding HER1 or HER3 ligands. This indicates that RB200h does not nonspecifically bind growth factors, but is highly specific for binding HER1 or HER3 ligands. The data, taken together, show that HER1 and HER3 ECDs in RB200h are functional in ligand binding ability as their natural counterparts.

### Example 17

### Ligand Binding Abilities of HER1 and HER3 in RB200h are Mutually Independent

To investigate whether ligand binding sites on HER1 and HER3 in RB200h are independent of each other, competition studies of Eu-EGF binding to RB200h was done in the presence of HER3 ligands (NRG1β1) and vice versa, competition of Eu-NRG1β1 by unlabeled EGF. The experiment was conducted as described in Example 14.

The data show that in the case Eu-EGF binding only unlabeled EGF, HB-EGF, or TGF-α, but not NRG1β1 competed with Eu-EGF binding to RB200h. Similarly, only unlabeled NGR1β1 competed Eu-NRG-1beta1 binding but not EGF. Taken together, the data show that the HER1 ligand binding site binds its ligands independent of the HER3 ligand binding site. The converse is also true, that is, HER-3 ligand binding site can bind its ligands independent of HER1 ligand binding site.

### Example 18

### Hermodulin Inhibits Cell Proliferation in Monolayer Cultures and in Soft-Agar

Because RB200h binds both EGF (HER1 ligand) and NRG1β1 (HER3 ligand) and inhibits the growth factor stimulated HER family protein tyrosine phosphorylation, it might also inhibit cell proliferation. This was tested by conducting monolayer cell proliferation studies with or without RB200h.

The soft agar colony growth assays were performed based on the method described by Hudziak et al (1987), except that the assay was done in 24-well plates with 1.5 ml of 0.5% agarose in culture medium with 10% fetal bovine serum as the base layer and the top layer containing the cells was 0.5 ml of 0.25% agarose in 10% fetal bovine serum. Compounds were added to the top layer. The colony growth was allowed to occur at 37° C. in a humidified incubator with 5% CO₂ and 95% air. At approximately every 3-days, 50 μl/well sterile water was added to prevent drying. The cell colonies were stained with 1.0 ml/well of 0.001% crystal violet in water overnight at 4° C. The cell colonies were counted using a microscope.

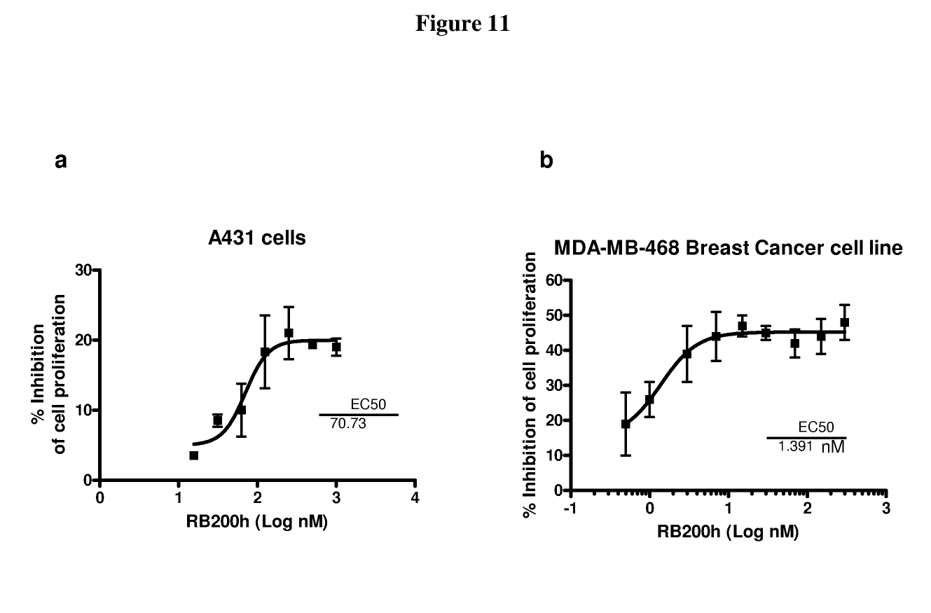

The hermodulin RB200h inhibited proliferation of A431 epidermoid cancer and MDA-MB-468 breast cancer cells in a dose dependent manner, with EC50 of 71 nM and 1.4 nM, respectively (FIG. 11). Several other tumor cell lines in monolayer culture were screened for sensitivity to RB200h. This study was expanded to include other randomly selected tumor cells. A diverse range of tumor cells, including skin, breast and lung cancer cells, are growth inhibited by RB200h (Table 29). However, some tumor cell lines including breast, lung, colon and gastric cancer cells are not sensitive to growth inhibition by RB200h (Table 29)

TABLE 29
RB200 Inhibits proliferation of a diverse range of
tumor cells in monolayer culture

| Tumor Cell Line | Tumor type | RB200 Efficacy |
|---|---|---|
| A431 | Epidermoid | +++ |
| MDA-MB-468 | Breast | ++ |
| SK-BR-3 | Breast | +++ |
| BT-474 | Breast | ++ |
| ZR-75-1 | Breast | + |
| A549 | Lung | ++ |
| H1437 | Lung | +++ |
| H1975 | Lung | + |
| SUM149 | Breast | + |
| MCF-7 | Breast | − |
| T47D | Breast | − |
| HT-29 | Colon | − |
| N-87 | Gastric | − |
| Calu-6 | Lung | − |
| H2122 | Lung | − |
| H358 | Lung | − |
| HCC4006 | Lung | − |

− = <10%; + = 10 to 20%; ++ = 21 to 30%; +++ = 31 to 50% growth inhibition of cells by RB200

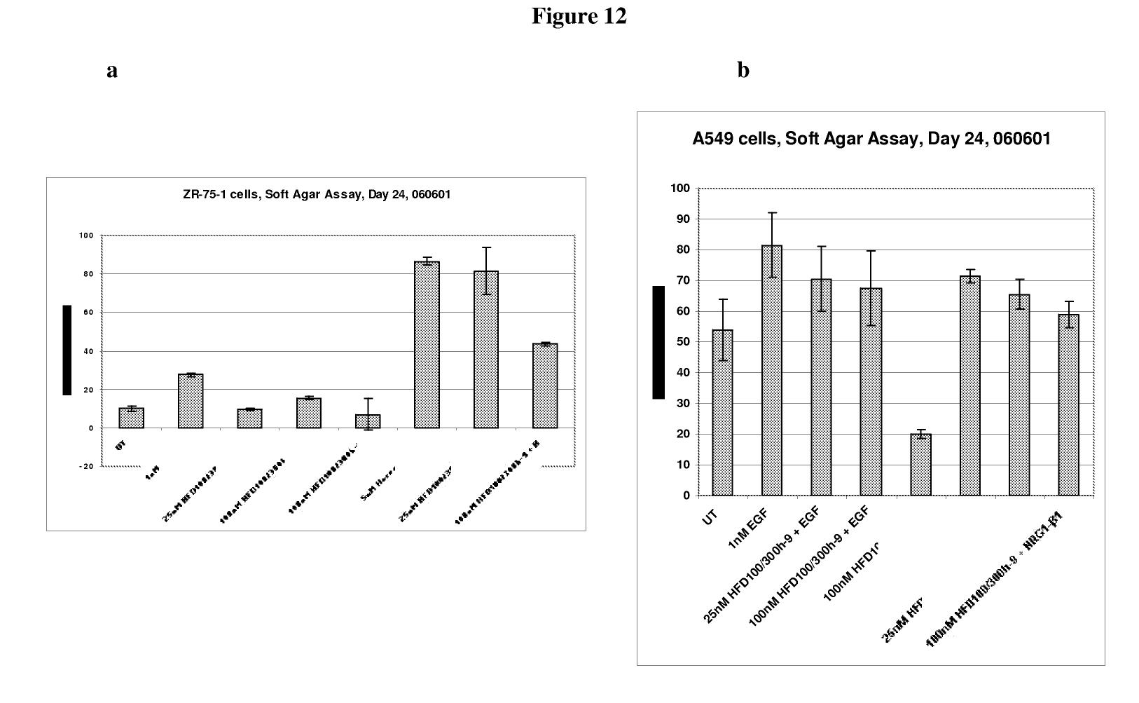

RB200h was also tested for its ability to inhibit anchorage-independent growth by soft-agar colony growth assay. Two tumor cell lines, ZR-75-1 breast cancer and A549 lung cancer cells, sensitive to growth inhibition by RB200h in monolayer growth were tested in the soft-agar assay. The ZR-75-1 cells grew poorly in soft agar, but were stimulated to form colonies with either EGF (HER1 ligand) or NRG1β1 (HER3 ligand), the latter growth factor was more efficacious, producing 9-fold stimulation whereas EGF caused 3-fold stimulation of colony growth (FIG. 12a). RB200h inhibited both EGF or NRG1β1 stimulated soft agar colony growth of ZR-75-1 cells, suggesting that RB200h is behaving like a ligand trap for these growth factors (FIG. 12a). A549 lung cancer cells readily formed colonies in soft agar, but could be stimulated by NRG1β1 or EGF by 1.3-fold and 1.4-fold, respectively compared with no growth factor treatment (FIG. 12b). This level of colony growth stimulation is much less than that observed for ZR-75-1 cells. RB200h treatment of A549 cells led to approximately 65% inhibition of colony growth in the absence of growth factors (FIG. 12b). However, RB200h did not produce statistically significant inhibition of EGF or NRG1β1 treated colony growth (FIG. 12b). This latter finding might be due to the fact that A549 cells readily formed colonies in soft agar without added growth factors and that addition of EGF or NRG1β1 caused only marginal (˜1.3-fold) stimulation in colony growth, thus they were not dependent on these ligands for colony growth. Taken, together, the data show that RB200h inhibits cell proliferation both by acting as growth factor ligand trap and by non-ligand trap mechanisms.

### Example 19

### Studies on RB200h Blocking EGF- or NRG1β1-Induced Cell Proliferation in Serum-Free Medium

To further test the hypothesis that RB200h is a HER1- or HER3-ligand trap, studies were conducted to determine whether RB200h inhibits EGF or NRG1β1 stimulated cell proliferation.

Cell proliferation studies were conducted in serum-free medium as indicated. Cells were plated in 96-well tissue culture plates (Falcon #35-3075, Becton Dickinson, N.J.) at 2000 to 6000 cells per well, as appropriate for a cell line, and then grown overnight (15 to 18 hrs). For the cell proliferation studies done in serum containing medium, the cells were then treated with or without compounds and allowed to grow for 3 to 5 days. The effect of RB200h on growth factor (EGF or NRG1β1) stimulated proliferation was done under serum-free growth conditions as follows. After plating cells in serum, the cells were grown overnight (15 to 20 hrs), then the cells were switched to serum-free medium and grown for 24 to 48 hrs (serum-starvation). They were then treated with the growth factors or LPA and with or without RB200h and grown for 3 to 5 days. Cell proliferation was quantified by crystal violet dye method as described previously (Sugarman et al., 1987). Briefly, the culture medium was decanted, the cells washed once with PBS, followed by addition of 50 μl/well 0.5% (w/v) crystal violet dye (Sigma-Aldrich, St Loius, Mo.) in methanol and incubation for 20 min. The plates were washed with water 3-times and then air-dried overnight. The cell-bound dye was eluted with 100 μl/well Sorenson's buffer (25 mM sodium citrate in 50% ethanol) for 15 min on a plate shaker. The plate was then read on a plate reader at 540 nm wavelength for absorbance, which was directly proportional to the amount of cells in the well.

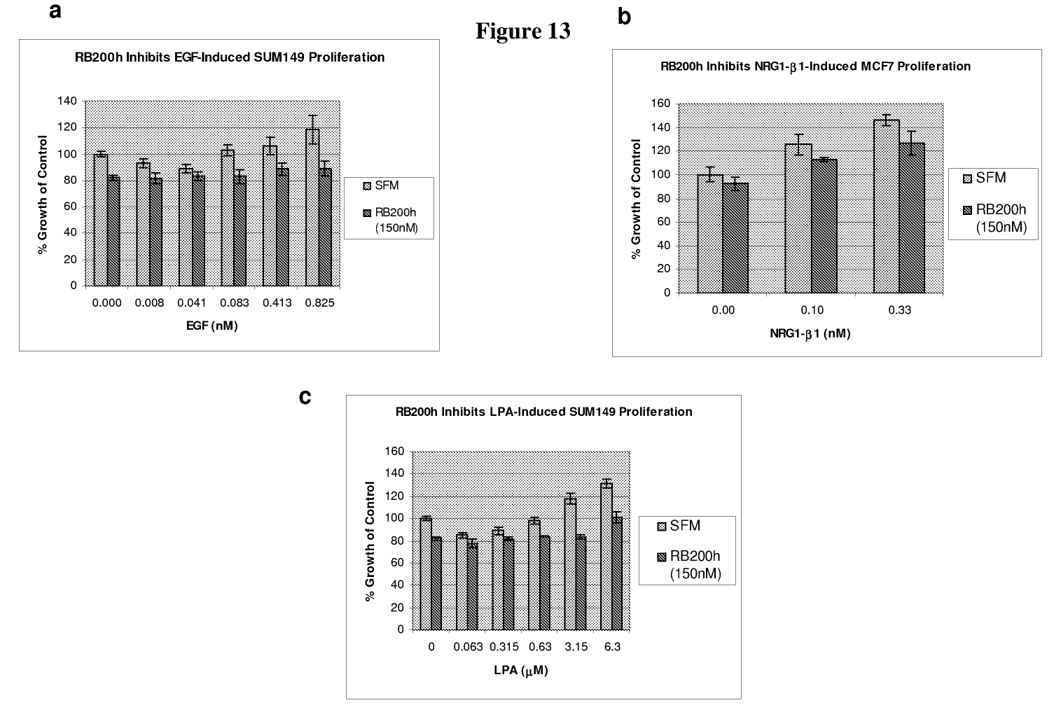

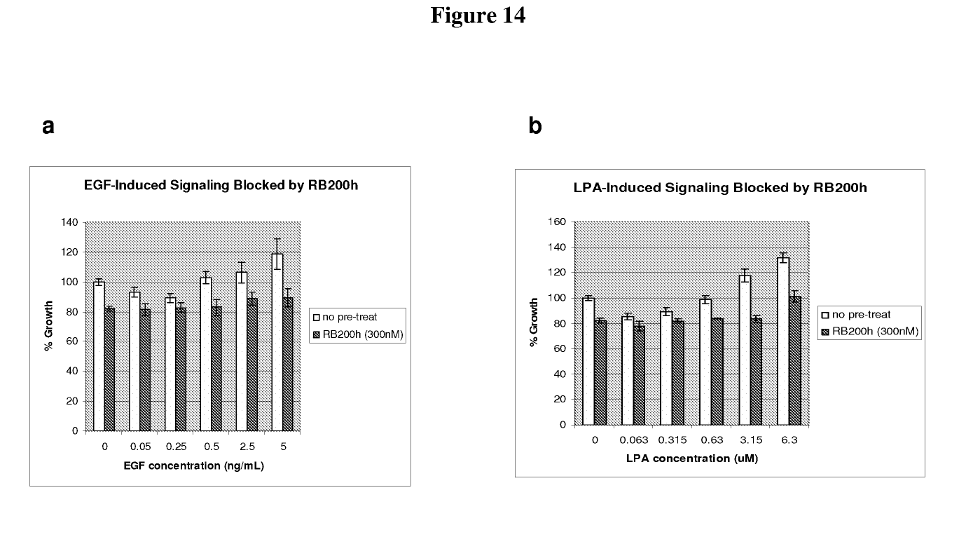

EGF stimulated the proliferation of SUM 149 cells. This EGF-stimulated proliferation was completely blocked by RB200h (FIGS. 13a and 14a). MCF-7 cells which have been reported to respond to NRG1β1 (Lewis, G D et al., Cancer Res. 1996, 56:1457-65) were treated with NRG1b1 (FIG. 13b). The growth factor produced a dose-dependent stimulation MC7 cell proliferation in serum-free condition. This NRG1β1 stimulated cell proliferation was completely blocked by RB200h.

Taken together, the antagonism of ligand stimulated proliferation data suggest that RB200h is a ligand trap for both HER1 and HER3 ligands.

### Example 20

### Hermodulin Inhibits GPCR Ligand Stimulated Cell Proliferation

An important source of growth factors for tumor cells is derived via GPCR ligand activation of ADAM metalloproteinases, which clip transmembrane bound growth factors such as, amphiregulin, HB-EGF or TGF-α, with eventual release of these growth factors (Huovila, A J et al., TIBS 2005, 30: 413-422). The growth factors thus generated are available then in either paracrine or in autocrine manner to stimulate proliferation of tumor cells. Because RB200h binds both HER1 and HER3 ligands, it may block this source of growth factors to tumor cells and lead to growth inhibition of the tumor cells. This hypothesis was tested using SUM149 breast cancer cells reported to be amphiregulin (AR) autocrine producing and AR-dependent cells (Willmarth, N E and Ethier, SP. J. Biol. Chem. 2006, 281: 37728-37737).

The cell proliferation was conducted as described in Example 19. The effect of GPCR ligand LPA stimulated proliferation was done under serum-free growth.

Treatment of SUM 149 cells with lysophosphatidic acid (LPA) led to a dose dependent increase in cell proliferation (FIGS. 13b and 14b) This LPA stimulated proliferation was completely blocked by RB200h (FIGS. 13b and 14b), consistent with the notion that the Hermodulin acts as a growth factor ligand trap for GPCR activated release of growth factors.

### Example 21

### Hermodulin is Synergistic with Tyrosine Kinase Inhibitors

Biologic agents directed at HER family proteins have shown synergistic response in inhibiting cell proliferation when combined with tyrosine kinase inhibitors directed at HER1 or HER2 kinase (Mendelsohn, J and Baselga, J. Semin. Oncol. 2006, 33: 369-385). Thus, we conducted combination studies with RB200h and tyrosine kinase inhibitors Gefitinib (Iressa), Erlotinib (Tarceva) which are FDA approved EGFR kinase inhibitors, and with tyrphostin AG 825, a HER2 kinase inhibitor, in monolayer cell proliferation assay.

Cell proliferation studies were conducted in either serum containing or in serum-free medium as indicated. Cells were plated in 96-well tissue culture plates (Falcon #35-3075, Becton Dickinson, N.J.) at 2000 to 6000 cells per well, as appropriate for a cell line, and then grown overnight (15 to 18 hrs). For the cell proliferation studies done in serum containing medium, the cells were then treated with or without compounds and allowed to grow for 3 to 5 days. For cell proliferation studies done in serum-free growth conditions. After plating cells in serum, the cells were grown overnight (15 to 20 hrs), then the cells were switched to serum-free medium and grown for 24 to 48 hrs (serum-starvation). Compounds such as RB200h, IRS, Irressa, Gefitinib, Erlotinib, and AG-825, were then applied simultaneously in duplicate and serial twofold dilutions were performed. Cell proliferation was quantified by crystal violet dye method as described previously (Sugarman et al., 1987). Briefly, the culture medium was decanted, the cells washed once with PBS, followed by addition of 50 μl/well 0.5% (w/v) crystal violet dye (Sigma-Aldrich, St Loius, Mo.) in methanol and incubation for 20 min. The plates were washed with water 3-times and then air-dried overnight. The cell-bound dye was eluted with 100 μl/well Sorenson's buffer (25 mM sodium citrate in 50% ethanol) for 15 min on a plate shaker. The plate was then read on a plate reader at 540 nm wavelength for absorbance, which was directly proportional to the amount of cells in the well.

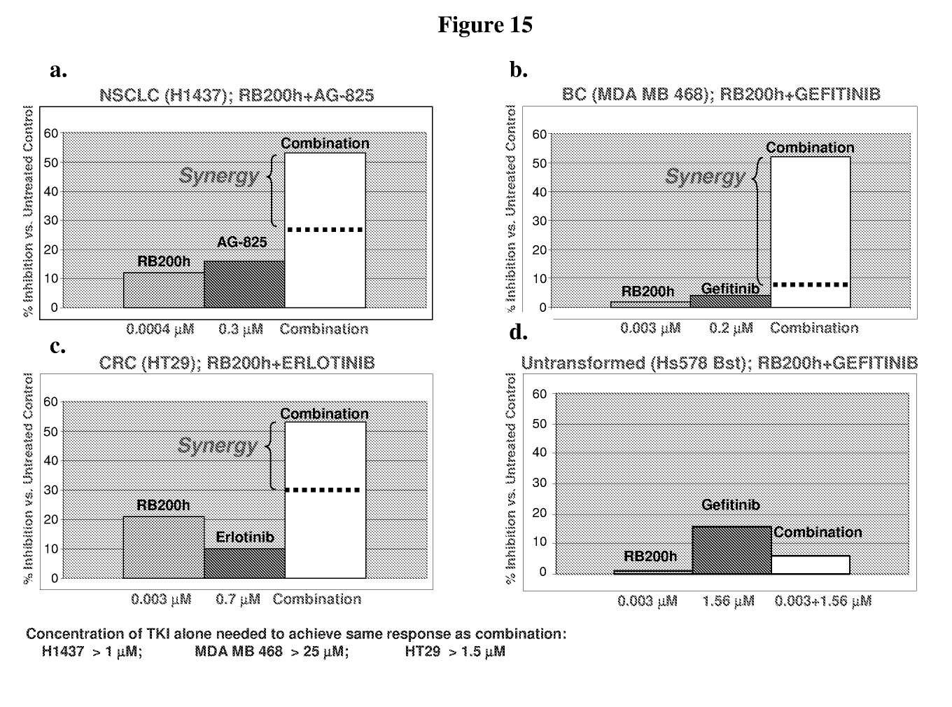

In NSCLC (H1437) cells, RB200h or AG 825 alone inhibited cell growth to low levels. These tumor cells are resistant to EGFR and HER2 kinase inhibitors. RB200h and AG 825 in combination produced a marked synergy (FIG. 15a). The synergy data was analyzed by CalcuSyn (Biosoft, Cambridge UK) a program specifically designed for objective determination of synergy in drug combinations studies (T-C Chou and P. Talalay; Trends Pharmacol. Sci 4, 450-454). Using the assay data, the CalcuSyn program generates a parameter called combination index (CI). When CI is less than 1.0 there is synergy between two compounds, CI of 1 means there is additive response and CI of greater than 1 indicates there is antagonism between the compounds. For AG-825 combination with RB200h was synergistic at all concentrations tested with a CI of 0.20 in NSCLC H1437 cells (FIG. 15a).

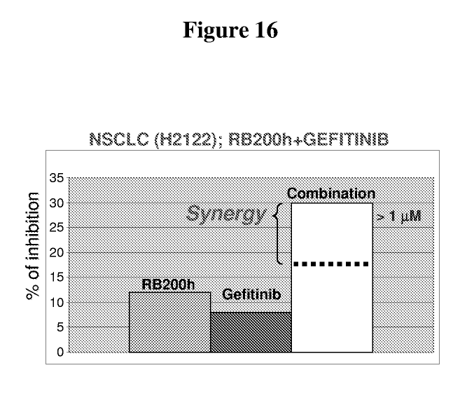

Another tyrosine kinase inhibitor, Gefitinib, directed towards EGFR was also highly synergistic with RB200h, with C.I of 0.20 in a breast cancer cell line MDA-MB-468 (FIG. 15b). This synergy with RB200h was also observed with Erlotinib, which is another FDA approved EGFR kinase inhibitor (FIG. 15c). Rb200h was also found to act synergistically with Erlotinib NSCLC cells H2122 (FIG. 16). In contrast, in normal cells, such as Hs578 Bst, RB200h had no significant inhibition of cell proliferation and also there was no synergy between RB200h and Gefitinib (FIG. 15d). Synergy between RB200h and either AG-825 or with Iressa is seen several other tumor cell lines.

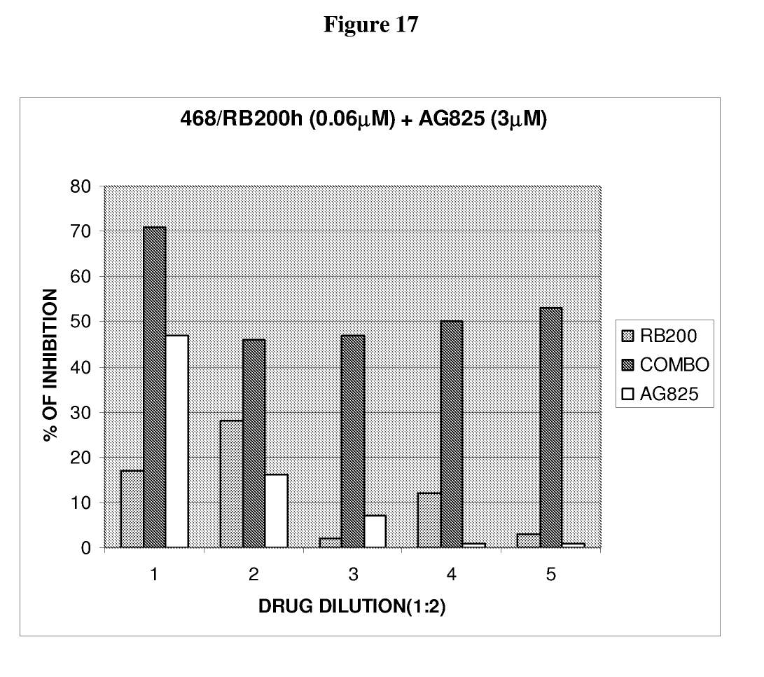

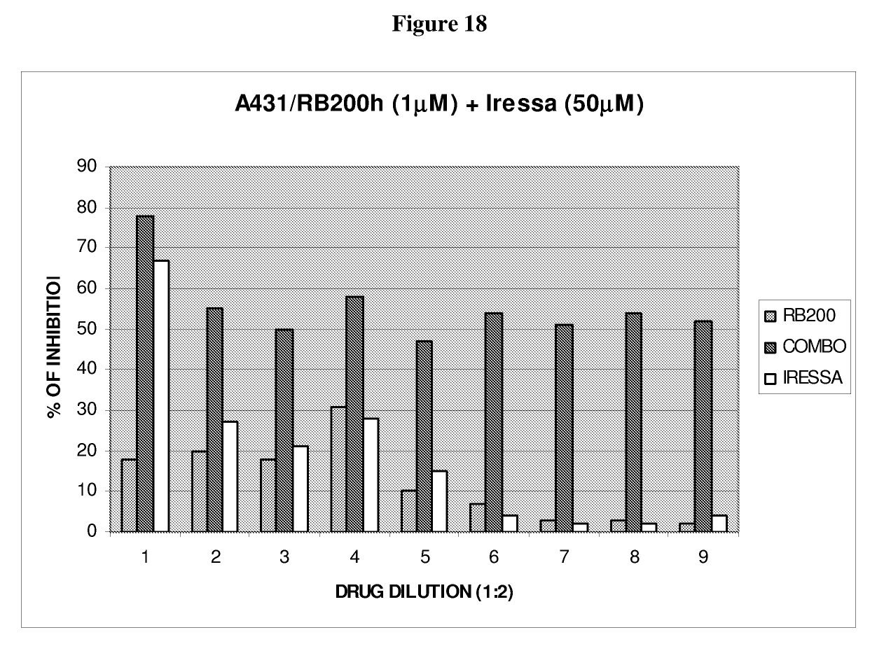

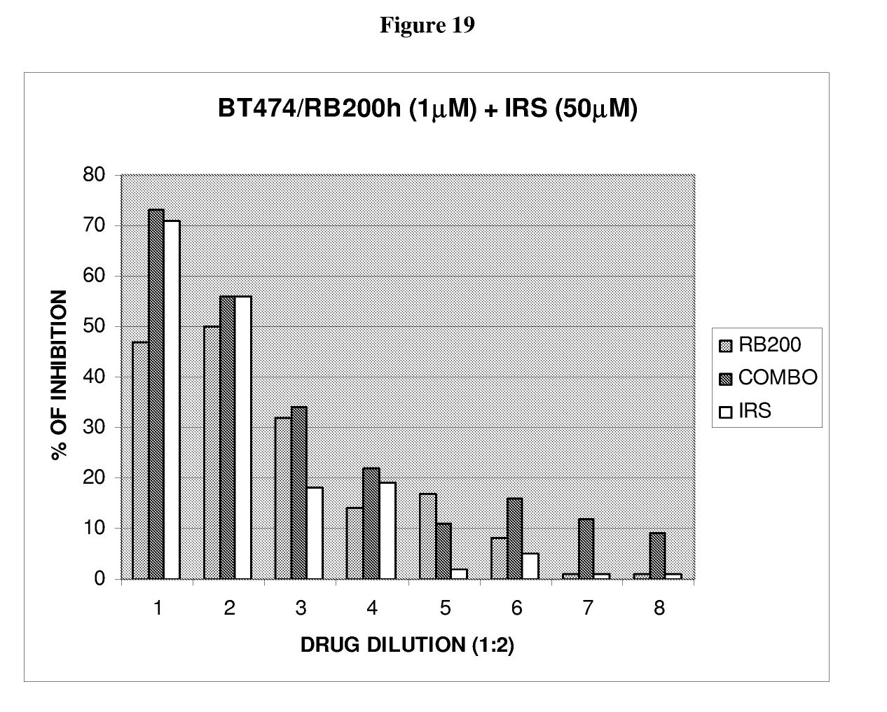

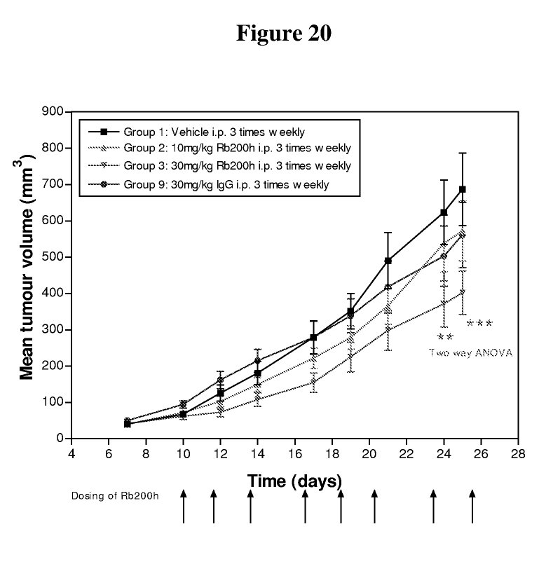

FIGS. 17-20 show that serial dilutions of RB200h and AG825 in A431 cells, RB200h and Irressa in A431 cells, and RB200h and IRS in BT474 acts synergistically to inhibit cell proliferation compared to RB200h or the tyrosine kinase inhibitor. In some cells the synergy is strong whereas in others there is weak synergy (Table 30).

TABLE 30
RB200 is Highly Synergistic with Tyrosine Kinase Inhibitors

| Tumor Cell Line | Tumor type | RB200 + AG825 | RB200 + Iressa |
|---|---|---|---|
| A431* | Epidermoid | +++ | +++++ |
| MDA-MB-468* | Breast | ++++ | +++ |
| BT-474 | Breast | +++ | + |
| HT-29 | Colon | ++ | ++ |
| N-87 | Gastric | ++ | + |
| Calu-6 | Lung | +++ | ++ |
| H2122 | Lung | ++ | ++ |
| HCC827 | Lung | + | + |
| Calu-1 | Lung | + | + |

+ less than additive;
++ moderate synergy;
+++ Synergy;
++++ Strong synergy;
+++++ very strong synergy

Taken together, the data show that RB200h at very low doses synergizes growth inhibitory activities of tyrosine kinase inhibitors directed towards HER1 or HER2 kinases. This implies that RB200h may have its greatest utility as a therapeutic in combination with tyrosine kinase inhibitors directed towards HER1 or HER2 kinases, including in those patients with resistance to these kinase inhibitors.

### Example 22

### Hermodulin RB200h has In Vivo Antitumor Efficacy in A431 Human Tumor Xenograft Model

In vivo efficacy of RB200h was tested in A431 human tumor xenograft model using nude mice. General protocols that were used are given in this example along with some deviations from the general protocol that were used.

### Animals

Mice were obtained from the commercial suppliers (Harlan, UK). The mice were 4-6 weeks old at the start of the study. Mice were maintained in sterile isolators within a barriered unit illuminated by fluorescent lights set to give a 12 hour light-dark cycle (on 07.00, off 19.00), as recommended in the United Kingdom Home Office Animals (Scientific Procedures) Act 1986. The room was air-conditioned by a system designed to maintain an air temperature range of 23±2° C. Mice were housed in groups of 2 or 5 during the procedure in plastic cages (Techniplast UK) with irradiated bedding and provided with both nesting materials and environmental enrichment. Sterile irradiated 2019 rodent diet (Harlan Teckland UK, product code Q219DJ1R2) and autoclaved water was offered ad libitum.

### Pilot Toxicity Study

There were 3 groups of 2 mice as follows: Group 1: (n=2) 30 mg/kg RB200h i.p. three times weekly, Group 2: (n=2) 75 mg/kg IRESSA i.p. on days 1-5 cycled weekly, and Group 3: (n=2) 10 mg/kg RB200h i.p. three times weekly and 38 mg/kg IRESSA i.p. on days 1-5 cycled weekly.

### Therapeutic Evaluation

There were 8 groups as follows: Group 1: (n=10) Vehicle for RB200h i.p. three times weekly, Group 2: (n=10) 10 mg/kg RB200h i.p. three times weekly, Group 3: (n=10) 30 mg/kg RB200h i.p. three times weekly, Group 4: (n=10) Vehicle for IRESSA i.p. on days 1-5 cycled weekly, Group 5: (n=10) 38 mg/kg IRESSA i.p. on days 1-5 cycled weekly, Group 6: (n=10) 75 mg/kg IRESSA i.p. on days 1-5 cycled weekly, Group 7: (n=10) Vehicle for RB200h and IRESSA, and Group 8: (n=10) 10 mg/kg RB200h i.p. three times weekly and 38 mg/kg IRESSA i.p. on days 1-5 cycled weekly.

### Tumor Initiation

A431 cells were supplied by the PRECOS and maintained in vitro in RPMI 1640 culture medium (Gibco, Paisley, UK) containing 10% (v/v) heat inactivated foetal bovine serum (Sigma, Poole, UK) at 37° C. in 5% CO₂ and humidified conditions. Cells from sub-confluent monolayers were harvested with 0.025% EDTA, washed twice in the culture medium described above, and re-suspended in sterile phosphate buffered saline, pH 7.4 (PBS) for in vivo administration. Cells were injected subcutaneously into mice at 1×10⁷ cells in a volume of 100 μl.

### Tumor Monitoring

For the pilot toxicity study, mice were allocated to their treatment groups and treatment began on day 5 for 2 weeks in a dosing volume of 150 μl per injection. For the therapeutic study mice were allocated to their treatment groups and treatment began when mean tumor volume reached 50-100 mm³ and were dosed for 3 weeks in a dosing volume of 150 μl per injection. Tumor dimensions were recorded (calliper measurement of length and width and tumor cross-sectional area and volume calculated) three times weekly and body weight measured weekly.

### Termination

Each mouse remained in the study until terminated, or until necessitate removal of that mouse from the study. Animals were terminated if the tumor size becomes excessive or any adverse effects are noted. At termination the mice were anaesthetized (Hypnorm/Hynovel) and ˜1 ml blood removed by cardiac puncture, processed for plasma, frozen for both the pilot and therapeutic study. The mice were then terminated by an approved S1 method. For the therapeutic study, the tumors were excised, weighed, measured and fixed in formalin.

### Data and Statistical Analysis

Body weight data, tumor growth and final tumor weight were recorded and reported in spreadsheet and graphical format. Statistical analysis was performed if appropriate using Minitab.

Deviations from Pilot Study

Pilot toxicity study was terminated after 12 days of dosing in order to collect plasma samples 3 hours after dosing. An extra group for the therapeutic study was added by PRECOS as below. Group 9: 30 mg/kg human IgG i.p. 3 times weekly. Iressa was prepared by PRECOS in 10% DMSO & 5% Cremaphor in PBS. Tumors were initiated with 2×10⁶ cells per mouse for the therapeutic study. RB200h and IgG was dosed intravenously for the therapeutic study as requested by the sponsors. Due to adverse effects noted following the first dose in groups 2, 3 & 8 dosing was reverted back to i.p. for the remainder of the study. The study was terminated at day 26 due to ulceration of the tumor in a number of mice.

### Results

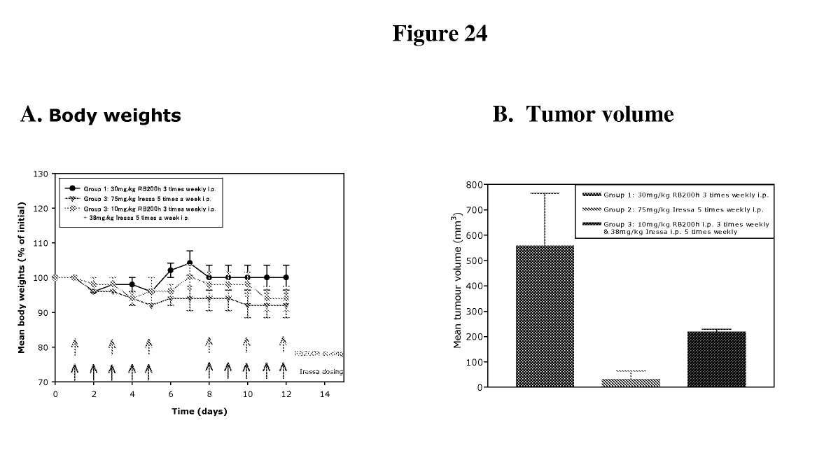

For the pilot toxicity study subcutaneous tumors were initiated with A431 cells as detailed in the protocol and dosing with Rb200h and/or Iressa was initiated in day 5. No adverse effects were seen in the A431 tumor bearing mice. The weights of the mice remained within an acceptable range throughout the toxicity study (FIG. 24A). The tumor volume was also measured prior to termination and the mean tumor size for group 1 was quite large. There was an inhibition of tumor size in Iressa treatment groups (groups 2 and 3)(FIG. 24B).

Due to the large size of the tumors present at 2 weeks following injection of 1×10⁷ cells, the cell number used to initiate the tumor was decreased to 2×10⁶ to increase the time frame of the study. The study was initiated over 2 days using 2 batches of cells and mice (Batch A and Batch B). At day 10, the mean tumor size reached 50-100 mm³ and dosing was initiated. Dosing for RB200h, IgG and RB200h vehicle was changed to intra-venous administration rather than intra-peritoneal. The first batch was dosed and an adverse reaction was observed in the RB200h treated mice in groups 2, 3 and 8. In group 3 30 mg/kg RB200h, the highest concentration used in this study, one of the mice did not recover The remaining mice were observed and recovered after 1 hour. Although RB200h has been administered i.v. previously, the RB200h batch and tumor model were different from that used in this study. The endotoxin levels were also lower in this batch than previously used. RB200h was warmed to 37° C. before i.v. dosing and 2 mice in group 3 were dosed and observed. As before the mice developed a red/purple colouration after 10 minutes and recovered after 1 hour. The dosing was therefore switched back to i.p. for the remaining mice and no further reactions were seen.

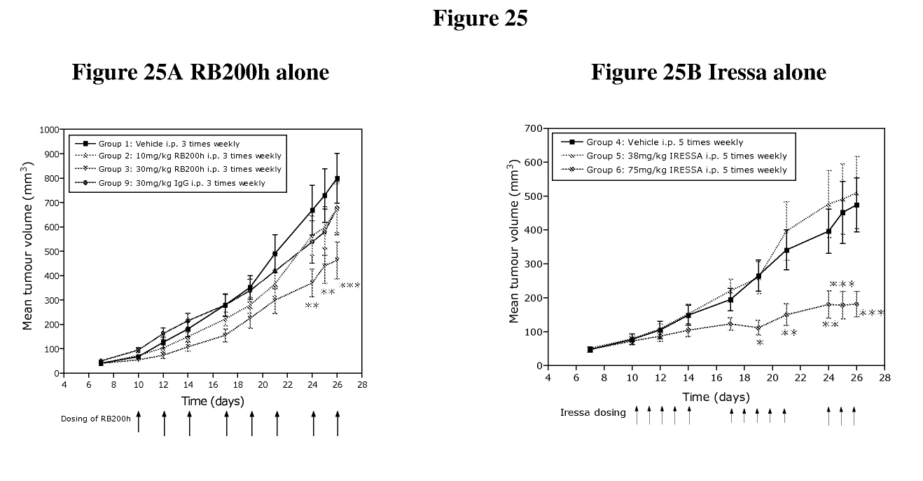

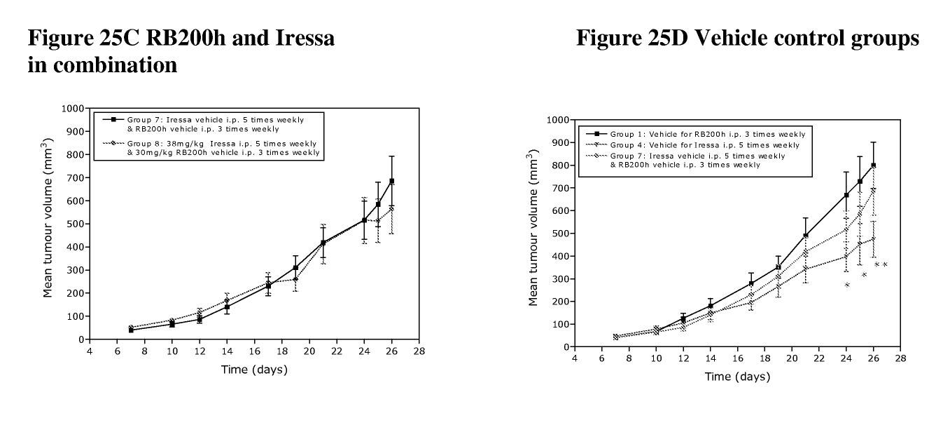

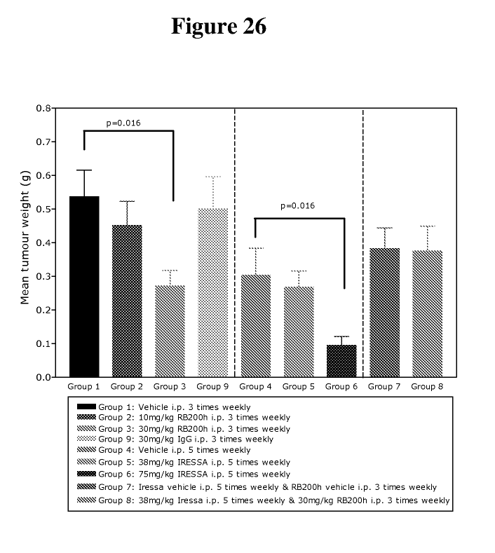

The tumor size was monitored throughout the study and the mean per group plotted over time is shown in FIGS. 25A-D. The data is also summarised in the following Table 40. The final tumor weight was also measured and the mean per group is shown in FIG. 26. The data is also summarised in Table 40.

The higher dose of RB200h alone (30 mg/kg, Group 3) significantly reduced the tumor growth rate in comparison to the vehicle group (p<0.05, Two way Anova). The final tumor weight was also significantly reduced by 50% (p=0.016, One way ANOVA, FIG. 26). In comparison, 10 mg/kg RB200h did not significantly attenuate A431 tumor growth, although there was a trend to decreasing the tumor size by approximately 15-20%. An equivalent dose of human IgG to that of RB200h (30 mg/kg, Group 9) was also included by PRECOS as a protein control. No effect on tumor growth was found with IgG.

The higher dose of Iressa (75 mg/kg, Group 6) significantly decreased the tumor growth rate (FIG. 2, p<0.001, Two way ANOVA), whereas 38 mg/kg Iressa did not in comparison to the vehicle group (group 4). The final tumor weight was also reduced by 69% when treated with 75 mg/kg Iressa (p=0.016, One way Anova, FIG. 26). The vehicle for Iressa (group 4) also had an inhibitory influence from the vehicle group for Rb200h over time (FIG. 25D, p<0.05, two way ANOVA) and reduced the final tumor size by 43% (FIG. 26) although this was not significant. In combination, 10 mg/kg RB200h and 38 mg/kg Iressa (group 8) did not influence the growth of A431 tumors (FIGS. 25C and 26). The vehicle group (Group 7) was found to reduce the tumor size by 30% but this was not found to be significant.

TABLE 40

| | | Tumor volume (mm³) | | | | | | | | | | Tumor |
|---|---|---|---|---|---|---|---|---|---|---|---|---|
| Group | | Day 7 | Day 10 | Day 12 | Day 14 | Day 17 | Day 19 | Day 21 | Day 24 | Day 25 | Day 26 | weight (g) |
| 1 | Mean | 40.4 | 67.2 | 125.6 | 180.8 | 279.0 | 351.5 | 490.2 | 668.7 | 728.7 | 799.4 | 0.536 |
| | SEM | 3.7 | 7.7 | 22.1 | 32.1 | 45.8 | 48.7 | 77.2 | 101.8 | 110.2 | 101.8 | 0.079 |
| 2 | Mean | 39.2 | 72.2 | 102.5 | 149.3 | 221.5 | 278.5 | 365.7 | 564.3 | 596.1 | 676.7 | 0.451 |
| | SEM | 3.8 | 6.2 | 14.5 | 19.0 | 28.0 | 32.5 | 51.1 | 81.2 | 86.4 | 99.7 | 0.072 |
| 3 | Mean | 40.0 | 53.7 | 72.5 | 107.7 | 154.3 | 225.0 | 298.1 | 370.3* | 440.4* | 462.5* | 0.270* |
| | SEM | 3.8 | 5.9 | 12.3 | 18.2 | 26.1 | 41.1 | 54.8 | 56.6 | 73.0 | 75.2 | 0.047 |
| 4 | Mean | 47.4 | 77.9 | 105.1 | 149.0 | 194.8 | 265.7 | 341.3 | 396.9* | 452.0* | 473.9* | 0.303 |
| | SEM | 7.5 | 15.3 | 25.6 | 29.3 | 33.0 | 46.1 | 58.6 | 64.9 | 91.2 | 79.2 | 0.081 |
| 5 | Mean | 51.2 | 79.4 | 108.8 | 152.6 | 220.6 | 259.6 | 396.9 | 477.0 | 491.1 | 510.1 | 0.267 |
| | SEM | 4.5 | 7.5 | 13.2 | 28.7 | 34.5 | 47.1 | 86.1 | 98.4 | 103.8 | 106.3 | 0.049 |
| 6 | Mean | 48.6 | 72.5 | 87.1 | 104.3 | 123.1 | 112.1# | 150.1# | 180.5# | 178.6# | 181.9# | 0.095# |
| | SEM | 5.6 | 10.8 | 15.7 | 19.0 | 18.5 | 21.1 | 31.9 | 40.1 | 40.2 | 37.1 | 0.026 |
| 7 | Mean | 39.3 | 65.3 | 85.4 | 140.1 | 229.5 | 310.4 | 418.9 | 516.1 | 584.3 | 685.5 | 0.382 |
| | SEM | 5.8 | 11.3 | 16.0 | 30.1 | 41.4 | 50.6 | 64.3 | 83.0 | 96.3 | 106.3 | 0.062 |
| 8 | Mean | 51.8 | 82.1 | 115.0 | 167.5 | 244.0 | 259.2 | 411.7 | 514.5 | 513.3 | 563.3 | 0.375 |
| | SEM | 3.0 | 9.3 | 19.0 | 31.3 | 45.4 | 50.4 | 85.4 | 99.5 | 94.0 | 105.7 | 0.074 |
| 9 | Mean | 49.8 | 94.5 | 161.9 | 214.8 | 279.1 | 338.5 | 418.0 | 538.5 | 578.2 | 676.2 | 0.501 |
| | SEM | 8.2 | 10.5 | 23.9 | 31.6 | 44.8 | 46.6 | 71.8 | 87.4 | 94.6 | 109.1 | 0.096 |

*Statistical significance from group 1
#Statistical significance from group 4

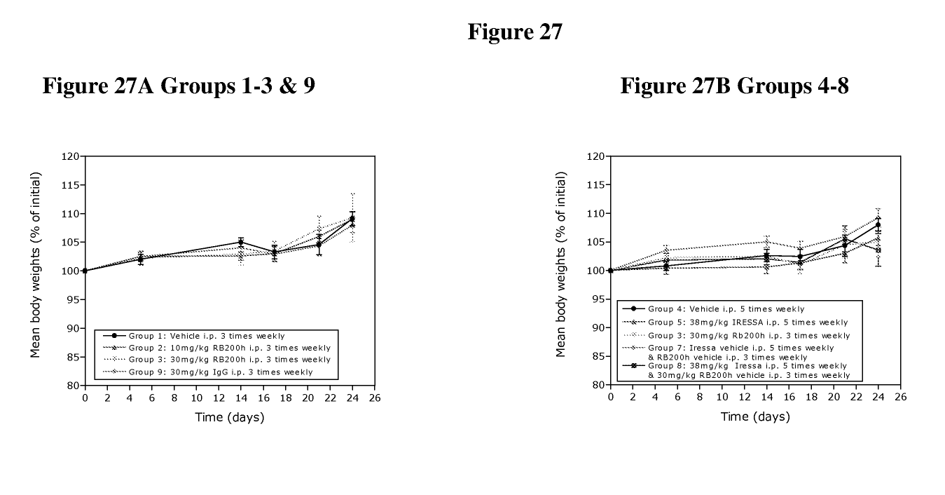

The mouse body weights were also monitored for the duration of the study and increase gradually as expected for the age of the mice (FIG. 27).

### Discussion

The objective of these experiments in this Example was to evaluate the effect of RB200h alone and in combination with Iressa in the A431 subcutaneous xenograft model. A431 epidermoid carcinomas are reported to express high levels of EGFR and Her2 (Ono M, et al., 2006. Clin Cancer Res. 12(24):7242-51) and have been used in the pre-clinical evaluation of Iressa (Wakeling A E, et al., 2002. Cancer Res. 62(20):5749-54), a selective inhibitor of EGFR tyrosine kinase domain, which is currently available in the clinic in the US for the treatment of NSCLC. RB200h is a ligand trap molecule specifically designed for pan-Her expressing tumors and therefore the A431 xenograft model was selected in order to evaluate RB200h.

Initial pilot toxicity studies showed i.p. administration to be well tolerated in mice bearing this tumor, however when the route was changed to i.v. adverse reactions were observed. This was not seen in mice bearing ZR75-1 which were dosed with RB200h i.v. (P130: Pilot toxicity study of RB200h in nude mice bearing ZR75-1 subcutaneous tumors). The endotoxin levels were also reported to be less in the batch used for the current study. The resultant effect may therefore be due to variation in alternative parameters in the batch preparations of RB200h and/or tumor type.

A dose of 30 mg/kg RB200h was found to significantly attenuate A431 tumor growth whereas 10 mg/kg did not. Similarly the top dose of 75 mg/kg Iressa significantly reduced tumor growth whereas 38 mg/kg did not. When the lower doses of the RB200h and Iressa were combined, no attenuation of tumor growth was observed. The higher doses were not combined in this study. Although Iressa had a greater therapeutic effect than RB200h the dose of Iressa used in this study was close to the MTD whereas the MTD for RB200h has not yet been determined.

Further dose escalating studies are done to determine the MTD of RB200h in line with clinically achievable doses to determine maximum therapeutic response. Another set of experiment tests for the influence of RB200h in other models such as subcutaneous ZR75-1 (high Her2 expressing breast cancer cell line), and the MDA-MB231 (high EGFR expressing breast cancer cell line) bone metastasis model. The BT20 breast cancer cell line expresses high levels of both EGFR and Her2 and would therefore be useful for the evaluation of RbB200h.

### Example 23

### Engineering for Higher Ligand Binding Affinity and Capacity panHER Ligand Traps: Structure-Based Mutagenesis of HER1 ECD

Although RB200h exhibited relatively high binding affinity for HER1 or HER3 ligands at approximately 10 nM, cells bind HER1 ligands such as EGF or TGF-a at Kd of approximately 0.3 to 3 nM and that for HER3 ligand, NRG1β1 is approximately 0.1 nM to 7.0 nM (Holmes et al; Slikowski et al; Pinkas-Kramarski et al, 1996). This suggests that tumor cells have higher affinity towards the HER1 or HER3 ligands than does RB200h, which has Kd for EGF or NRG1-β1 binding of approximately 10 nM. Thus, the intent was to design a higher affinity panHER ligand traps than observed with RB200h. This was done first by computer modeling using published co-crystal structure of EGF bound to EGFR (HER1) to optimize high affinity HER1/Fc towards its ligands. Crystal structure of the complex of human epidermal growth factor and receptor extracellular domains (Ogiso H et al. Cell (2002) 775-787) was used for computer-based optimization of the ligand-receptor interaction. The three-dimensional protein structures were from the Research Collaboratory for Structural Bioinformatics (RCSB)'s Protein Data Bank (http://www.rcsb.org/pdb). The designed optimization of ligand-receptor interaction was based on the physio-chemical proterties and classification of amino acids such as charged, polar, aromatic, etc. Also considered were residue volume, surface area, solvent accessibilities, etc. PAM250 matrix was used to aid for the prediction of amino acid substitution (W. A Pearson, Rapid and Sensitive Sequence Comparison with FASTP and FASTA, in Methods in Enzymology, ed. R. Doolittle (ISBN 0-12-182084-X, Academic Press, San Diego) 183(1990)63-98; and also M. O. Dayhoff, ed., 1978, Atlas of Protein Sequence and Structure, Vol. 5).

### A. High-Throughput Mutagenesis

This was followed by single amino acid substitutions through mutagenesis, followed by expression and screening of clones for ligand binding activities towards EGF, HB-EGF, TGF-alpha, and amphiregulin (AR). A mutant with substitution in threonine at position 39 in HER1 to serine, called T39S, was predicted by modeling studies to give rise to high affinity, was screened and found to bind EGF, TGF-alpha, and HB-EGF. This HER1/Fc T39S mutant is called HFD120. Besides HFD120, several other mutants were made.

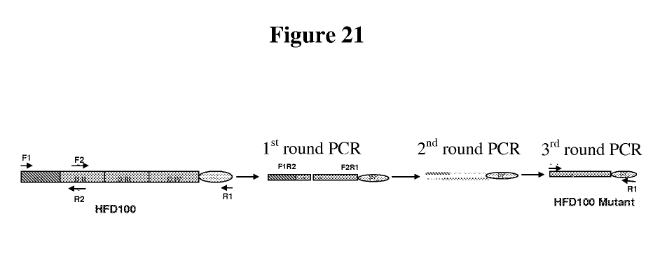

Overlapping PCR was performed using Elongase (Invitrogen) and pfu polymerase (Stratagene) to introduce the designed point mutations into HFD100 (the template) (FIG. 21 and Table 31).

Forward primer used was EGFR-F1: 5′-AATTCGTACG ACCGCCACC ATG GGA CCCTCCGGGACGGCC-3′ and reverse primer used was EGFR650-R1: GGGGACCACTTTGT ACAAGAAAGCTGGGT CTA GGA CGG GAT CTT AGG CCC A

1st round PCR: HFD100 was used as PCR template. PCR was performed using Elongase and pfu polymerase with primersEGFR-F1 and EGFRmu_R2. The PCR conditions were 94° C. 2 min, 94° C. 45 sec, 60° C. 45 sec, 68° C. 3 min for 26 cycles. EGFR-R1, the conditions were 94° C. 2 min, 94° C. 45 sec, 60° C. 45 sec, 68° C. 3 min for 26 cycles. After the amplification, PCR products were separated on 1% Agarose gel and purified using Qiagen gel purification Kit. (Qiagen).

2nd round PCR: The 1st round PCR products were mixed by molar ratio 1 to 1. PCR was performed using Elongase and pfu polymerase and the condition of 9° C. 2 min, 94° C. 45 sec, 57° C. 45 sec, 68° C. 30 min for 8 cycles.

3rd round PCR: The 2nd round PCR products were used as template. PCR was performed using Elongase and pfu polymerase with primers EGFR-F1 and EGFR-R1. The PCR conditions were 94° C. 2 min, 94° C. 45 sec, 60° C. 45 sec, 68° C. 3 min for 26 cycles. PCR products were separated on 1% Agarose gel and purified using Qiagen get purification kit. Purified PCR products were subcloned into p221DONR vector.

TABLE 31
EGFR mu Primers Used for
Mutational Analysis of HFD100

| Primer Name | Pos. bp | Well | Primer Sequence |
|---|---|---|---|
| EGFRmu01_R2 | 138 | A01 | CTC TGG AGG CTG AGA AAA |
| | | | TGT TCT TCA AAA GTG CCC |
| | | | AAC TGC G |
| EGFRmu02_R2 | 137 | A02 | CTC TGG AGG CTG AGA AAA |
| | | | TGA TTT TCA AAA GTG CCC |
| | | | AAC TGC G |
| EGFRmu03_R2 | 137 | A03 | CTC TGG AGG CTG AGA AAA |
| | | | TGT TGT TCA AAA GTG CCC |
| | | | AAC TGC G |
| EGFRmu04_R2 | 124 | A04 | TGA GAA AAT GAT CTT CAA |
| | | | AAG TGT TCA ACT GCG TGA |
| | | | GCT TGT TAC |
| EGFRmu05_R2 | 124 | A05 | CTC TGG AGG CTG AGA AAA |
| | | | TGT TCT TCA AAA GTG TTC |
| | | | AAC TGC GTG AGC TTG TTA |
| | | | C |
| EGFRmu06_R2 | 121 | A06 | AAA ATG ATC TTC AAA AGT |
| | | | GCC CAC CTG CGT GAG CTT |
| | | | GTT ACT CG |
| EGFRmu07_R2 | 121 | A07 | GAA AAT GAT CTT CAA AAG |
| | | | TGC CAA TCT GCG TGA GCT |
| | | | TGT TAC TC |
| EGFRmu08_R2 | 277 | A08 | GAA TTC GCT CCA CTG TGT |
| | | | TGA CGG CAA TGA GGA CAT |
| | | | AAC CAG |
| EGFRmu09_R2 | 277 | A09 | GAA TTC GCT CCA CTG TGT |
| | | | TGA TGG CAA TGA GGA CAT |
| | | | AAC CAG |
| EGFRmu10_R2 | 205 | A10 | AAA GAT CAT AAT TCC TCT |
| | | | GCA CCC AGG TAA TTT CCA |
| | | | AAT TCC CA |
| EGFRmu11_R2 | 338 | A11 | CTG CTA AGG CAT AGG AAT |
| | | | TTT CCC AGT ACA TAT TTC |
| | | | CTC TGA TGA T |
| EGFRmu12_R2 | 342 | A12 | GAC TGC TAA GGC ATA GGA |
| | | | ATT ATC GTA GTA CAT ATT |
| | | | TCC TCT GA |
| EGFRmu13_R2 | 340 | B01 | ACT GCT AAG GCA TAG GAA |
| | | | TTT TGG TAG TAC ATA TTT |
| | | | CCT CTG ATG |
| EGFRmu14_R2 | 367 | B02 | GGT TTT ATT TGC ATC ATA |
| | | | GTT AGC TAA GAC TGC TAA |
| | | | GGC ATA GGA |
| EGFRmu15_R2 | 367 | B03 | GGT TTT ATT TGC ATC ATA |
| | | | GTT AGT TAA GAC TGC TAA |
| | | | GGC ATA GGA |
| EGFRmu16_R2 | 128 | B04 | GGC TGA GAA AAT GAT CTT |
| | | | CAA ATT TGC CCA ACT GCG |
| | | | TGA GCT T |
| EGFRmu17_R2 | 128 | B05 | GGC TGA GAA AAT GAT CTT |
| | | | CAA ATT GGC CCA ACT GCG |
| | | | TGA GCT T |
| EGFRmu18_R2 | 129 | B06 | GGC TGA GAA AAT GAT CTT |
| | | | CAA AAA TGC CCA ACT GCG |
| | | | TGA GCT |
| EGFRmu19_R2 | 128 | B07 | GGC TGA GAA AAT GAT CTT |
| | | | CAA AAT CGC CCA ACT GCG |
| | | | TGA GCT T |
| EGFRmu20_R2 | 128 | B08 | GGC TGA GAA AAT GAT CTT |
| | | | CAA AAT AGC CCA ACT GCG |
| | | | TGA GCT T |
| EGFRmu21_R2 | 128 | B09 | GGC TGA GAA AAT GAT CTT |
| | | | CAA AAC CGC CCA ACT GCG |
| | | | TGA GCT T |
| EGFRmu22_R2 | 128 | B10 | GGC TGA GAA AAT GAT CTT |
| | | | CAA AAA GGC CCA ACT GCG |
| | | | TGA GCT T |
| EGFRmu23_R2 | 145 | B11 | GAA CAT CCT CTG GAG GCT |
| | | | GGC AAA ATG ATC TTC AAA |
| | | | AGT GCC CA |
| EGFRmu24_R2 | 145 | B12 | TTG AAC ATC CTC TGG AGG |
| | | | CTC CAA AAA TGA TCT TCA |
| | | | AAA GTG CCC |
| EGFRmu25_R2 | 149 | C01 | TTA TTG AAC ATC CTC TGG |
| | | | AGG GTG AGA AAA TGA TCT |
| | | | TCA AAA GTG |
| EGFRmu26_R2 | 148 | C02 | TAT TGA ACA TCC TCT GGA |
| | | | GGA GGA GAA AAT GAT CTT |
| | | | CAA AAG TGC |
| EGFRmu27_R2 | 145 | C03 | GTT ATT GAA CAT CCT CTG |
| | | | GAG GAG GGC AAA ATG ATC |
| | | | TTC AAA AGT GCC C |
| EGFRmu28_R2 | 145 | C04 | GTT ATT GAA CAT CCT CTG |
| | | | GAG TTG GGC AAA ATG ATC |
| | | | TTC AAA AGT GCC C |
| EGFRmu29_R2 | 148 | C05 | TTA TTG AAC ATC CTC TGG |
| | | | AGG GCG AGA AAA TGA TCT |
| | | | TCA AAA GTG C |
| EGFRmu30_R2 | 145 | C06 | TTA TTG AAC ATC CTC TGG |
| | | | AGG GCG TAA AAA TGA TCT |
| | | | TCA AAA GTG CCC A |
| EGFRmu31_R2 | 145 | C07 | TTA TTG AAC ATC CTC TGG |
| | | | AGG GCG TTA AAA TGA TCT |
| | | | TCA AAA GTG CCC |
| EGFRmu32_R2 | 118 | C08 | TGA TCT TCA AAA GTG CCC |
| | | | AAC TCC GTG AGC TTG TTA |
| | | | CTC GTG CC |
| EGFRmu33_R2 | 118 | C09 | TGA TCT TCA AAA GTG CCC |
| | | | AAC GAC GTG AGC TTG TTA |
| | | | CTC GTG C |
| EGFRmu34_R2 | 118 | C10 | GAT CTT CAA AAG TGC CCA |
| | | | ACT TCG TGA GCT TGT TAC |
| | | | TCG TGC |
| EGFRmu35_R2 | 118 | C11 | ATG ATC TTC AAA AGT GCC |
| | | | CAA GTA CGT GAG CTT GTT |
| | | | ACT CGT G |
| EGFRmu36_R2 | 115 | C12 | CTT CAA AAG TGC CCA ACT |
| | | | GCG AGA GCT TGT TAC TCG |
| | | | TGC CTT |
| EGFRmu37_R2 | 116 | D01 | CTT CAA AAG TGC CCA ACT |
| | | | GCT TGA GCT TGT TAC TCG |
| | | | TGC CTT |
| EGFRmu38_R2 | 115 | D02 | CTT CAA AAG TGC CCA ACT |
| | | | GCT CGA GCT TGT TAC TCG |
| | | | TGC CTT |
| EGFRmu39_R2 | 115 | D03 | ATC TTC AAA AGT GCC CAA |
| | | | CTG ATA GAG CTT GTT ACT |
| | | | CGT GCC |
| EGFRmu40_R2 | 100 | D04 | ACT GCG TGA GCT TGT TAC |
| | | | TCT GGC CTT GGC AAA CTT |
| | | | TCT TTT C |
| EGFRmu41_R2 | 1300 | D05 | CGA CTG CAA GAG AAA ACT |
| | | | GAC GAT GTT GCT TGG TCC |
| | | | TGC CG |
| EGFRmu42_R2 | 1300 | D06 | ACG ACT GCA AGA GAA AAC |
| | | | TGA TTA TGT TGC TTG GTC |
| | | | CTG CCG |
| EGFRmu43_R2 | 1393 | D07 | GCA TAG CAC AAA TTT TTG |
| | | | TTT CGT GAA ATT ATC ACA |
| | | | TCT CCA TC |
| EGFRmu44_R2 | 1393 | D08 | TTT GCA TAG CAC AAA TTT |
| | | | TTG TTA TGT GAA ATT ATC |
| | | | ACA TCT CCA TC |
| EGFRmu45_R2 | 146 | D09 | TTG AAC ATC CTC TGG AGG |
| | | | CTT TGA AAA TGA TCT TCA |
| | | | AAA GTG CC |
| EGFRmu46_R2 | 1109 | D10 | AAA TGC CAC CGG CAG GAT |
| | | | GCG GAG ATC GCC ACT GAT |
| | | | GGA |
| EGFRmu47_R2 | 1120 | D11 | GAG TCA CCC CTA AAT GCC |
| | | | AGC GGC AGG ATG TGG AGA |
| | | | TCG |
| EGFRmu48_R2 | 1126 | D12 | TGT GAA GGA GTC ACC CCT |
| | | | ATG TGC CAC CGG CAG GAT |
| | | | GTG |
| EGFRmu49_R2 | 1132 | E01 | GAG TAT GTG TGA AGG AGT |
| | | | CAG CCC TAA ATG CCA CCG |
| | | | GCA |
| EGFRmu50_R2 | 1132 | E02 | GAG TAT GTG TGA AGG AGT |
| | | | CAT TCC TAA ATG CCA CCG |
| | | | GCA G |
| EGFRmu51_R2 | 1226 | E03 | CCG TCC TGT TTT CAG GCC |
| | | | ATT CCT GAA TCA GCA AAA |
| | | | ACC CT |
| EGFRmu52_R2 | 1226 | E04 | CCG TCC TGT TTT CAG GCC |
| | | | AAT CCT GAA TCA GCA AAA |
| | | | ACC CT |
| EGFRmu53_R2 | 1228 | E05 | GTC CGT CCT GTT TTC AGG |
| | | | CTC AGC CTG AAT CAG CAA |
| | | | AAA CC |
| EGFRmu54_R2 | 1330 | E06 | GTA ATC CCA AGG ATG TTA |
| | | | TGT CCA GGC TGA CGA CTG |
| | | | CAA GA |
| EGFRmu55_R2 | 1472 | E07 | CTG TTT TCA CCT CTG TTG |
| | | | CTT TTA ATT TTG GTT TTC |
| | | | TGA CCG G |
| EGFRmu56_R2 | 1459 | E08 | TCT GTT GCT TAT AAT TTT |
| | | | GGT TTC CTG ACC GGA GGT |
| | | | CCC AAA C |
| EGFRmu57_R2 | 1459 | E09 | TCT GTT GCT TAT AAT TTT |
| | | | GGT TTG CTG ACC GGA GGT |
| | | | CCC AAA C |
| EGFRmu58_R2 | 1475 | E10 | GCA GCT GTT TTC ACC TCT |
| | | | GTT TTT TAT AAT TTT GGT |
| | | | TTT CTG ACC G |
| EGFRmu59_R2 | 166 | E11 | CCA AGG ACC ACC TCA CAG |
| | | | TTT TCG AAC ATC CTC TGG |
| | | | AGG CTG |
| EGFRmu60_R2 | 160 | E12 | CCA CCT CAC AGT TAT TGA |
| | | | ACA GCC TCT GGA GGC TGA |
| | | | GAA AAT |
| EGFRmu61_R2 | 160 | F01 | CCA AGG ACC ACC TCA CAG |
| | | | TTT TCG TAC AGC CTC TGG |
| | | | AGG CTG AGA AAA |
| EGFRmu62_R2 | 127 | F02 | GAG GCT GAG AAA ATG ATC |
| | | | TTC AGC ATC GCC CAA CTG |
| | | | CGT GAG CTT |
| EGFRmu63_R2 | 127 | F03 | TCT GGA GGC TGA GAA AAT |
| | | | GAT TTT CAG CAT CGC CCA |
| | | | ACT GCG TGA GCT T |
| EGFRmu64_R2 | 95 | F04 | TGA GCT TGT TAC TCG TGC |
| | | | CTG GGC AAA CTT TCT TTT |
| | | | CCT CCA |
| EGFRmu65_R2 | 283 | F05 | TGC AGG TTT TCC AAA GGA |
| | | | ATT GTC GAA AAT TCG TTG |
| | | | AGG GCA ATG AGG ACA |
| EGFRmu66_R2 | 314 | F06 | GTA CAT ATT TCC TCT GAT |
| | | | GAT CCG CAG GTT TTC CAA |
| | | | AGG AAT TC |
| EGFRmu67_R2 | 329 | F07 | AAG GCA TAG GAA TTT TCG |
| | | | TAG ACC TGA GTT CCT CTG |
| | | | ATG ATC TGC AGG |
| EGFRmu68_R2 | 364 | F08 | CGG TTT TAT TTG CAT CAT |
| | | | AGT TTA ACA TGA CTG CTA |
| | | | AGG CAT AGG AAT |
| EGFRmu69_R2 | 407 | F09 | CAT GCA GGA TTT CCT GTA |
| | | | AAT TTG TCA GGC GCA GCT |
| | | | CCT TCA GTC CGG |
| EGFRmu70_R2 | 448 | F10 | CGT TGC ACA GGG CAG GGT |
| | | | TCT TTT CGA TCC GCA CGG |
| | | | CGC CAT GCA |
| EGFRmu71_R2 | 460 | F11 | TGC TCT CCA CGT TGC ACA |
| | | | GTT TAT CGT TGT TGC TGA |
| | | | ACC GCA CG |
| EGFRmu72_R2 | 472 | F12 | CCA CTG GAT GCT CTC CAC |
| | | | GTG GCA CAG GGC AGG GTT |
| | | | GTT G |
| EGFRmu73_R2 | 202 | G01 | AGA TCA TAA TTC CTC TGC |
| | | | ACA AGG ACA ATT TCC AAA |
| | | | TTC CCA AGG AC |
| EGFRmu74_R2 | 202 | G02 | AGA AGG AAA GAT CAT AAT |
| | | | TCC TCC CCG TAA GGA CAA |
| | | | TTT CCA AAT TCC CAA GGA |
| | | | C |
| EGFRmu75_R2 | 286 | G03 | GTT TTC CAA AGG AAT TCG |
| | | | CTC AAA TGT GTT GAG GGC |
| | | | AAT GAG GA |
| EGFRmu76_R2 | 286 | G04 | TGC AGG TTT TCC AAA GGA |
| | | | ATT GAC TCA AAT GTG TTG |
| | | | AGG GCA ATG AGG A |
| EGFRmu77_R2 | 283 | G05 | TGC AGG TTT TCC AAA GGA |
| | | | ATT GTC GAA AAT TCG TTG |
| | | | AGG GCA ATG AGG ACA |
| EGFRmu78_R2 | 364 | G06 | CGG TTT TAT TTG CAT CAT |
| | | | AGT TAA ACA TGA CTG CTA |
| | | | AGG CAT AGG AAT |
| EGFRmu79_R2 | 364 | G07 | CTT CAG TCC GGT TTT ATT |
| | | | TGC ATT ATA GTT AAA CAT |
| | | | GAC TGC TAA GGC ATA GGA |
| | | | ATT |
| EGFRmu80_R2 | 448 | G08 | AGG GCA GGG TTG TTG CTG |
| | | | ATC CGC ACG GCG CCA TGC |
| | | | A |
| EGFRmu81_R2 | 448 | G09 | CCA CGT TGC ACA GGG CAG |
| | | | GCT TGT TGC TGA TCC GCA |
| | | | CGG CGC CAT GCA |
| EGFRmu82_R2 | 103 | G10 | CCA ACT GCG TGA GCT TGT |
| | | | TAA GCG TGC CTT GGC AAA |
| | | | CTT TCT |
| EGFRmu83_R2 | 103 | G11 | CCA ACT GCG AGA GCT TGT |
| | | | TAA GCG TGC CTT GGC AAA |
| | | | CTT TCT T |
| EGFRmu84_R2 | 127 | G12 | TCC TCT GGA GGC TGA GAA |
| | | | ATT GAT TTT CAG CAT CGC |
| | | | CCA ACT GCG TGA GCT T |
| EGFRmu85_R2 | 127 | H01 | CAT CCT CTG GAG GCT GAG |
| | | | ATA TTG ATT TTC AGC ATC |
| | | | GCC CAA CTG CGT GAG CTT |
| EGFRmu86_R2 | 124 | H02 | GGC TGA GAA AAT GAT CTT |
| | | | CAA AAC CGT TCA ACT GCG |
| | | | TGA GCT TGT TAC |
| EGFRmu87_R2 | 124 | H03 | AGG CTG AGA AAA TGA TCT |
| | | | TCA TAA CCG TTC AAC TGC |
| | | | GTG AGC TTG TTA C |
| EGFRmu88_R2 | 124 | H04 | GAG GCT GAG AAA ATG ATC |
| | | | TTC ATA ATT GTT CAA CTG |
| | | | CGT GAG CTT GTT AC |
| EGFRmu89_R2 | 1471 | H05 | GCA GCT GTT TTC ACC TCT |
| | | | GTT TTT TTG AAT TTT GGT |
| | | | TTT CTG ACC GGA G |
| EGFRmu90_R2 | 1297 | H06 | CGA CTG CAA GAG AAA ACT |
| | | | GAC GAA CTT GCT TGG TCC |
| | | | TGC CGC G |
| EGFRmu91_R2 | 507 | H07 | ACA TGT TGC TGA GAA AGT |
| | | | CAC CCC TGA CTA TGT CCC |
| | | | GCC ACT |
| EGFRmu92_R2 | 514 | H08 | GTG GTT CTG GAA GTC CAT |
| | | | CAC GAT CTC GGC GTC ACG |
| | | | GTC ACT GCT GAC TAT GTC |
| | | | C |
| EGFRmu93_R2 | 532 | H09 | GCA GCT GCC CAG GTG GTT |
| | | | GTC GCC TTT CAC CGA CAT |
| | | | GTT GCT GAG AAA GTC |
| EGFRmu94_R2 | 118 | H10 | TGA GAA AAT GAT CTT CAA |
| | | | AAG TGT CCA AGT ACG TGA |
| | | | GCT TGT TAC TCG TGA |
| EGFRmu95_R2 | 115 | H11 | GAT CTT CAA AAG TGC CCA |
| | | | ACT CAT AGA GCT TGT TAC |
| | | | TCG TGC CTT |
| EGFRmu96_R2 | 337 | H12 | GAC TGC TAA GGC ATA GGA |
| | | | ATT ATC GTG GTA CAT ATT |
| | | | TCC TCT GAT GAT CTG |
| EGFRmu01_F2 | 1725 | A01 | CGC AGT TGG GCA CTT TTG |
| | | | AAG AAC ATT TTC TCA GCC |
| | | | TCC AGA G |
| EGFRmu02_F2 | 1726 | A02 | CGC AGT TGG GCA CTT TTG |
| | | | AAA ATC ATT TTC TCA GCC |
| | | | TCC AGA G |
| EGFRmu03_F2 | 1726 | A03 | CGC AGT TGG GCA CTT TTG |
| | | | AAC AAC ATT TTC TCA GCC |
| | | | TCC AGA G |
| EGFRmu04_F2 | 1739 | A04 | GTA ACA AGC TCA CGC AGT |
| | | | TGA ACA CTT TTG AAG ATC |
| | | | ATT TTC TCA |
| EGFRmu05_F2 | 1739 | A05 | GTA ACA AGC TCA CGC AGT |
| | | | TGA ACA CTT TTG AAG AAC |
| | | | ATT TTC TCA GCC TCC AGA |
| | | | G |
| EGFRmu06_F2 | 1736 | A06 | CGA GTA ACA AGC TCA CGC |
| | | | AGG TGG GCA CTT TTG AAG |
| | | | ATC ATT TT |
| EGFRmu07_F2 | 1736 | A07 | GAG TAA CAA GCT CAC GCA |
| | | | GAT TGG CAC TTT TGA AGA |
| | | | TCA TTT TC |
| EGFRmu08_F2 | 1586 | A08 | CTG GTT ATG TCC TCA TTG |
| | | | CCG TCA ACA CAG TGG AGC |
| | | | GAA TTC |
| EGFRmu09_F2 | 1586 | A09 | CTG GTT ATG TCC TCA TTG |
| | | | CCA TCA ACA CAG TGG AGC |
| | | | GAA TTC |
| EGFRmu10_F2 | 1658 | A10 | TGG GAA TTT GGA AAT TAC |
| | | | CTG GGT GCA GAG GAA TTA |
| | | | TGA TCT TT |
| EGFRmu11_F2 | 1525 | A11 | ATC ATC AGA GGA AAT ATG |
| | | | TAC TGG GAA AAT TCC TAT |
| | | | GCC TTA GCA G |
| EGFRmu12_F2 | 1521 | A12 | TCA GAG GAA ATA TGT ACT |
| | | | ACG ATA ATT CCT ATG CCT |
| | | | TAG CAG TC |
| EGFRmu13_F2 | 1519 | B01 | CAT CAG AGG AAA TAT GTA |
| | | | CTA CCA AAA TTC CTA TGC |
| | | | CTT AGC AGT |
| EGFRmu14_F2 | 1496 | B02 | TCC TAT GCC TTA GCA GTC |
| | | | TTA GCT AAC TAT GAT GCA |
| | | | AAT AAA ACC |
| EGFRmu15_F2 | 1496 | B03 | TCC TAT GCC TTA GCA GTC |
| | | | TTA ACT AAC TAT GAT GCA |
| | | | AAT AAA ACC |
| EGFRmu16_F2 | 1735 | B04 | AAG CTC ACG CAG TTG GGC |
| | | | AAA TTT GAA GAT CAT TTT |
| | | | CTC AGC C |
| EGFRmu17_F2 | 1735 | B05 | AAG CTC ACG CAG TTG GGC |
| | | | CAA TTT GAA GAT CAT TTT |
| | | | CTC AGC C |
| EGFRmu18_F2 | 1734 | B06 | AGC TCA CGC AGT TGG GCA |
| | | | TTT TTG AAG ATC ATT TTC |
| | | | TCA GCC |
| EGFRmu19_F2 | 1735 | B07 | AAG CTC ACG CAG TTG GGC |
| | | | GAT TTT GAA GAT CAT TTT |
| | | | CTC AGC C |
| EGFRmu20_F2 | 1735 | B08 | AAG CTC ACG CAG TTG GGC |
| | | | TAT TTT GAA GAT CAT TTT |
| | | | CTC AGC C |
| EGFRmu21_F2 | 1735 | B09 | AAG CTC ACG CAG TTG GGC |
| | | | GGT TTT GAA GAT CAT TTT |
| | | | CTC AGC C |
| EGFRmu22_F2 | 1735 | B10 | AAG CTC ACG CAG TTG GGC |
| | | | CTT TTT GAA GAT CAT TTT |
| | | | CTC AGC C |
| EGFRmu23_F2 | 1718 | B11 | TGG GCA CTT TTG AAG ATC |
| | | | ATT TTG CCA GCC TCC AGA |
| | | | GGA TGT TC |
| EGFRmu24_F2 | 1718 | B12 | GGG CAC TTT TGA AGA TCA |
| | | | TTT TTG GAG CCT CCA GAG |
| | | | GAT GTT CAA |
| EGFRmu25_F2 | 1722 | C01 | CAC TTT TGA AGA TCA TTT |
| | | | TCT CAC CCT CCA GAG GAT |
| | | | GTT CAA TAA |
| EGFRmu26_F2 | 1722 | C02 | GCA CTT TTG AAG ATC ATT |
| | | | TTC TCC TCC TCC AGA GGA |
| | | | TGT TCA ATA |
| EGFRmu27_F2 | 1718 | C03 | GGG CAC TTT TGA AGA TCA |
| | | | TTT TGC CCT CCT CCA GAG |
| | | | GAT GTT CAA TAA C |
| EGFRmu28_F2 | 1718 | C04 | GGG CAC TTT TGA AGA TCA |
| | | | TTT TGC CCA ACT CCA GAG |
| | | | GAT GTT CAA TAA C |
| EGFRmu29_F2 | 1722 | C05 | GCA CTT TTG AAG ATC ATT |
| | | | TTC TCG CCC TCC AGA GGA |
| | | | TGT TCA ATA A |
| EGFRmu30_F2 | 1718 | C06 | TGG GCA CTT TTG AAG ATC |
| | | | ATT TTT ACG CCC TCC AGA |
| | | | GGA TGT TCA ATA A |
| EGFRmu31_F2 | 1718 | C07 | GGG CAC TTT TGA AGA TCA |
| | | | TTT TAA CGC CCT CCA GAG |
| | | | GAT GTT CAA TAA |
| EGFRmu32_F2 | 1745 | C08 | GGC ACG AGT AAC AAG CTC |
| | | | ACG GAG TTG GGC ACT TTT |
| | | | GAA GAT CA |
| EGFRmu33_F2 | 1745 | C09 | GCA CGA GTA ACA AGC TCA |
| | | | CGT CGT TGG GCA CTT TTG |
| | | | AAG ATC A |
| EGFRmu34_F2 | 1745 | C10 | GCA CGA GTA ACA AGC TCA |
| | | | CGA AGT TGG GCA CTT TTG |
| | | | AAG ATC |
| EGFRmu35_F2 | 1745 | C11 | CAC GAG TAA CAA GCT CAC |
| | | | GTA CTT GGG CAC TTT TGA |
| | | | AGA TCA T |
| EGFRmu36_F2 | 1742 | C12 | AAG GCA CGA GTA ACA AGC |
| | | | TCT CGC AGT TGG GCA CTT |
| | | | TTG AAG |
| EGFRmu37_F2 | 1743 | D01 | AAG GCA CGA GTA ACA AGC |
| | | | TCA AGC AGT TGG GCA CTT |
| | | | TTG AAG |
| EGFRmu38_F2 | 1742 | D02 | AAG GCA CGA GTA ACA AGC |
| | | | TCG AGC AGT TGG GCA CTT |
| | | | TTG AAG |
| EGFRmu39_F2 | 1742 | D03 | GGC ACG AGT AAC AAG CTC |
| | | | TAT CAG TTG GGC ACT TTT |
| | | | GAA GAT |
| EGFRmu40_F2 | 1763 | D04 | GAA AAG AAA GTT TGC CAA |
| | | | GGC CAG AGT AAC AAG CTC |
| | | | ACG CAG T |
| EGFRmu41_F2 | 563 | D05 | CGG CAG GAC CAA GCA ACA |
| | | | TCG TCA GTT TTC TCT TGC |
| | | | AGT CG |
| EGFRmu42_F2 | 563 | D06 | CGG CAG GAC CAA GCA ACA |
| | | | TAA TCA GTT TTC TCT TGC |
| | | | AGT CGT |
| EGFRmu43_F2 | 470 | D07 | GAT GGA GAT GTG ATA ATT |
| | | | TCA CGA AAC AAA AAT TTG |
| | | | TGC TAT GC |
| EGFRmu44_F2 | 470 | D08 | GAT GGA GAT GTG ATA ATT |
| | | | TCA CAT AAC AAA AAT TTG |
| | | | TGC TAT GCA AA |
| EGFRmu45_F2 | 1717 | D09 | GGC ACT TTT GAA GAT CAT |
| | | | TTT CAA AGC CTC CAG AGG |
| | | | ATG TTC AA |
| EGFRmu46_F2 | 754 | D10 | TCC ATC AGT GGC GAT CTC |
| | | | CGC ATC CTG CCG GTG GCA |
| | | | TTT |
| EGFRmu47_F2 | 743 | D11 | CGA TCT CCA CAT CCT GCC |
| | | | GCT GGC ATT TAG GGG TGA |
| | | | CT |
| EGFRmu48_F2 | 737 | D12 | CAC ATC CTG CCG GTG GCA |
| | | | CAT AGG GGT GAC TCC TTC |
| | | | ACA |
| EGFRmu49_F2 | 731 | E01 | TGC CGG TGG CAT TTA GGG |
| | | | CTG ACT CCT TCA CAC ATA |
| | | | CTC |
| EGFRmu50_F2 | 731 | E02 | CTG CCG GTG GCA TTT AGG |
| | | | AAT GAC TCC TTC ACA CAT |
| | | | ACT C |
| EGFRmu51_F2 | 637 | E03 | AGG GTT TTT GCT GAT TCA |
| | | | GGA ATG GCC TGA AAA CAG |
| | | | GAC GG |
| EGFRmu52_F2 | 637 | E04 | AGG GTT TTT GCT GAT TCA |
| | | | GGA TTG GCC TGA AAA CAG |
| | | | GAC GG |
| EGFRmu53_F2 | 635 | E05 | GGT TTT TGC TGA TTC AGG |
| | | | CTG AGC CTG AAA ACA GGA |
| | | | CGG AC |
| EGFRmu54_F2 | 633 | E06 | TCT TGC AGT CGT CAG CCT |
| | | | GGA CAT AAC ATC CTT GGG |
| | | | ATT AC |
| EGFRmu55_F2 | 391 | E07 | CCG GTC AGA AAA CCA AAA |
| | | | TTA AAA GCA ACA GAG GTG |
| | | | AAA ACA G |
| EGFRmu56_F2 | 404 | E08 | GTT TGG GAC CTC CGG TCA |
| | | | GGA AAC CAA AAT TAT AAG |
| | | | CAA CAG A |
| EGFRmu57_F2 | 404 | E09 | GTT TGG GAC CTC CGG TCA |
| | | | GCA AAC CAA AAT TAT AAG |
| | | | CAA CAG A |
| EGFRmu58_F2 | 388 | E10 | CGG TCA GAA AAC CAA AAT |
| | | | TAT AAA AAA CAG AGG TGA |
| | | | AAA CAG CTG C |
| EGFRmu59_F2 | 1697 | E11 | CAG CCT CCA GAG GAT GTT |
| | | | CGA AAA CTG TGA GGT GGT |
| | | | CCT TGG |
| EGFRmu60_F2 | 1703 | E12 | ATT TTC TCA GCC TCC AGA |
| | | | GGC TGT TCA ATA ACT GTG |
| | | | AGG TGG |
| EGFRmu61_F2 | 1703 | F01 | TTT TCT CAG CCT CCA GAG |
| | | | GCT GTA CGA AAA CTG TGA |
| | | | GGT GGT CCT TGG |
| EGFRmu62_F2 | 1736 | F02 | AAG CTC ACG CAG TTG GGC |
| | | | GAT GCT GAA GAT CAT TTT |
| | | | CTC AGC CTC |
| EGFRmu63_F2 | 1736 | F03 | AAG CTC ACG CAG TTG GGC |
| | | | GAT GCT GAA AAT CAT TTT |
| | | | CTC AGC CTC CAG A |
| EGFRmu64_F2 | 1768 | F04 | TGG AGG AAA AGA AAG TTT |
| | | | GCC CAG GCA CGA GTA ACA |
| | | | AGC TCA |
| EGFRmu65_F2 | 1580 | F05 | TGT CCT CAT TGC CCT CAA |
| | | | CGA ATT TTC GAC AAT TCC |
| | | | TTT GGA AAA CCT GCA |
| EGFRmu66_F2 | 1549 | F06 | GAA TTC CTT TGG AAA ACC |
| | | | TGC GGA TCA TCA GAG GAA |
| | | | ATA TGT AC |
| EGFRmu67_F2 | 1534 | F07 | CCT GCA GAT CAT CAG AGG |
| | | | AAC TCA GGT CTA CGA AAA |
| | | | TTC CTA TGC CTT |
| EGFRmu68_F2 | 1499 | F08 | ATT CCT ATG CCT TAG CAG |
| | | | TCA TGT TAA ACT ATG ATG |
| | | | CAA ATA AAA CCG |
| EGFRmu69_F2 | 1456 | F09 | CCG GAC TGA AGG AGC TGC |
| | | | GCC TGA CAA ATT TAC AGG |
| | | | AAA TCC TGC ATG |
| EGFRmu70_F2 | 1415 | F10 | TGC ATG GCG CCG TGC GGA |
| | | | TCG AAA AGA ACC CTG CCC |
| | | | TGT GCA ACG |
| EGFRmu71_F2 | 1403 | F11 | CGT GCG GTT CAG CAA CAA |
| | | | CGA TAA ACT GTG CAA CGT |
| | | | GGA GAG CA |
| EGFRmu72_F2 | 1391 | F12 | CAA CAA CCC TGC CCT GTG |
| | | | CCA CGT GGA GAG CAT CCA |
| | | | GTG G |
| EGFRmu73_F2 | 1661 | G01 | GTC CTT GGG AAT TTG GAA |
| | | | ATT GTC CTT GTG CAG AGG |
| | | | AAT TAT GAT CT |
| EGFRmu74_F2 | 1661 | G02 | GTC CTT GGG AAT TTG GAA |
| | | | ATT GTC CTT ACG GGG AGG |
| | | | AAT TAT GAT CTT TCC TTC |
| | | | T |
| EGFRmu75_F2 | 1577 | G03 | TCC TCA TTG CCC TCA ACA |
| | | | CAT TTG AGC GAA TTC CTT |
| | | | TGG AAA AC |
| EGFRmu76_F2 | 1577 | G04 | TCC TCA TTG CCC TCA ACA |
| | | | CAT TTG AGT CAA TTC CTT |
| | | | TGG AAA ACC TGC A |
| EGFRmu77_F2 | 1580 | G05 | TGT CCT CAT TGC CCT CAA |
| | | | CGA ATT TTC GAC AAT TCC |
| | | | TTT GGA AAA CCT GCA |
| EGFRmu78_F2 | 1499 | G06 | ATT CCT ATG CCT TAG CAG |
| | | | TCA TGT TTA ACT ATG ATG |
| | | | CAA ATA AAA CCG |
| EGFRmu79_F2 | 1499 | G07 | AAT TCC TAT GCC TTA GCA |
| | | | GTC ATG TTT AAC TAT AAT |
| | | | GCA AAT AAA ACC GGA CTG |
| | | | AAG |
| EGFRmu80_F2 | 1415 | G08 | TGC ATG GCG CCG TGC GGA |
| | | | TCA GCA ACA ACC CTG CCC |
| | | | T |
| EGFRmu81_F2 | 1415 | G09 | TGC ATG GCG CCG TGC GGA |
| | | | TCA GCA ACA AGC CTG CCC |
| | | | TGT GCA ACG TGG |
| EGFRmu82_F2 | 1760 | G10 | AGA AAG TTT GCC AAG GCA |
| | | | CGC TTA ACA AGC TCA CGC |
| | | | AGT TGG |
| EGFRmu83_F2 | 1760 | G11 | AAG AAA GTT TGC CAA GGC |
| | | | ACG CTT AAC AAG CTC TCG |
| | | | CAG TTG G |
| EGFRmu84_F2 | 1736 | G12 | AAG CTC ACG CAG TTG GGC |
| | | | GAT GCT GAA AAT CAA TTT |
| | | | CTC AGC CTC CAG AGG A |
| EGFRmu85_F2 | 1736 | H01 | AAG CTC ACG CAG TTG GGC |
| | | | GAT GCT GAA AAT CAA TAT |
| | | | CTC AGC CTC CAG AGG ATG |
| EGFRmu86_F2 | 1736 | H02 | GTA ACA AGC TCA CGC AGT |
| | | | TGA ACG GTT TTG AAG ATC |
| | | | ATT TTC TCA GCC |
| EGFRmu87_F2 | 1736 | H03 | GTA ACA AGC TCA CGC AGT |
| | | | TGA ACG GTT ATG AAG ATC |
| | | | ATT TTC TCA GCC T |
| EGFRmu88_F2 | 1736 | H04 | GTA ACA AGC TCA CGC AGT |
| | | | TGA ACA ATT ATG AAG ATC |
| | | | ATT TTC TCA GCC TC |
| EGFRmu89_F2 | 392 | H05 | CTC CGG TCA GAA AAC CAA |
| | | | AAT TCA AAA AAA CAG AGG |
| | | | TGA AAA CAG CTG C |
| EGFRmu90_F2 | 566 | H06 | CGC GGC AGG ACC AAG CAA |
| | | | GTT CGT CAG TTT TCT CTT |
| | | | GCA GTC G |
| EGFRmu91_F2 | 1356 | H07 | AGT GGC GGG ACA TAG TCA |
| | | | GGG GTG ACT TTC TCA GCA |
| | | | ACA TGT |
| EGFRmu92_F2 | 1349 | H08 | GGA CAT AGT CAG CAG TGA |
| | | | CCG TGA CGC CGA GAT CGT |
| | | | GAT GGA CTT CCA GAA CCA |
| | | | C |
| EGFRmu93_F2 | 1331 | H09 | GAC TTT CTC AGC AAC ATG |
| | | | TCG GTG AAA GGC GAC AAC |
| | | | CAC CTG GGC AGC TGC |
| EGFRmu94_F2 | 1745 | H10 | GCA CGA GTA ACA AGC TCA |
| | | | CGT ACT TGG ACA CTT TTG |
| | | | AAG ATC ATT TTC TCA |
| EGFRmu95_F2 | 1748 | H11 | AAG GCA CGA GTA ACA AGC |
| | | | TCT ATG AGT TGG GCA CTT |
| | | | TTG AAG ATC |
| EGFRmu96_F2 | 1526 | H12 | CAG ATC ATC AGA GGA AAT |
| | | | ATG TAC CAC GAT AAT TCC |
| | | | TAT GCC TTA GCA GTC |

Confirmed HFD100 mutants in pDONR221 were subcloned into pcDNA3.2-DEST expression vector by LR reaction.

### B. Protein Expression and Secretion

The HFD100-mutants in pcDNA3.2-DEST expression vector were expressed in 293T cells using Lipofectamin 2000-mediated transient gene expression (Invitrogen) following the manufacturer's instruction. Conditioned media were collected 48 hours after transfection. A volume of 15 ul of the conditioned media was analyzed by Western blotting. The Western blots were probed with anti-Fc antibody to check the protein expression and secretion. Duoset Human EGFR ELISA Kit (R&D System) was used to diertermine the recombinant HFD100-mutants in the conditioned media. ELISA plates are coated with 0.4 ug/ml anti-EGFR antibody at room temperature for over night. Coated plates were washed 3 times in PBS+0.05% Tween 20, blocked with PBS/1% BSA at RT for 2 hrs, and washed 3 times again in PBS+0.05% Tween-20. The condition media were initially diluted at 1:1000, and were further diluted at a ratio of 1:2. The diluted conditioned medied (CM) were applied to the plates for ELISA detection following the manufacturer's instruction.

### C. Ligand Binding Screening

EU-labeled EGF binding: Plates were coated with 5 ug /ml of anti-Fc antibody at RT for overnight. After coating plates were washed 3 times in PBS/0.05% Tween 20, and were blocked with PBS/1% BSA at RT for 2 hrs. After blocking, plates were washed 3 times with PBS/0.05% Tween 20. Recombinant proteins in conditioned media (20 ng) were diluted with 1× DELFIA binding buffer, and were added to the plates (100 μl/well. Plates were incubated at RT for 2 hrs. This was followed by 3 washes with 120 ul/well of ice cold DELFIA wash buffer. Subsequently, EU-EGF (0.5 nM) in DELFIA binding buffer was added to each well (100 μl/well) and plates were incubated at RT for 2 hrs. Plates were washed 3 times with ice-cold DELFIA wash buffer (120 μl/well).

DELFIA Enhancement Solution (110 μl/well) was added to each well, and plates were further incubated at RT for 20 min. After the incubation, the plates were read by an Envision (PerkinElmer) to detect the time-resolved fluorescence.

### D. TGF_ and HB-EGF Binding

A TGF_ or HB-EGF ELISA Kit (R&D System) was modified for the ligand binding assays. Plates were coated with 1 μg/ml anti-Fc antibody (Sigma) at RT for overnight, and blocked with PBS/1% BSA at RT for 2 hrs. Blocked plates were incubated with 20 ng of HFD100-mutants protein at RT for 2 hrs. Plates were washed and further incubated with 5-50 nM TGF_ or 5 nM HB-EGF, respectively, in 100 μl/well of binding buffer ((PBS/1% BSA) at RT for 2 hrs. Plates were washed, and further incubated at RT for 2 hrs with 300 ng/ml of biotinylated goat anti-human TGF_ or biotinylated goat anti-human HB-EGF antibody. Streptavidin-HRP (1:200 dilution) was subsequently added to the plates and a substrate solution was applied 20 min later for color development. Plates were read by a microplate reader to determine the values at OD 650 nm.

### E. Results

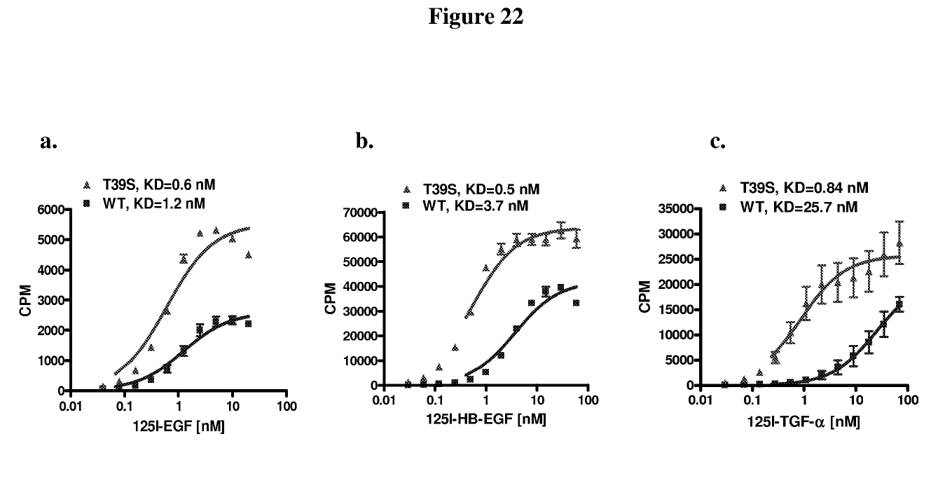

Detailed ligand binding studies revealed that the HFD120 bound the HER1 ligands with higher affinity than the wild type (HFD100). Compared with the wild type (HFD100), the HFD120 mutant gave 2-fold higher affinity for EGF, 7-fold improved affinity for HB-EGF, and greater than 30-fold improved affinity for TGF-alpha (FIGS. 22a-c and Table 32).

TABLE 32
Binding Affinity
Binding Affinity (KD = nM)

| Growth Factor | HFD100 | T39S | Fold Improvement |
|---|---|---|---|
| EGF | 1.2 | 0.6 | 2 |
| HB-EGF | 3.7 | 0.5 | 7 |
| TGF-β | 25.7 | 0.8 | >30 |

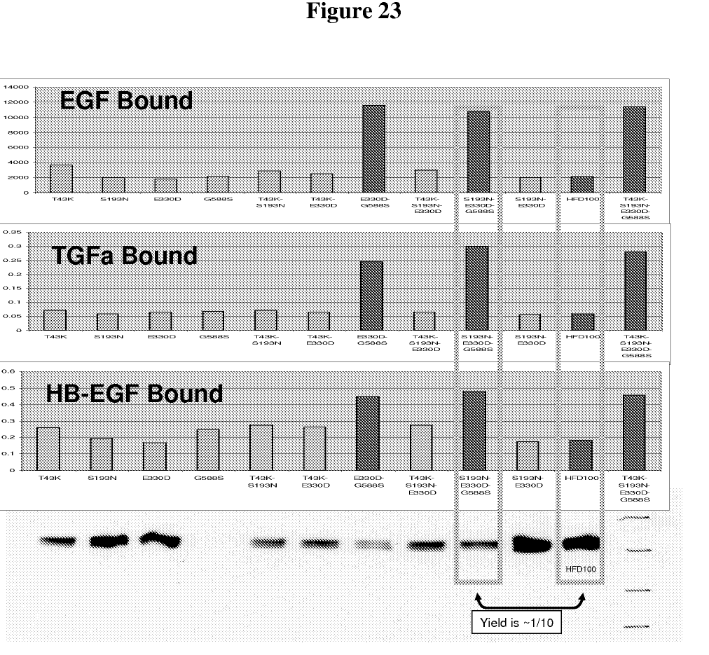

One mutant called T43K/S193N/E330D/G588S, besides designed T43K mutation had random PCR introduced mutations. This quad mutant had substantially increased HER1 ligand binding activities (FIG. 23). This mutant was systematically changed to give rise to two other HER1 mutants called called S193N/E330D/G588S and E330D/G588S, both bound HER1 ligands EGF, HB-EGF and TGF-alpha to substantially increased levels compared with the wild type (HFD100); however, the S193N/E330D/G588S gave higher secretion level of protein than did E330D/G588S (FIG. 23).

### Example 24

### Engineering for Higher Ligand Binding Affinity and Capacity panHER Ligand Traps: Hermodulins with Increased Ligand Binding Capacity

Besides the HFD120, which has high affinity for HER1 ligands (discussed above in Example 18), a heterodimeric HER1/Fc:HER3/Fc construct called RB220h was made with the T39S mutation in the HER1 arm. This T39S mutation is same as in HFD120. HFD120 was expressed and purified as in HFD100 in Examples 2 and 3. The hermodulins were also expressed as mixtures comprising homodimers and heterodimers, called RB620 is the mixture cell expression system makes as HFD120, HFD300, and RB220h. See Table 33. RB620 was expressed as described in Example 2 and purified as described for RB600 in Example 3.

TABLE 33
Hermodulin Compositions

| Molecule | |
|---|---|
| Name | Elements |
| HFD100 | Her1/Fc homodimer |
| HFD120 | Her1/Fc homodimer with mutation in HER1 T39S |
| HFD300 (also called | Her3/Fc homodimer |
| HFD300h) | |
| RB200h | Purified Her1/Fc-Her3/Fc heterodimer |
| RB220 | RB200h with enhanced Her1 component (Her1 with |
| | T39S mutation) |
| RB600 (RB-mix) | Her1/Fc homodimer |
| | Her3/Fc homodimer |
| | Her1-Her3 heterodimer |
| RB620 | RB600 with enhanced Her1 component (Her1 with |
| | T39S mutation) |
| RB630 | RB600 with enhanced Her1 component and |
| | enhanced Her3 component |

The HER1 or HER3 ligand binding activities (capacity) of the mutants were compared with the wild type constructs. Comparing either the homodimers HFD100 versus HFD120 (mutant construct) or RB200h versus RB220h (mutant construct), the mutants, which contain the T39S mutation in HER1, have approximately 2.5-fold EGF binding capacity than their wild type counterpart (Tables 34, 35, 38, 39). Also, the data show that the mix, either as RB600 or as RB620 have better, 3- to 10-fold higher HER1 ligand EGF binding capacity (Tables 35, 38 and 39).

With respect to HER3 ligand (NRG1β1) binding capacities of wild type and mutant hermodulins, the difference less pronounced than observed for EGF binding. First, the heterodimer RB200h has approximately 1.6-fold higher NRG1β1 binding capacity than the mix RB600. The NRG1β1 binding capacities of the mutant heterodimer (RB220h) or the mutant mix RB620 is approximately the same (See Tables 38 and 39). However, interesting finding is that when NRG1β1 binding activities of heterodimers either the wild type RB200h or the mutant (RB220h) is compared with the HER3 homodimer (HFD300), the HER3 homodimer HFD300 has only 30% binding activity of the heterodimers (See Tables 36 and 37).

TABLE 34
Relative EGF Binding Activities.

| Protein | Relative Binding to EGF | HFD % |
|---|---|---|
| HFD100-63 | 1 | >99% |
| HFD120-1 | 1.8 | >99% |
| RB200H-X.C | 1 | <0.5% |
| RB220h-1 | 2.47 | <0.5% |

TABLE 35
Relative EGF Binding Activities.

| | Relative Binding to | | |
|---|---|---|---|
| Protein | EGF | HFD1xx % | RB2xxh % |
| RB600-1 | 1 | 64% | 27% |
| RB200h-X.C | 0.11 | <0.5% | |
| RB602-1 | 1 | 37% | 46% |
| RB220h-1 | 0.29 | <0.5% | |

TABLE 36
Relative NRG1β1 Binding Activities.

| | Relative Binding to | | |
|---|---|---|---|
| Protein | NRG-β1 | HFD2xxh % | HFD300 % |
| RB600-1 | 1 | 27% | 9% |
| RB200h-X.C | 1.57 | 95% | 5% |
| RB602-1 | 1.68 | 46% | 17% |
| RB220h-1 | 1.76 | >98% | <2% |

TABLE 37
Relative NRG1β1 Binding Activities.

| | Specific Binding | | |
|---|---|---|---|
| Protein | to NRG-β1 | purity % | RB2xxh % |
| HFD100-63 | 0.016 | >98% | |
| HFD120-1 | 0.032 | >98% | |
| RB300-1 | 0.676 | 75% | 25% |

TABLE 38
Ligand Binding Specific Activities: RB200h vs RB600.

| | fmol | | fmol | | |
|---|---|---|---|---|---|
| | EGF/fmol | | NRG/fmol | | EGF:NRG |
| | RB200 | SD | RB200 | SD | Ratio |
| RB200h- | 0.153 | 0.008 | 0.302 | 0.013 | 0.508 |
| 65/67/70/72 | | | | | |
| RB220h-1 | 0.051 | 0.003 | 0.407 | 0.025 | 0.125 |
| RB620-1 | 0.174 | 0.008 | 0.387 | 0.031 | 0.449 |
| RB200h-XC | 0.031 | 0.001 | 0.363 | 0.006 | 0.086 |
| RB600-1 | 0.283 | 0.024 | 0.231 | 0.009 | 1.223 |

TABLE 39
Ligand Binding Specific Activities: RB200h vs RB600.

| Protein | fmol EGF/mg RB | fmol NRG/mg RB |
|---|---|---|
| RB200h | 0.16 × 10⁶ | 1.91 × 10⁶ |
| RB600 | 1.50 × 10⁶ | 1.22 × 10⁶ |
| RB220h | 0.27 × 10⁶ | 2.14 × 10⁶ |
| RB620 | 0.91 × 10⁶ | 2.04 × 10⁶ |

1. A multimer, comprising:
a) a first chimeric polypeptide that is selected from either:
i) a chimeric polypeptide that contains a full-length extracellular domain (ECD) from HER1 receptor linked directly or indirectly via a linker to a multimerization domain, or
ii) a chimeric polypeptide that contains less than the full length of the ECD of HER1, HER2, HER3 or HER4 receptor linked directly or indirectly via a linker to a multimerization domain, wherein the ECD contains at least a sufficient portion of subdomains I and/or III to bind to a ligand of the receptor and a sufficient portion of the ECD, including a sufficient portion of subdomain II, to dimerize with a cell surface receptor, unless the ECD in the chimeric polypeptide is from a HER2 receptor, then it also contains all or part of domain IV, including a sufficient portion or all of modules 2-5 of subdomain IV to effect dimerization with a cell surface receptor; and
b) a second chimeric polypeptide linked directly or indirectly via a linker to a multimerization domain, and that contains at least a sufficient portion of an ECD of a cell surface protein to bind to ligand therefor and/or to dimerize with a cell surface receptor, wherein the multimerization domains in the first and second chimeric polypeptides are complementary or the same, with the proviso that if the first chimeric polypeptide is a full length HER1 ECD, then the second chimeric polypeptide does not contain an ECD from HER2 or if it does, the HER2 ECD is less than full length and the sufficient portion for receptor dimerization includes a sufficient portion of domain IV to effect dimerization, whereby:
the chimeric polypeptides form a multimer; and
the resulting multimer binds to additional ligands compared to the first chimeric polypeptide or a homodimer thereof and/or dimerizes with more cell surface receptors than the first chimeric polypeptide or a homodimer thereof.

2. The multimer of claim 1, wherein the ECD of one or both of the first and second chimeric polypeptide is a hybrid ECD that contains subdomains from at least two different cell surface receptor ECDs.

3. The multimer of claim 1, wherein the first chimeric polypeptide contains less than the full length of the ECD of HER2, HER3 or HER4.

4. The multimer of claim 1, wherein the first chimeric polypeptide contains less than the full length of the ECD of HER3 or HER4

5. The multimer of claim 1 that is a heteromultimer, wherein the ECD portion of the second chimeric polypeptide is from a different cell surface receptor from HER1.

6. The multimer of claim 5, wherein the ECD in the second chimeric polypeptide is from HER3 or HER4.

7. The multimer of claim 1, wherein the ECD domain of the second chimeric polypeptide contains a full length ECD.

8. The multimer of claim 1, wherein the ECD domain of the second chimeric polypeptide contains at least a sufficient portion of subdomains I, II and III to bind to its ligand and to dimerize with a cell surface receptor.

9. The multimer of claim 1, wherein the second chimeric polypeptide contains less than a full-length ECD, and includes a sufficient portion of domains I and III to bind to its ligand.

10. The multimer of claim 1, wherein the second chimeric polypeptide contains less than a full-length ECD, and includes a sufficient portion of the ECD to dimerize with a cell surface receptor.

11. The multimer of claim 1, wherein the multimerization domain is selected from among an immunoglobulin constant region (Fc), a leucine zipper, complementary hydrophobic regions, complementary hydrophilic regions, compatible protein-protein interaction domains, free thiols that forms an intermolecular disulfide bond between two molecules, and a protuberance-into-cavity and a compensatory cavity of identical or similar size that form stable multimers.

12. The multimer of claim 1, wherein the multimerization domain is an Fc domain or a variant thereof that effects multimerization.

13. The multimer of claim 12, wherein the Fc domain is from an IgG, IgM or an IgE.

14. The multimer of claim 1, wherein the cell surface receptor is a cognate receptor to an ECD or subdomain of the ECD of the multimer.

15. The multimer of claim 1, wherein the ECD of the second chimeric polypeptide is selected from among HER2, HER 3, HER4, IGF1-R, VEGFR, a FGFR, a TNFR, a PDGFR, a MET, a Tie, a RAGE, an EPH receptor and a T cell receptor

16. The multimer of claim 15, wherein the ECD of the second chimeric polypeptide is selected from among VEGFR1, FGFR2, FGFR4, IGF1-R and Tie1.

17. The multimer of claim 2, wherein the ECD of the second chimeric polypeptide is an intron fusion protein which is linked to the multimerization domain.

18. A multimer of claim 2, wherein the second ECD is a full length HER2, HER3 or HER4 or a sufficient portion of thereof for receptor dimerization with a cell surface receptor and/or for binding to a ligand for a cell surface receptor.

19. A multimer of claim 2, wherein the second ECD is from a receptor tyrosine kinase other than HER1.

20. The multimer of claim 2 that binds to at least three, four, five, six or seven different ligands.

21. The multimer of claim 20, wherein the ligand is selected from among EGF, TGF-α, amphiregulin, HB-EGF, β-cellulin, epiregulin and an additional ligand that binds to the ECD of a cell surface receptor other than HER1.

22. The multimer of claim 21, wherein the additional ligand is selected from among neuregulin-1, neuregulin-2, neuregulin-3 and neuregulin-4.

23. The multimer of claim 1, wherein:
the first chimeric polypeptide contains either i) a full length ECD from HER1 or ii) a portion thereof sufficient to bind to ligand and/or to dimerize; and
the second chimeric polypeptide contains all or a portion of the ECD of HER3 or HER4 sufficient to bind to ligand and/or to dimerize.

24. The multimer of claim 1, wherein the multimerization domain in each chimeric polypeptide is selected from among an immunoglobulin constant region (Fc), a leucine zipper, complementary hydrophobic regions, complementary hydrophilic regions, compatible protein-protein interaction domains, free thiols that forms an intermolecular disulfide bond between two molecules, and a protuberance-into-cavity and a compensatory cavity of identical or similar size that form stable multimers, whereby the chimeric polypeptides interact in a back-to-back configuration whereby the ECD of both chimeric polypeptides is available for dimerization with a cell surface receptor.

25. The multimer of claim 23 or claim 24, wherein the multimerization domain is an Fc domain.

26. The multimer of claim 25, wherein the Fc domain is from an IgG, IgM or an IgE.

27. The multimer of claim 1, that comprises at least two chimeric polypeptides, wherein:
the first chimeric polypeptide contains all or part of the ECD of HER1; and
the second chimeric polypeptide contains all or part of the ECD of HER3 or HER4.

28. The multimer of claim 1, wherein a constituent chimeric polypeptide is a fusion polypeptide.

29. The multimer of claim 1, wherein chimeric polypeptides a) and b) are fusion polypeptides.

30. The multimer of claim 1, wherein a constituent chimeric polypeptide is formed by chemical conjugation.

31. The multimer of claim 1, wherein chimeric polypeptides a) and b) are formed by chemical conjugation.

32. The multimer of claim 1, wherein the multimerization domain of at least one chimeric polypeptide is linked directly to the ECD.

33. The multimer of claim 1, wherein the multimerization domain of at least one chimeric polypeptide is linked via a linker to the ECD.

34. The multimer of claim 32, wherein the multimerization domains of all constituent chimeric polypeptides are linked directly to each respective ECD.

35. The multimer of claim 33, wherein the multimerization domains of all of the constituent chimeric polypeptides are linked to each respective ECD via a linker.

36. The multimer of claim 33 or claim 35, wherein the linker is a chemical linker or a polypeptide linker.

37. The multimer of claim 1 that is a heterodimer.

38. The multimer of claim 1 that is a heterodimer that contains the component chimeric polypeptides in a back-to-back configuration, whereby the ECD in each chimeric polypeptide is available for dimerization with a cell surface receptor.

39. A heteromultimer, comprising:
an extracellular domain (ECD) from one HER receptor; and
an ECD from a second receptor, wherein:
at least one of the ECDs is a HER ECD and contains subdomains I, II and III and part, but not all of subdomain IV;
subdomain IV includes at least module 1; and
the ECDs are different.

40. The heteromultimer of claim 39, wherein the second ECD is from a cell surface receptor.

41. The heteromultimer of claim 39 wherein one HER is HER1 and the other is HER3 or HER4.

42. The heteromultimer of claim 39, wherein the dimerization domain of at least one ECD in the heteromultimer is available for dimerization with a cell surface receptor.

43. The heteromultimer of claim 39, wherein each ECDs is linked directly or via a linker to a multimerization domain, whereby the multimerization domains of at least two ECDs interact to form the heteromultimer.

44. The heteromultimer of claim 43, wherein the multimerization domain is selected from among an immunoglobulin constant region (Fc), a leucine zipper, complementary hydrophobic regions, complementary hydrophilic regions, compatible protein-protein interaction domains, free thiols that forms an intermolecular disulfide bond between two molecules, and a protuberance-into-cavity and a compensatory cavity of identical or similar size that form stable multimers.

45. The heteromultimer of claim 43 or claim 44, wherein the multimerization domain is an Fc domain.

46. The multimer of claim 45, wherein the Fc domain is from an IgG, IgM or an IgE.

47. The heteromultimer claim 40, wherein the cell surface receptor is a cognate receptor to an ECD or subdomain of the ECD of the heteromultimer.

48. The heteromultimer of claim 38, wherein the second ECD is from a receptor selected from among HER2, HER 3, HER4, IGF1-R, VEGFR, a FGFR, a TNFR, a PDGFR, a MET, a Tie, a RAGE, an EPH receptor and a T cell receptor.

49. The heteromultimer of claim 48, wherein the ECD is selected from among VEGFR1, FGFR2, FGFR4, IGFR1 and Tie1.

50. A hybrid extracellular domain (ECD), comprising:
all or part of at least domains I, II and III of an ECD of one or more cell surface receptor, wherein:
at least two of the domains are from ECDs of different cell surface receptors;
the hybrid ECD contains a sufficient portion of domain I or III from one or more ECDs of a cell surface receptor to bind ligand, and a sufficient portion of an ECD of a cell surface receptor, including a sufficient portion of domain II, to dimerize with a cell surface receptor when the hybrid ECD is linked to a multimerization domain.

51. The hybrid ECD of claim 50, wherein the cell surface receptor is a member of the HER family.

52. The hybrid ECD of claim 50, wherein domain I is from HER1, domain II is from HER2, and domain III is from HER3.

53. A chimeric polypeptide, comprising the hybrid ECD of claim 50 linked directly or via a linker to a multimerization domain.

54. The chimeric polypeptide of claim 53, wherein the multimerization domain is selected from among an immunoglobulin constant region (Fc), a leucine zipper, complementary hydrophobic regions, complementary hydrophilic regions, compatible protein-protein interaction domains, free thiols that forms an intermolecular disulfide bond between two molecules, and a protuberance-into-cavity and a compensatory cavity of identical or similar size that form stable multimers.

55. The chimeric polypeptide of claim 53 or claim 54, wherein the multimerization domain is an Fc domain.

56. The chimeric polypeptide of claim 55, wherein the Fc domain is from an IgG, an IgM or an IgE.

57. A multimer, comprising at least two chimeric polypeptides of claim 50.

58. A heteromultimer, comprising:
all or part of the extracellular domain (ECD) from HER1 receptor; and
all or part of the ECD from HER3 or HER4 receptor, wherein:
the part includes at least subdomains I, II and III.

59. A nucleic acid molecule, comprising a sequence of nucleic acids encoding a least one chimeric polypeptide in the heteromultimer of claim 1, a chimeric polypeptide of claim 95, or a heteromultimer comprising such chimeric polypeptide, or encoding the hybrid ECD of claim 50.

60. A vector, comprising the nucleic acid of claim 59.

61. An isolated cell, comprising the nucleic acid molecule of claim 59 or the vector of claim 60.

62. A pharmaceutical composition comprising, a multimer, heteromultimer chimeric polypeptide or polypeptide of claim 1 or the nucleic acid molecule of claim 59, or a cell of claim 61.

63. The pharmaceutical composition of claim 59 that is formulated for single dosage administration.

64. The pharmaceutical composition that is formulated for local, topical or systemic administration.

65. A method of treating a cancer, an inflammatory disease, an angiogenic disease or a hyperproliferative disease, comprising administering a therapeutically effective amount of a pharmaceutical composition of claim 62.

66. The method of claim 65, wherein the cancer is pancreatic, gastric, head and neck, cervical, lung, colorectal, endometrial, prostate, esophageal, ovarian, uterine, glioma, bladder, renal or breast cancer.

67. The method of claim 65, where the disease is a proliferative disease.

68. The method of claim 67, wherein the proliferative disease involves proliferation and/or migration of smooth muscle cells, or is a disease of the anterior eye, or is a diabetic retinopathy, or psoriasis.

69. The method of claim 65, wherein the disease is restenosis, ophthalmic disorders, stenosis, atherosclerosis, hypertension from thickening of blood vessels, bladder diseases, and obstructive airway diseases.

70. A method for treating cancer, comprising:
administering a pharmaceutical composition of claim 62 and another anticancer agent.

71. The method of claim 70, wherein the anti-cancer agent is radiation therapy and/or a chemotherapeutic agent.

72. The method of claim 70, wherein the anti-cancer agent is a tyrosine kinase inhibitor or an antibody.

73. The method of claim 72, wherein the anti-cancer agent is a quinazoline kinase inhibitor, an antisense or siRNA or other double-stranded RNA molecule, or an antibody that interacts with a HER receptor, an antibody conjugated to a radionuclide or cytotoxin.

74. The method of claim 73, wherein the anti-cancer agent is Gefitinib, Tykerb, Panitumumab, Eroltinib, Cetuximab, Trastuzimab, Imatinib, a platinum complex or a nucleoside analog.

75. A method of treatment of a HER receptor-mediated disease, comprising:
testing a subject with the disease to identify which HER receptors are expressed or overexpressed; and
based upon the results, selecting a multimer that targets at least two HER receptors.

76. The method of claim 75, wherein the disease is cancer.

77. The method of claim 76, wherein the cancer is pancreatic, gastric, head and neck, cervical, lung, colorectal, endometrial, prostate, esophageal, ovarian, uterine, glioma, bladder or breast cancer.

78. A polypeptide selected from among,
(SEQ ID NO. 405)
CSQFLRGQECVEECRVLQGLPREYVNARHCLPCHPECQPQNGSVTCFGPE
ADQCVACAHYKDPPF;
A target polypeptide in Domain II (DII) of a Her receptor family member

| | HER family | | | | | | | |
|---|---|---|---|---|---|---|---|---|
| Pep. # | HER3 | # | HER4 | # | HER1 | # | HER2 | # |
| | | | | | | | | |
|---|---|---|---|---|---|---|---|---|
| 1.1.5 | CWGPGSEDCQ | 62 | CWGPTENNCQ | 63 | CWGAGEENCQ | 64 | CWGESSEDCQ | 65 |
| 2.1.1 | LTKTICAPQCNG | 66 | LTRTVCAEQCDG | 67 | LTKIICAQQCSG | 68 | LTRTVCAGGCA | 69 |
| 1.1.1 | NPNQCCH | 70 | YVSDCCH | 71 | SPSDCCH | 72 | LPTDCCH | 73 |
| 1.1.2 | ECAGGCSGPQDTDCFAC | 74 | ECAGGCSGPKDTDCFAC | 75 | QCAAGCTGPRESDCLVC | 76 | QCAAGCTGPKNSDCLAC | 77 |
| 1.1.6 | SGACVPRCPQPL | 78 | SGACVTQCPQTF | 79 | EATCKDTCPPLM | 80 | SGICELHCPALV | 81 |
| 1.1.3 | CPHNFVV | 82 | CPHNFVV | 83 | CPRNYVV | 84 | CPYNYLS | 85 |
| 2.1.4 | DQTSCVRACPPD | 86 | DSSSCVRACPSS | 87 | DHGSCVRACGAD | 88 | DHGSCVRACGAD | 89 |
| 1.1.4 | MEVDKNGLK | 90 | MEVEENGIK | 91 | YEMEEDGVR | 92 | QEVTAEDGTQ | 93 |

And
A target polypeptide in Domain IV (DIV) of a Her receptor family member

| | HER family | | | | | | | |
|---|---|---|---|---|---|---|---|---|
| Pep. # | HER3 | # | HER4 | # | HER1 | # | HER2 | # |
| | | | | | | | | |
|---|---|---|---|---|---|---|---|---|
| 1.2.1 | LCSSGGCWGPGP | 94 | LCSSDGCWGPGP | 95 | LCSPEGCWGPEP | 96 | LCARGHCWGPGP | 97 |
| 1.2.5 | SCRNYSRGGV | 98 | SCRRFSRGRI | 99 | SCRNVSRGRE | 100 | NCSQFLRGQE | 101 |
| 1.2.2 | CNFLNGEPREF | 102 | CNLYDGEFREF | 103 | CNLLEGEPREF | 104 | CRVLQGLPREY | 105 |
| 1.2.6 | ANHEAECF | 106 | ENGSICV | 107 | VENSECI | 108 | VNARHCL | 109 |
| 1.2.7 | TATCNGS | 110 | LLTCHGP | 111 | NITCTGR | 112 | SVTCFGP | 113 |
| 1.2.3 | GSDTCAQCAHFRDGPHCV | 114 | GPDNCTKCSHFKDGPNCV | 115 | GPDNCIQCAHYIDGPHCV | 116 | EADQCVACAHYKDPPFCV | 117 |
| 2.2.1 | IYKYPDVQN | 118 | IFKYADPDR | 119 | VWKYADAGH | 120 | IWKFPDEEG | 121 |

among SEQ ID Nos. 54-61, which are target polypeptides for ligand binding.

79. A method for identifying candidate molecules that interact with HER receptors;
a) contacting a test molecule or collection thereof, with a polypeptide of at least about 6 amino or 6 amino acids up to about 50 amino acids or 50 amino acids based upon regions in domains II and IV or I and III that are involved in any of dimerization, ligand binding and/or tethering; and
b) identifying and selecting any test molecule that interacts with one or more of the polypeptides.

80-88. (canceled)

89. An isolated antibody that specifically interacts with a polypeptide of claim 78.

90-94. (canceled)

95. A chimeric polypeptide, comprising:
an ECD or portion thereof sufficient for ligand binding and/or receptor dimerization; and
a multimerization domain,
wherein the ECD or portion thereof is selected from selected from among HER2-530 (SEQ ID No. 14), HER2-595 (SEQ ID No. 16), HER2-650 (SEQ ID No 18), HER3-500 (SEQ ID No.20), P85HER3 (SEQ ID No. 22), HER3-519 (SEQ ID No. 24), HER3-621 (SEQ ID No. 26), HER4-485 (SEQ ID No. 28), HER4-522 (SEQ ID No.30), HER4-650 (SEQ ID No. 32), HER1 ECE as set forth as amino acids 25-645 of SEQ ID No. 414 a polypeptide set forth in any of SEQ ID Nos. 32, 34, 127, 141, 146, 148, 159 and 54-125 and allelic and species variants of any of the aforementioned ECDs.

96. A heteromultimer, comprising two or more chimeric polypeptides, wherein:
the ECDs are selected from among HER1-501 set forth in SEQ ID No. 10 and HER1-621 set forth in SEQ ID No 12 or a portion sufficient for ligand binding and/or receptor dimerization, a chimeric polypeptide of claim 95 and allelic or species variants thereof of any of the aforementioned polypeptides; and
each of the chimeric polypeptides is linked directly or indirectly via linkers to a multimerization domain.

97. The chimeric polypeptide of claim 95 or a heteromultimer of claim 96, wherein the multimerization domain is polypeptide is selected from among an immunoglobulin constant region (Fc), a leucine zipper, complementary hydrophobic regions, complementary hydrophilic regions, compatible protein-protein interaction domains, free thiols that forms an intermolecular disulfide bond between two molecules, and a protuberance-into-cavity and a compensatory cavity of identical or similar size that form stable multimers, whereby the chimeric polypeptides interact in a back-to-back configuration whereby the ECD of both chimeric polypeptides is available for dimerization with a cell surface receptor.

98. The chimeric polypeptide or heteromultimer of claim 97, wherein the multimerization domain is an Fc domain.

99. The chimeric polypeptide or heteromultimer of claim 98, wherein the Fc domain is from an IgG, IgM or an IgE.

100. An isolated polypeptide, comprising a amino acid residues as set forth in any of SEQ ID Nos. 127, 141, 146, 148, 153, 155, 157, 159, 297 and 299.

101. A chimeric polypeptide comprising a polypeptide of claim 100 and a multimerization domain or a polypeptide of claim 151 or claim 152.

102. A heteromultimer, comprising a chimeric polypeptide of claim 101.

103. The heteromultimer of claim 102, comprising a second polypeptide that is HER ECD or portion thereof sufficient for ligand binding and/or receptor dimerization.

104. The heteromultimer of claim 39, wherein both ECDs are HER ECDs.

105. The multimer of claim 17, wherein the intron fusion protein is a herstatin, or variant thereof.

106. A multimer of claim 1, comprising at least two chimeric polypeptides.

107. A chimeric polypeptide, comprising an ECD or portion thereof of a HER1 receptor linked to a multimerization domain, wherein:
the ECD or portion thereof comprises a modification whereby the ECD binds to an additional ligand compared to the unmodified ECD or portion thereof.

108. A chimeric polypeptide, comprising all or a portion of amino acids 25-645 of SEQ ID No. 114 or a sequence having at least about 70, 80, 90, 95% sequence identity thereto but comprises a mutation of Ser to Phe at a position corresponding to 442 of SEQ ID No. 114, linked to a multimerization domain.

109. The chimeric polypeptide of claim 107 or 108, wherein the multimerization domain is selected from among is selected from among an immunoglobulin constant region (Fc), a leucine zipper, complementary hydrophobic regions, complementary hydrophilic regions, compatible protein-protein interaction domains, free thiols that forms an intermolecular disulfide bond between two molecules, and a protuberance-into-cavity and a compensatory cavity of identical or similar size that form stable multimers.

110. The chimeric polypeptide of any of claims 107, wherein the multimerization domain is an Fc domain or a variant thereof that effects multimerization.

111. The chimeric polypeptide of claim 110, wherein the Fc domain is from an IgG, IgM or an IgE.

112. The chimeric polypeptide of any of claims 107, wherein the ECD is from a HER1 receptor.

113. The chimeric polypeptide of any of claims 107, wherein the modification corresponds to modification at position S442 or a corresponding position of an HER receptor.

114. The chimeric polypeptide of claim 113, wherein the modification is in the ECD of a HER1 receptor, whereby the HER1 ECD intereacts with NRG-2β.

115. The multimer of claim 114, wherein the modification is, or corresponds to S442F in Seq. ID No. 2.

116. The chimeric polypeptide of claim 107 that comprises a sufficient portion of the ECD of the modified HER1 to interact with EGF and NRG-2β.

117. The multimer of claim 1, wherein:
the ECD is a modified ECD;
the modification alters ligand binding or other activity of the ECD or full-length receptor containing such ECD compared to the unmodified ECD or full-length receptor.

118. The multimer of claim 1, wherein:
the ECD is not modified to alter ligand binding or other activity

119. The multimer of claim 15, wherein the modification alters ligand binding.

120. The multimer of claim 119, wherein the modification corresponds to modification at position S442 or a corresponding position of an HER receptor.

121. The multimer of claim 120, wherein the modification is in the ECD of a HER1 receptor, whereby the HER1 ECD intereacts with NRG-2β.

122. The multimer of claim 121, wherein the modification is, or corresponds to S442F in SEQ ID No. 2

123. The multimer of claim 117 that comprises an ECD or portion thereof from HER1 and from HER3 or HER4, whereby the resulting multimer interacts with ligands for at least two HER receptors.

124. The multimer of claim 117 that comprises an ECD or portion thereof from HER1 and from HER3 or HER4, whereby the resulting multimer interacts with ligands for at least three HER receptors.

125. The multimer of claim 117 that is a dimer.

126. The multimer of claim 117 that comprises an Fc multimerization domain.

127. The heteromultimer of claim 39, wherein a domain or part thereof from an ECD contains a mutation in the domain that alters ligand binding or specificity; the mutation alters ligand binding or other activity of the ECD or full-length receptor containing such ECD compared to the unmodified ECD or full-length receptor, whereby the heteromultimer exhibits the altered ligand binding or specificity.

128. The heteromultimer of claim 127, wherein the modification alters ligand binding.

129. The heteromultimer of claim 128, wherein the modification corresponds to modification at position S442 or a corresponding position of a HER receptor.

130. The heteromultimer of claim 129, wherein the modification is in the ECD of a HER1 receptor, whereby the HER1 ECD intereacts with NRG-2β.

131. The heteromultimer of claim 130, wherein the modification is, or corresponds to or S442F.

132. The heteromultimer of claim 127 that comprises an ECD or portion thereof from HER1 and from HER3 or HER4, whereby the resulting ECD can interact with ligands for at least two HER receptors.

133. The heteromultimer of claim 127 that comprises an and ECD or portion thereof from HER1 and from HER3 or HER4, whereby the resulting hybrid can interact with ligands for at least three HER receptors.

134. The heteromultimer of claim 127 that comprises an Fc multimerization domain.

135. The hybrid ECD of claim 50, comprising a domain or portion thereof from an ECD that contains a mutation in the domain that alters ligand binding or specificity;
the mutation alters ligand binding or other activity of the ECD or full-length receptor containing such ECD compared to the unmodified ECD or full-length receptor, wherein the hybrid ECD exhibits the altered ligand binding or specificity.

136. The hybrid ECD of claim 135, wherein the modification alters ligand binding.

137. The hybrid ECD of claim 136, wherein the modification corresponds to modification at position S442 or a corresponding position of an HER receptor.

138. The hybrid ECD of claim 137, wherein the modification is in the ECD of a HER1 receptor, whereby the HER1 ECD intereacts with NRG-2β.

139. The hybrid ECD of claim 138, wherein the modification is, or corresponds to or is S442F.

140. The hybrid ECD of claim 135 that comprises an ECD or portion thereof from HER1 and from HER3 or HER4, whereby the resulting ECD can interact with ligands for at least two HER receptors.

141. The hybrid ECD of claim 135 that comprises an and ECD or portion thereof from HER1 and from HER3 or HER4, whereby the resulting hybrid can interact with ligands for at least three HER receptors.

142. The hybrid ECD of claim 135 that comprises an Fc multimerization domain.

143. The heteromultimer of claim 58, wherein a domain or part thereof from an ECD contains a mutation in the domain that alters ligand binding or specificity;
the mutation alters ligand binding or other activity of the ECD or full-length receptor containing such ECD compared to the unmodified ECD or full-length receptor, whereby the heteromultimer exhibits the altered ligand binding or specificity.

144. The heteromultimer of claim 143, wherein the modification alters ligand binding.

145. The heteromultimer of claim 144, wherein the modification corresponds to modification at position S442 or a corresponding position of an HER receptor.

146. The heteromultimer of claim 145, wherein the modification is in the ECD of a HER1 receptor, whereby the HER1 ECD intereacts with NRG-2β.

147. The heteromultimer of claim 146, wherein the modification is, or corresponds to or S442F.

148. The heteromultimer of claim 143 that comprises an ECD or portion thereof from HER1 and from HER3 or HER4, whereby the resulting ECD can interact with ligands for at least two HER receptors.

149. The heteromultimer of claim 143 that comprises an ECD or portion thereof from HER1 and from HER3 or HER4, whereby the resulting hybrid can interact with ligands for at least three HER receptors.

150. The heteromultimer of claim 143 that comprises an Fc multimerization domain.

151. A chimeric polypeptide, comprising a multimerization domain linked directly or indirectly via a linker to the polyeptide set forth as amino acids 25-645 of SEQ ID No. 414 or a portion thereof sufficient to effect ligand binding to at least two different ligand.

152. The polypeptide of claim 151, wherein the multimerization domain is selected from among an immunoglobulin constant region (Fc), a leucine zipper, complementary hydrophobic regions, complementary hydrophilic regions, compatible protein-protein interaction domains, free thiols that forms an intermolecular disulfide bond between two molecules, and a protuberance-into-cavity and a compensatory cavity of identical or similar size that form stable multimers, whereby the chimeric polypeptides interact in a back-to-back configuration whereby the ECD of both chimeric polypeptides is available for dimerization with a cell surface receptor.

153. A composition comprising a mixture of heteromultimers and homomultimers wherein the heteromultimer comprises an ECD or portion thereof from HER1 and another ECD or portion thereof from HER3 and wherein the homomultimers comprise an ECD or portion thereof from HER1 or an ECD or portion thereof from HER3.

154. A pharmaceutical composition comprising the composition of claim 153 formulated for topical, oral, systemic, or local administration.

155. A method for treating cancer, an inflammatory disease, an angiogenic disease or a hyperproliferative disease, comprising administering a therapeutically effective amount of a composition of claim 153 or 154.

156. The method of claim 155, wherein the cancer is pancreatic, gastric, head and neck, cervical, lung, colorectal, endometrial, prostate, esophageal, ovarian, uterine, glioma, bladder, renal or breast cancer.

157. The method of claim 155, where the disease is a proliferative disease.

158. The method of claim 157, wherein the proliferative disease involves proliferation and/or migration of smooth muscle cells, or is a disease of the anterior eye, or is a diabetic retinopathy, or psoriasis.

159. The method of claim 155, wherein the disease is restenosis, ophthalmic disorders, stenosis, atherosclerosis, hypertension from thickening of blood vessels, bladder diseases, and obstructive airway diseases.
